(19) United States
(12) Patent Application Publication
(10) Pub. No.: US 2025/0120813 A1
(43) Pub. Date: Apr. 17, 2025

(54) HEART VALVE PROSTHESES AND RELATED METHODS

(71) Applicant: InValve Therapeutics, Inc., Louisville, KY (US)

(72) Inventors: Anthony PAOLITTO, Mont-Royal (CA); Paul A. SPENCE, Aventura, FL (US); Valerio VALENTINI, Montreal (CA)

(73) Assignee: InValve Therapeutics, Inc., Louisville, KY (US)

(21) Appl. No.: 18/752,629

(22) Filed: Jun. 24, 2024

Related U.S. Application Data
(63) Continuation of application No. PCT/US2022/082183, filed on Dec. 21, 2022.
Application No. PCT/US2022/082183 is a continuation-in-part of application No. PCT/US2021/040053, filed on Jul. 1, 2021.
(60) Provisional application No. 63/292,464, filed on Dec. 22, 2021.
(60) Provisional application No. 63/046,841, filed on Jul. 1, 2020.

(51) Int. Cl. A61F 2/24

(52) U.S. Cl. CPC A61F 2/2463; A61F 2220/0016; A61F 2220/0025; A61F 2250/0006

(57) ABSTRACT

Disclosed are numerous embodiments of selective occlusion devices (SODs) that can be disposed in native heart valves in conjunction with leaflet clips to remediate regurgitation of the native valve.

### CROSS-REFERENCE TO RELATED APPLICATIONS

This application is a continuation of International Patent Application No. PCT/US2022/082183, entitled “Heart Valve Prostheses and Related Methods,” filed Dec. 21, 2022, which claims priority to and the benefit of U.S. Provisional Patent Application No. 63/292,464, entitled “Heart Valve Prostheses and Related Methods,” filed Dec. 22, 2021, the disclosure of which is incorporated herein by reference in its entirety.

International Patent Application No. PCT/US2022/082183 is a continuation-in-part of International Patent Application No. PCT/US2021/040053, entitled “Heart Valve Prostheses and Related Methods,” filed Jul. 1, 2021, which claims priority to U.S. Provisional Patent Application No. 63/046,841, entitled “Mitral Valve Prostheses and Related Methods,” filed Jul. 1, 2020, the disclosure of each of which is incorporated herein by reference in its entirety.

International Patent Application No. PCT/US2022/082183 is related to U.S. Pat. No. 10,912,646, entitled “Methods, Apparatus and Devices to Treat Heart Valves” (“the '646 Patent”), the disclosure of which is incorporated by reference herein in its entirety.

### BACKGROUND

Heart valve incompetence, in various forms and affecting various valves of the heart (e.g., the aortic valve, tricuspid valve, pulmonary valve and mitral valve), has led to a growing area of research and development designed to improve heart valve functionality. Although any one or more of these native heart valves may be compromised due, for example, to congenital disorders or, more often, disease conditions, the mitral valve has received particular attention. Regurgitation of blood flow through a heart valve, such as a mitral valve, involves the backward flow of blood through the valve when the valve is supposed to be fully closed (i.e., full coaptation of the native leaflets). A diseased or otherwise compromised mitral valve will often allow regurgitated blood flow from the left ventricle into the left atrium during cardiac systole. This causes the amount of blood ejected from the left ventricle during cardiac systole to be reduced, leading to less than optimal “ejection fraction” for the patient. Thus, the patient may experience a lower quality of life due to this inefficiency of their heart or, worse, a life-threatening condition.

Surgical techniques as well as transvascular or catheter-based techniques for treatment of mitral valve incompetence have been developed and, for example, include mitral annuloplasty, attachment of the native anterior mitral leaflet to the native posterior mitral leaflet, chordal replacement and even complete mitral valve replacement. Similar approaches have been developed for treatment of tricuspid valve incompetence.

In many cases, mitral valve regurgitation is related not to congenital defects in the mitral valve leaflets but to changes in the coaptation of the leaflets over time due to heart disease. In these situations, the native mitral leaflets are often relatively normal, but they nevertheless fail to prevent regurgitation of blood from the left ventricle into the left atrium during cardiac systole. Instead of the native anterior and posterior leaflets properly mating or coapting together completely during cardiac contraction or systole, one or more gaps between the native leaflets cause mitral regurgitation. Similar issues are encountered with tricuspid valves.

A current, commonly used technique for reducing mitral valve regurgitation is an edge-to-edge approximation or repair procedure that involves the attachment of the native mitral valve anterior leaflet to the native mitral valve posterior leaflet using a clip structure. The use of the edge to edge mitral repair procedure is increasing rapidly to treat mitral regurgitation. Abbott has the MitraClip™ on the market and Edwards has recently introduced the PASCAL device. The MitraClip™ fastens or clips the anterior mitral leaflet to the posterior mitral leaflet, while the PASCAL performs the same function with the addition of a material between the native leaflets providing certain advantages for the procedure.

MitraClip™ procedures currently use about two clips per procedure and mitral regurgitation remains in many patients who undergo treatment. The native anterior and posterior mitral leaflets have gaps between them in systole resulting in persistent mitral regurgitation even after clipping them together. Clinical studies show improved patient outcomes with the clip but many patients remain quite ill and require ongoing strict medical supervision. Abbott has also developed the TriClip™ for clipping the native leaflets of tricuspid valves.

The '646 Patent discloses devices attached to an edge-to-edge mitral clipping device to prevent any residual leak. These devices and methods sealed the space between the native mitral leaflets in systole and allowed for filling of the left ventricle in diastole. Some devices were fixed in shape and others had moving components or leaflets that closed the residual gap in systole and allowed blood to enter the LV in diastole.

One particularly promising variation disclosed in the '646 Patent was a bileaflet valve that could be positioned and attached to the edge to edge clip. A number of variations on this solution are shown including (but not limited to) FIGS. 5-11, 15-29 and 35 of the '646 Patent, also included herein.

Each of these variations requires the development of a new type of valve-generally a bileaflet valve that fills the gap between the native leaflets in systole and moves to allow blood to enter the left ventricle (LV) in diastole. This valve will require considerable testing and development before it is available for clinical use. There is no such similar bileaflet device on the market. So, there is a development risk and a regulatory risk that this new device may fail. It is also possible that a bi-leaflet device may not be widely welcomed by physicians who have been accustomed to tri-leaflet valves for more than 50 years.

The tri-leaflet stented valve is proven effective and safe. It has been the mainstay of surgical tissue valves for over 50 years and millions of valves with a tri-leaflet construction have been implanted in patients with very good long-term outcomes. In the last decade hundreds of thousands of stented valves carrying three leaflets have been successfully used in patients who have received catheter based heart valve replacement procedures. It would be very useful to consider using two proven technologies (the edge to edge device and the tri-leaflet stented valve) to treat mitral regurgitation. The combination of these will reduce time to market and as well as regulatory and adoption risk, in addition to clinical advantage.

Many doctors are now very accomplished in performing the mitral clipping procedure (bringing the anterior and posterior leaflets together with a clip such as the MitraClip™) and the tricuspid clipping procedure (which can have several variations on bringing together the anterior, septal, and/or posterior leaflets with a clip such as the MitraClip™). They are also confident in the reliability and with their ability to deliver a stented valve. Building a device that takes advantage on these proven implants and skill sets will be well received by doctors and safer for patients who have doctors performing a familiar procedure.

It would be useful to further address these and other problems or challenges associated with heart valve incompetence.

### SUMMARY

Disclosed are numerous embodiments of selective occlusion devices (SODs) that can be disposed in native heart valves in conjunction with leaflet clips to remediate regurgitation of the native valve.

Additional features, aspects and/or advantages will be recognized and appreciated upon further review of a detailed description of the illustrative embodiments taken in conjunction with the accompanying drawings.

### BRIEF DESCRIPTION OF THE DRAWINGS

FIG. 1A is a schematic view illustrating a system constructed in accordance with one illustrative embodiment.

FIG. 1B is a schematic perspective view of a native left atrium and mitral valve, similar to FIG. 1A, but illustrating installation of the catheter delivered selective occlusion device.

FIG. 1C is a schematic perspective view similar to FIG. 1B, but illustrating the membrane of the selective occlusion device in place over the frame structure.

FIG. 2A is a cross-sectional view taken transversely through the selective occlusion device along line 2A-2A of FIG. 3A when the heart cycle is in systole.

FIG. 2B is a cross-sectional view similar to FIG. 2A, during the systole phase of the heart cycle, but taken along line 2B-2B of FIG. 3A.

FIG. 2C is a cross-sectional view similar to FIG. 2B, but illustrating the native mitral valve and the selective occlusion device while in the diastole phase of the heart cycle.

FIG. 3A is a top view of the native mitral valve and the selective occlusion device while the heart is in the systole phase.

FIG. 3B is a top view similar to FIG. 3A, but illustrating the device and native mitral valve while the heart is in the diastole phase.

FIG. 4A is a perspective view of the device as shown in the previous figures, with the membrane of the device removed for clarity, and showing only the frame structure in solid lines.

FIG. 4B is a perspective view similar to FIG. 4A, but illustrating the membrane applied to the frame structure of the device.

FIG. 5A is a schematic perspective view, partially sectioned similar to FIG. 1A, but illustrating a catheter-based or transcatheter delivery and implantation system constructed in accordance with another embodiment.

FIG. 5B is a view similar to FIG. 5A, but illustrating a subsequent step in the method, in which the native mitral leaflets have been captured and clipped together.

FIG. 5C is a sectional view similar to FIGS. 5A and 5B, but illustrating the frame of the selective occlusion device implanted and attached to the clip structure, with the flexible membrane removed for clarity.

FIG. 5D is a view similar to FIG. 5C, but illustrating the flexible membrane of the device in place on the frame structure.

FIG. 6A is a perspective view of the frame structure and attached clip structure shown in FIGS. 5A through 5C.

FIG. 6B is a perspective view similar to FIG. 6A, but illustrating another embodiment of a collapsible and expandable frame structure.

FIG. 7A is a cross sectional view of the native mitral valve and selective occlusion device of FIG. 6B, with the heart in the diastole phase.

FIG. 7B is a cross sectional view similar to FIG. 7A, but illustrating the selective occlusion device and the mitral valve when the heart is in the systole phase.

FIG. 8 is a side view with the heart in cross-section at the location of the native mitral valve, illustrating the selective occlusion device of FIGS. 7A and 7B, with the membrane in broken lines for clarity, and the device implanted.

FIG. 9 is a perspective view illustrating another embodiment of a selective occlusion device, showing the frame structure in solid lines and the flexible membrane in broken lines for clarity.

FIG. 10A is a schematic perspective view similar to FIGS. 1A and 5A, but illustrating another embodiment of a catheter-based system for delivering and implanting a selective occlusion device coupled with a pre-installed mitral valve leaflet clip structure.

FIG. 10B is a view similar to FIG. 10A, but illustrating a subsequent step during the method.

FIG. 10C is a perspective view, with the heart sectioned at the native mitral valve, illustrating the implantation of the selective occlusion device, but with the flexible membrane removed for clarity.

FIG. 11A is a perspective view showing another alternative embodiment of a selective occlusion device with the flexible membrane removed for clarity.

FIG. 11B is a perspective view showing another alternative embodiment of a selective occlusion device with the flexible membrane removed for clarity.

FIG. 11C is a front top perspective view of the device of FIG. 11A or 11B implanted in the native mitral valve.

FIG. 11D is a front view of the device in FIGS. 11A through 11C.

FIG. 11E is a transverse cross section of FIG. 11D.

FIG. 12A is a perspective view of another alternative embodiment of a selective occlusion device implanted in the native mitral valve, which is shown in cross-section similar to previous figures.

FIG. 12B is a cross-sectional view of the heart, taken at the native mitral valve, and showing the selective occlusion device of FIG. 12A in side elevation.

FIG. 12C is a view similar to FIG. 12B, but illustrating another alternative embodiment of a selective occlusion device implanted in a native mitral valve.

FIG. 12D is another view similar to FIG. 12C, but illustrating another alternative embodiment of a selective occlusion device implanted in the native mitral valve.

FIG. 13A is a transverse cross-sectional view taken through the mitral valve and generally through one of the selective occlusion elements of FIGS. 12A through 12D, to show sealing during systole.

FIG. 13B is a view similar to FIG. 13A, but showing the selective occlusion element and the mitral valve when the heart is in the diastole phase.

FIG. 13C is a view similar to FIG. 13B, but showing another embodiment of the selective occlusion element.

FIG. 14A is a perspective view of another alternative embodiment of a selective occlusion device and mitral valve clip structure.

FIG. 14B is a perspective view of another alternative embodiment of a selective occlusion device and mitral valve clip structure.

FIG. 14C is a perspective view of another alternative embodiment of a selective occlusion device and mitral valve clip structure.

FIG. 15A is a perspective view of another alternative embodiment of a selective occlusion device with the flexible membrane of the device broken away for clarity.

FIG. 15B is a perspective view similar to FIG. 15A, but further illustrating a flexible membrane on the frame structure.

FIG. 15C is a side elevational view of the selective occlusion device shown in FIGS. 15A and 15B with the flexible membrane removed for clarity.

FIG. 15D is a side elevation view similar to FIG. 15C, but illustrating the flexible membrane applied to the frame structure.

FIG. 15E is a top view of the device shown in FIGS. 15A through 15D, but illustrating the membrane cross-sectioned to show the membrane shape in the expanded or filled condition when the heart is in the systole phase.

FIG. 16A is a perspective view of a system and of the heart, similar to FIG. 5A, but illustrating another alternative embodiment of a catheter-based system and method for implanting a selective occlusion device and a clip structure in the native mitral valve.

FIG. 16B is a perspective view similar to FIG. 16A, but illustrating a subsequent step in the method.

FIG. 16C is a view similar to FIG. 16B, but illustrating another subsequent step in the method.

FIG. 16D is a perspective view illustrating the implanted selective occlusion device in the mitral valve of the patient.

FIG. 17A is a side cross-sectional view of the native mitral valve and of the selective occlusion device of FIGS. 16A through 16D being implanted and secured to the mitral valve clip structure.

FIG. 17B is a side cross-sectional view similar to FIG. 17A, but illustrating a subsequent step in the method.

FIG. 17C is a side cross-sectional view similar to FIG. 17B, but illustrating another subsequent step in the method in which the apparatus is fully implanted.

FIG. 18A is a cross sectional view of the selective occlusion device, as shown in FIGS. 16A through 16D and 17A through 17C, with the device and mitral valve shown when the heart is in the diastole phase.

FIG. 18B is a view similar to FIG. 18A, but illustrating the device and the native mitral valve when the heart is in the systole phase.

FIG. 19 is a top view schematically illustrating a representation for the shape of the selective occlusion device when implanted in a native mitral valve having an anatomical curvature.

FIG. 20 is a perspective view of a selective occlusion device constructed in accordance with another alternative embodiment.

FIG. 21A is a side cross-sectional view taken generally lengthwise along a central portion of the device shown in FIG. 20.

FIG. 21B is a top view of the device shown in FIG. 21A.

FIG. 21C is a cross-sectional view of the device shown in FIG. 21B.

FIG. 22A is a perspective view of a catheter-based system and method according to another alternative embodiment being performed on a native mitral valve, shown in a schematic cross-sectioned portion of the heart.

FIG. 22B is a view similar to FIG. 22A, but illustrating a subsequent step in the method.

FIG. 22C is a view similar to FIG. 22B, but illustrating another subsequent step in the method.

FIG. 22D is a perspective view illustrating the fully implanted apparatus in the native mitral valve, resulting from the method shown in FIGS. 22A through 22C.

FIG. 22E is a view similar to FIG. 22D, but illustrating an alternative frame structure attached to the selective occlusion device.

FIG. 22F is a view similar to FIG. 22E, but illustrating another alternative frame structure.

FIG. 22G is a view similar to FIG. 22F, but illustrating another alternative frame structure.

FIG. 23A is a cross-sectional view of a native mitral valve and another embodiment of a heart valve repair apparatus, shown with the heart in the systole phase.

FIG. 23B is a view similar to FIG. 23A, but illustrating the apparatus and the mitral valve when the heart is in the diastole phase.

FIG. 24 is a side cross-sectional view of another alternative embodiment of a heart valve repair apparatus implanted in a native mitral valve.

FIG. 25A is a cross-sectional view of another alternative embodiment of a heart valve repair apparatus.

FIG. 25B is a cross-sectional view of another alternative embodiment of a heart valve repair apparatus implanted in a native mitral valve.

FIG. 26A is another alternative embodiment of a selective occlusion device shown in cross-section.

FIG. 26B is a schematic view illustrating the device of FIG. 26A implanted in a native mitral valve.

FIG. 26C is a perspective view illustrating the device of FIGS. 26A and 26B implanted in a native mitral valve.

FIG. 26D is a cross-sectional view of another alternative heart valve repair apparatus implanted in a native mitral valve.

FIG. 26E is a cross-sectional view of another alternative heart valve repair apparatus implanted in a native mitral valve.

FIG. 27A is a perspective view of another alternative selective occlusion device.

FIG. 27B is a lengthwise cross-sectional view of the device shown in FIG. 27A, schematically illustrating blood flow during the systole phase of the heart.

FIG. 27C is a transverse cross-sectional view illustrating the device of FIGS. 27A and 27B during systole.

FIG. 28A is a perspective view illustrating another alternative embodiment of another apparatus including a selective occlusion device together with a mitral valve clip structure.

FIG. 28B is a lengthwise cross-sectional view illustrating the device and clip structure shown in FIG. 28A.

FIG. 28C is a transverse cross-sectional view illustrating the device of FIGS. 28A and 28B.

FIG. 29A is a cross-sectional view of a selective occlusion device and clip structure schematically illustrating blood flow between the interior membrane wall surfaces during the heart systole phase.

FIG. 29B is a cross sectional view of the apparatus of FIG. 29A implanted in the native mitral valve and illustrating the device and the mitral valve when the heart is in the systole phase.

FIG. 30 is a perspective view illustrating the mitral valve in cross-section and the fully implanted selective occlusion device and clip structure.

FIG. 31 is a perspective view of another alternative embodiment illustrating a prosthetic heart valve and leaflet clip structures.

FIG. 32A is a side elevational view of the prosthetic heart valve of FIG. 31, partially fragmented to show the prosthetic heart valve and leaflet clip structures.

FIG. 32B is a side elevational view with the native heart valve in cross-section, illustrating an initial portion of the implantation procedure associated with the prosthetic heart valve of FIGS. 31 and 32A.

FIG. 32C is a view similar to FIG. 32B, but illustrating a subsequent step in the method.

FIG. 32D is a view similar to FIG. 32C, but illustrating a subsequent step in the method.

FIG. 32E is a view similar to FIG. 32D, but illustrating the fully implanted prosthetic heart valve clipped to the native heart valve leaflets and expanded into an implanted condition.

FIG. 33 is a perspective view of another alternative embodiment of a prosthetic heart valve and native leaflet clip structure.

FIG. 34A is a side elevational view of the prosthetic heart valve illustrated in FIG. 33.

FIG. 34B is a view of the prosthetic heart valve of FIG. 34A implanted in a native heart valve.

FIG. 35A is a cross sectional view similar to FIG. 29B, but illustrating another illustrative embodiment of a heart valve repair apparatus implanted in a mitral valve and showing the systole phase of the heart cycle.

FIG. 35B is a cross sectional view similar to FIG. 35A, but illustrating the apparatus and mitral valve when the heart cycle is in the diastole phase.

FIGS. 36A and 36B are illustrations of the anatomy of a native mitral valve and native tricuspid valve, respectively.

FIG. 37A is a schematic illustration of a native mitral valve.

FIGS. 37B to 37D are schematic illustrations of a native mitral valve after a clipping procedure with one more clips engaged with the native leaflets.

FIGS. 38A to 38F are schematic illustration of a native tricuspid valve after a clipping procedure with one or more clips engaged with the native leaflets.

FIGS. 39A and 39B are schematic illustrations of a prosthetic valve according to an embodiment, in side view and top view, respectively.

FIGS. 40A and 40B are schematic illustrations of the prosthetic valve of FIGS. 39A and 39B, shown disposed in a native mitral valve, in side view and top view, respectively.

FIG. 41 is a flow chart of a method of delivery of the prosthetic valve of FIGS. 39A and 39B, according to an embodiment.

FIGS. 42A and 42B are a perspective partial view, a partial cross-sectional side view of a prosthetic valve according to an embodiment.

FIG. 42C is a perspective view of the prosthetic valve of FIGS. 42A and 42B shown disposed in a native mitral valve.

FIGS. 42D to 42F are partial end cross-sectional views showing variants of the clip connector of the prosthetic valve of FIGS. 42A to 42C.

FIG. 43 is a partial cross-sectional side view of a prosthetic valve according to an embodiment.

FIG. 44 is a partial cross-sectional side view of a prosthetic valve according to an embodiment.

FIGS. 45A to 45C are partial cross-sectional side views of a prosthetic valve according to an embodiment, illustrating a process for expanding a limb of the prosthetic valve.

FIGS. 46A to 46C are top, side, and partial cross-sectional side views, respectively, of a prosthetic valve according to an embodiment.

FIGS. 47A to 47D are top, side, end, and exploded end views of a prosthetic valve, according to an embodiment, disposed in a native mitral valve.

FIG. 48 is a top of a flow control device similar to that of the prosthetic valve of FIGS. 47A to 47D, according to an embodiment.

FIGS. 49A and 49B are a top view and a side view, respectively, of a prosthetic valve, according to an embodiment.

FIG. 50 is a side view of a prosthetic valve, according to an embodiment.

FIGS. 51A and 51B are a top view and a partial cross-sectional end view, respectively, of a prosthetic valve, according to an embodiment.

FIG. 51C to 51F are perspective views of a components of the flow control device of the prosthetic valve of FIGS. 51A and 51B.

FIGS. 52A and 52B are schematic illustrations of a prosthetic valve according to an embodiment, in side view and top view, respectively.

FIGS. 53A and 53B are schematic illustrations of the prosthetic valve of FIGS. 52A and 52B, shown disposed in a native mitral valve, in side view and top view, respectively.

FIG. 54 is a flow chart of a method of delivery of the prosthetic valve of FIGS. 52A and 52B, according to an embodiment.

FIGS. 55A to 55C are side perspective, top, and top perspective view of a prosthetic valve according to an embodiment, disposed in a centrally-clipped mitral valve.

FIGS. 56A and 56B are top views of a prosthetic valve according to an embodiment, disposed in a centrally clipped mitral valve, and FIGS. 56C to 56I illustrate mechanisms and procedures for securing the prosthetic valve to the clip in the mitral valve.

FIGS. 57A and 57B are top and end views, respectively, of a prosthetic valve according to an embodiment, shown disposed in an eccentrically-clipped mitral valve.

FIGS. 58A and 58B are top and end views, respectively, of a prosthetic valve according to an embodiment, shown disposed in an eccentrically-clipped mitral valve.

FIGS. 59A and 59B are top and end views, respectively, of a prosthetic valve according to an embodiment, shown disposed in an eccentrically-clipped mitral valve.

FIGS. 60A and 60B are perspective top and side views, respectively, of a prosthetic valve according to an embodiment, shown disposed in an eccentrically-clipped mitral valve.

FIGS. 60C and 60D are perspective top views of the prosthetic valve of FIGS. 60A and 60B, illustrating alternative heart tissue tethers.

FIG. 61A is a top view of a prosthetic valve according to an embodiment, shown disposed in an eccentrically-clipped mitral valve, and FIG. 61B is a top perspective view of the clip of FIG. 61A.

FIG. 62 is a top view of a prosthetic valve according to an embodiment, shown disposed in a mitral valve clipped with two eccentrically-placed clips.

FIG. 63 is a top view of a prosthetic valve according to an embodiment, shown disposed in a tricuspid valve clipped with two clips in a triple orifice clipping procedure.

FIGS. 64A and 64B are top and top perspective views, respectively, of a prosthetic valve according to an embodiment, shown in FIG. 64A disposed in a tricuspid valve clipped with three clips.

FIG. 65A is a cross-sectional perspective view of a delivery system for clips and for the prosthetic valve of FIGS. 64A and 64B, and FIGS. 65B to 65D illustrate delivery of the clips to the tricuspid valve resulting in the clipped tricuspid valve shown in FIG. 64A

FIG. 66 is a top view of a prosthetic valve according to an embodiment, shown disposed in a tricuspid valve that has been clipped with three clips in a bicuspidization procedure.

FIGS. 67A to 67C illustrate a heart tissue tether for a clip, and a process for delivering and deploying the tether and the clip.

FIGS. 68A and 68B are schematic illustrations of a selective occluding device (SOD) according to an embodiment, in a side view and a top view, respectively.

FIGS. 69A and 69B are schematic illustrations of the SOD of FIGS. 68A and 68B disposed in a native heart valve, in a side view and a top view, respectively.

FIG. 70 is a perspective view of an SOD disposed in a native mitral valve, according to an embodiment.

FIG. 71 is a perspective view of an SOD disposed in a native mitral valve, according to an embodiment.

FIG. 72 illustrates a device and a method for repairing or salvaging an SOD previously implanted in a mitral valve, according to an embodiment.

FIG. 73 illustrates another device and a method for repairing or salvaging an SOD previously implanted in a mitral valve, according to an embodiment.

FIG. 74 is a schematic illustration of a native mitral valve in which a single clip is disposed centrally to approximate the edges of the native leaflets, according to an embodiment.

FIGS. 75A and 75B are top views of an SOD disposed in the native mitral valve of FIG. 74, during systole, with the SOD shown unshaded and shaded, respectively, according to an embodiment.

FIGS. 75C and 75D are a side view and a partial, detail side view, respectively, of the SOD of FIGS. 75A and 75B.

FIGS. 76A and 76B are partial perspective, exploded and assembled views, respectively, of an occluder arm pivot of the SOD of FIGS. 75A and 75B, engaged with a clip.

FIG. 76C is a side view of the occluder arm pivot and clip of FIGS. 76A and 76B.

FIGS. 77A and 77B are perspective views of a method of coupling the occluder arm pivot and clip of FIGS. 76A-C.

FIG. 78A is a perspective view of an occluder arm pivot, clip connector, and clip, according to an embodiment.

FIG. 78B is an assembled, cross-sectional view of the occluder arm pivot, clip connector, and clip of FIG. 78A.

FIG. 79 is a perspective view of a clip being delivered to a mitral valve having an annuloplasty ring, according to an embodiment.

FIG. 80 is a perspective view of an SOD being delivered to a mitral valve having an annuloplasty ring and a clip, according to an embodiment.

FIG. 81A is a perspective view of an SOD that includes two clips, according to an embodiment.

FIGS. 81B and 81C illustrate the SOD of FIG. 81A disposed in an SOD delivery catheter, according to an embodiment.

FIGS. 81D and 81E are top views of the SOD of FIG. 81A disposed in a native mitral valve, during diastole and systole, respectively.

FIG. 82A is a perspective view of an SOD that includes two clips, according to an embodiment.

FIG. 82B illustrates the SOD of FIG. 82A disposed in an SOD delivery catheter, according to an embodiment.

FIG. 83A is a perspective view of an SOD and a pair of clips to which the SOD can be coupled, according to an embodiment.

FIGS. 83B and 83C illustrate further details of the SOD and clip connectors for coupling the SOD of FIG. 83A to the pair of clips, according to an embodiment.

FIG. 83D is a top view of the SOD coupled to the pair of clips, as shown in FIG. 83A.

FIG. 84 is a top view of an SOD coupled to a pair of clips, according to an embodiment.

FIGS. 85A and 85B are top views of an SOD disposed in a native mitral valve, during diastole and systole, respectively, according to an embodiment.

FIGS. 85C and 85D are cross-sectional side views of the SOD of FIGS. 85A and 85B, during diastole and systole, respectively.

FIG. 85E is schematic elevation view of the SOD of FIGS. 85A and 85B.

FIGS. 86A and 86B are top and cross-sectional side views, respectively, of an SOD disposed in a native mitral valve, according to an embodiment.

FIGS. 87A and 87B are a cross-sectional side and elevation view, respectively, of an SOD disposed in a native mitral valve, according to an embodiment.

FIGS. 87C and 87D are top views of the SOD of FIGS. 87A and 87B during diastole and systole, respectively.

FIGS. 88A and 88B are top views of an SOD disposed in a native mitral valve, during diastole and systole, respectively, according to an embodiment.

FIG. 88C is an elevation view of the SOD of FIGS. 88A and 88B.

FIG. 89 is a top view of an SOD disposed in a native mitral valve, according to an embodiment.

FIG. 90A is a top view of an SOD with an integrated clip, disposed in a native mitral valve, with the SOD in a stowed configuration, according to an embodiment.

FIGS. 90B and 90C are top views of the SOD of FIG. 90A, with the SOD in a deployed configuration, during diastole and systole, respectively.

FIGS. 90D-H show steps of a method of deploying the SOD of FIGS. 90A-C.

FIGS. 91A and 91B are perspective views of an SOD having two occluders, shown disposed in a native mitral valve, during systole and diastole, respectively according to an embodiment.

FIGS. 91C and 91D are cross-sectional views of the SOD of FIGS. 91A and 91B, respectively.

FIGS. 91E to 91H illustrate a membrane support frame of an occluder of the SOD of FIGS. 91A to 91D.

FIGS. 92A and 92B are perspective views of an SOD, according to an embodiment.

FIG. 92C illustrates steps of a method for attaching the SOD of FIGS. 92A and 92B, to a clip, according to an embodiment.

FIG. 92D illustrates a clip coupled to native leaflets.

FIGS. 92E and 92F are side views of the SOD of FIGS. 92A and 92B, coupled to the clip and disposed in a native mitral valve, respectively.

FIG. 92G is a top view of the SOD of FIGS. 92A and 92B.

FIGS. 92H to 92K illustrate the clip connector of the SOD of FIGS. 92A and 92B, coupling to a delivery catheter.

FIGS. 93A and 93B are perspective views of an SOD having a tubular clip connector for coupling to the clip, according to an embodiment.

FIGS. 94A and 94B are schematic illustrations of a selective occluding device (SOD) according to an embodiment, in a side view and a top view, respectively.

FIGS. 95A and 95B are schematic illustrations of the SOD of FIGS. 94A and 94B disposed in a native heart valve, in a side view and a top view, respectively.

FIG. 96A is a schematic illustration of an SOD with a ventricular connector, disposed in a native mitral valve, according to an embodiment, and FIGS. 96B and 96C are illustrations of the SOD of FIG. 96A taken along line 96B-96B of FIG. 96A. FIG. 96D is a schematic illustration of the SOD of FIGS. 96A to 96C, but with an annulus connector, disposed in a native mitral valve, according to an embodiment, and FIGS. 96E and 96F are illustrations of the SOD taken along line 96E-96E of FIG. 96D.

FIG. 97A is a schematic illustration of an SOD disposed in a native mitral valve, according to an embodiment, and FIGS. 97B to 97D are illustrations of the SOD of FIG. 96A taken along line 97B-97B of FIG. 97A.

FIGS. 98A and 98B are perspective views of an SOD, shown disposed in FIG. 98A in a native mitral valve, according to an embodiment.

FIG. 98C is a cross-sectional view of the occluder of the SOD of FIGS. 98A and 98B, taken along line 98C-98C of FIG. 98A, during different stages of the cardiac cycle.

FIG. 98D is a bottom view of the occluder of the SOD of FIGS. 98A and 98B, taken along line 98D-98D of FIG. 98B, during different stages of the cardiac cycle.

FIG. 98E is a detail view of the SOD of FIGS. 98A and 98B, showing the attachment of the membrane to the support frame.

FIG. 98F is a top view of the SOD of FIGS. 98A and 98B, shown during systole.

FIGS. 99A to 99C illustrate an SOD with an alternate movable membrane configuration and alternate ventricular tissue anchor, according to an embodiment.

FIG. 99D is a detail view of the tissue anchor of FIGS. 99A to 99C.

FIGS. 99E and 99F are detail views of alternate membrane attachments, according to embodiments.

FIGS. 100A to 100D illustrate an implementation of a membrane spacing element, according to an embodiment.

FIGS. 101A to 101D illustrate another implementation of a membrane spacing element, according to an embodiment.

FIGS. 102A to 102D illustrate another implementation of a membrane spacing element, according to an embodiment.

FIGS. 103A to 103G illustrate another implementation of a membrane spacing element, according to an embodiment.

FIGS. 104A to 104D illustrate another implementation of a membrane spacing element, according to an embodiment.

FIG. 105A illustrates an SOD (such as the embodiments shown in FIGS. 101A to 101D and FIGS. 103A to 103G) disposed in a native mitral valve.

FIG. 105B illustrates an SOD (such as the embodiment shown in FIGS. 102A to 102D) disposed in a native mitral valve.

FIG. 105C illustrates an SOD (such as the embodiment shown in FIGS. 101A to 101D) disposed in a native mitral valve.

FIGS. 106A to 106C illustrate a tissue anchoring clip for an SOD, according to an embodiment, and FIGS. 106D to 106F illustrate delivery of multiple tissue anchoring clips.

FIGS. 107A to 107C illustrate a multiprong tissue anchor for an SOD, according to an embodiment.

FIGS. 108A to 108D illustrate a papillary muscle anchor for an SOD, according to an embodiment.

FIGS. 109A and 109B illustrate an anchoring plate ventricular connector for an SOD, according to an embodiment.

FIGS. 110A to 110E illustrate an occluder for an SOD with a backing strip on the flexible membranes to implement a membrane spacing element, according to an embodiment.

FIGS. 111A to 111E illustrate an occluder for an SOD with a rib stiffener on the flexible membranes to implement a membrane spacing element, according to an embodiment.

FIGS. 112A to 112E illustrate an occluder for an SOD with an inlet stiffener on the flexible membranes to implement a membrane spacing element, according to an embodiment.

FIGS. 113A to 113D illustrate an occluder for an SOD with a stent frame on the flexible membranes to implement a membrane spacing element, according to an embodiment.

FIGS. 114A to 114H illustrate an occluder for an SOD with membranes formed of polymeric material, according to an embodiment.

FIGS. 115A to 1115G illustrate an occluder for an SOD in which the membrane spacing element is implemented with vortex generating features on the membranes, according to an embodiment.

FIGS. 116A to 116D illustrate a method for implanting a clip and an SOD as part of an integrated procedure, according to an embodiment.

FIGS. 117A to 117F illustrate a method for implanting an SOD sequentially after delivering a clip, according to an embodiment.

FIGS. 118A to 118E illustrate a method for implanting an SOD using two guide wires after delivering a clip, according to an embodiment.

FIG. 119 illustrates an SOD with an annulus connector, according to an embodiment.

FIG. 120 illustrates an SOD with a prosthetic valve occluder, according to an embodiment.

FIGS. 121A to 121C illustrate a method for implanting an prosthetic valve or prosthetic valve occluder after a flexible membrane occluder, according to an embodiment.

FIGS. 122A to 122D illustrate various leaflet clips configured for engagement with a SOD delivered after the clip, according to embodiments.

FIG. 123 is a schematic illustration of various types of structures or connectors that may be used to secure an occluder of a SOD to various cardiac tissues and to a clip CL, according to embodiments.

FIGS. 124A to 124C illustrate an SOD with a prosthetic valve occluder and a clip connector that is implemented as a helical docking frame or helical anchor, according to an embodiment.

### DETAILED DESCRIPTION

The detailed description herein serves to describe non-limiting embodiments or examples involving various inventive concepts and uses reference numbers for ease of understanding these examples. Common reference numbers between the figures refer to common features and structure having the same or similar functions, as will be understood. While various figures will have common reference numbers referring to such common features and structure, for purposes of conciseness, later figure descriptions will not necessarily repeat a discussion of these features and structure.

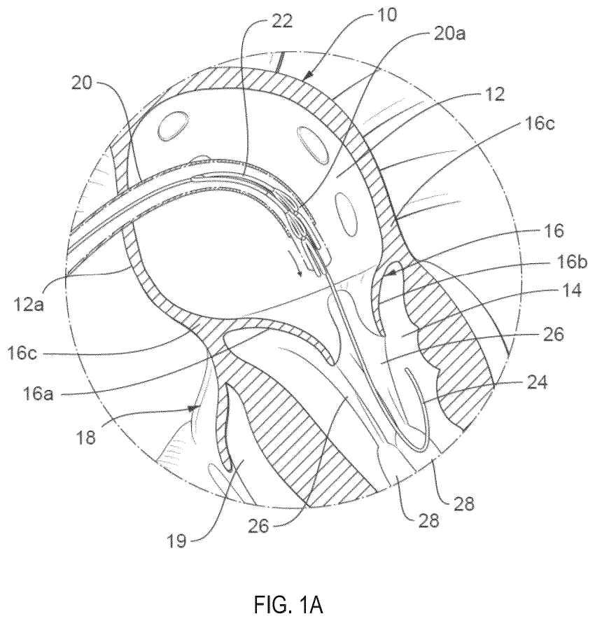

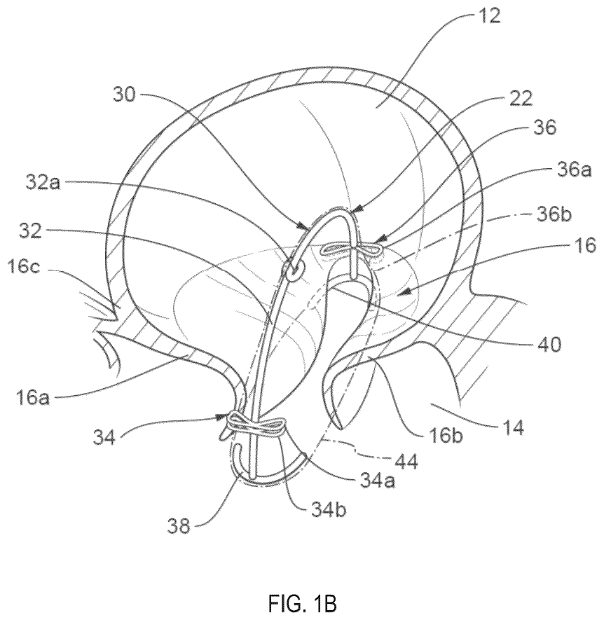

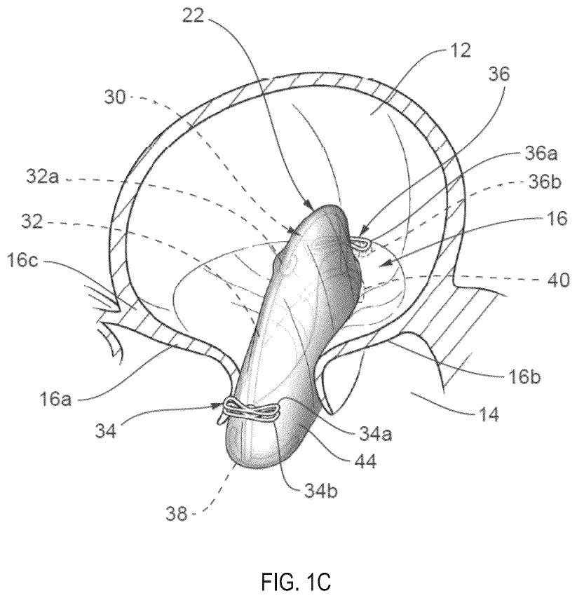

Referring first to FIG. 1A, a native heart 10 is shown and includes a left atrium 12, a left ventricle 14, and a native mitral valve 16, which controls blood flow from the left atrium 12 to the left ventricle 14. The tricuspid valve 18 is also shown in communication with the right ventricle 19. The mitral valve 16 includes an anterior leaflet 16a, a posterior leaflet 16b and a native valve annulus 16c. When the mitral valve 16 is functioning properly, it will open to allow blood flow from the left atrium 12 into the left ventricle 14 during the diastole portion of the heart cycle. When the heart 10 contracts during systole, the anterior and posterior native mitral leaflets 16a, 16b will fully coapt or engage with one another to stop any blood flow in the reverse direction into the left atrium 12 and blood in the left ventricle 14 will be ejected efficiently and fully through the aortic valve (not shown). A catheter 20 carries a collapsed selective occlusion device 22 along a guide wire 24. In this illustrative procedure, the catheter 20 is delivered transeptally across the inter-atrial septum 12a. It will be appreciated that any other transcatheter approach, or other surgical approaches of various levels of invasiveness, may be used instead. The patient may or may not be on bypass and the heart may or may not be beating during the procedure. As further shown in FIG. 1A, the native mitral leaflets 16a, 16b are supported by chordae tendineae 26 attached to papillary muscles 28. As schematically illustrated in FIG. 1A, the anterior and posterior native mitral leaflets 16a, 16b may not properly coapt or engage with one another when the heart cycle is in systole. Insufficient coaptation of the leaflets 16a, 16b leads to blood flow out of the left ventricle 14 in a backward direction, or in regurgitation, through the mitral valve 16 into the left atrium 12 instead of fully through the aortic valve (not shown).

Now referring to FIG. 1A in conjunction with FIGS. 1B and 1C, the selective occlusion device 22 has been fully extruded or extended from the distal end 20a of the catheter 20, and transformed from the collapsed position or condition shown in FIG. 1A within the catheter 20, to the expanded condition shown in FIGS. 1B and 1C. As further shown in FIGS. 1B and 1C, the selective occlusion device 22 comprises a collapsible and expandable frame structure 30. The frame structure 30 is comprised of a curved frame member 32 generally extending across the native mitral valve 16 while being supported or stabilized at the native annulus 16c. The selective occlusion device 22 is formed in a manner allowing it to be collapsed for delivery as shown in FIG. 1A, but expanded to the exemplary form shown in FIGS. 1B and 1C. This may be accomplished in many ways. For example, the frame structure 30 may be comprised of flexible polymers, metals such as super-elastic or shape memory metals or other materials. The selective occlusion device 22 may, for example, expand into a preformed shape through the use of shape memory materials. The frame structure 30 may be covered partially or completely by fabrics such as the Dacron, Teflon and/or other covering materials such as used in the manufacture of prosthetic cardiac valves or other implants. More specifically, the frame structure 30 includes a curved frame member 32 which, in this embodiment, and/or other embodiments, extends from one commissure to the other. The frame member 32 may instead extend from other portions of the heart tissue generally located at the annulus region. At opposite ends, the frame structure 30 is supported by respective first and second non-penetrating annulus connectors 34, 36. As an example of a non-penetrating annulus connector, these connectors are configured with respective upper and lower connector elements 34a, 34b and 36a, 36b. These connector elements 34a, 34b and 36a, 36b respectively sandwich or capture annulus tissue therebetween at each commissure. The connector elements 34a, 34b and 36a, 36b are each shown as “butterfly-type” connectors that may be slipped or inserted into place with native leaflet tissue sandwiched or secured therebetween. It will be appreciated that other tissue trapping connectors may be used instead, and/or other penetrating or non-penetrating connectors. Non-penetrating connectors are advantageous because they cause no damage that would otherwise occur due to penetrating connectors, and they allow for position adjustment. The frame structure 30 further includes first and second membrane support members 38, 40 at opposite ends configured to be located in the left ventricle 14 to support a flexible membrane 44 in a slightly open condition. Together with the frame structure 30, the flexible membrane 44 forms a selective occlusion device that works in conjunction with the native mitral valve leaflets 16a, 16b to control blood flow through the mitral valve 16. The flexible membrane 44, in this embodiment acts as a prosthetic heart valve by moving in coordination with the leaflets 16a, 16b as will be described below. In other embodiments, the selective occlusion device need not have any moving part that moves in conjunction with the leaflets 16a, 16b. The flexible membrane 44 is secured at opposite portions of the frame structure 30 to the support members 38, 40 in any suitable manner, such as adhesive, mechanical securement, suturing, fasteners, etc. As further shown, a considerable portion at a lower margin of the flexible membrane 44 is not attached to the frame structure 30. The membrane support members 38, 40 are short, curved members and remaining membrane portions at the lower margin of the flexible membrane 44 are not directly attached to any frame portion. This allows the flexible membrane to billow, expand or inflate outward as will be discussed further below during systole to engage with the native leaflets 16a, 16b and prevent regurgitation of blood flow in a reverse direction through the mitral valve 16 when the heart cycle is in systole.

The flexible membrane 44 may be formed of various types of thin, flexible materials. For example, the materials may be natural, synthetic or bioengineered materials. Materials may include valve tissue or pericardial tissue from animals, such as cows and pigs, or other sources. Synthetic materials such as ePTFE, Dacron, Teflon or other materials or combinations of materials may be used to construct the flexible membrane 44. Flexibility of the frame structure 30 together with the flexibility of the flexible membrane 44 provides for operation of the selective occlusion device 22 and the manners contemplated herein, and may also help prevent failure due to fatigue from repeated cycling movement of the selective occlusion device 22 in the heart 10. It will be appreciated that FIG. 1B shows the flexible membrane 44 removed for a clear view of the frame structure 30, and in this FIG. the flexible membrane 44 is in broken lines, while in FIG. 1C the flexible membrane 44 is shown in solid lines, with the heart cycle in systole and the flexible membrane 44 fully engaging the native leaflets 16a, 16b to reduce regurgitation of blood flow through the mitral valve 16. The flexible membrane 44 may be sutured to the frame structure 30 using techniques employed by the prosthetic heart valve industry for the manufacture of prosthetic aortic and mitral valves. The frame may be made from one or more layers of material, such as super-elastic or shape memory material and the membrane 44 may be suitably secured. One manner may be trapping the flexible membrane 44 between layers of the frame structure 30. To retain the membrane 44 in place, fabric covering(s) (not shown) over a metallic frame may aid in attaching the membrane 44 to the frame structure 30.

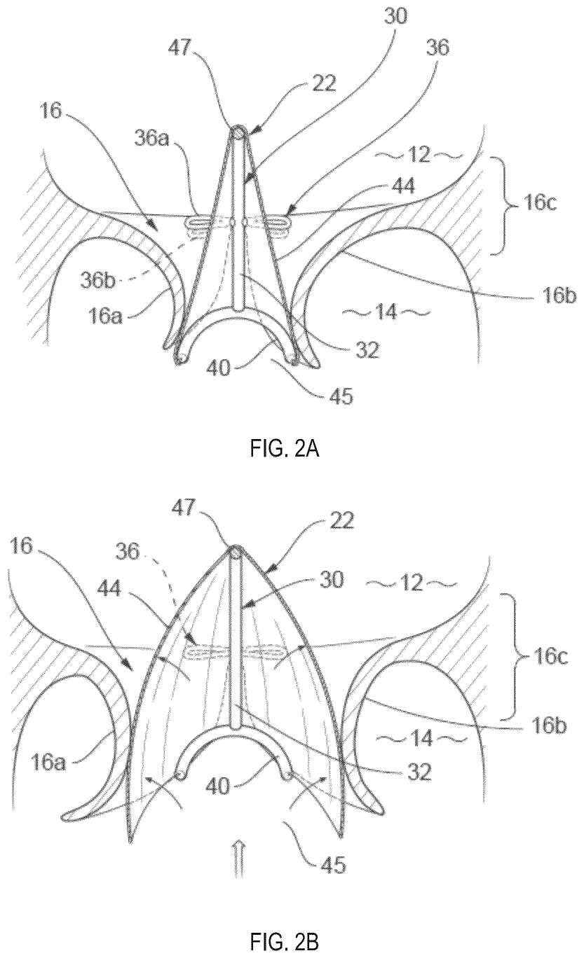

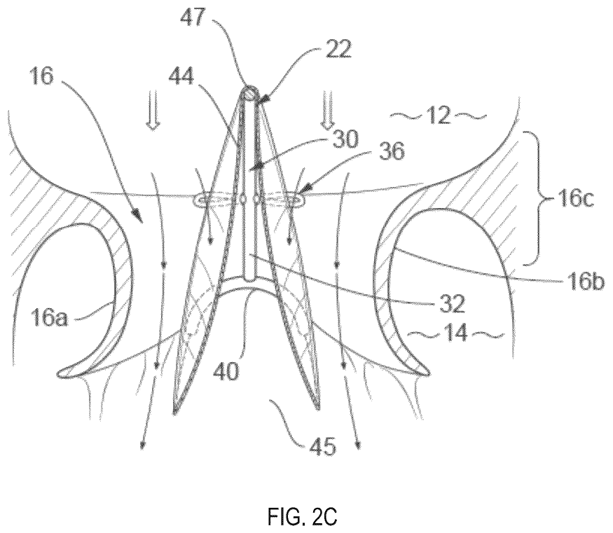

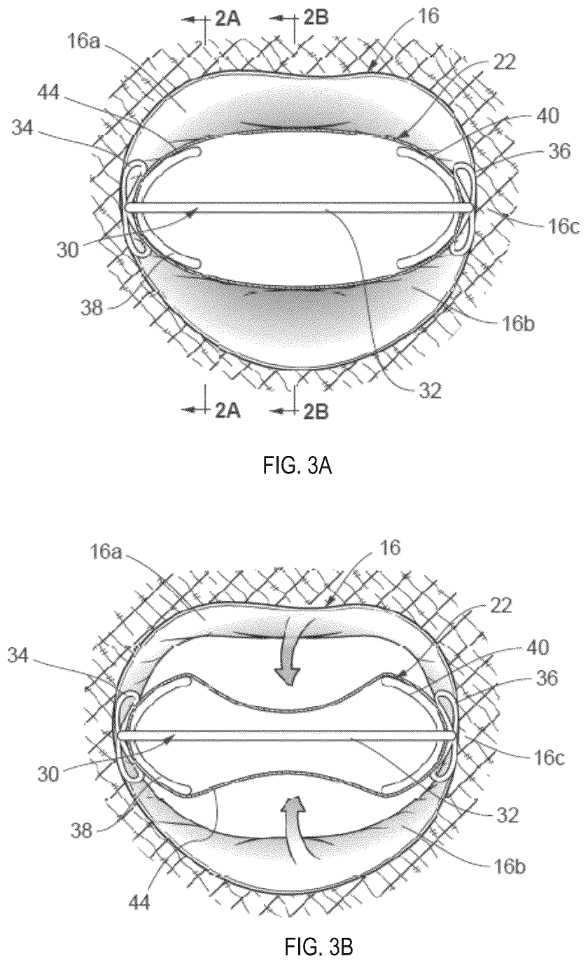

FIGS. 2A, 2B and 2C are transverse cross-sections through the selective occlusion device 22 and the mitral valve 16 shown in FIGS. 1A through 1C. FIG. 2A illustrates the device 22 in a cross section along line 2A-2A of FIG. 3A, while FIG. 2B shows the selective occlusion device 22 in cross section along line 2B-2B of FIG. 3A, with each of these two FIGS. showing the heart cycle in systole. FIGS. 3A and 3B are top views respectively showing the systole and diastole conditions, but not illustrating the hinge 32a that may be provided to assist with folding during delivery. FIG. 2C is similar to FIG. 2B but showing the selective occlusion device 22 when the heart cycle is in diastole. In systole (FIGS. 2A, 2B and 3A), which is when the native mitral valve 16 is supposed to fully close to prevent blood flow back into the left atrium 12, the pressurized blood will flow through the open end 45 of the flexible membrane and be prevented from flowing through the closed end 47, at least to any substantial degree. As will be appreciated from a review of some embodiments, a small vent may be provided in the flexible membrane. Because the flexible membrane billows or expands outwardly in the direction of the arrows shown in FIG. 2B, the native mitral leaflets 16a, 16b will seal against or coapt with the flexible membrane 44 to prevent blood flow regurgitation. In this manner, native mitral leaflets 16a, 16b that would not otherwise properly seal together or coapt will seal in systole against the flexible membrane 44. To ensure coaptation, one or more portions of the flexible membrane 44 adjacent to frame structure 30 will move away from the adjacent frame structure into contact with the native leaflet(s) 16a, 16b. In other words, only a portion of the lower margin of the flexible membrane 44 is affixed to frame structure 30. As further shown in FIG. 2B, there may be extra membrane material adjacent the membrane support members 38, 40 to allow for the expanded membrane condition. As further shown in FIGS. 2C and 3B, when the heart cycle is in diastole and blood flow needs to occur from the left atrium 12 into the left ventricle 14 (during the filling portion of the heart cycle), the blood will push past the flexible membrane 44 and the flexible membrane 44 will move into a collapsed or contracted condition while the native mitral leaflets 16a, 16b move apart or away from each other in the opposite direction to facilitate blood flow in the direction of the arrows. The arch-shaped membrane support members 38, 40 maintain a separation between lower margins or edges of the flexible membrane 44 to force blood to fill the inside or interior of the membrane 44 during systole through the open end 45, causing the membrane 44 to expand or billow outward so that the membrane 44 fills the gap between the native mitral valve leaflets 16a, 16b. The arch-shaped or curved support members 38, 40, and/or other portions of the frame structure 30, may be formed using a central wire and a fabric cover around the wire. Other constructions are possible as well, such as using soft, sponge-like material, and fabrics in conjunction with more structurally supportive material such as metal and/or plastic. The filling and emptying of the flexible membrane 44 through the open end 45 can ensure that there is washing or rinsing of the underside of the membrane 44 with each heartbeat to prevent clot formation, and any resulting embolization of clot material.

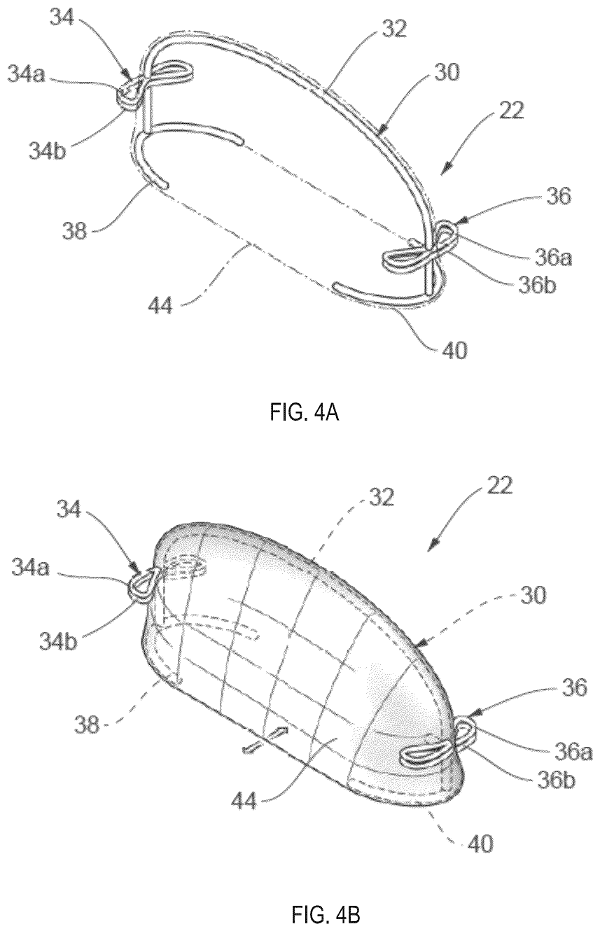

FIGS. 4A and 4B are respectively similar to FIGS. 1B and 1C, but illustrate the selective occlusion device 22 isolated from the native mitral valve 16 (FIGS. 1B and 1C).

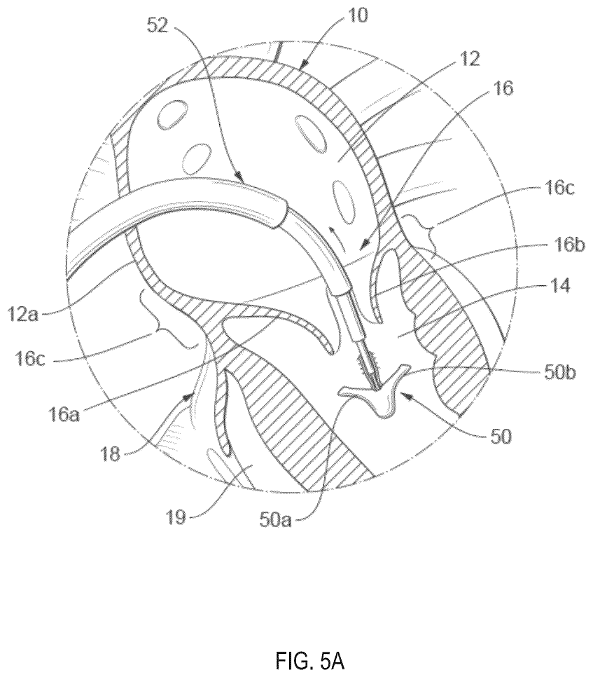

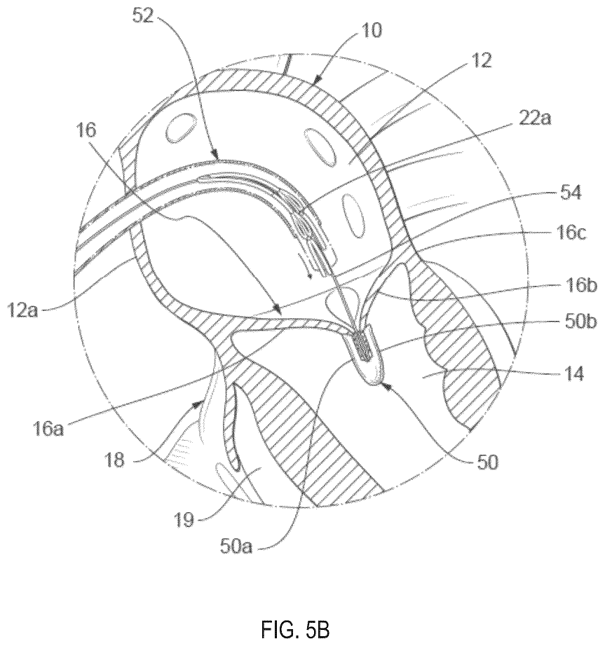

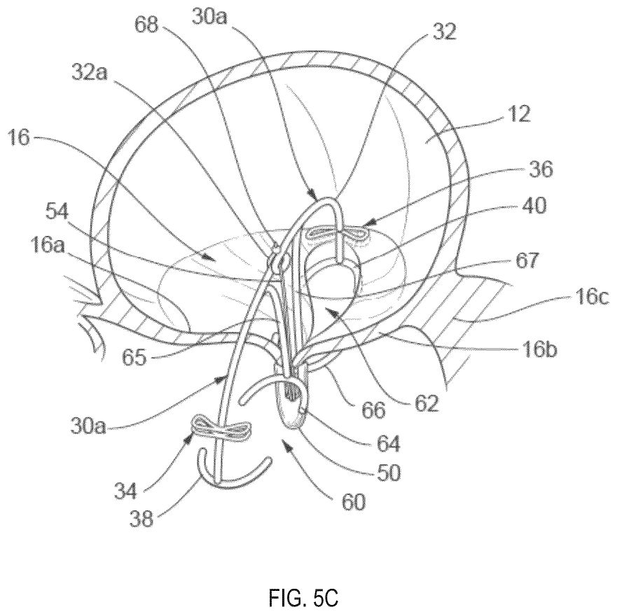

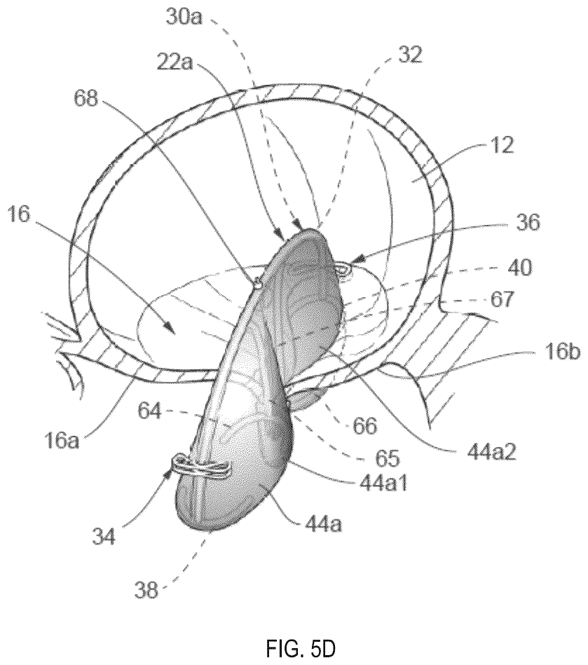

FIGS. 5A through 5D illustrate another embodiment of a selective occlusion device 22a. As previously stated, all like reference numerals between the various embodiments and FIGS. refer to like structure and function except to the extent described herein. Some reference numerals will have a suffix modification such as a letter (e.g., “22a”), or a prime mark (e.g., 90′), indicating a modification to the like structure which will be discussed and/or apparent from a review of the drawings. To be more concise, redundant descriptions of like structure and function between the various FIGS. will not be made or will be kept to a minimum. This embodiment is particularly suited to achieve beneficial effects for those mitral valve repairs involving clipping or otherwise securing one native leaflet margin to another. It will be appreciated, though, that clips or other anchors (herein generically referred to as clip structures) may be applied to only one leaflet margin, and more than one clip or anchor may be used. Often, mitral valve repair is made with a clip structure 50 having first and second clip elements 50a, 50b movable toward each other from an open condition to a closed position. The clip structure 50 is typically applied in a transcatheter procedure using a suitable catheter assembly 52. A representative and illustrative clip structure 50 is shown in these FIGS. for clipping together margins of the native leaflets 16a, 16b near a central location of each margin. The beginning of the procedure is shown in FIG. 5A with the catheter assembly 52 directed transeptally into the left atrium 12 through the inter-atrial septum 12a and into the mitral valve 16 and to the left ventricle 14. A portion of the margin of each leaflet 16a, 16b is captured by the clip structure 50 and then clipped and firmly secured together as shown in FIG. 5B. At least one of the elements 50a, 50b moves toward the other in a clipping or clamping action to change from an open condition to a closed condition. A wire, suture or other tensile member or connector 54 is coupled to the clip structure 50. At or near the end of the clipping step of the method, a selective occlusion device 22a in the form of a frame structure 30a and flexible membrane 44a (FIG. 5D) is introduced through the catheter or catheters 52 in a manner similar to the method described above with respect to the first embodiment. The selective occlusion device 22a is guided by the suture, wire or other tensile member 54 affixed and extending from the clip structure 50.

As further shown in FIG. 5C, this embodiment of the device 30a, 44a includes two sections 60, 62. This embodiment advantageously utilizes the clip structure 50 as an anchoring mechanism for assisting with securing the device 30a, 44a in place and implanted as a selective occlusion device 22a in the native mitral valve 16. The two sections 60, 62 are employed in a manner described above in connection with the single section embodiment of the device 30, 44. As will be appreciated from a review of FIGS. 5C and 5D, a modified frame structure 30a is employed to support a modified flexible membrane 44a. More specifically, the flexible membrane 44a includes corresponding sections 44al and 44a2. These may be formed from one or more distinct pieces of membrane material. In addition, third and fourth membrane support members 64, 66 are provided to support the flexible membrane sections 44al and 44a2 in manners similar and analogous to the manner that support members 38, 40 support and function in the first illustrative embodiment discussed above. An arc-shaped frame member 32 is shown similar to the first embodiment spanning across the native valve 16. Vertical support members 65, 67 extend from the frame member 32 and couple with the membrane support members 64, 66. As another option, the frame member 32 may be eliminated and the vertical members 65, 67 or other structure could be joined together in the central region of the device 22a.

As further shown best in FIG. 5C, the suture or wire 54 couples the clip structure 50 to the frame structure 30a, such as by using a crimp element or other securement 68 generally at hinge 32a. It will be appreciated that other securement methods and structures may be used instead to secure the clip structure 50 to the frame structure 30a. The clip structure 50 and the frame structure 30a may take other forms than the illustrative forms shown and described herein. Use of the clip structure 50 securing the frame structure 30a in addition to the non-penetrating and/or other connectors such as generally at the native annulus 16c provides for an overall secure implant. The clip structure 50 and one or more annulus connectors will provide opposing forces that firmly secure the frame structure 30a and flexible membrane 44a generally therebetween. The two separate selective occlusion or flow control sections 44a1, 44a2 are separated from each other by the clip structure 50. The attachment of the selective occlusion device 22a to the native mitral valve 16 may be a direct connection between the flexible membrane 44a and the native leaflets 16a, 16b (see below). Another option is that instead of the single arch-type frame member 32, the two side-by-side sections 60, 62 of the frame structure 30a may be otherwise coupled together near the center of the selective occlusion device 22a to avoid the need for a continuous frame member 32 spanning across the native mitral valve 16. Still further modifications are possible, while retaining advantages of a clip structure used in combination with a selective occlusion device. For example, the selective occlusion device may be configured as a frame structure and flexible membrane affixed around a continuous perimeter portion of the frame structure.

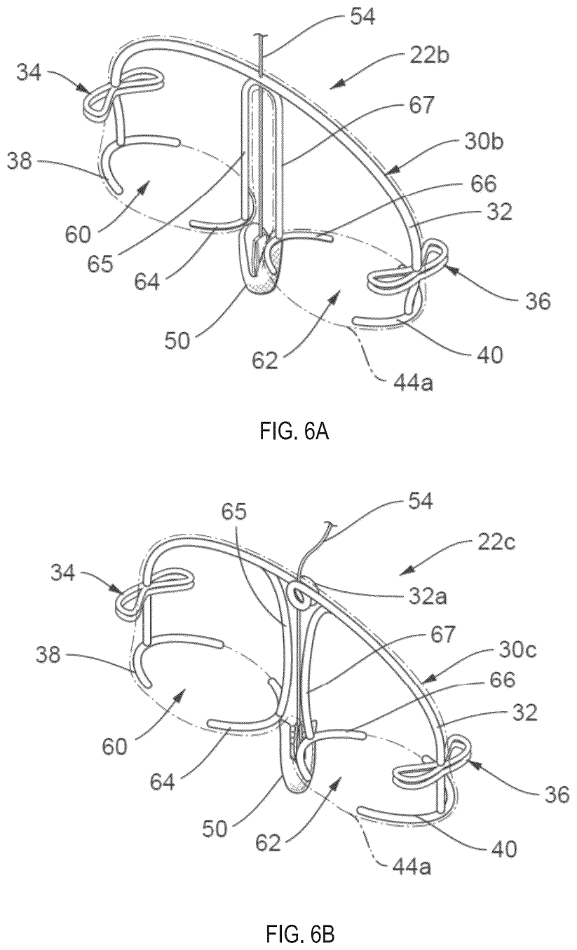

FIGS. 6A and 6B illustrate additional embodiments of selective occlusion devices 22b and 22c. In these FIGS. the flexible membrane 44a is shown in broken lines so that the respective frame structures 30b, 30c are more clearly shown. In the illustrative embodiment of FIG. 6A, the central hinge has been eliminated and the suture or wire 54 extends directly through the frame member 32. As with all embodiments, the devices 22b, 22c and any associated components, such as the frame structures 30b, 30c, may be made flexible enough and foldable into a collapsed condition for catheter delivery purposes. Again, a crimp element (not shown) or any other fixation manner may be used to secure the wire or suture 54 in tension against the frame structure 30b, 30c. FIG. 6B illustrates an embodiment of the selective occlusion device 22c slightly different from the embodiment of FIG. 6A in that the flexible membrane 44a, shown in broken lines, is folded inwardly at the region of the clip structure 50. As shown in FIG. 6A, and as one additional option, the flexible membrane 44a may be more distinctly attached to the frame members as shown by the broken lines extending upwardly against the vertical frame members 65, 67.

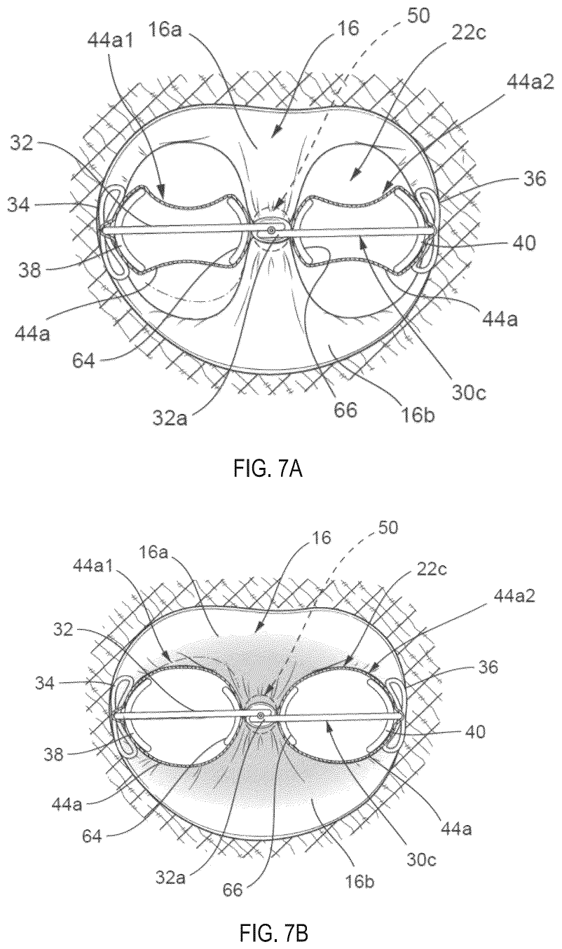

FIGS. 7A and 7B are top views illustrating selective occlusion device 22c, such as shown in FIG. 6B having separate sections 44al and 44a2 secured in place and implanted within a native mitral valve 16. FIG. 7A shows the selective occlusion device 22c when the heart cycle is in diastole, and FIG. 7B shows the selective occlusion device 22c when the heart cycle is in systole. The function of a multi-section apparatus, such as with devices 22a, 22b, 22c, is similar to the function of the single section selective occlusion device 22 discussed above in connection with the first illustrative embodiment, except that with the native mitral valve itself separated into two sections by the clip structure 50, the separate flexible membrane sections 44al and 44a2 independently function to contract or collapse in diastole (FIG. 7A) and billow, expand or inflate outwardly in systole (FIG. 7B) due to the forceful introduction of blood flow when the heart cycle is in systole. The effect or result is similar to that described above in connection with, for example, FIGS. 3A and 3B, but with the dual effect of correcting any misalignment or lack of coaptation between the native mitral leaflets 16a, 16b on each side of the clip structure 50. In this manner, blood flow is allowed in diastole as shown in FIG. 7A past the native mitral leaflets 16a, 16b which have spread or expanded outwardly and also past the two section flexible membrane 44a which has collapsed inwardly or away from the native mitral leaflets 16a, 16b. Reverse or regurgitated blood flow is at least reduced, if not reduced to essentially zero (prevented), during systole as the flexible membrane 44a expands or inflates to contact or engage the native mitral leaflets 16a, 16b creating a fluid seal.

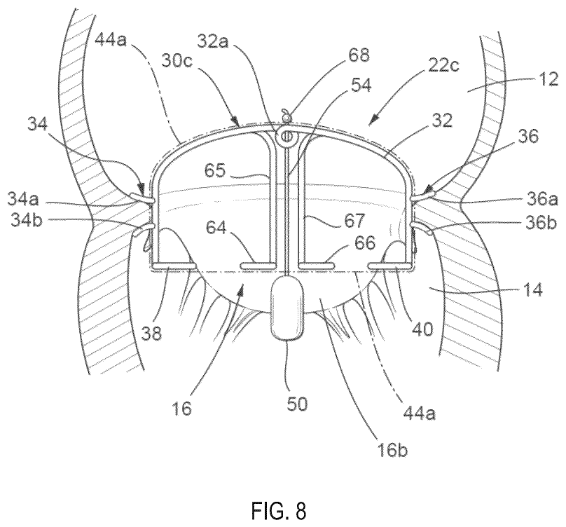

FIG. 8 shows a side view of the selective occlusion device 22c shown in FIG. 7B, but with the flexible membrane 44a shown in broken lines for clarity. The selective occlusion device 22c is securely implanted in the mitral valve 16 between annulus connectors 34, 36 generally at an upper location and a clip structure 50 at a lower location. Again, different connector and/or clip configurations may be used than those shown and described, and different numbers of connectors and clip structures may be used. The clip structure or structures may be secured to each leaflet 16a, 16b simultaneously as shown, or may be secured separately to a single leaflet 16a and/or 16b. Although the tensile member 54 is shown to have a particular length to connect between the clip structure 50 and the frame member 32, a tensile member or other type of connection of any necessary longer or shorter extent may be used instead. In some cases, the clip structure 50 may be directly affixed to the frame structure 30.

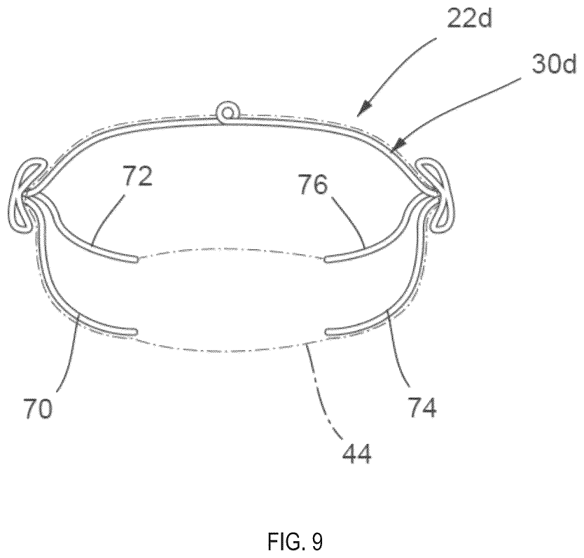

FIG. 9 illustrates a selective occlusion device 22d constructed according to an illustrative embodiment, in which an alternatively configured frame structure 30d is used and coupled with a flexible membrane 44 (shown in broken lines for clarity. Particularly, lower supporting members 70, 72, 74, 76 have a different configuration for guiding the shape of the flexible membrane 44. The flexible membrane 44 may be securely attached to the lower supporting members 70, 72, 74, 76 along their entire lengths, or along a portion of their lengths, or not at all if they are otherwise held in place during diastole in a suitable manner. The lower margins of the flexible membrane 44 are allowed to billow or expand outwardly and may be detached from the lower supporting members 70, 72, 74, 76 along at least substantial portions to allow this expanding or billowing action to take place. In addition, the entire frame structure 30d and/or only the lower supporting members 70, 72, 74, 76 may be highly flexible to allow this expansion or billowing action to take place when the heart cycle is in systole, as previously described.

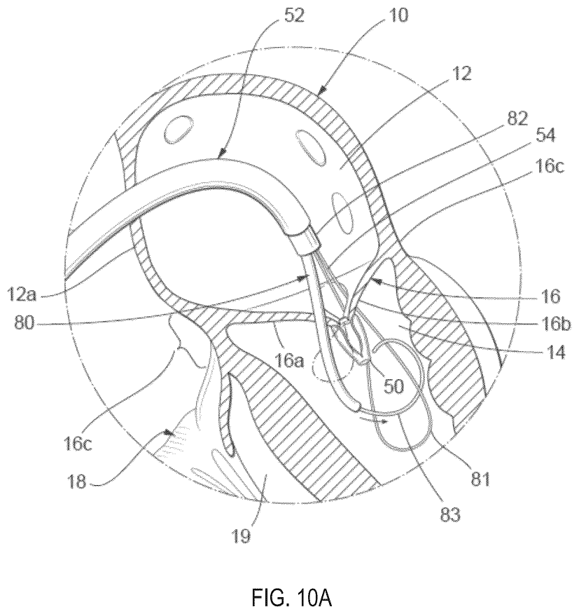

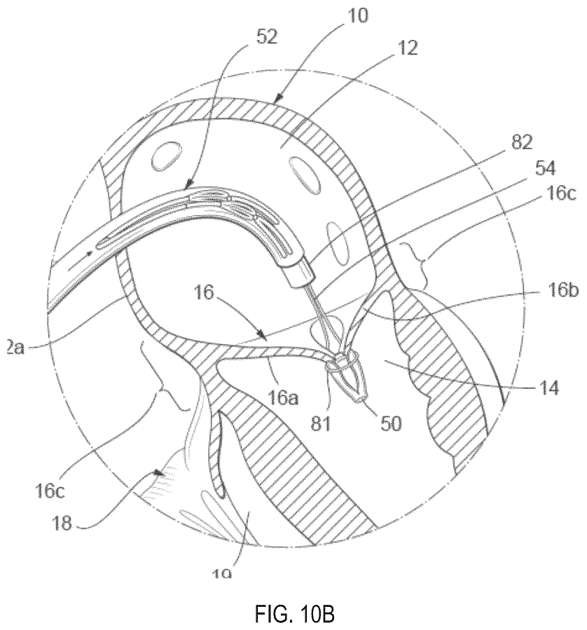

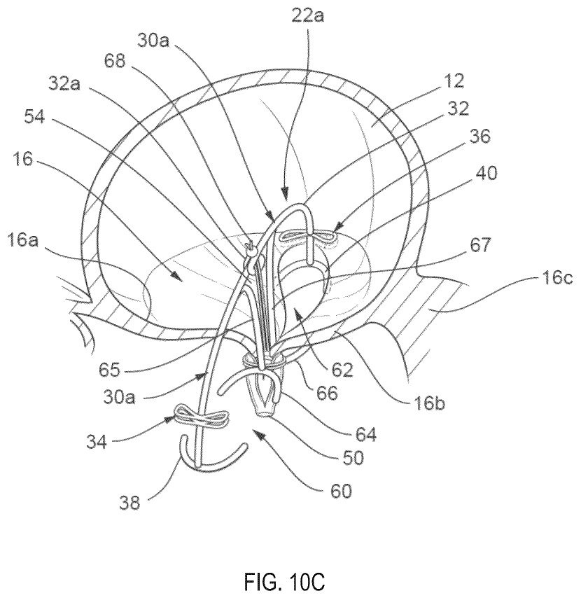

FIGS. 10A, 10B and 10C show another illustrative embodiment in which a transcatheter system 52 is used and, specifically, a clip structure capturing device 80 is used to help secure the selective occlusion device 22a in place. This may be particularly useful when applying a selective occlusion device such as according to the present disclosure to a previously implanted mitral clip structure 50. The clip structure 50 may be of any type or configuration. In cases where the clip structure 50 has failed to properly repair the mitral valve 16, or the mitral valve function has degraded over time, despite the clip repair procedure, this embodiment assists with the capturing of the previously implanted clip structure 50 and implantation of a selective occlusion device, such as frame structure 30a and flexible membrane 44a. In this regard, and as shown in FIGS. 10A and 10B, a lasso or suture loop device 81 is deployed from a catheter 82 and captures the clip structure 50 with assistance from a guide device 83. The suture, wire or other tensile member 54 that extends upwardly through the mitral valve 16 may be a part of the suture loop device 81 in this embodiment and may then be used as generally described above to guide and securely affix selective occlusion device 22a, to the clip structure 50, as shown in FIG. 10C. For clarity, the flexible membrane 44a has not been shown in FIG. 10C.

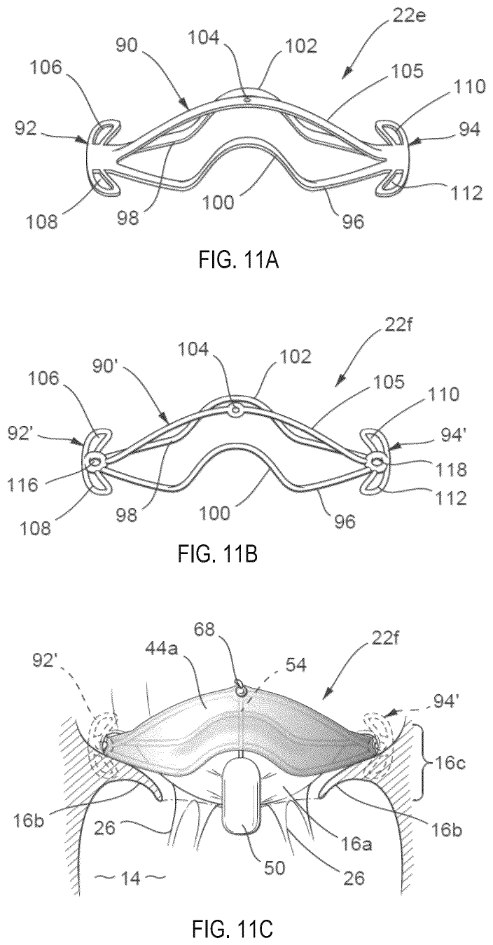

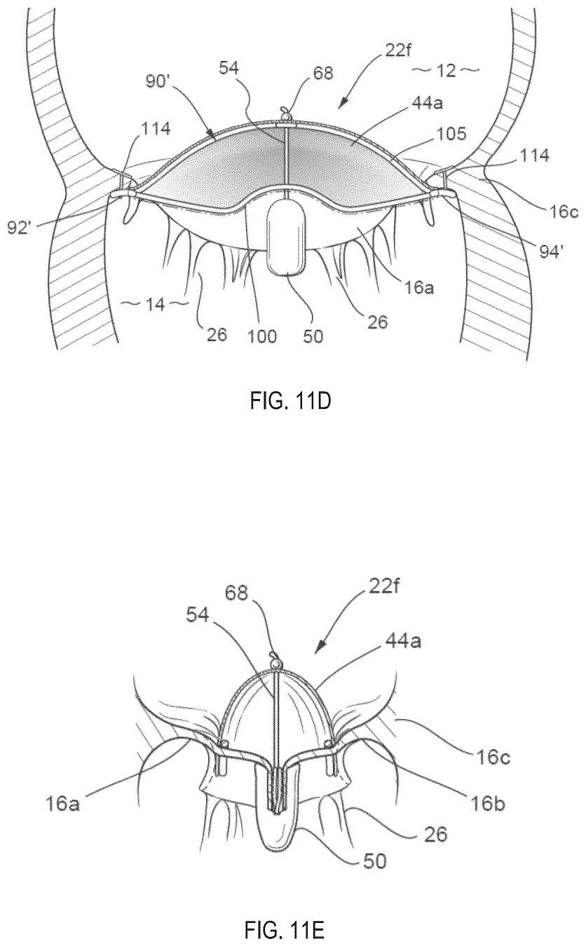

FIGS. 11A and 11B illustrate two additional embodiments of selective occlusion devices 22e, 22f, without showing the flexible membranes, that may be used to prevent blood flow regurgitation through a heart valve such as, by way of example, the mitral valve 16. In these embodiments, a flexible membrane 44a (FIGS. 11C through 11E) may be secured over a frame structure 90, 90′ from one end to the other, such as between two non-penetrating annulus connectors or, in other embodiments, penetrating connector portions 92, 94, 92′, 94′. Advantageously, there are two spaced apart elongate frame members 96, 98 extending between the connectors 92, 94, 92′, 94′, each having an upward bend or hump 100, 102 creating a recessed space. As shown in FIG. 11C the flexible membrane 44a is carried on this frame structure 90, 90′ and may be secured to the frame members 96, 98 along all or some of the lengths thereof. This can leave a desired portion of the flexible membrane 44a at the lower margin of the frame structures 90, 90′ unsecured and able to expand or billow in outward direction during systole, generally as described above in prior described embodiments or in later described embodiments. This outward expansion or billowing action will allow the flexible membrane 44a to better contact or engage the natural leaflet tissue during systole to prevent regurgitation of blood flow. This will also allow for more exchange of blood beneath or within the flexible membrane to prevent blood stagnation and the resulting possibility of clotting which may embolize and cause stroke or other complications. The humps 100, 102 in each of the lower, spaced apart support members 96, 98 accommodate the clip structure 50 and generally receive that portion of the mitral valve 16 fastened together at the A2/P2 junction. A central connection element, such as a hole 104, is provided in a central frame member 105 and allows a wire, suture or other tensile member 54 to attach the frame structure 90, 90′ to the clip structure 50. The central frame member connects the annulus connectors 92, 94 and 92′, 94′ together and arches over and across the mitral valve 16 in a manner similar to frame member 32. Suitable configurations of the frame structure 90, 90′ may be used, such as any of those previously described, for accommodating one or more clip structures and forming a plurality of separate flexible membrane sections, for example, with one section on each side of a clip structure 50. FIGS. 11A and 11B also show another way of attaching a frame structure generally at the native annulus 16c with one or more holes 106, 108, 110, 112 to engage with a suitable fixation element or anchor 114 (FIG. 11D). The embodiment of FIG. 11D includes two additional fixation holes 116, 118 for receiving fasteners. In some embodiments such as shown in FIG. 11D, penetrating anchors may be used, such as rivets, T-bars, pledgets, or other fixation elements, although the benefits of non-penetrating connectors in accordance with this disclosure would be desirable, such as for purposes of allowing self-adjustment and reduced tissue damage.

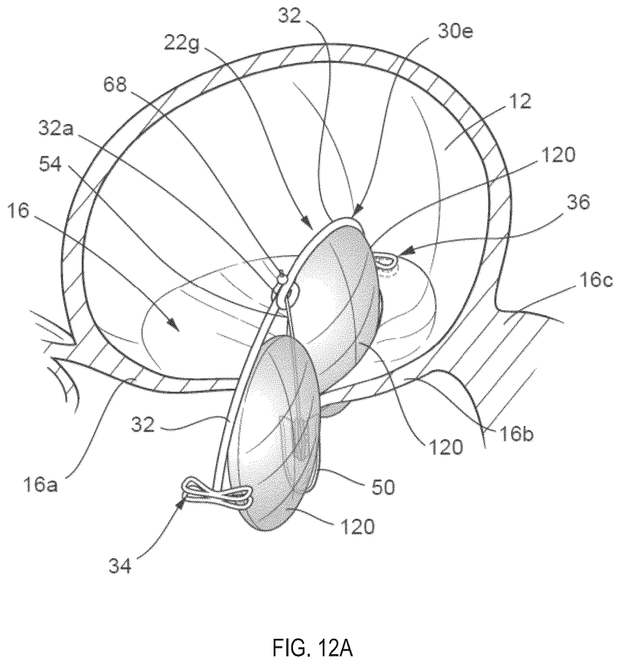

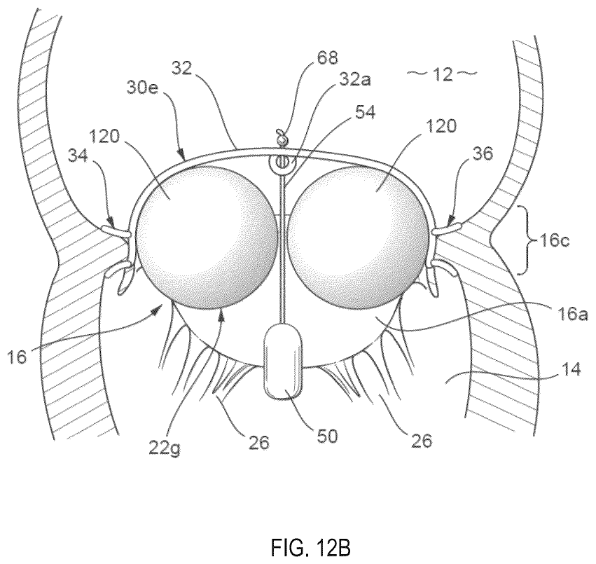

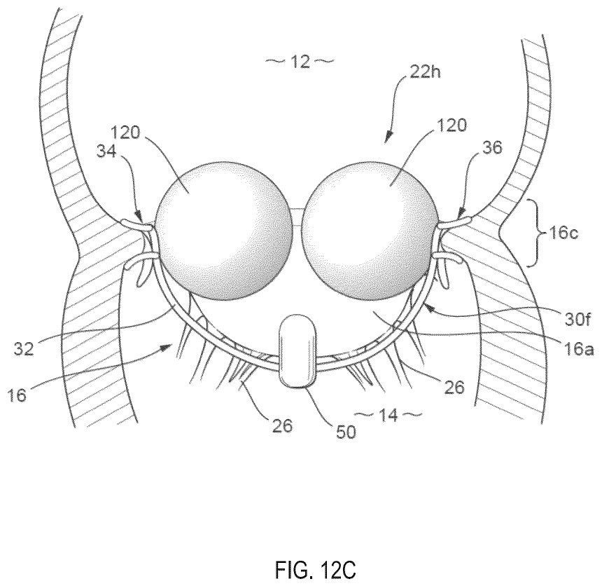

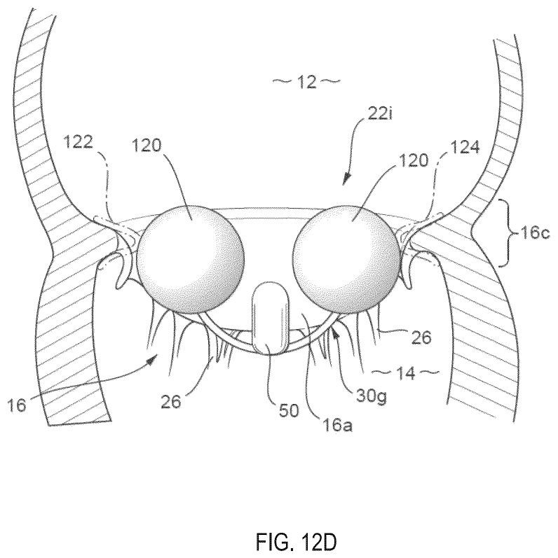

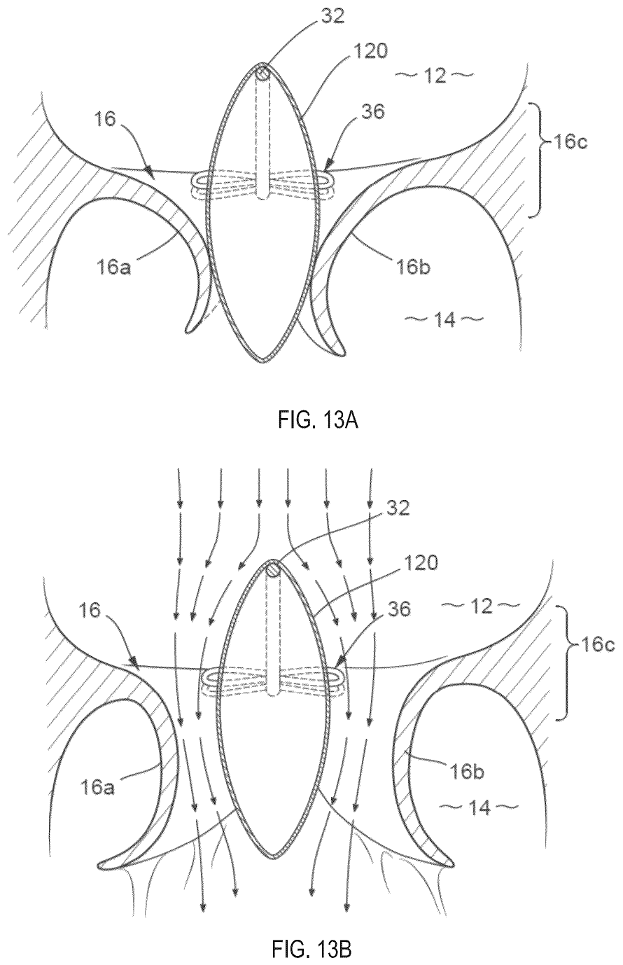

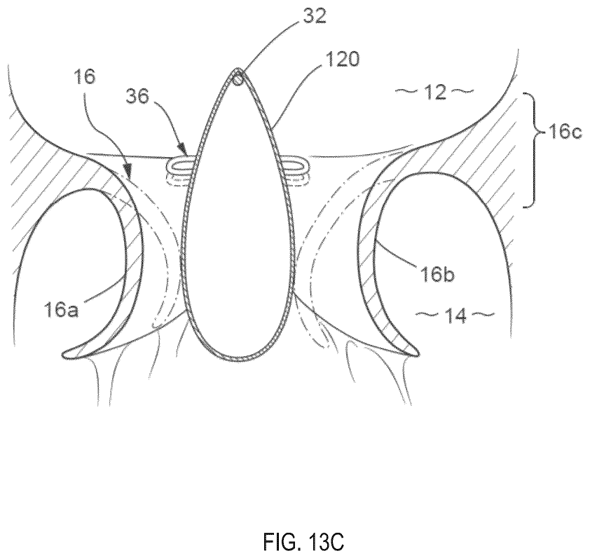

FIGS. 12A and 12B illustrate another illustrative embodiment of a selective occlusion device 22g. Rather than employing a flexible membrane, this apparatus includes at least one rigid occlusion element 120. This embodiment is more specifically configured to operate in conjunction with mitral valve leaflets 16a, 16b that have been affixed together at a central location along their margins with a clip structure 50 such as a clip structure previously described. Therefore, two selective occlusion elements 120 are provided for reasons analogous to the two section flexible membrane embodiments described herein. The selective occlusion elements 120 are “rigid” in use within the mitral valve 16 in that they are static and need not flex inwardly or outwardly to engage and disengage the native mitral leaflets 16a, 16b during the systole and diastole portions of the heart cycle. Instead, these disk-shaped elements 120 retain their shape and are sized and located in the native mitral valve 16 such that the native mitral leaflets 16a, 16b engage the elements 120 during systole and disengage the elements 120 during diastole. This selective or cyclical interaction is shown in FIGS. 13A and 13B, to be described further below. The device 22g shown in FIGS. 12A and 12B includes a frame structure 30e that is configured to extend generally across the native mitral valve 16, with a frame member 32 and hinge 32a as generally described in previous embodiments, along with non-penetrating annulus connectors 34, 36 as also previously described. Further, the clip structure 50 is secured to the frame structure 30e with a crimp element 68 and a suture, wire or other tensile member 54, such as in one of the previously described manners. In this way, the first and second rigid, selective occlusion elements 120 are respectively disposed on opposite sides of the native mitral valve 16 and on opposite sides of the clip structure 50 to selectively include the openings in the native mitral valve 16 formed when the clip structure 50 is affixed to each leaflet 16a, 16b bringing central portions of the two leaflet margins together either in direct contact with each other or in contact with a spacer (not shown) disposed between the movable clip elements. In this embodiment, the frame structure 30e is formed with a curved or arch-type frame member 32 configured to extend over the native mitral valve 16 in the left atrium 12.

The selective occlusion device 22g is shown when the heart cycle is in systole in FIGS. 12A, 12B and 13A. The native anterior and posterior mitral valve leaflets 16a, 16b are shown being forced inwardly toward each other. There is no blood leak or regurgitation because the static occlusion elements 120 fill any residual gap between the anterior and posterior leaflets 16a, 16b. The elements 120 do not need to be of the depicted shape. Any shape of space filling would be sufficient if the gap between the two leaflets 16a, 16b is filled by the elements 120. The best shape could be determined at least partly by studying the shape of the gap between the native mitral valve leaflets 16a, 16b in systole after a clip structure 50 has been applied. The optimal shape for the elements 120 for a particular patient anatomy may even be custom manufactured for that patient with rapid manufacturing techniques. Advantages of using rigid/static element(s) 120 include their ability to withstand repeated cycling forces perhaps better than a design that relies on one or more moving valve elements that may be more susceptible to fatigue.

FIG. 12B more particularly shows a cut away view of the mitral valve 16 from commissure to commissure. At the commissures, the anchors or connectors 34, 36 are shown on each side-both above and below the leaflets 16a, 16b. Centrally, there is a clip structure 50 or other attachment that anchors to the mitral valve leaflets 16a, 16b either individually or together. A tensile or other connecting member 54 extends up from the clip attachment component 50 and attaches to the frame member 32 which extends across the valve 16 from commissure to commissure.

The frame structure 30e can be constructed of a metal material such as stainless steel or Nitinol. Nitinol or other shape memory or super-elastic material may be preferred as this can be collapsed for delivery via a catheter device inside the heart, and then expanded inside the heart for implantation.

The element(s) 120 may be constructed in a number of ways and have various shapes. They could be composed of a frame of metal such as Nitinol that could be collapsed for catheter delivery. The metal frame could be covered by a plastic material or other artificial material like silicone or Teflon or polyurethane. Animal or human pericardium and animal or human heart valve material or any of the materials typically used for heart valve leaflet construction could be used to cover the frame structure 30e. A synthetic material or bioengineered material could also be used to cover the frame structure 30e.

The inside of the static occlusion elements 120 could be hollow. Or, a bladder or sac could be inside to fill the hollow interior space of the element(s) 120. The bladder could be filled with air or any gas or a liquid such as saline, sterile water, blood, antibiotic or antiseptic fluid, polymer or curable fluid material. The use of a bladder to fill the inside of the element 120 could eliminate the need or reduce the need for a frame associated with the element 120.

The selective occlusion device 22g has commissural and leaflet attachments to anchor it in position. It would also be possible to create this apparatus without a leaflet attachment. For example, the attachment could be at the commissures only. It would not be necessary to have a clip structure 50 and a member connected to the frame member 32. In this case there would not need to be two occluding elements 120. A single occlusion element 120 could be used to fill any gap between the two leaflets 16a, 16b. The shape of course would be different-likely an oval surface to extend between the commissures. The frame of such an element could be similar to that previously shown and described in connection with the first embodiment or another configuration.

FIG. 12C shows another illustrative embodiment or variation of a selective occlusion device 22h mounted inside the heart to the native mitral valve 16. There are two selective occluding elements 120 attached to a frame structure 30f. The frame structure 30f is engaged with a clip structure 50 that is attaching the anterior and posterior leaflets 16a, 16b together centrally, e.g., near the A2/P2 junction. The frame structure 30f is stabilized by connectors 34, 36 at the commissures and annulus region 16c of the valve 16.

The embodiment of FIG. 12C is similar to that shown in FIGS. 12A and 12B. The difference here is that the support frame member 32 is not located above the elements 120 but below the elements 120. In other embodiments the support frame member 32 is located above the selective occlusion device and been directed to the left atrium. In this embodiment, the supporting frame member 32 is biased downward and toward the left ventricle, generally below the mitral valve 16. Also, in this embodiment, the frame member 32 can be directly connected to the clip structure 50 that attaches the two leaflets 16a, 16b and the frame structure 30f together. This may allow a procedure where the entire device is implanted at one time. The clip structure 50, with the selective occlusion device elements 120 coupled to frame structure 30f, could be delivered by a catheter (not shown). The clip structure 50 (with or without exposing the rest of the device) could be extruded outside the delivery catheter inside the heart 10. The clip structure 50 may then be closed on the native mitral valve anterior and posterior leaflets 16a, 16b. The remainder of the selective occlusion device 22h could be then released from the delivery catheter-placing the entire device in position. This may simplify the procedure to one step.

It is also important to note that in prior embodiments the frame structure has been above the clip structure 50, and in this embodiment, the frame structure 30f is below. It is also possible to have both an upper and a lower support frame structure (such as by combining two arc-shaped supports in one device). It would also be possible to join upper and lower arc support or frame members, so the support or frame structure is a complete loop or circle. This may provide further structural strength to the system.

FIG. 12D is a side elevational view schematically illustrating another illustrative embodiment of a selective occlusion device 22i including first and second rigid or static selective occlusion elements 120 coupled with a frame structure 30g. In this embodiment, the rigid selective occlusion elements 120 are directly coupled to the frame structure 30g, which may be a frame member 32 coupled with the clip structure 50. As in previous embodiments, the clip structure 50 may directly couple respective margins of the anterior and posterior mitral leaflets 16a, 16b, or may couple these leaflet margins together against an intermediate spacer (not shown). This may be used to correctly orient and locate the rigid selective occlusion elements 120 on opposite sides of the clip structure 50 and within the side-by-side openings of the native mitral valve 16 created by the central clip structure 50. Optionally, additional connectors 122, 124 shown in broken lines may be used to help secure the rigid selective occlusion elements 120 in place at the commissures of the mitral valve 16.

FIGS. 13A and 13B schematically illustrate, in cross section, the functioning of the rigid, selective occlusion elements 120 shown in FIGS. 12A through 12D. Specifically, when the heart cycle is in systole the native mitral leaflets 16a, 16b will close against the rigid selective occlusion elements 120 to provide a fluid seal against regurgitation of blood flow. As shown in FIG. 13B, during diastole, the mitral valve leaflets 16a, 16b will spread apart and disengage from the rigid selective occlusion elements 120 to allow blood flow from the left atrium 12 into the left ventricle 14 between the rigid selective occlusion elements 120 and the respective native leaflets 16a, 16b. The one or more elements 120 fill any gap between the anterior and posterior leaflets 16a, 16b. When mitral regurgitation occurs due to failure of complete leaflet coaptation, the leaflets 16a, 16b are frequently pulled apart from each other in the plane of the valve 16 (here left-right). However, the situation may become more complex because the leaflets 16a, 16b tend to be pulled down into the ventricle 14 as well as apart from each other as mitral regurgitation becomes more severe over time. So, an up/down gap may also occur with one leaflet 16a or 16b sitting at a higher plane than the other leaflet 16a, 16b.

The advantage to a convexly curved outer surface of the element(s) 120 is that this surface can be shaped to adapt to a wide variety of defects that may occur between the anterior and posterior leaflets 16a, 16b. An outer, convexly curved surface of the element(s) 120 can accommodate leaflet gaps that are in the plane of the valve 16 (left right in the Figure) and perpendicular to the plane of the valve 16 (up and down in the Figure).

The selective occlusion device 22g is symmetric on each side. The elements 120 could also be constructed so that they are asymmetrical, i.e., not identical on opposite sides. For example, the posterior leaflet 16b may be more retracted into the left ventricle 14 than the anterior leaflet 16a. It may be useful to have adjustments in the element 120 on the side facing the posterior leaflet 16b to fill the gap left by a retracted posterior leaflet 16b. The element 120 may be constructed to be more prominent on the side of the element 120 adjacent to the posterior leaflet 16b than on the side adjacent or facing the anterior leaflet 16a. One or more elements 120 may be adjustable in shape, such as by an adjustable level of inflation to a hollow interior of the element 120 or other method, to accommodate any need to fill a gap between the leaflets 16a, 16b that would otherwise cause regurgitation.

Custom made or custom size elements 120 could also be made depending on the shape of the gap. A gap could be determined by echocardiography or CT and appropriately sized and shaped filling elements 120 could be selected based on measurements obtained with imaging. The valve defect that needs repair may be more shaped as a cylinder and a cylinder or pyramid-cylinder shape may be better to stop blood regurgitation than a lens or disc shape for the element(s) 120.

The margins of the element(s) 120 facing the oncoming flow of blood from the left atrium 12 has a tapering surface. This will allow the blood to flow smoothly into the left ventricle and avoid blood damage or hemolysis and to promote complete and unimpeded filling of the left ventricle 14. The edge of the element(s) 120 inside the left ventricle 14 also demonstrates a taper similar to the inflow region of the element(s) 120. When the heart begins to contract, blood will be ejected back toward the element(s) 120 and the native leaflets 16a, 16b will begin to move toward the element(s) 120 to produce a complete seal-preventing regurgitation of blood during systole.

An additional option is provided and illustrated in FIG. 13C. The rigid selective occlusion element(s) 120 may be formed in a fluid efficient manner, such as a teardrop shape or other hemodynamic shape to prevent undesirable blood flow patterns and damage or hemolysis as the blood flows past the elements 120 in between the element 120 and the respective mitral leaflets 16a, 16b.

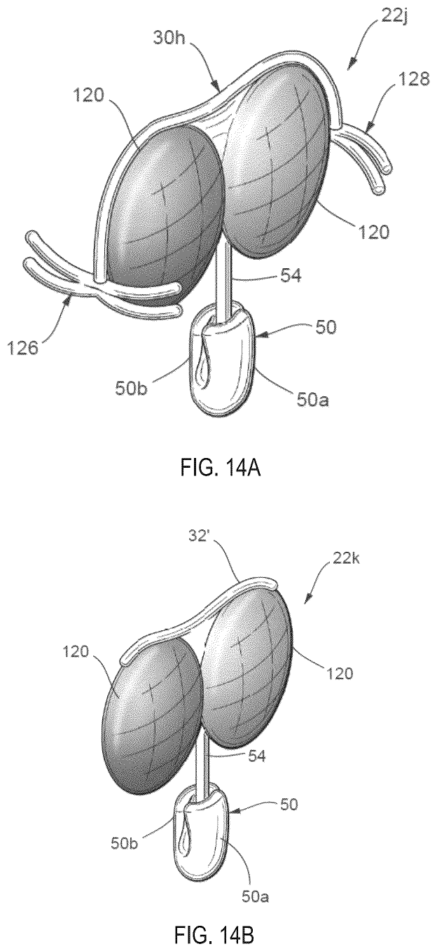

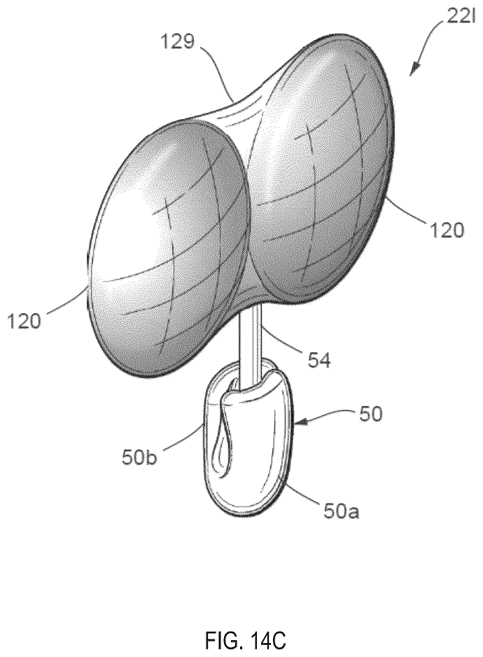

FIGS. 14A, 14B and 14C illustrate additional embodiments of selective occlusion devices 22j, 22k, 221 that utilize rigid or static selective occlusion elements 120. These elements 120 function as discussed above in connection with FIGS. 12A through 12D and FIGS. 13A, 13B. In FIG. 14A the rigid or static selective occlusion elements 120 are coupled to a frame structure 30h that is secured along top margins of the elements 120. At each end of the frame structure 30h respective commissure connectors 126, 128 are provided that include connecting elements which operate the same as the butterfly type elements previously described by sandwiching mitral tissue or other heart tissue therebetween. Additional securement is provided by the clip structure 50 and a suitable tensile element or other connector 54, such as also previously described.

FIG. 14B illustrates an embodiment of a selective occlusion device 22k in the form of rigid or static elements 120 that are again generally disc shaped and secured together by a frame member 32′, a tensile element or connector 54 and a connected clip structure 50.

FIG. 14C illustrates an embodiment of a selective occlusion device 221 in which the rigid selective occlusion elements 120 are secured together by fabric or other structure 129, and further secured through a tensile member or other connector 54 to a clip structure 50 which secures the selective occlusion device 221 to the native mitral valve 16 through a clipping action as previously described.

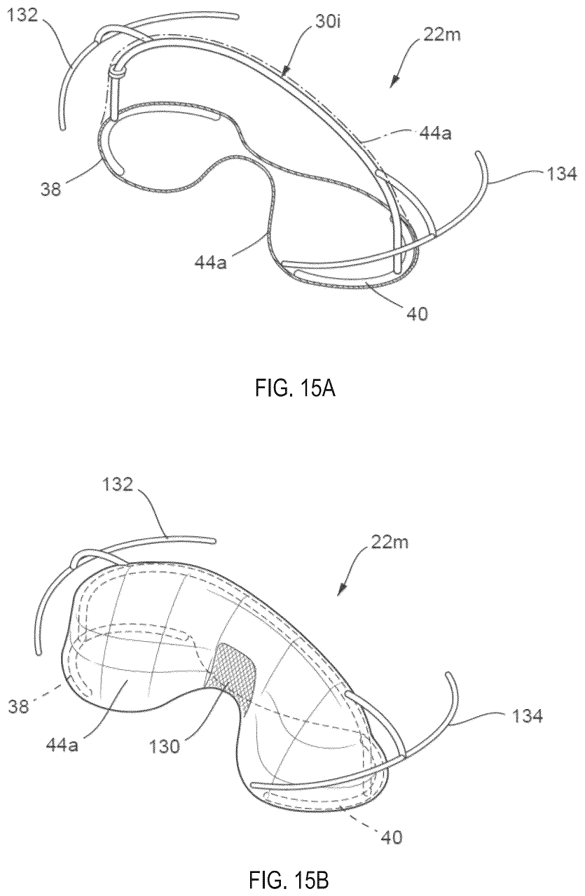

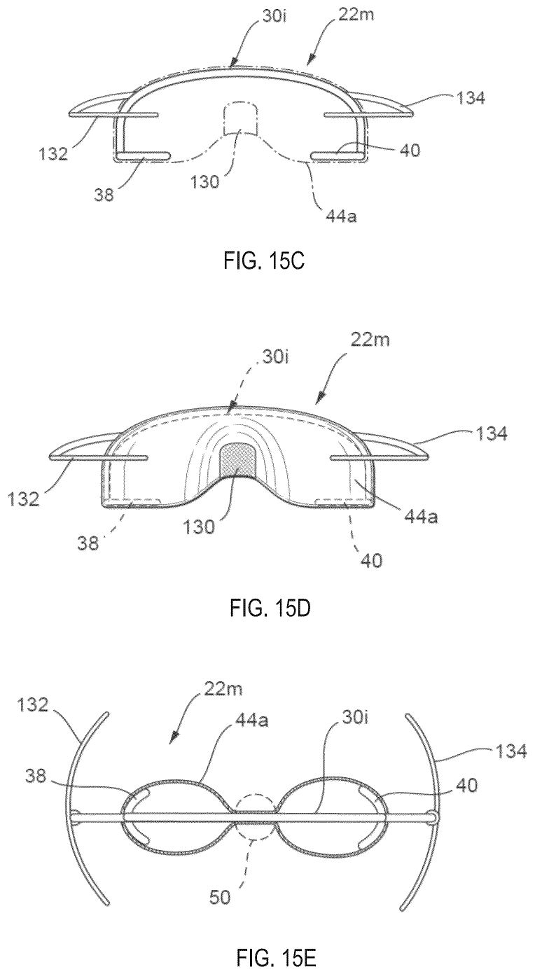

FIGS. 15A through 15E illustrate another embodiment of a selective occlusion device 22m including a flexible membrane 44a and a frame structure 30i. The flexible membrane 44a is secured to frame structure 30i that is also preferably flexible for reasons such as previously described. This embodiment is similar to previous embodiments utilizing flexible membranes 44a in conjunction with a mitral valve clip structure 50, but includes a central reinforced area such as a fabric area 130 allowing the native leaflet margin tissue to be a clipped against the reinforced fabric area 130 directly. The clip structure 50 is shown in broken lines in FIG. 15E. In this alternative, the native mitral tissue is not directly contacting abutting native mitral tissue but instead contacts and is secured against the reinforced central fabric area 130 of the flexible membrane 44a. This fabric or other reinforcing material 130 may, for example, be useful in situations where the remainder of the flexible membrane is formed from more delicate material such as biologic material. Annulus connectors 132, 134 are provided and rest against an upper portion of the annulus 16c as generally shown in other Figures, such that the clip structure 50 (not shown in this embodiment) secures the selective occlusion device 22m to the reinforced, central area 130 from below, and the annulus connectors 132, 134 secure the selective occlusion device 22m from above by bearing against or otherwise coupling to the native annulus 16c.

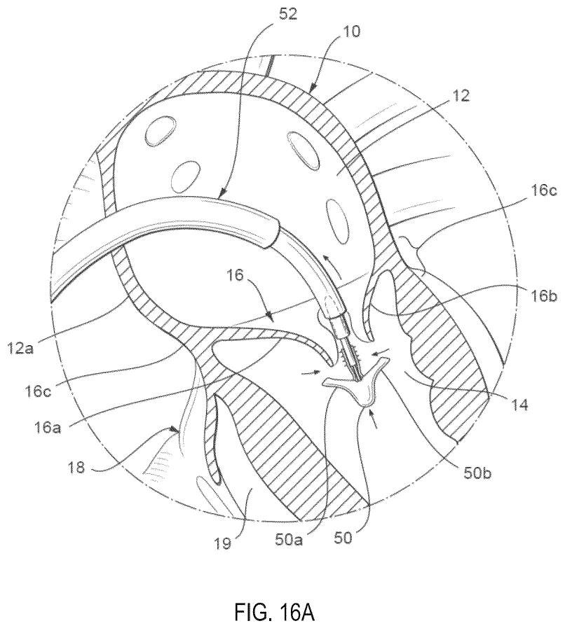

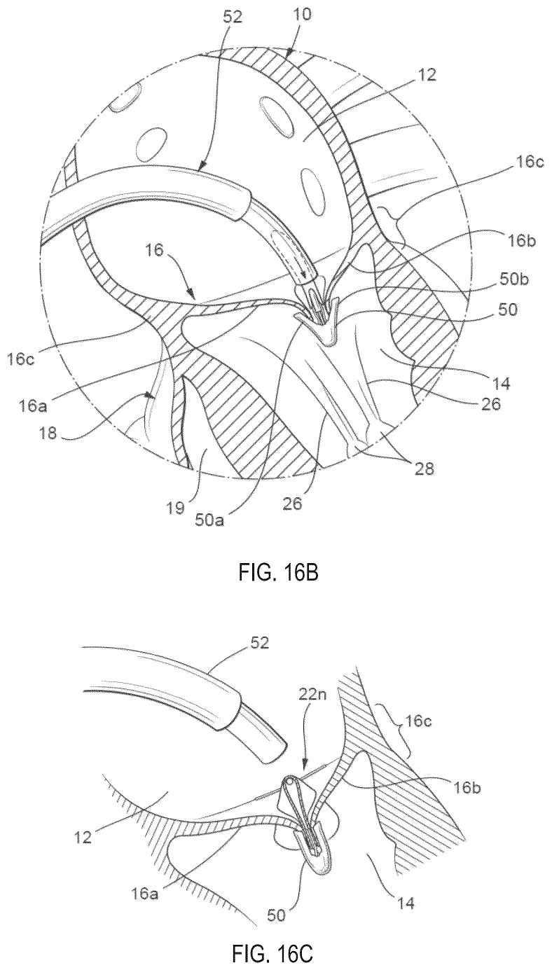

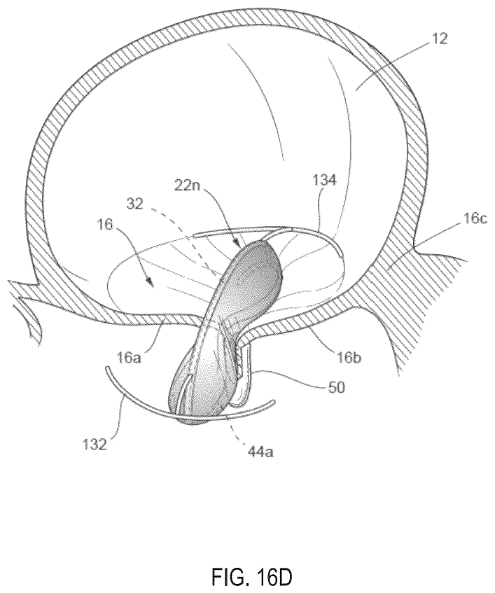

FIGS. 16A through 16D illustrate another illustrative embodiment of a transcatheter delivered selective occlusion device 22n combined with a clip structure 50. Again, the clip structure 50 is used to affix a lower central margin portion of one leaflet 16a to a lower central margin portion of the opposing leaflet 16b, generally as previously described. Again, this clipping action may be for purposes of clipping the anterior leaflet 16a directly in contact with the posterior leaflet 16b at the central location, or clipping the anterior and posterior leaflets 16a, 16b against an intermediate spacer. In this embodiment, the selective occlusion device is coupled with the clip structure 50 delivered through one or more catheters 52. As shown in FIGS. 16A and 16B, the catheter assembly 52 is delivered transeptally into the left atrium 12 and downwardly through the native mitral valve 16 although other approaches may be used instead in the various embodiments. The clip structure 50 is extruded from the catheter assembly distal end and, in the open condition shown in FIG. 16A captures the leaflet margin portions as shown in FIG. 16B and is actuated to move one or both clip elements 50a, 50b together into the position shown in FIG. 16C to secure the central leaflet margin portions together. The remaining portion of the selective occlusion device 22n is then extruded from the distal end of the catheter assembly 52 as shown in FIG. 16C. As shown in FIG. 16D the selective occlusion device 22n, which may be, as illustrative examples, of the type shown in FIG. 16D or any of the types otherwise shown and described herein, or even other configurations contemplated hereby, self-expands into the mitral valve location. Operation of the selective occlusion device 22n may be generally as described herein, and securement of the device 22n occurs generally between the clip structure 50 and respective annulus connectors 132, 134. Specifically, as previously discussed, the annulus connectors 132, 134 provide a downward force for securing the device 22n generally at the annulus 16c, while the clip structure 50 provides an upward force to generally secure the selective occlusion device 22n therebetween in place in the native mitral valve 16.

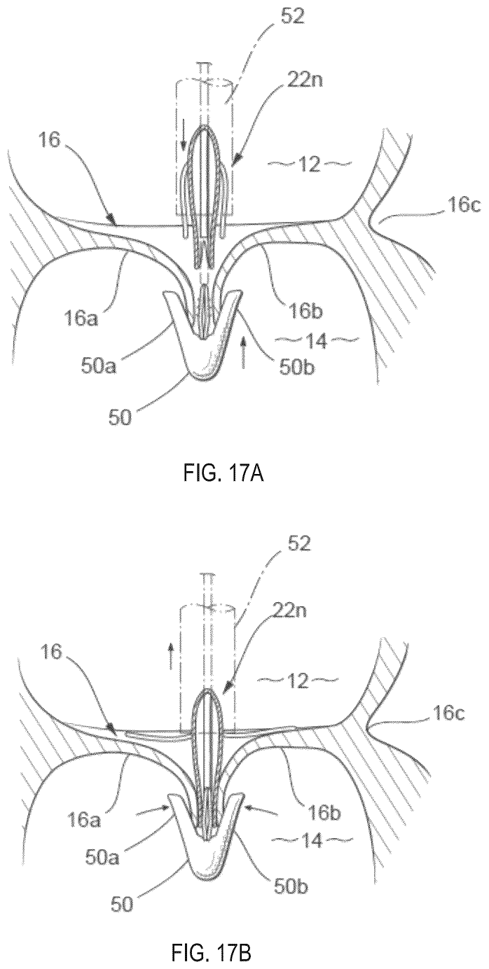

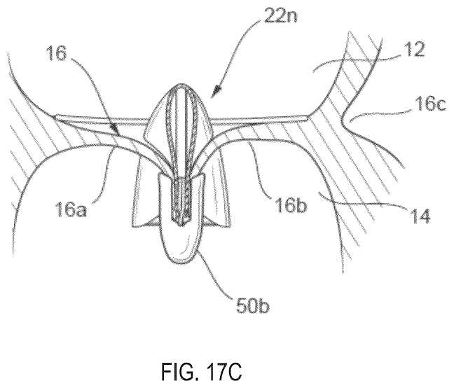

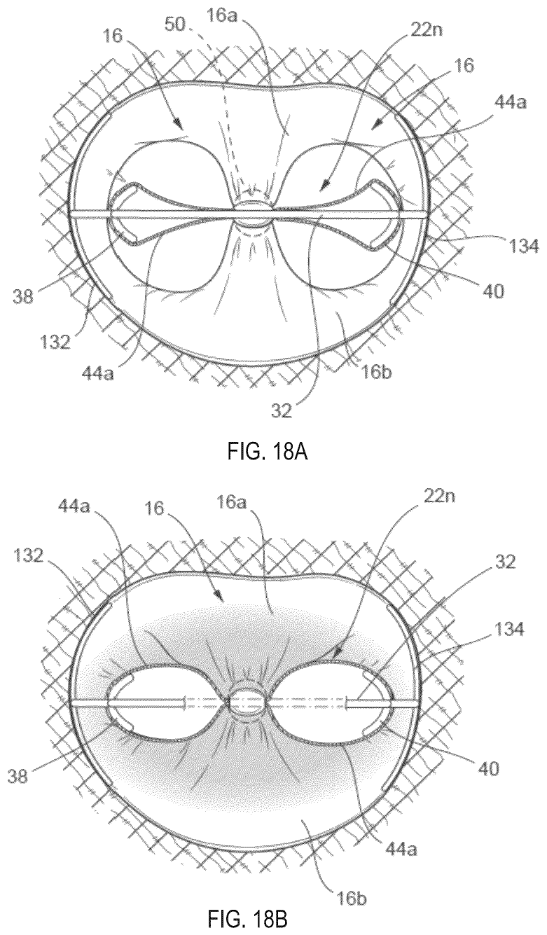

FIGS. 17A through 17C illustrate an embodiment of an apparatus for transcatheter delivery and implantation. In this embodiment, the clip structure 50 is delivered below the mitral valve 50 generally as previously described, and the selective occlusion device 22n is delivered to a location above the native mitral valve 16. The selective occlusion device 22n is inserted into the mitral valve 16 and between the native leaflets 16a, 16b, and also between the clip elements as shown in the method proceeding from FIG. 17A to 17B. Once in position as shown in FIG. 17B, at least one of the clip elements is moved toward the other clip element to clip or clamp the leaflet margins together, as previously described, and also to clamp a lower central portion of the selective occlusion device 22n and, particularly, the flexible membrane 44a in this embodiment, such that the leaflet margins are secured together at the same time as the selective occlusion device 22n is secured and implanted in place within the native mitral valve 16. As shown in FIG. 17C, the selective occlusion device 22n is fully extruded from the catheter assembly, whereupon it self-expands into position in the native mitral valve 16 and functions as otherwise generally discussed herein. More particularly, FIGS. 18A and 18B illustrate the diastole and systole portions, respectively, of the heart cycle with the apparatus secured in place as described in connection with FIGS. 17A through 17C. In FIG. 18A, during diastole, blood flow is allowed between the native mitral leaflets 16a, 16b and the flexible membrane 44a, while in systole the flexible membrane 44a, in each section, fills with blood and thereby expands or inflates as the mitral leaflets 16a, 16b move toward one another and against the flexible membrane 44a to form a fluid seal preventing regurgitation of blood flow from the left ventricle 14 into the left atrium 12 of the heart 10.

FIG. 19 is an anatomical view from above the native mitral valve 16 with the selective occlusion device 22n superimposed to show another representation for the configuration in which the selective occlusion device 22n is curved and flexes in accordance with the natural curvature of the mitral valve 16.

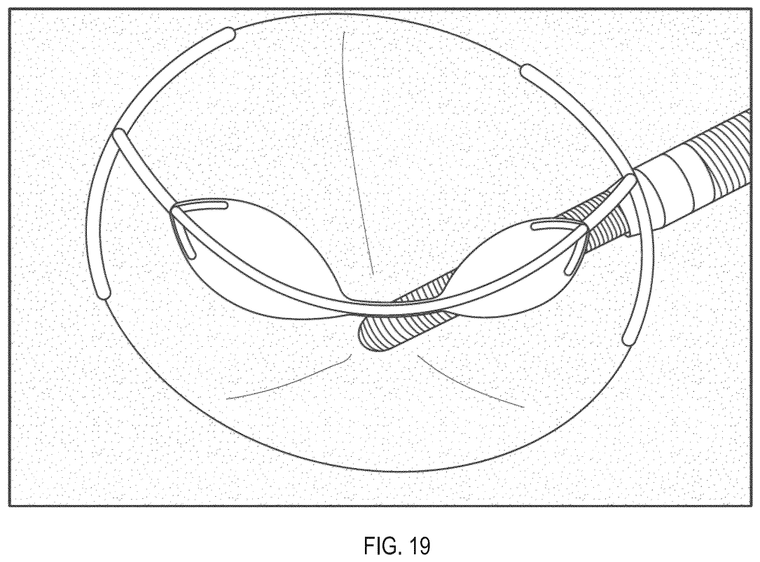

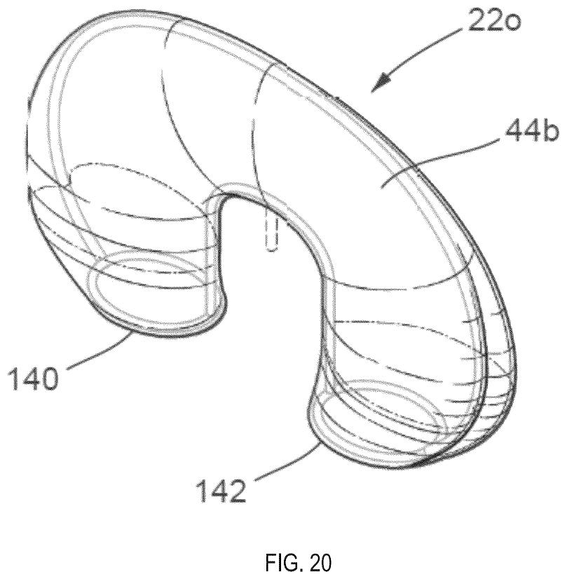

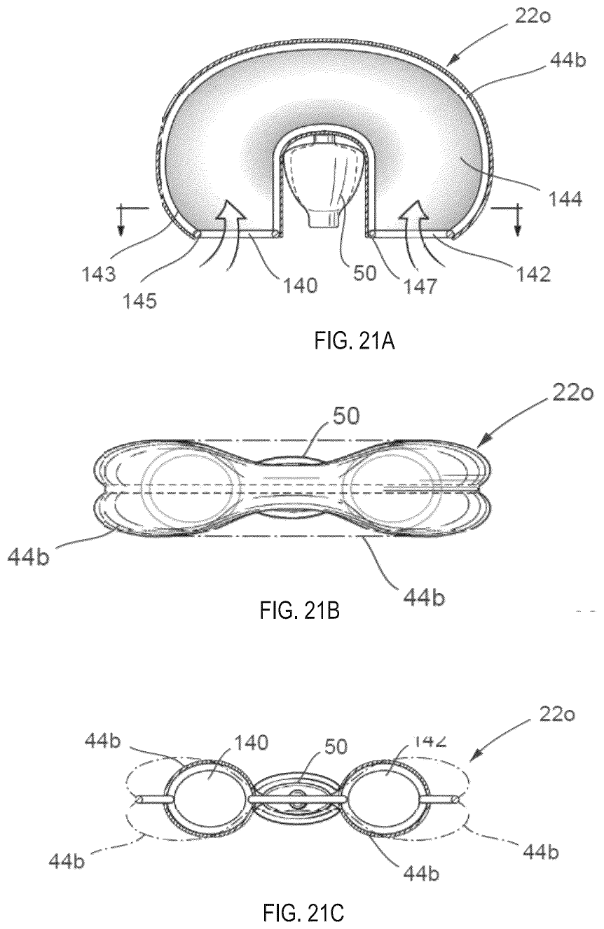

FIGS. 20, 21A, 21B and 21C illustrate another embodiment for a selective occlusion device 220 and apparatus (combining the device 220 with a clip structure 50), in which the selective occlusion device 220 is configured generally as a two section device, but with the sections in fluid communication as best shown in FIG. 21A. A clip structure 50 is secured to the selective occlusion device 220 at a position between respective open ends 140, 142 of the sections. The clip structure 50 is used in the same manner as previously described. The flexible membrane 44b is supported by a flexible but strong frame structure 143, which may be formed in any manner contemplated herein, such as for allowing transcatheter delivery and implantation. The open ends 140, 142 are defined by hoop or ring portions 145, 147 of the frame structure 143. The hollow interior 144 of a flexible membrane 44b receives blood flow in the systole portion of the heart cycle and fluid communication between the two openings 140, 142 ensures better rinsing or washing during the heart cycle to reduce the chances of blood clots.

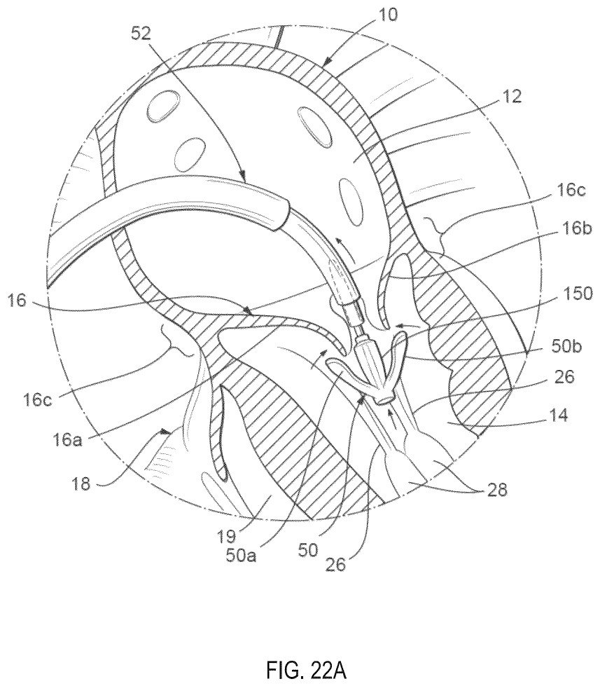

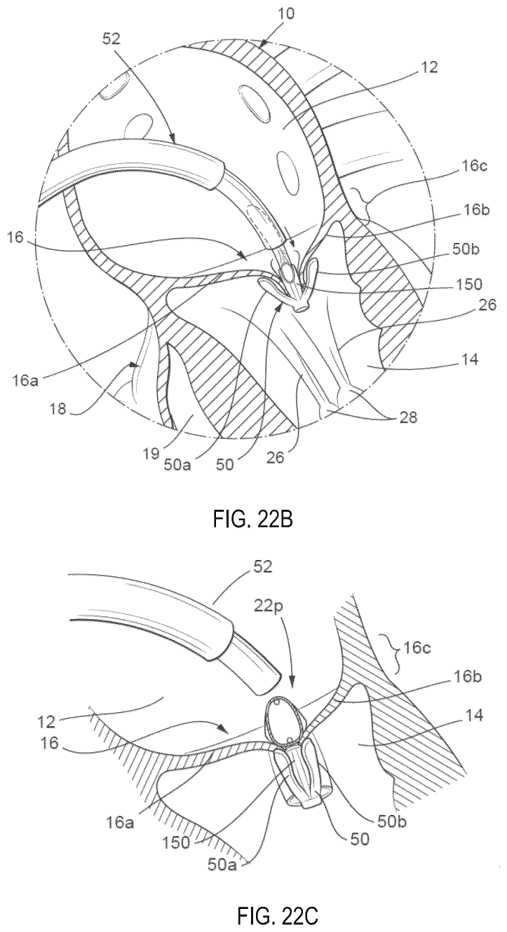

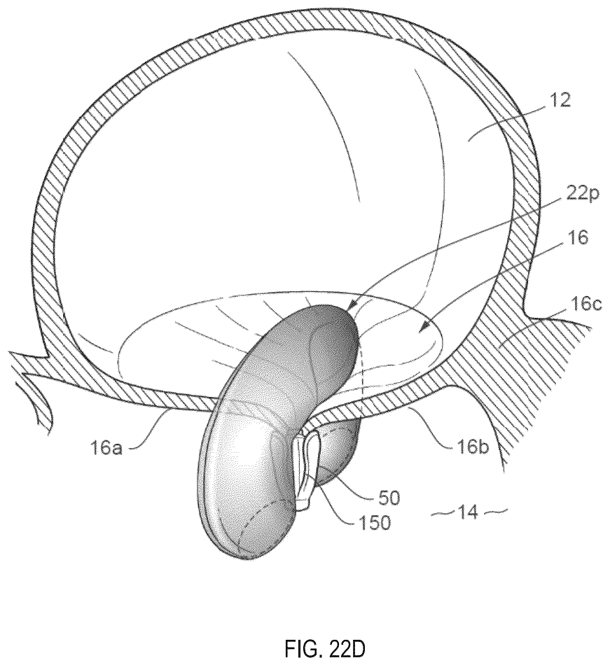

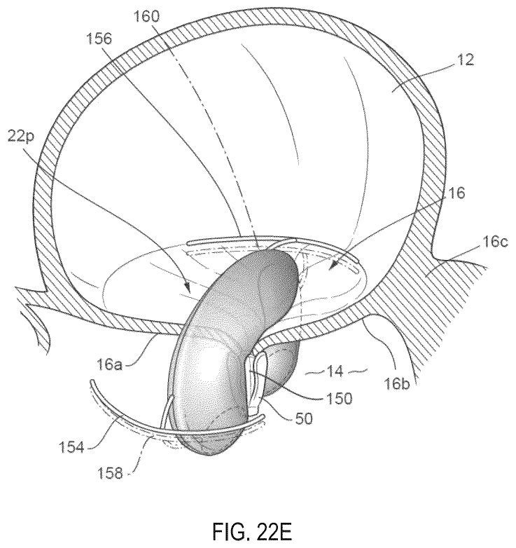

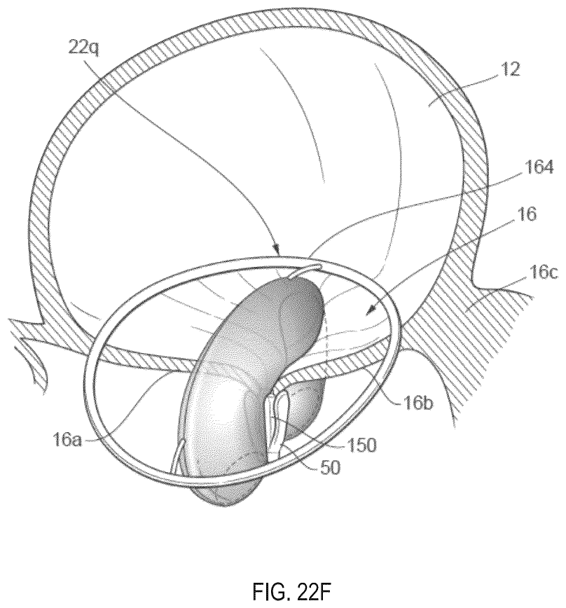

FIGS. 22A through 22D illustrate another embodiment of an apparatus for transcatheter delivery and implantation of a clip structure 50 coupled with a selective occlusion device 22p. A difference with this embodiment is that the clip structure 50 clips the native mitral leaflets 16a, 16b against a central or intermediate spacer 150, instead of directly into contact with each other. The procedure is generally shown in FIGS. 22A through 22C in which the clip structure 50 is first extruded from the transeptally directed catheter assembly 52 generally at a location below the mitral leaflets 16a, 16b. The leaflets 16a, 16b are captured against the intermediate spacer 150, as shown in FIG. 22B. The leaflets 16a, 16b are secured firmly against the spacer 150 as shown in FIG. 22C by moving at least one of the clip elements 50a, 50b toward the other. In this embodiment, each clip element 50a, 50b is moved toward the central or intermediate spacer 150 to clamp leaflet tissue against the spacer 150. The selective occlusion device 22p, in this illustrative embodiment, is already secured to the clip structure 50 when it is extruded from the catheter assembly 52 as illustrated in FIG. 22C whereupon the selective occlusion device 22p self-expands into the implanted condition shown in FIG. 22D. It will be appreciated that the selective occlusion device 22p may be extruded and implanted as a separate component, as well as coupled to the clip structure 50 in a suitable manner, instead of being extruded in an already assembled form from the catheter or catheters 52.

FIG. 22E illustrates another embodiment, similar to that shown in FIG. 22D, but further illustrating respective annulus connectors 154, 156 as part of the selective occlusion device 22p in the form of frame members that bear against heart tissue generally at the annulus 16c in the left atrium 12 and, additionally or optionally, frame members or connectors 158, 160 (shown in broken lines) coupled with the selective occlusion device 22p and located in the left ventricle 14 abutting the annulus 16c from below. Use of both sets of annulus connectors 154, 156, 158, 160 results in sandwiching the heart tissue therebetween for better securement.

FIG. 22F illustrates another embodiment of a device 22q, similar to FIG. 22E, but illustrating a single annular connector 164 generally encircling the native mitral valve 16 formed as part of the selective occlusion device and anchoring the selective occlusion device 22q in the native mitral valve 16 securely, preventing rocking in any direction but allowing flexibility. As with all embodiments, the frame members may be formed of any desired material, such as flexible wire-like materials formed from polymers and/or flexible metals including super-elastic or shape memory materials. This can help achieve overall goals of the embodiments of flexibility for collapsed delivery and improved operation during implanted use, as well as resistance against failure due to fatigue in this application involving continuous cycling in the heart.

FIG. 22G illustrates another embodiment of a device 22r. The selective occlusion device 22r may be as described in connection with any other embodiment, but for illustrative purposes, is shown with a hollow flexible membrane 44b, while the frame structure has been modified as shown. The frame structure includes a generally annular frame member 170 such as described and shown in connection with FIG. 22F, but including raised portions 170a, 170b relative to other portions. The raised portions 170a, 170b are configured to be located adjacent and above the commissures of the native mitral valve 16 and are connected with a central frame member 32 extending generally across the native mitral valve 16 and formed as part of the selective occlusion device 22r such as with another connecting frame member 172. Such frame members at the annulus, as with all embodiments, may be above the annulus, below the annulus, or frame members/connectors may be above and below the annulus to sandwich tissue therebetween.

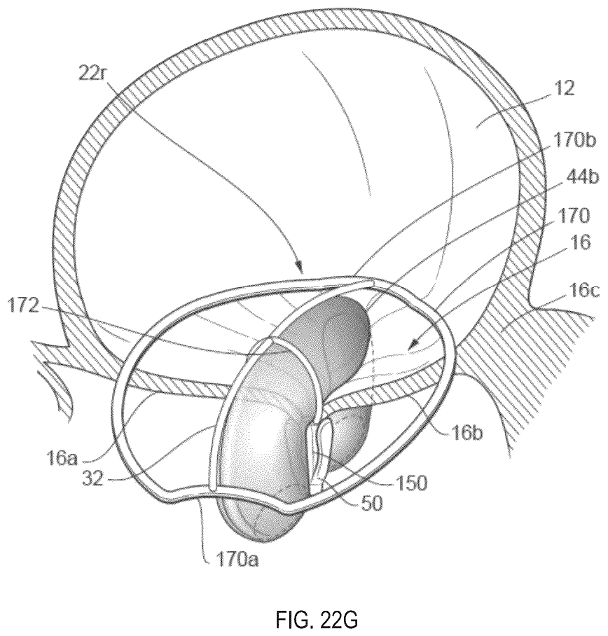

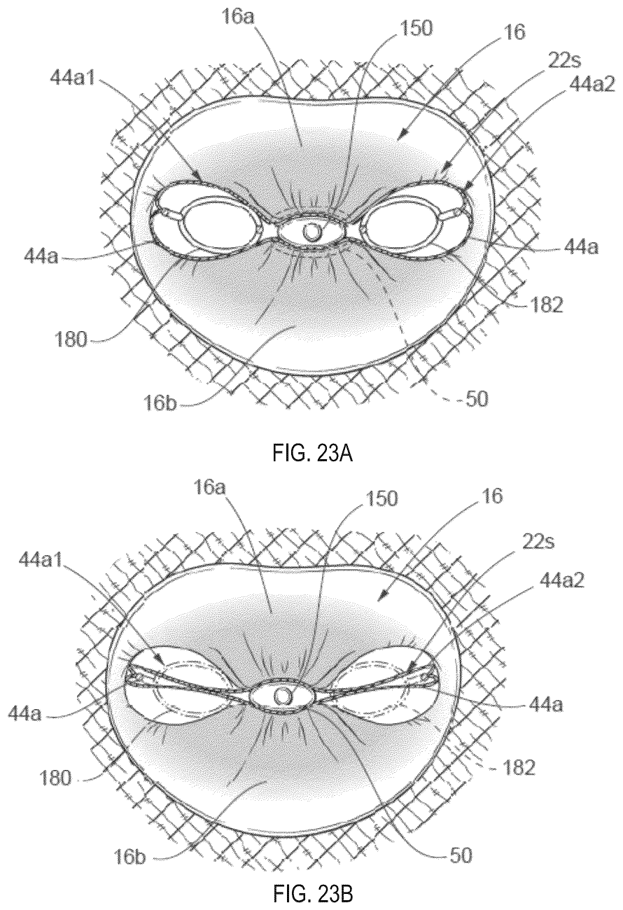

FIGS. 23A and 23B schematically illustrate a selective occlusion device 22s coupled with a central clip 50 including a spacer 150 implanted in a mitral valve 16. FIG. 23A illustrates the device 22s and the mitral valve 16 when the heart cycle is in systole, while FIG. 23B illustrates the mitral valve 16 and the selective occlusion device 22s when the heart is in diastole. The frame structure includes respective hoops or rings 180, 182 as shown in solid lines in FIG. 23A and broken lines in FIG. 23B. These define the openings 140, 142. A benefit of this frame configuration is that the frame will not contact the commissures during repeated heart cycling. The device, like other embodiments allows blood flow from the left atrium to the left ventricle in diastole but prevents blood flow during systole.

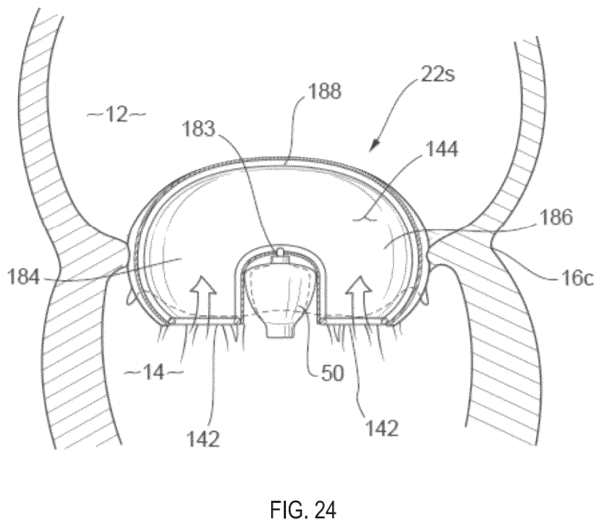

FIG. 24 is a cross-sectional view schematically illustrating the mitral valve 16 and the implanted selective occlusion device 22s, coupled with a central clip structure 50 such as at a coupling 183. The selective occlusion device 22s is of a type with a hollow interior 144 having two fluid communicating sections 184, 186 and respective first and second openings 140, 142 and a closed end 188. Fluid communication between sections 184, 186 allows for better rinsing and washing action and reduced chance of clotting.

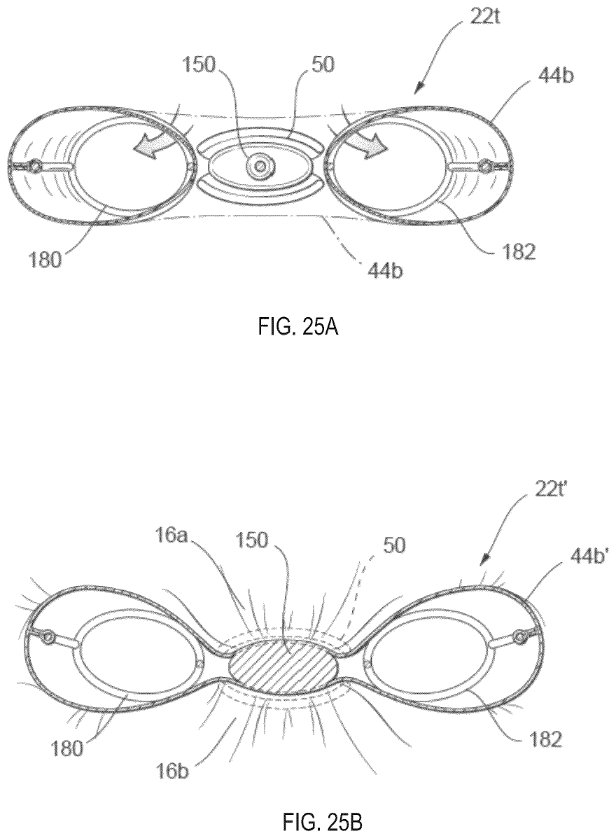

FIGS. 25A and 25B are schematic views of a selective occlusion device 22t, 22t′ including a flexible membrane 44b, 44b′ with FIGS. 25A and 25B showing the selective occlusion devices 22t, 22t′ when the heart cycle is in systole. The difference between the two devices 22t, 22t′ is that the flexible membrane 44b′ is integrated into the spacer 150 of the clip structure 50, while the flexible membrane 44b is not. Flexible membrane 44b and/or another portion, such as a frame portion, of device 22t may be otherwise coupled to clip structure 50 such as in the manner shown in FIG. 24 or another suitable manner.

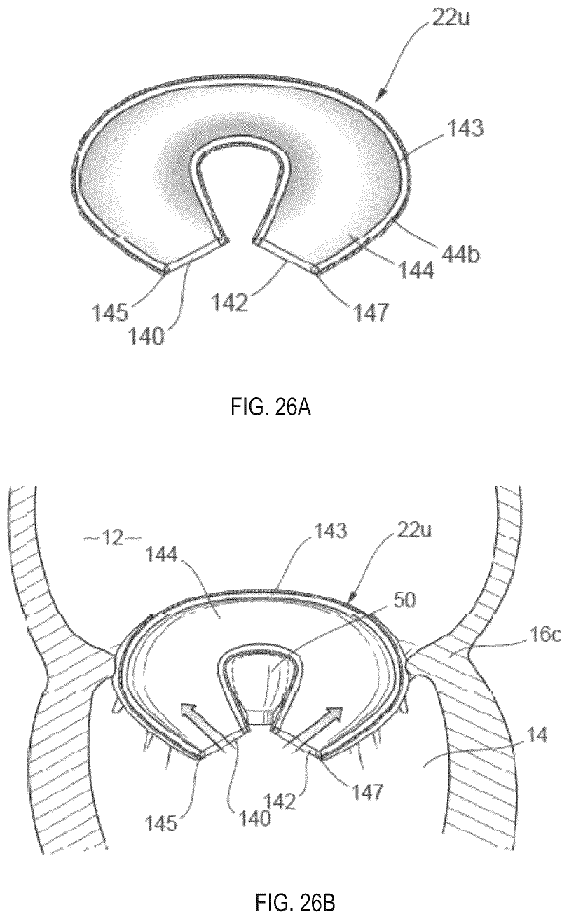

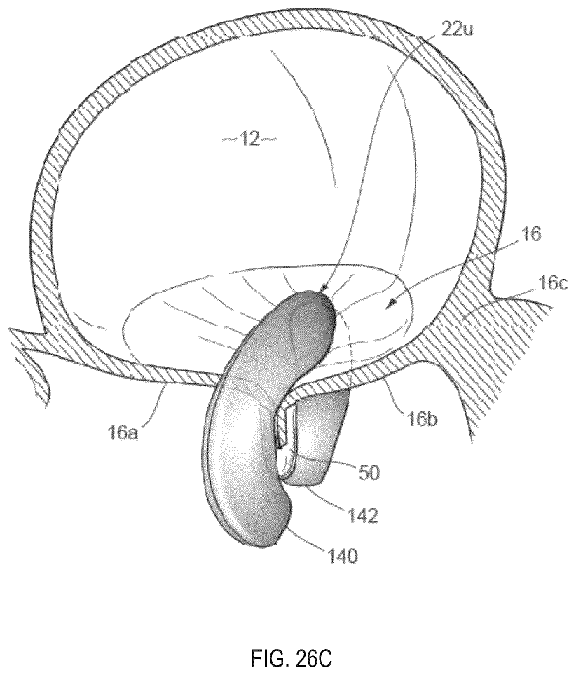

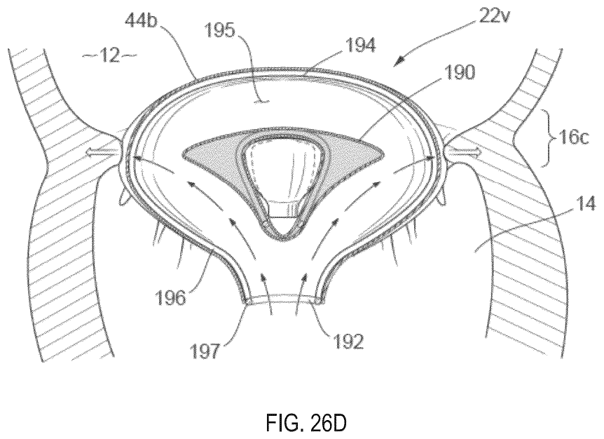

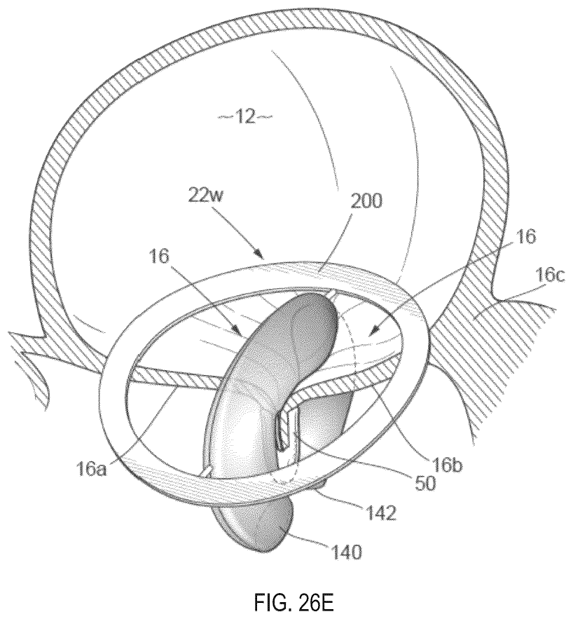

FIGS. 26A, 26B and 26C schematically illustrate another illustrative embodiment of an apparatus including a central clip structure 50 (FIG. 26B) and a selective occlusion device 22u. The selective occlusion device 22u, as with previous devices shown and described herein, is a hollow fluid communicating structure having a flexible membrane 44b and allowing blood flow into the hollow interior 144 defined by the flexible membrane 44b in systole, as shown in FIGS. 26B and 26C. In diastole, the flexible membrane 44b collapses inwardly, as previously shown and described, to allow blood flow past the selective occlusion device 22u and between the native mitral leaflets 16a, 16b from the left atrium 12 into the left ventricle 14. In this embodiment, the orientation of openings 140, 142 and shape of the device 22u force blood flow, in systole, toward the commissure regions as shown by the arrows. These forces help retain the device 22u in place, in addition to any other securement such as the clip structure 50. In this way, rocking of the device 22u may be reduced and the device 22u can be more stable during implantation and use. These inlets 140, 142 are angled acutely away from the central clip structure 50 as shown in FIG. 26B.

FIG. 26D illustrates another embodiment of a selective occlusion device 22v in which a suitable baffle structure 190 is provided within the selective occlusion device 22v for directing blood flow outwardly as shown by the arrows toward the connecting locations between the device 22v and the mitral annulus 16c. This helps to produce securement force and stabilization of the device 22v in the implanted condition. A single opening 192 is provided for in flow during systole and the device 22v includes a closed end 194 and a hollow interior 195, such that the device 22v fills with blood during systole and collapses to expel the blood during diastole as previously shown and described. A frame structure 196 is provided to support a flexible membrane 44b, generally as previously described, except that the frame structure is shaped and configured differently so as to form the single opening 192 defined by a hoop or ring frame member 197. It will be appreciated that the shapes and configurations of these structures may be modified from those shown in these illustrative examples.

FIG. 26E is an embodiment of a device 22w that may be configured as previous embodiments have been described, in terms of the selective occlusion device 22w, but which includes a generally annular or circular frame 200 structure that is a flat element for securing the apparatus in place in the mitral valve 16. The frame structure 200 is shown to rest and/or be secured in the left atrium 12 abutting against heart tissue generally proximate the mitral annulus 16c. However, it will be appreciated that such a structure could be secured in other manners, and that an additional lower support may be provided to sandwich heart tissue therebetween.

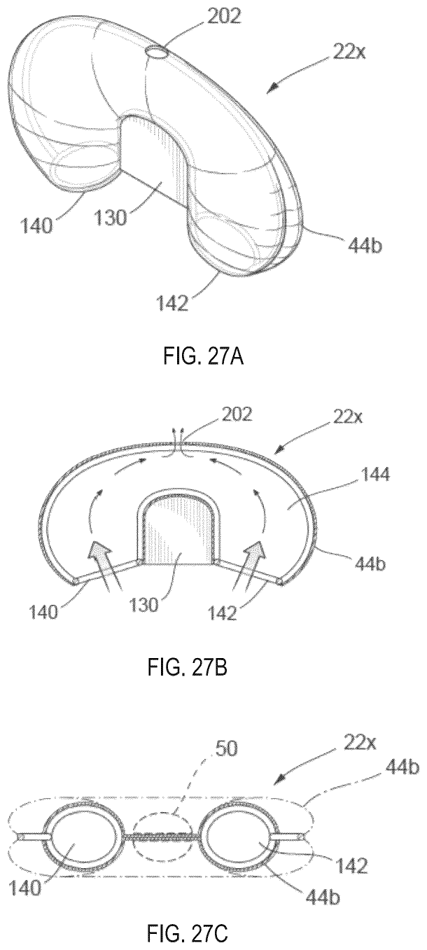

FIGS. 27A through 27C illustrate another embodiment of a selective occlusion device 22x which may be constructed in accordance with previous described embodiments, but including at least one small vent 202 opposite to the two openings 140, 142 of the flexible membrane 44b. The vent 202 is not large enough to result in any significant regurgitation or leakage of blood in systole. To the extent that the vent 202 does not allow any significant regurgitation of blood, this end of the flexible membrane is closed while the opposite end includes at least one and, in this embodiment two openings 140, 142. Otherwise, this embodiment of the flexible membrane 44b operates and functions for purposes and in ways as previously shown and described. One or more vents 202 may, for example, provide a pressure relief to reduce the forces against the device 22x during high pressure systole portions of the heart cycle.

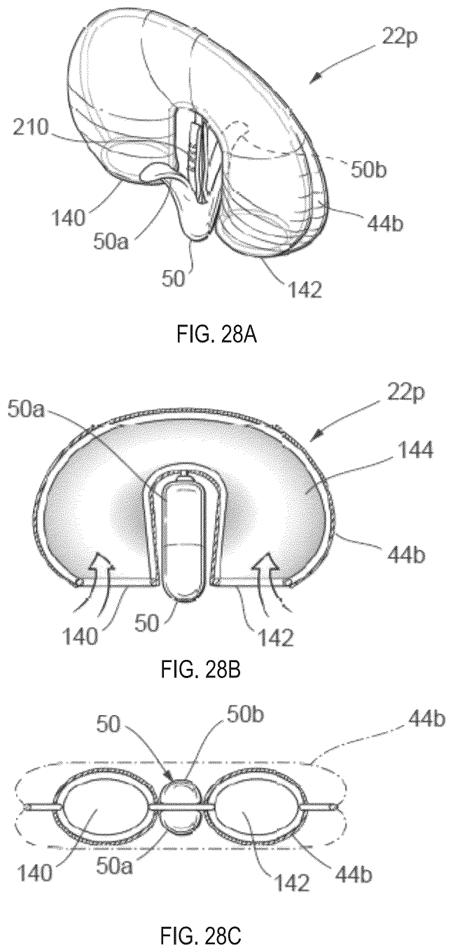

FIGS. 28A through 28C illustrate another embodiment of an apparatus comprised of a central clip structure 50 and the previously described selective occlusion device 22p. In this embodiment, the clip structure 50 includes a central gripping structure 210 which may have tines or other knurled, roughened or frictional surfaces. This will assist with clamping and retaining mitral leaflet margin tissue between the respective clip elements 50a, 50b and the selective occlusion device 22p. The clip structure 50 is secured to the selective occlusion device 22p, such as via the central gripping element 210. FIGS. 28B and 28C further illustrate that the selective occlusion device 22p operates in the same manner, for example, as described above with fluid communication between two generally adjacent openings 140, 142 for increased washing and rinsing.

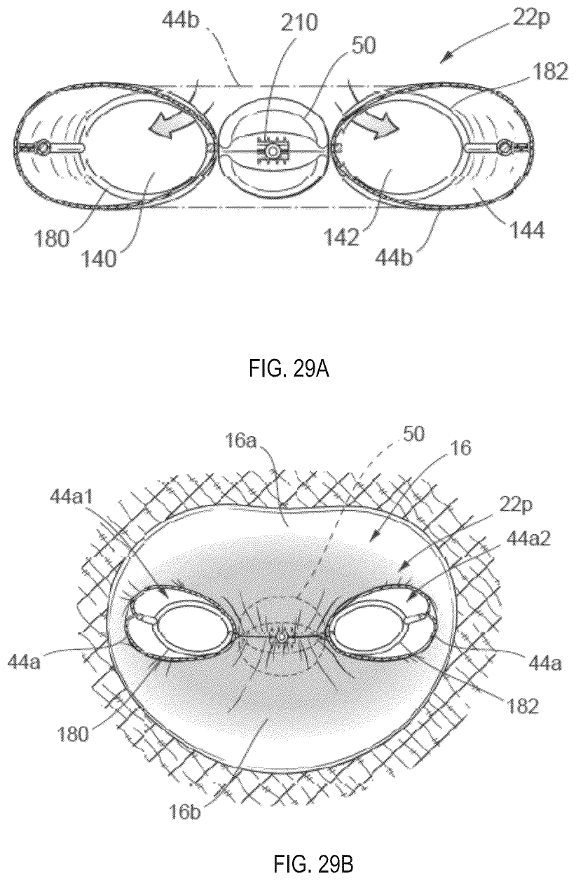

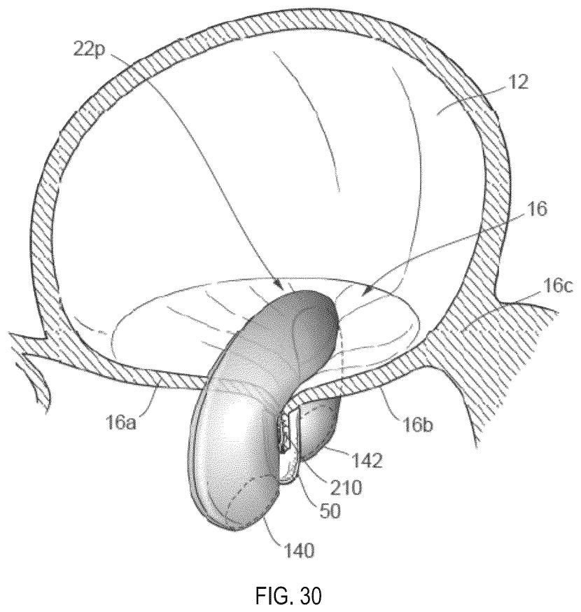

FIGS. 29A, 29B and 30 illustrate the apparatus shown in FIGS. 28A through 28C in operation after being implanted in the mitral valve 16. Specifically, blood enters the selective occlusion device 22p through the open ends 140, 142 and fills the interior 144 defined by the flexible membrane 44b, whereupon the flexible membrane 44b expands or inflates to engage in contact with the native mitral leaflets 16a, 16b forming a fluid seal that prevents regurgitation of blood flow during systole (FIGS. 29A and 29B). This is shown in FIG. 29B with the anatomy of the mitral valve 16 further shown and the native leaflet tissue contacting the outside surfaces of the flexible membrane 44b during systole.

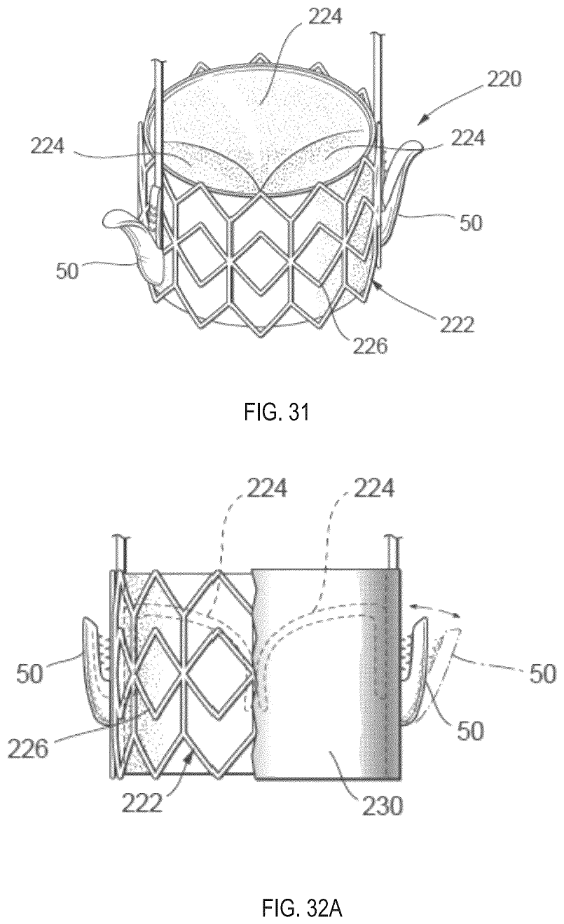

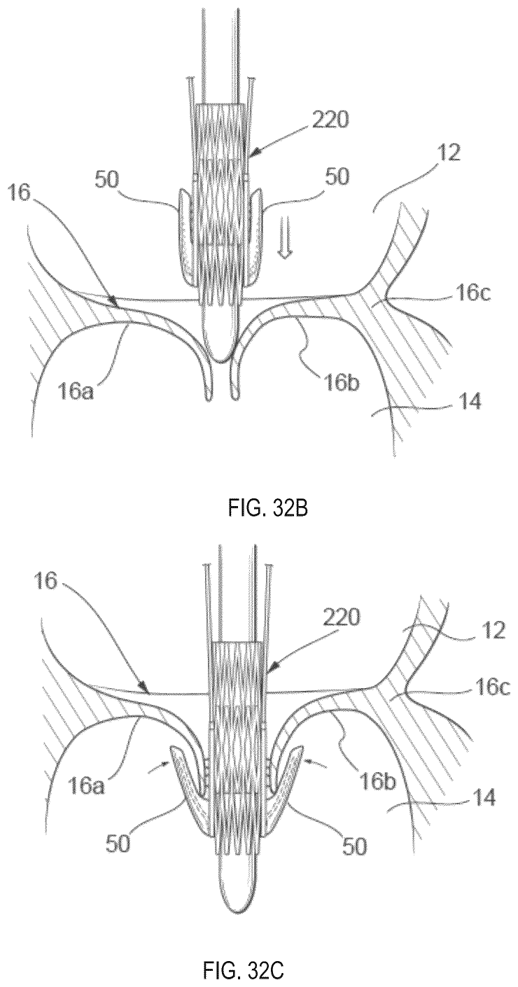

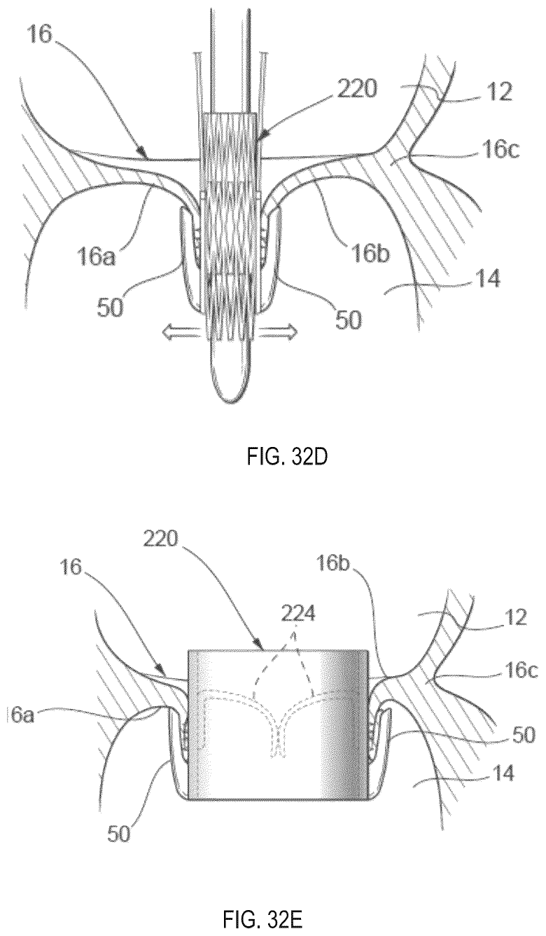

FIG. 31 illustrates another embodiment showing an expandable prosthetic heart valve 220, which may be comprised of a generally cylindrical outer or peripheral frame structure 222 and coupled with interior prosthetic leaflets 224 that open and close to control blood flow therethrough. This is different from the other versions of a selective occlusion device which have at least one movable valve element (e.g., the flexible membrane that operates in conjunction with a native mitral leaflet), in that this prosthetic heart valve 220 does not operate in conjunction with the native leaflet to control blood flow. Instead, the prosthetic leaflets 224 control blood flow through the prosthetic valve 220. Coupled to the frame structure 222 are clip structures 50 or elements that directly couple the expandable prosthetic heart valve 220 to heart valve leaflets, such as the mitral valve leaflets 16a, 16b as previously shown and described. FIG. 32A is a side elevational view partially fragmented to show the internal stent structure 226 exposed underneath an outer covering 230, which may be natural, synthetic, biologic, bioengineered, or any other suitable medical grade material useful for cardiac devices of this type.

FIGS. 32B through 32E illustrate the succession of steps used to implant the prosthetic valve 220 of FIGS. 31 and 32A. In particular, this apparatus may be implanted through a transcatheter procedure, or a more invasive procedures such as a surgical procedure or keyhole type or other less invasive procedure. The collapsed or folded apparatus 220 is inserted between the mitral valve leaflets 16a, 16b as shown in FIG. 32B, the clip structures 50 are used to capture the lower margins of the mitral leaflets 16a, 16b (FIG. 32C) and clamp them as shown in FIG. 32D. The expandable prosthetic heart valve 220 is then expanded against the native mitral leaflets 16a, 16b as shown in FIG. 32E to secure the implanted prosthetic heart valve 220 in place within the native mitral valve 16. The prosthetic leaflets 224 then open and close, respectively during diastole and systole to allow and prevent the flow of blood through the prosthetic heart valve 220.

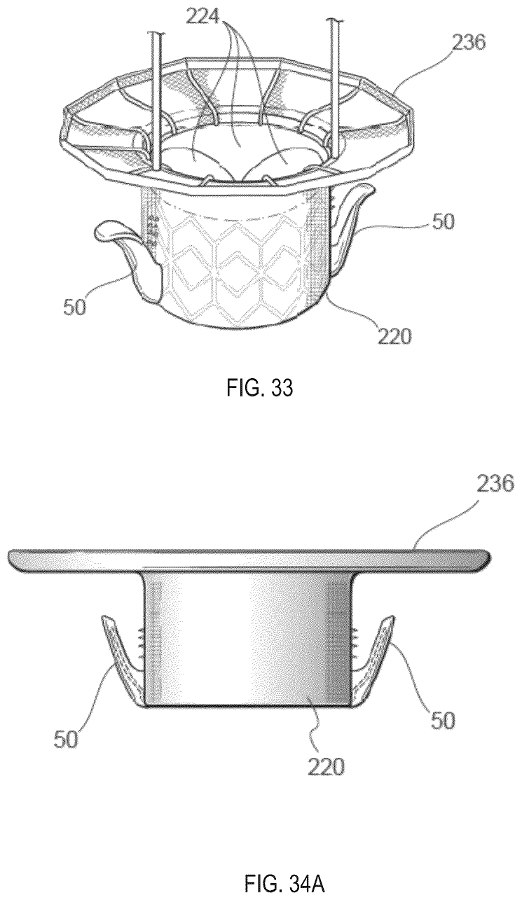

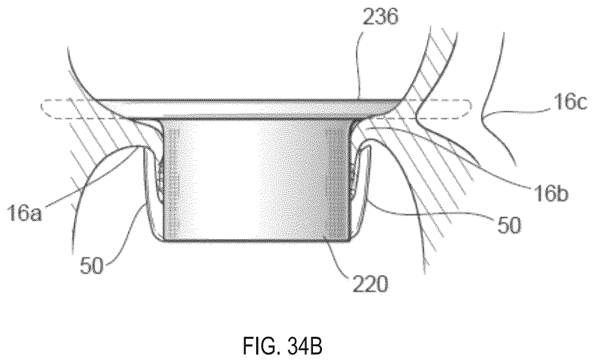

FIG. 33 illustrates another embodiment, similar to the previous embodiment shown in FIG. 32, but adding an upper flange element 236 that helps secure the prosthetic heart valve 220 by stabilizing the heart valve 220 within the left atrium 12. In this regard the flange 236 is mounted above the native mitral valve 16. The flange 236 may abut against heart tissue in the lower portion of the left atrium 12. FIG. 34A is a side elevational view of the prosthetic heart valve 220 shown in FIG. 33. FIG. 34B is an illustration of the prosthetic heart valve 220 shown secured in place within the native mitral valve 16.

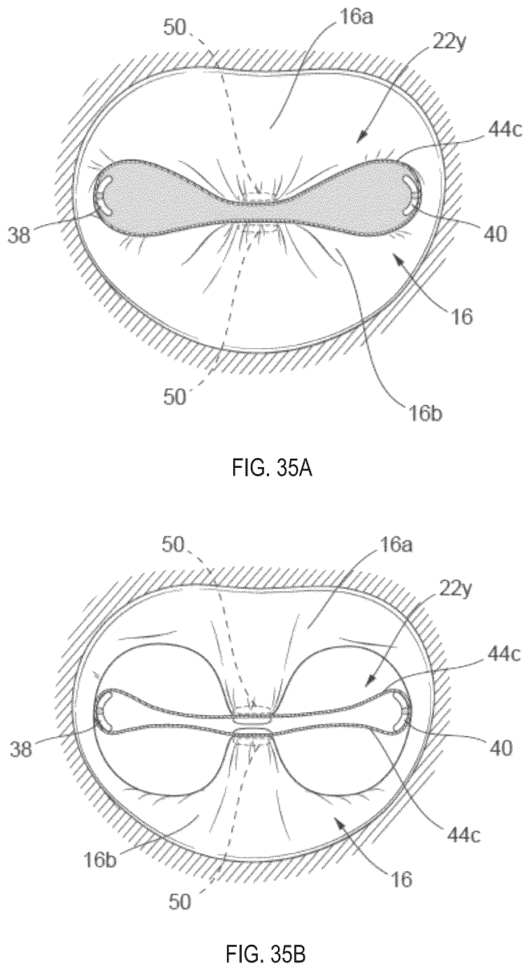

FIGS. 35A and 35B show another embodiment of a selective occlusion device 22y mounted in a native mitral valve 16, as viewed in cross section. This embodiment includes a flexible membrane 44c with an open end facing the left ventricle 14, as in other embodiments, and receiving blood flow from below when the heart cycle is in systole (FIG. 35A). In this portion of the heart cycle, the flexible membrane 44c expands against the native leaflets 16a, 16b to reduce regurgitation as previously discussed. In diastole, the flexible membrane collapses and expels the blood therein (FIG. 35B). Blood then travels in the reverse direction, generally, through the mitral valve 16 by flowing between the native leaflets 16a, 16b and outer surfaces of the collapsed membrane 44c. A difference between this embodiment and others is that multiple clip structures 50 are used to secure the selective occlusion device 22y directly to the leaflets 16a, 16b. The leaflets 16a, 16b are not clipped to each other. It will be appreciated that even further clip structures 50 may be used in this embodiment as well as others. In this embodiment, a clip structure 50 secures one side of the flexible membrane 44c to the anterior leaflet 16a and another clip structure 50 secures the flexible membrane 44c to the posterior leaflet 16b.

As described above with reference to FIGS. 31 to 34B, the flow of blood through a native valve can be controlled by a prosthetic valve that is engaged with the native valve apparatus by coupling the prosthetic valve to, and between, each of the native leaflets, e.g. by a clip that engages each leaflet and fixes it with respect to the frame of the prosthetic valve. Prosthetic valves, such as those used in transcatheter aortic valve implantation (“TAVI”) or transcatheter aortic valve replacement (“TAVR”) procedures, have proven to be reliable and effective. Prosthetic valves such as the CoreValve Evolut valve offered by Medtronic and the Sapien valve offered by Edwards Lifesciences are representative. They have metal stent or frame bodies, which may be balloon-expandable (e.g. cobalt chromium) or self-expanding (e.g. Nitinol) that support a tri-leaflet prosthetic valve set (typically formed of animal tissue such as pericardium or native animal leaflets).

As described in more detail in the following embodiments, prosthetic valves can also be used to control the flow of blood through a native heart valve on which an edge-to-edge approximation procedure is performed (e.g. with a clip such as the MitraClip™ or PASCAL), which procedure alters the native valve orifice between the native valve leaflets. For ease of illustration and explanation in the following description, the native valve is a mitral valve, i.e. a bileaflet valve with an anterior leaflet and a posterior leaflet, but the devices and procedures described below can also be used, or adapted for use, with other native valves, such as the tricuspid, that have three native leaflets.

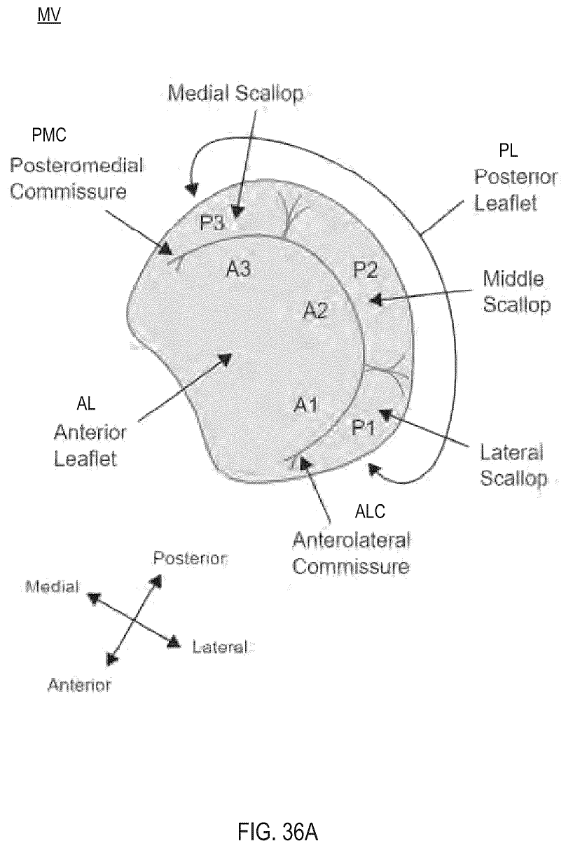

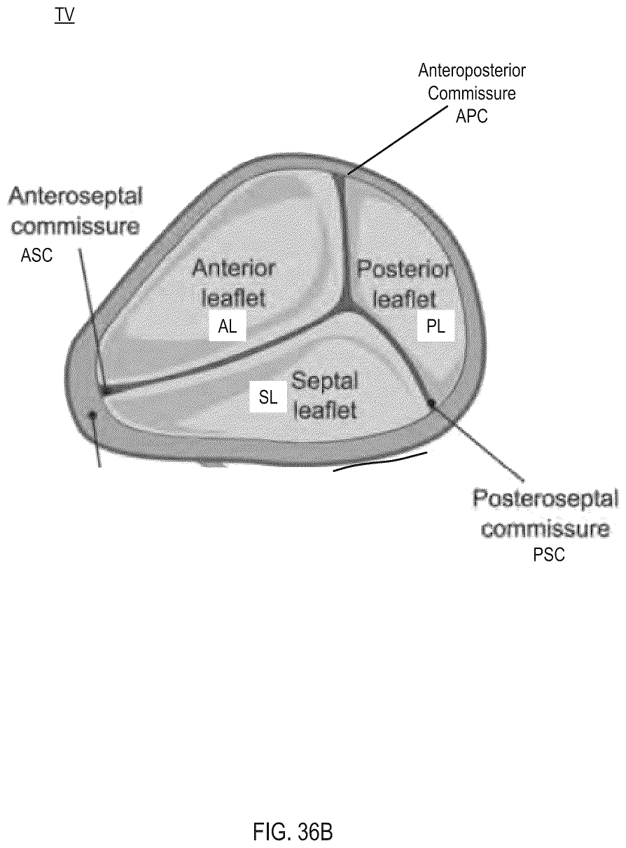

For reference, FIG. 36A illustrates a native mitral valve MV, with a posterior leaflet PL and anterior leaflet AL. The posterior leaflet PL has three segments or scallops: P1 (anterior or medial scallop); P2 (middle scallop); and P3 (posterior or lateral scallop). The anterior leaflet AL has three corresponding segments: A1 (anterior segment); A2 (middle segment); and A3 (posterior segment). The corresponding segments or scallops of the anterior leaflets coapt with each other to prevent retrograde flow through the valve (from the left ventricle LV into the left atrium LA) during systole—in FIG. 36A, the leaflets are shown coapted, i.e. they are in the position they assume during systole. The two leaflets AL and PL meet at two commissures—the posteromedial commissure PMC and the anterolateral commissure ALC. The leaflets extend from the mitral valve's annulus, MVA (not shown in FIG. 36A).

For further reference, FIG. 36B illustrates a native tricuspid valve MV, with a posterior leaflet PL, an anterior leaflet AL, and a septal leaflet SL. In FIG. 36B, the leaflets are shown coapted, i.e. they are in the position they assume during systole. The leaflets meet at three commissures: the anterior leaflet AL meets the septal leaflet SL at the anteroseptal commissure ASC; the septal leaflet SL and the posterior leaflet PL meet at the posteroseptal commissure PSC, and the posterior leaflet PL meets the anterior leaflet AL at the anteroposterior leaflet APC.

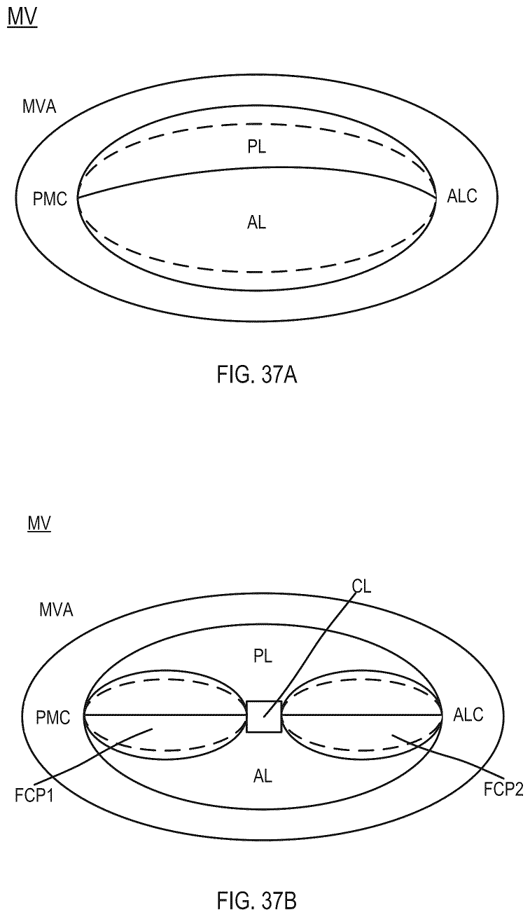

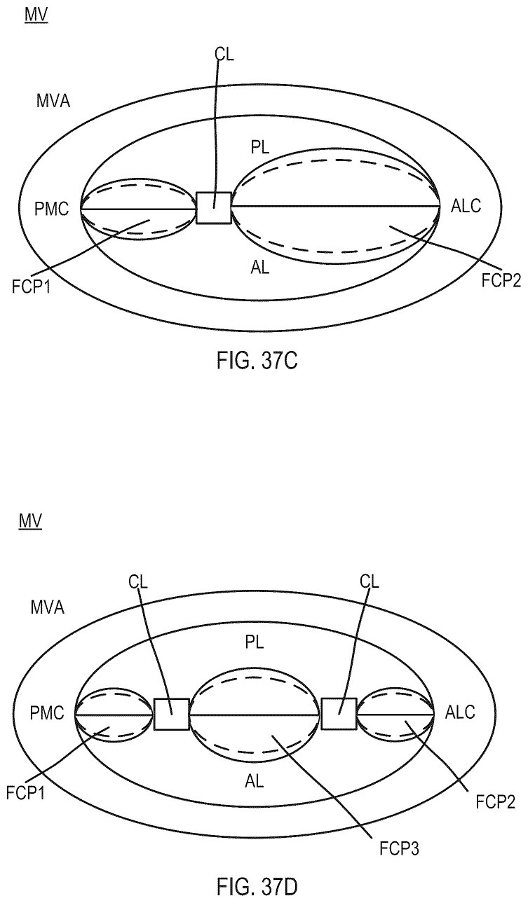

For further reference, a native mitral valve MV is shown schematically in FIG. 37A. In this figure, the edges of the leaflets AL and PL are shown in solid lines when the heart is in systole, i.e. the leaflet edges are coapted against each other and (for a competent native valve) block retrograde blood flow, and are shown in dashed lines with the heart is in diastole, i.e. the leaflets are spaced, permitting antegrade blood flow from the left atrium LA to the left ventricle LV.

FIGS. 37B to 37D schematically illustrate a native mitral valve MV on which an edge-to-edge approximation is performed with one or more clips. As shown in FIG. 37B, a single clip CL has been disposed centrally to approximate the edges of the anterior leaflet AL and posterior leaflet PL at their respective A2 and P2 segments. This has created two flow control portions through which blood can flow during diastole-FCP1, which is bounded by the anterior leaflet AL, posterior leaflet PL, clip CL, and posteromedial commissure PMC, and FCP2, which is bounded by the anterior leaflet AL, posterior leaflet PL, clip CL, and anterolateral commissure ALC. Similarly, as shown in FIG. 37C, a single clip CL has been disposed off-center in the native leaflets. Two flow control portions-FCP1 and FCP2—are created, but they are of substantially different sizes. In the extreme case of off-center or eccentric clipping, the smaller flow control portion (e.g. FCP1 in FIG. 37C) may be of insignificant or negligible size to warrant treatment. As such, a single, larger flow control portion may result with the placement of a single clip CL. As shown in FIG. 37D, two clips have been disposed spaced from each other to approximate the edges of the anterior leaflet AL and posterior leaflet PL. This has created three flow control portions through which blood can flow during diastole-FCP1, which is bounded by the anterior leaflet AL, posterior leaflet PL, clip CL, and posteromedial commissure PMC; FCP2, which is bounded by the anterior leaflet AL, posterior leaflet PL, clip CL, and anterolateral commissure ALC; and FCP3, which is bounded by the anterior leaflet AL, posterior leaflet PL, and the two clips CL.

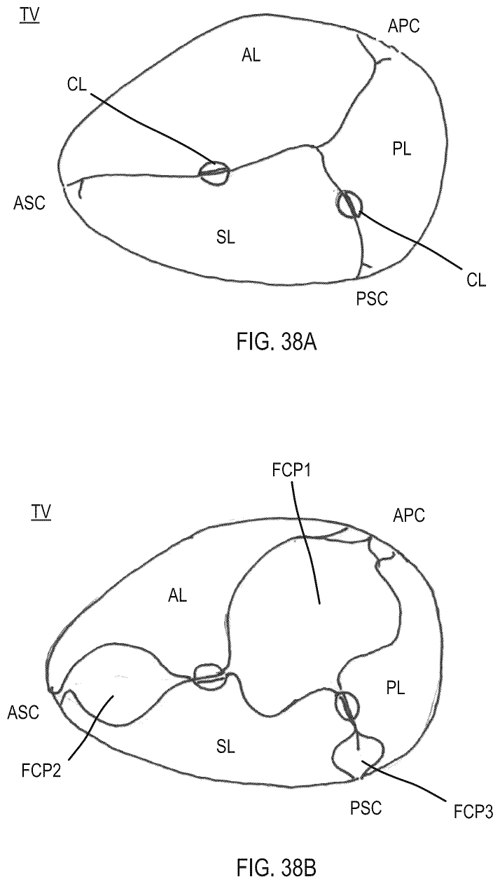

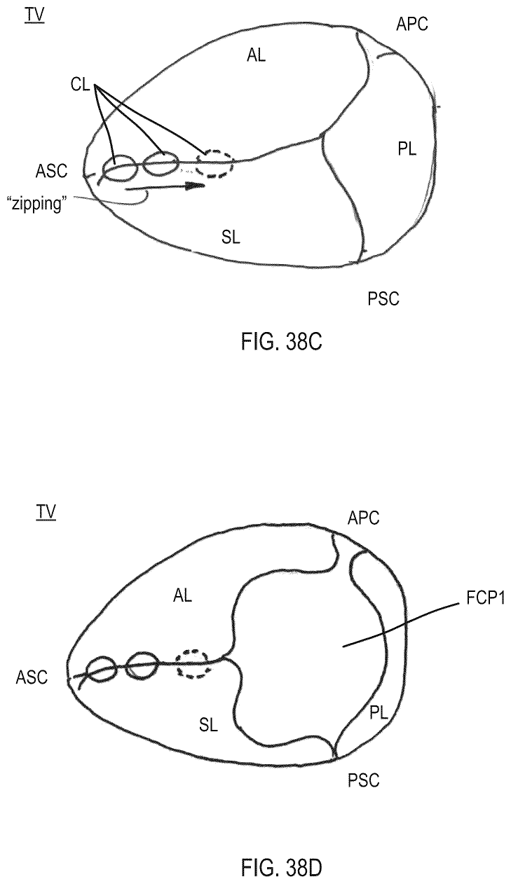

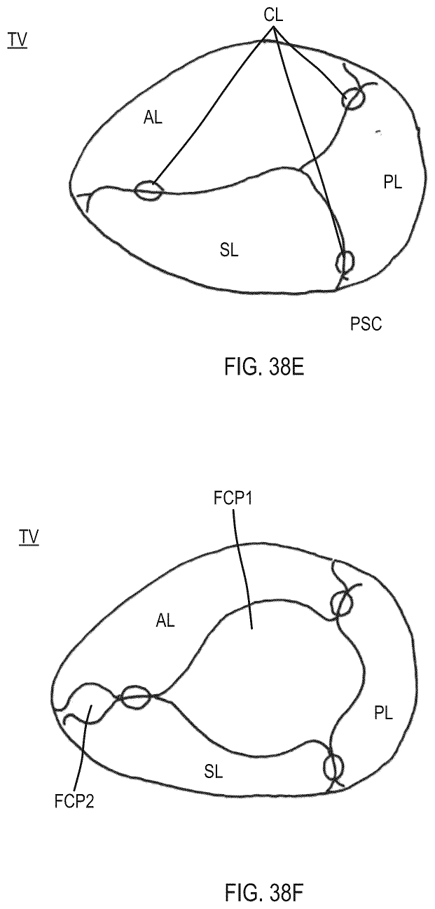

FIGS. 38A to 38F schematically illustrate a native tricuspid valve TV on which an edge-to-edge approximation is performed. FIGS. 38A and 38B illustrate a native tricuspid valve TV on which a “triple orifice” clipping technique has been performed with two clips CL (such as the TriClip™-FIG. 38A illustrates tricuspid valve TV during systole, and FIG. 38B illustrates tricuspid valve TV during diastole. One clip CL joins the anterior leaflet AL and the septal leaflet SL, while the other clip CL joins the posterior leaflet PL and the septal leaflet SL. This clipping procedure has created three flow control portions through which blood can flow during diastole-FCP1, which is bounded by the anterior leaflet AL, posterior leaflet PL, septal leaflet SL, both clips CL, and anteroposterior commissure APC; FCP2, which is bounded by the anterior leaflet AL, septal leaflet SL, one clip CL, and anteroseptal commissure ASC; and FCP3, which is bounded by the posterior leaflet PL, septal leaflet SL, one clip CL, and posteroseptal commissure PSC.

FIGS. 38C and 38D illustrate a native tricuspid valve TV on which a “bicuspidization” clipping technique has been performed with two or more clips CL-FIG. 38C illustrates tricuspid valve TV during systole, and FIG. 38D illustrates tricuspid valve TV during diastole. All of the clips CL joins the anterior leaflet AL and the septal leaflet SL. This clipping procedure has created one large flow control portion through which blood can flow during diastole-FCP1, which is bounded by the anterior leaflet AL, posterior leaflet PL, septal leaflet SL, one of the clips CL, anteroposterior commissure APC, and posteroseptal commissure PSC.

FIGS. 38E and 38F illustrate a native tricuspid valve TV on which a “three clip variant” clipping technique has been performed with three clips CL-FIG. 38E illustrates tricuspid valve TV during systole, and FIG. 38F illustrates tricuspid valve TV during diastole. One clip CL joins the anterior leaflet AL and the septal leaflet SL, one joins the joins the septal leaflet SL and posterior leaflet PL, and one joins the posterior leaflet PL and the anterior leaflet AL. This clipping procedure also creates one large flow control portion through which blood can flow during diastole-FCP1, which is similar to, but smaller than, the opening of the native tricuspid valve before the clipping procedure. Thus, the flow control portion FCP 1 is bounded by the anterior leaflet AL, posterior leaflet PL, and septal leaflet SL, but instead of being bounded by the three native commissures, it is bounded by the three clips CL.

As discussed above, the goal of an edge-to-edge approximation procedure, using one or more clips (such as the MitraClip™, TriClip™, or PASCAL) is to repair a native valve that is not adequately preventing retrograde flow during systole, i.e. is experiencing regurgitation. The clipping procedure can reduce, or ideally eliminate, such regurgitation. However, experience has shown that regurgitation can still occur in one or more of the flow control portions FCP created by the clipping procedure, either immediately after the procedure or over time (e.g. with expansion of the heart and correspondingly the size of the annulus of the native valve, or retraction of the native leaflets). In the embodiments described above, a selective occlusion device may be disposed in the one or more regurgitant flow control portions to reduce or eliminate regurgitation. The selective occlusion device may be engaged with the clip(s) to maintain, or aid in maintaining, the device in the desired position with respect to the native valve and the flow control portions. The selective occlusion device may also be supported with respect to the native valve with the aid of one or more structures that engage with the annulus of the native valve and/or other structure of the native valve apparatus. In some embodiments described below, a prosthetic valve may be disposed in the one or more regurgitant flow control portions. Devices and systems incorporating such prosthetic valves may employ similar structures and techniques for engaging with the clip and/or the native valve apparatus to maintain the prosthetic valve(s) in position. In other embodiments described below, a pseudo-valve may be disposed in the one or more regurgitant flow control portions.

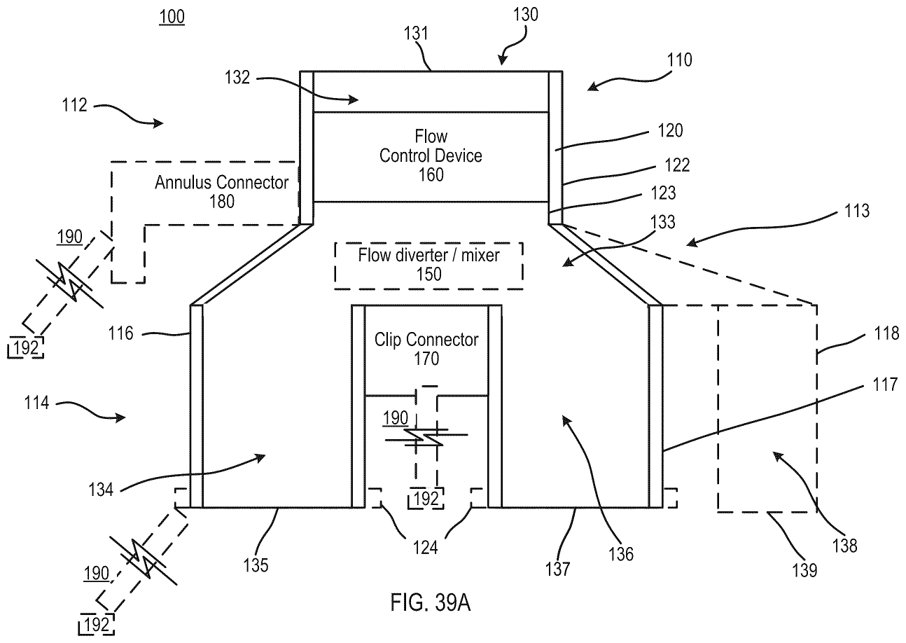

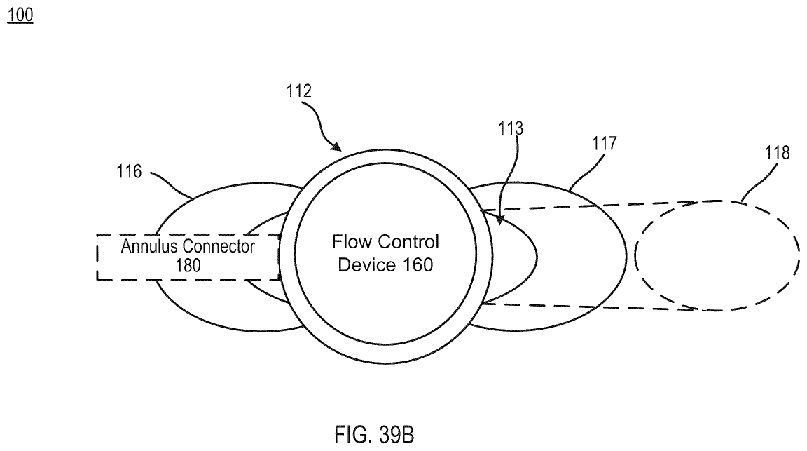

An embodiment of a prosthetic valve 100 is illustrated schematically in a side view and top view, respectively, in FIGS. 39A and 39B. In the following description, some of the reference numbers use are the same as those in the preceding description. The reference numbers used below are intended to be internally consistent, so no correspondence of structures or functions for elements with the same reference number in the preceding and following description should be inferred. As shown in FIGS. 39A and 39B, prosthetic valve 100 includes a body 110 with an inlet portion 112, a transition portion 113, and an outlet portion 114. Outlet portion 114 includes a first limb 116 and a second limb 117, and may optionally include a third limb 118. Body 110 defines a flow passage 130 therethrough that includes a flow control passage 132 in the inlet portion 112, a branching or transition passage 133 in transition portion 113, a first limb passage 134 in the first limb 116 and a second limb passage 136 in second limb 117, and may optionally include a third limb passage 138 in optional third limb 118.

All of the portions of the flow passage 130 are in fluid communication with each other and can conduct fluid, e.g. blood, from an inlet 131 at the entrance to the flow passage 130, through the flow control passage 132, through the transition passage 133, and through the first limb passage 134 out of a first outlet 135 at the exit to the first limb passage 134, the second limb passage 136 out of a second outlet 137 at the exit to the second limb passage 136, and optionally through the optional third limb passage 138 and out of a third outlet 139 at the exit to the optional third limb passage 138.

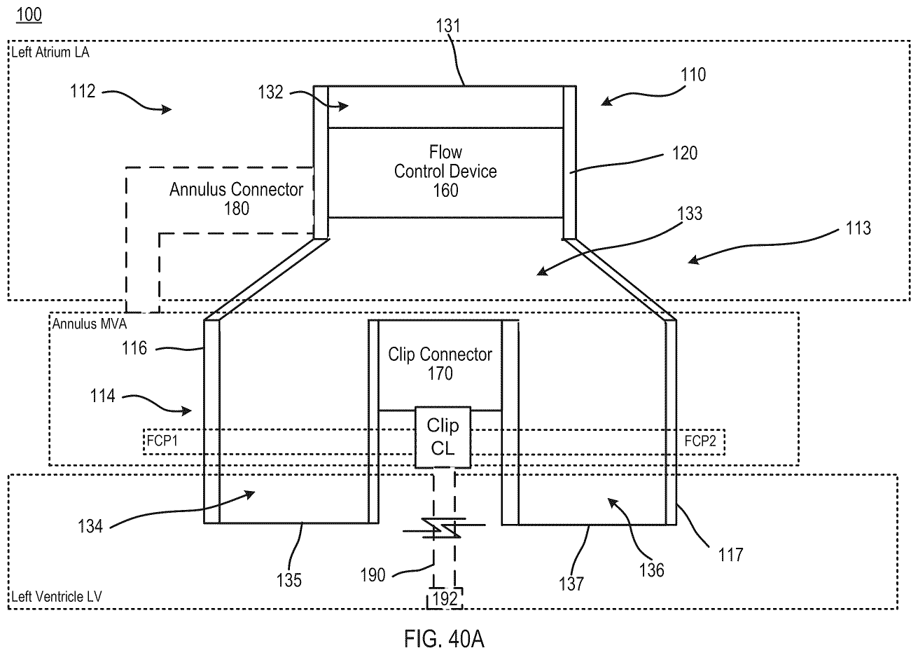

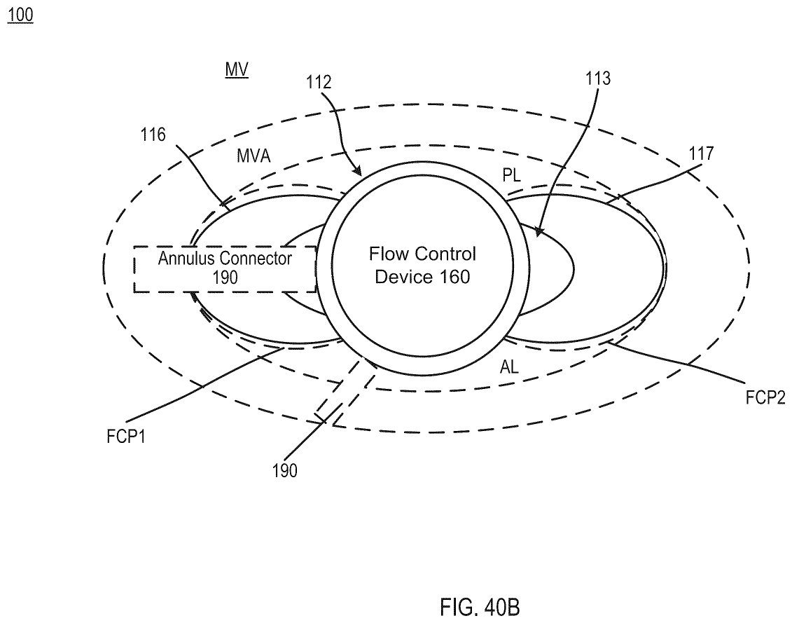

Flow through the flow passage 130, and in particular through flow control passage 132, is controlled by flow control device 160. Flow control device 160 can be constructed, and function, similar to known prosthetic valves described above, and may be implemented as a tri-leaflet valve with three leaflets. Other valve constructions may be suitable, including valves with fewer than three leaflets, which may coapt against fixed structures in the valve in addition to, or instead of, coapting against other leaflet(s), as described in more detail below in particular embodiments. As shown schematically in FIGS. 39A to 40B, flow control device 160 may be cylindrical, with a circular cross section. Flow control device 160 may be mounted to inlet portion 112 of body 110 and disposed so that all flow through flow control passage 132 must pass through flow control device 160. Flow control device 160 is configured to permit fluid to flow therethrough in the direction from the inlet 131 to the outlets 135, 137, and optionally 139, but to prevent fluid to flow in the opposite direction.

It is well known that tissue valves can fail, and it is also known that this problem can be solved by delivering another tissue-based stent valve inside a failed valve. Thus, it is contemplated that if flow control device 160 fails, a new tri-leaflet valve can be placed inside flow control device 160.

Prosthetic valve 100 also includes a clip connector 170 that is part of, or coupled to, body 110, and is configured to engage with a clip such as those described above, and thereby to retain prosthetic valve 100 in operative relationship with a native heart valve to which the clip is attached. In particular, clip connector 170 is configured to carry fluid dynamic load applied to prosthetic valve 100 during the cardiac cycle of the heart to the clip CL and thence to the native leaflets, annulus, and surrounding heart tissue to resist displacement of the prosthetic valve. The largest loads to be carried tend to be during systole, and the displacement to be resisted is towards the atrium of the heart.

Clip connector can be implemented in a variety of configurations, including those described above in connection with numerous embodiments of selective occlusion devices to couple a frame structure (which can be analogized to body frame 120 and/or to annulus connector 180) to a clip structure, for example in FIGS. 5C-5D (with a tensile member 54), FIGS. 12C-12D (frame member 32 connected directly to the clip 50), FIGS. 14A-14C (with a rod-like connector 54), FIGS. 15A-15E and FIGS. 27A-27C (with the clip directly engaging a reinforced central fabric area 130 of the flexible membrane 44a or 44b).

Prosthetic valve 100 is shown disposed in a native heart valve in a side view and top view, respectively, in FIGS. 40A and 40B. Note that for convenience of illustration, prosthetic valve 100 is shown in FIGS. 40A and 40B without the optional third limb 118 and associated third limb passage 138 and third outlet 139, and the native heart valve is illustrated as a mitral valve MV. Mitral valve MV is shown with the anterior leaflet AL and posterior leaflet PL joined by a clip CL in an edge-to-edge approximation, similar to the mitral valve MV shown in FIG. 37B. Thus, mitral valve MV has two flow control portions-FCP1 and FCP2-defined between the clip, the leaflets, and the commissures of the mitral valve MV.

As shown in FIGS. 40A and 40B, prosthetic valve 100 can be disposed in mitral valve MV with inlet 131 disposed in the left atrium LA and the first outlet 135 and second outlet 137 disposed in the left ventricle LV. First limb 116 is shown disposed in flow control portion FCP1, and second limb 117 is shown disposed in flow control portion FCP2. Clip connector 170 is engaged with clip CL. Optional annulus connector 180 can be engaged with mitral valve annulus MVA. When prosthetic valve 100 is disposed in mitral valve MV, it is operable to reduce or eliminate regurgitation through flow control portions FCP1 and/or FCP2, i.e. to prevent retrograde flow of blood from the left ventricle to the left atrium during systole, but to allow blood to flow freely through prosthetic valve 100 from left atrium LA to left ventricle LV during diastole.

As noted above, prosthetic valve 100 can be used with other native heart valves, including the other atrioventricular valve, the tricuspid valve. For example, a prosthetic valve with the optional third limb may be useful for a tricuspid valve on which a triple orifice clipping technique has been used, with each of the three limbs being disposable in each of the three resulting flow control portions, respectively. However, in some instances it may be preferable to use a prosthetic valve that does not include the third limb in such a tricuspid valve, disposing each of two limbs in two of the three flow control portions, and allowing the third flow control portion to function only with the native leaflets.

The height of inlet portion 112 of body 100, or the collective height of inlet portion 112 and transition portion 113, may be of any suitable distance, though it is desirable that it not be so large that the inflow of blood into inlet 131 is impeded, i.e. sufficient room is left above and around inlet 131 inside the atrium of the heart for blood to freely enter.

The absolute and relative sizes (cross-sectional areas) of the flow control passage 132 (and flow control device 160) and of the first limb passage 134 and second limb passage 136 (and optional third limb passage 138) can be varied to optimize function, to match the anatomy, cardiac capacity, etc. of the heart, or to account for other relevant factors.

Each of the first limb 116 and the second limb 117 may be configured so that outer surfaces thereof engage in a substantially sealing relationship with the anterior leaflet and the posterior leaflet and thereby to reduce or prevent the flow of blood therebetween, during at least a portion of the pumping cycle of the heart. In some embodiments, each of the first limb and the second limb may be sized (e.g. perimeter) and configured (e.g. cross-sectional shape is circular, elliptical, ovoid, etc.) to substantially fill, or over-fill (stretch) the corresponding flow control passage, maintaining the edges of the leaflets in sealing relationship with the outer surfaces of the first limb and the second limb throughout the cardiac cycle, thus preventing flow between the limbs and the leaflets from the atrium to the ventricle during diastole and from the ventricle to the atrium during systole. In this configuration, substantially all blood flow from the atrium to the ventricle during diastole is therefore carried through the prosthetic valve (and thus through the flow control device), and blood flow from the ventricle to the atrium during systole (regurgitation) is substantially prevented (by the flow control device 160). This configuration offers several benefits. First, the native leaflets would move little or not at all during the cardiac cycle, which should reduce wear resulting from repetitive contact between the leaflets and the outer surfaces of the limbs of the prosthetic valve (there is little momentum on the native leaflet during impact with the limbs). The native leaflets are pliable and will tend to fill any irregular shapes or defects in closure. Second, complete valve sealing, i.e. prevention of regurgitation, should be assured. Finally, the heart tends to deteriorate over time in patients needing valve repair or replacement. For such patients, regurgitation should not occur again as the prosthetic valve assumes virtually full responsibility for the function of the native valve, and the residual valve tissue will be able to fill any gaps that may occur as the heart dilates (or alternatively any gaps that may occur as the valve leaflets retract with disease progression). These benefits are particularly applicable for a native valve to which an edge-to-edge clip has been applied. After the clip is applied the total opening size of the valve is limited to the area of the resultant flow control portion(s), which is a smaller area than that of the original opening of the native valve. Thus, the surface or orifice area that must be occluded by a valve is reduced and the load on the prosthetic valve is reduced. In many instances the clip can securely hold the load created by cardiac contraction, highest during systole.

In another configuration, the limbs could be sized to be smaller than the flow control portions, allowing a gap to be formed during diastole and permitting some of the blood flowing from the atrium to the ventricle to flow through the gap (in addition to the blood flowing through the flow passage and the flow control device). The limbs are preferable sized so that during systole the leaflets can sealingly engage the limbs' outer surface and prevent retrograde flow between the limbs and the leaflets.

The limbs of prosthetic valve 100 are shown schematically in FIGS. 39B and 40B as being elliptical in cross section. This is because the flow control portions of the native valve that result from leaflet clipping are likely to be oval or slit like. By shaping the limbs with a corresponding cross-section, they can better follow the shape of the flow control portions and fill the leak space. In some embodiments, the cross-sectional shape of the limbs may be rounder (circular or oval) near the clip with a teardrop (more V-shaped) extension toward the commissures. Although the limbs are shown schematically in FIGS. 39B and 40B as being approximately linear, or symmetrically arranged about a center line through the clip, as can be seen in FIG. 36A, there is a natural curve to the coaptation line of the native mitral valve leaflets. When looking down on the mitral valve with the anterior leaflet above, there is a upward curve to the line of closure. To conform to this anatomy, in some embodiments the limbs of the prosthetic valve could be arranged to follow the curve of the coaptation line.

Limbs 116, 117 (and optionally 118) are shown schematically in FIGS. 39B and 40B as being straight and being parallel with each other. However, in some embodiments the limbs may be straight but may be non-parallel, and may be angled towards or away from each other. In other embodiments they may not be straight, but may instead be arcuate, and thus the outlet portion 114 of body 110 may have a horseshoe shape, similar to the shape of the device shown in FIG. 26B. Similarly, although the space between limbs 116, 117 (and optionally 118) is illustrated as rectangular in the schematic illustrations in FIGS. 39A and 40A, this space can be arcuate or curved with a large radius of curvature, or may be sharper (more V shaped).

Limbs 116, 117 (and optionally 118) are shown schematically in FIGS. 39A to 40B as being generally tubular in shape. However, in some embodiments it may be useful for the limbs to flare outwardly (larger perimeter) towards their respective outlets, as this may encourage more blood to enter the flow passage 130 during systole and urge closed the leaflets of the flow control device 160. Thus, the outlet ends of the limbs could have a trumpet bell shape, for example.

Although limbs 116, 117 (and optionally 118) are shown schematically in FIGS. 39B and 40B as having ends (i.e. at outlets 135, 137 (and optionally 139)) that are flat, i.e. linear and orthogonal to a central vertical axis of prosthetic valve 100, in other embodiments the ends of the limbs can be of any other configuration, including angled and/or arcuate, provided that they can be reliably positioned in the native valve so that preferably the entirety of the outlets are below the locations where the native leaflets are sealingly engaged with the limbs 116, 117 (and optionally 188). The outlets may also have an outflow perimeter that is not planar but rather a scalloped perimeter. For instance, the portion of the outflow perimeter that engages the anterior leaflet may extend deeper within the ventricle than the corresponding portion that engages the posterior leaflet. As such, the upward surge of blood during systole will first come into contact with this deeper extending portion of the outlet and may ensure better systolic filling of the prosthetic valve 100.

Body 110 can be constructed with materials and techniques similar to known prosthetic heart valves such as those discussed above. For example, body 110 can have a body frame 120 formed of wires, struts, mesh, braids, or other suitable constructions, in metal (such as cobalt chrome, stainless steel, shape memory metals such as Nitinol, etc.), polymer, or other suitable material. Body frame 120 can be formed in a single, unitary piece formed in a Y shape, or it could be constructed by separate pieces that are joined together, e.g. one piece for each of the inlet portion 112, transition portion 113, first limb 116, second limb 117, and (optionally) third limb 118. In embodiments in which the body frame 120 is formed in separate pieces, the pieces could be delivered and implanted separately to make delivery easier, and then coupled together in place in the heart valve. As described in more detail below with reference to specific embodiments, body frame 120 does not necessarily extend to the outlet portion 114 of body 110. For example, a stiff graft (such as Dacron, Teflon etc. with or without coatings) could be used with no frame or with minimal frame.

The construction of limbs 116, 117, and (optionally) 118 could vary. In some embodiments, the portion of the body frame 120 in the limbs can be configured with a stent frame, with the potential for body covering 122 and/or body lining 123 to include, or the addition of, padding (formed of materials such as silicone and pericardium). It may be useful to make the limbs more complaint so that the limbs move with each heartbeat and reduce the wear when leaflet tissues contact the device. Thus, any or all of the limbs could be constructed similar to the arrangements described above for pseudo-valves, such as the embodiments shown in FIG. 5D, 7A-B, 15A-E, or 18A-B. In such embodiments, the limbs can be constructed with a frame that allows the superimposed biologically compatible covering (such as pericardium) to move back and forth with each cardiac cycle. In other embodiments, the limbs could be constructed similar to the occlusion devices shown in FIGS. 13A-B and FIGS. 25A-B, in which the occlusion device is rigid or static, and the native leaflets moves toward the limbs to seal against leak and prevent wear.

In some embodiments, the limbs of prosthetic valve can be configured to have their shapes be adjustable to improve the seal between the limbs and the native leaflets. For example, oval shaped balloons or oval shaped stents could be introduced to shape the limbs after the prosthetic valve 100 has been placed in the native valve. Such an approach could also be useful if the body covering 122 and/or body lining 123 on (or in) a limb wears out. A new body lining 123 could be applied from inside the limb, delivered through the flow passage 130 on a stent or a frame. This approach would be particularly useful if the limb is constructed with a segment in which there is little or no frame material.

Flow control device 160 is coupled to, and supported by, body frame 120 in inlet portion 112, or may optionally form some or all of the inlet portion of the body frame 120, and be coupled to the transition portion 113.

Body frame 120 can be covered on the outside with a body covering 122 and/or on the inside with a body lining 123, each of which may be formed from any suitable material that is biocompatible, sufficiently impermeable to fluids such as blood to form the flow passage 130 and maintain fluid within (or outside of) flow passage 130, and atraumatic to native valve tissue (leaflets, chordae, heart chamber walls, etc.) that contact the body 120. Suitable materials can include animal pericardium and synthetic materials such as Dacron (the latter material may be more suitable for covering areas of the body 120 that do not contact heart tissue as it can be somewhat abrasive).

Body covering 122 and/or body lining 123 may cover or line the entirety of body 120, or may be discontinuous, and cover only portions of body 120. Each may also be attached continuously to each area of body frame 120 that it covers or lines, but may also be attached around the periphery or margins of selected areas on the body 120, but not attached within those areas. This construction can allow blood to pass between, for example, struts in the body frame 120 and expand/balloon out the body covering 122 and/or body lining 123 so that it gently contacts the native valve leaflets. The native leaflets would contact against material of body covering 122 and/or body lining 123 (for example pericardium) that is backed by blood within flow passage 130 rather than against a solid portion of body frame 120. Body frame 120 could be formed with struts that are widely spaced in the region of contact to ensure the body covering 122 and/or body lining 123 will be expanded by blood. This could reduce considerably wear on native leaflet tissues.

As shown in FIG. 39A (but omitted from FIG. 40A for ease of illustration), body 110 can also include an outlet cuff 124 at the outflow ends of the limbs 116 and 117 (and optionally, not shown, on limb 118) that includes padding material to reduce the risk of injury to cardiac tissue that may contact those portions of the body 120 during the cardiac cycle. Such padding material could be any useful biocompatible material. Silicone, polyurethane, bio-polymers or bio-elastomers, Dacron, PTFE (Teflon) fabrics (often used in rolled or folded form in the sewing cuff of prosthetic valve sewing rings) are suitable options that are commonly used in valve structures. Such padding or damage reducing materials could be added to any part of prosthetic valve 100.

In some embodiments, features may be included in the flow passage 130 to guide the flow of fluid (e.g. blood) through prosthetic valve 100. For example, it may be useful to urge the fluid towards the lateral walls of flow passage 130, e.g. in the transition passage 133, similar to the flow diversion performed by the baffle 190 in FIG. 26D above. As described in connection with FIG. 26D, the force of the fluid flow directed to the sides of the prosthetic valve 100 may reduce the risk of rocking. It may alternatively, or additionally, be useful to mix the fluid (e.g. blood) flowing through flow passage 130, such as with a spiral component disposed in the transition passage 133, similar to the manner described with reference to FIG. 26D above. Mixing the fluid around a spiral may reduce the rocking on the prosthetic valve 100 by dissipating the energy and directing the flow centrally to the flow control component 160. Structure to perform the flow diversion and/or mixing is shown schematically in FIG. 39A as optional flow diverter/mixer 150 (flow diverter/mixer is omitted from FIG. 40A for ease of illustration).

Although clip CL is described above with reference to FIGS. 36A to 40B as being a commercially-available edge-to-edge leaflet clip such as the MitraClip or PASCAL, and prosthetic valve 100 being configured to engaged with such a clip after it has been used to clip the native leaflets, in some embodiments the clip CL may be configured differently than such commercially-available clips, and/or may be included as part of a system with prosthetic valve device 100 and configured to be delivered sequentially or concurrently with prosthetic valve 100 as part of a total valve repair/replacement procedure. As described above, prosthetic valve 100 is configured to be anchored to the clip CL, which in turn is coupled to the tissue of the anterior leaflet AL and posterior leaflet PL, and prosthetic valve 100 will carry a considerable fluid dynamic load during systole that must in turn be carried by the clip and the leaflets. Thus, an enlarged clip anchor or clip structure having wider paddles or multiple paddles may be useful. For example, the clip could be composed of two or three paddles (instead of the single paddle of some of the clip devices) for engagement with each of the one or more leaflets with which the clip is engaged, to increase the area of the leaflet(s) engaged by the clip(s). This allows the dynamic load to be distributed more evenly over a larger leaflet area (and over a greater number of underlying chordae tendineae that are attached to the engaged leaflet portions) . . .

Rather than relying on the clip connector (and thus clip CL, native valve leaflets, or other native valve tissue) to carry all of the fluid dynamic loads imposed on the prosthetic valve, in some embodiments those loads can be carried in part by other structures without putting the clip or the native leaflets in the load path. Thus, in some embodiments prosthetic valve can include an optional annulus connector 180 and/or an optional heart tissue tether 190.

As shown in FIGS. 39A to 40B, optional annulus connector 180 may be part of, or coupled to, body 110, and is configured to engage with an annulus (and/or other nearby tissue, including the atrial wall, the native leaflets, and/or the chordae tendineae) of a native heart valve to enhance the stability of the prosthetic valve 100 when placed in the native heart valve, e.g. to inhibit lateral rocking of the prosthetic valve, and or displacement away from the annulus towards the atrium (during systole) or the ventricle (during diastole). Annulus connector 180 may be implement similarly to annulus connectors described above with reference to various embodiments of selective occlusion devices, including: FIG. 22E (with annulus connectors 154 and 152 configured as elongate frame members that extend longitudinally from the frame of the selective occlusion device and can engage a peripheral portion of the mitral valve annulus, with connector 154 engageable with tissue on the atrial side of the annulus and connector 158 engageable with tissue on the ventricle side of the annulus); FIG. 22F (with a single circular annulus connector 164 coupled to the frame of the selective occlusion device and engageable with substantially the entire periphery of the atrial surface of the annulus, thus preventing rocking in any direction but allowing flexibility—this configuration could also be used for engagement with the ventricle side of the annulus); FIG. 26E (similar to FIG. 22F, but the with the circular annulus connector configured as a frame structure 200 that is a flat element that may be secured to the atrium side of the annulus, and may alternatively or additionally have a similar structure that may be secured to the ventricle side of the annulus). Annulus connector 180 may be configured with non-tissue penetrating members or with tissue penetrating members.

As shown in FIGS. 39A to 40B, one or more optional heart tissue tethers 190 may be coupled to body 110, clip connector 170, clip CL, and/or annulus connector 180. For ease of illustration, not all options are shown in all of the figures. Heart tissue tethers 190 may be elongate tension members implemented as metal wires, polymer sutures (of monofilament or braided construction), or other suitable, biocompatible materials with sufficient tensile strength to carried the desired portion of the fluid dynamic loads imposed on prosthetic valve 100. Each such tether may include a suitable anchoring mechanism by which the free end of the tether (opposite to the end connected to prosthetic valve 100) may be secured to the heart tissue. Such a tether anchor 192 can include any known mechanisms for securing tethers or sutures to tissue, including cardiac tissues, such as pins, screws, clips, suture loops, or enlarged structures (pledgets, disks) that may be disposed on the opposite side of a tissue wall from the body of the tether. The heart tissue tether(s) 190 could be coupled to heart tissue that includes various locations/structures in the ventricle, such as the apex of the ventricle, the ventricular septum, any other wall of the ventricle, one or more of the papillary muscles, one or more or the chordae tendineae, and/or the annulus of the native valve.

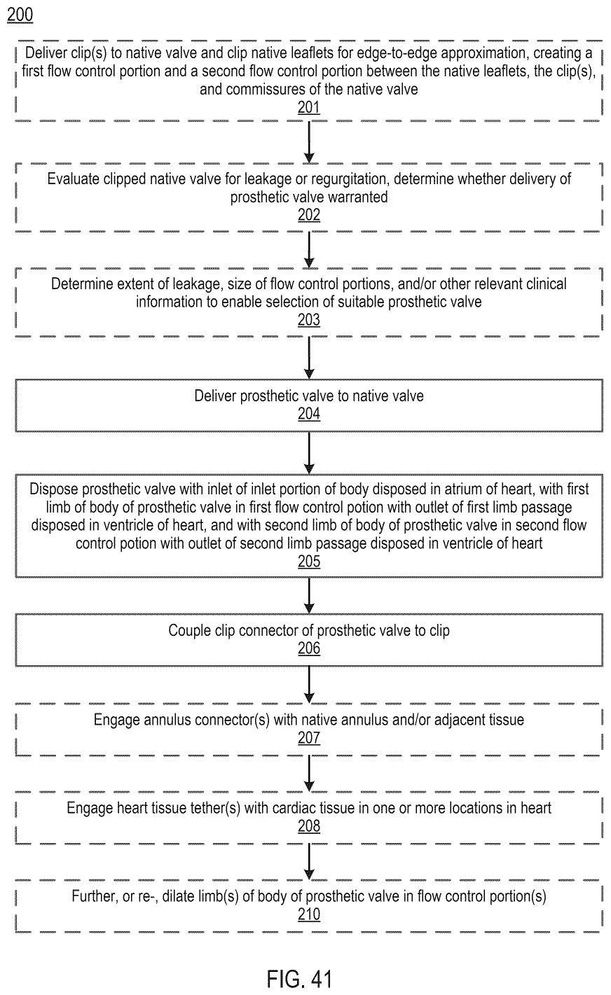

Prosthetic valve 100 may be delivered to and positioned in the native valve, and secured to the clip CL by the clip connector, to the annulus (and/or nearby tissue) by the annulus connectors, and or to other heart tissue by the heart tissue tether(s) in various methods and sequences. The delivery, positioning, and/or securement may also be performed as part of an integrated procedure with an edge-to-edge approximation procedure with clip(s) CL, sequentially after an edge-to-edge approximation during the same interventional process, or as a separate procedure on a patient who has previously been subjected to an edge-to-edge approximation procedure. Some options are described with reference to the method 200 shown in the flow chart in FIG. 41. At 201, one or more clips CL may optionally be delivered to the native valve, and used to clip the native leaflet for edge-to-edge approximation. As described above, this procedure can create two flow control portions, each bounded by the native leaflets, the (or a) clip, and a commissure of the native valve. As noted above, 201 may have been previously performed in a separate procedure, or may be performed as part of the same procedure as the subsequent portions of method 200. At 202, the clipped native valve can be evaluated for leakage or regurgitation, and a determination made as to whether any such leakage or regurgitation was sufficiently severe to warrant usage of prosthetic valve 100. At 203, the extent of the leakage or regurgitation, the size of the flow control portions, and/or other relevant clinical information could be determined (e.g. through imaging) to enable selection of a suitable prosthetic valve (e.g. size of the limbs 116, 117 (or optionally 118). At 204, the prosthetic valve 100 is delivered to the native valve, such as by known endovascular techniques, using a delivery catheter. At 205, the prosthetic valve 100 is disposed in the native valve with the inlet 131 of flow passage 130 disposed in the atrium of the heart, with the first limb 116 of body 110 of prosthetic valve 100 disposed in the first flow control portion FCP1, with the first outlet 135 of the flow passage 130 disposed in the ventricle of the heart, and with the second limb 117 of body 110 of prosthetic valve 100 disposed in the second flow control portion FCP2, with the second outlet 137 of the flow passage 130 disposed in the ventricle of the heart. At 206, clip connector 170 is coupled to clip(s) CL that are clipped to the native leaflets. In integrated procedures, clip connector 170 may be coupled to clip(s) CL before clip(s) CL are clipped to the native leaflets for the edge-to-edge approximation.

Optionally, at 207 annulus connector(s) 180 may be engaged with the native annulus (on the ventricle side and/or atrial side), and/or adjacent tissue. Although in the flow chart of FIG. 41 207 is shown as being after 206, in some embodiments the annulus connector(s) 180 may be engaged with the native annulus first, i.e. with the prosthetic valve in position in the native valve, and then the clip connector 170 may be coupled to clip(s) CL. Also optionally, at 208, one or more heart tissue tether(s) 190 may be engaged with cardiac tissue in one or more locations in the heart. Further optionally, at the completion of the method 200, or in a subsequent procedure, if some blood regurgitation is identified, and determined to arise from insufficient seal between the native leaflets in the flow control portion(s) of the native valve and the limb(s) 116, 117, then at 210, one or both of the limbs 116, 117 of prosthetic valve 100 may be further, or re-, dilated to reshape or increase the perimeter of the limb(s) and improve the seal with the native leaflets, as described in more detail below.

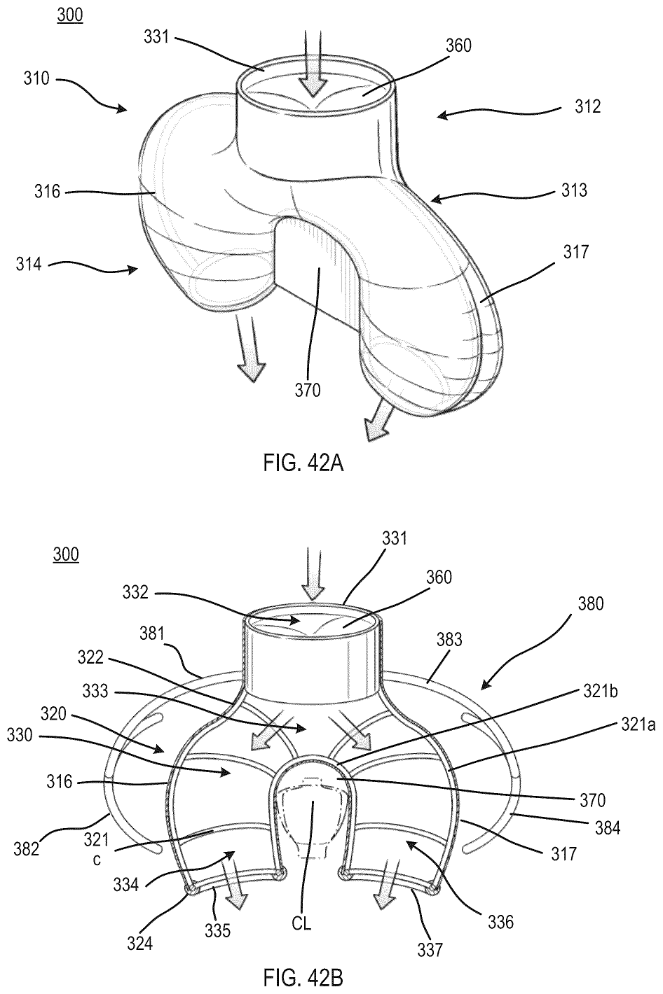

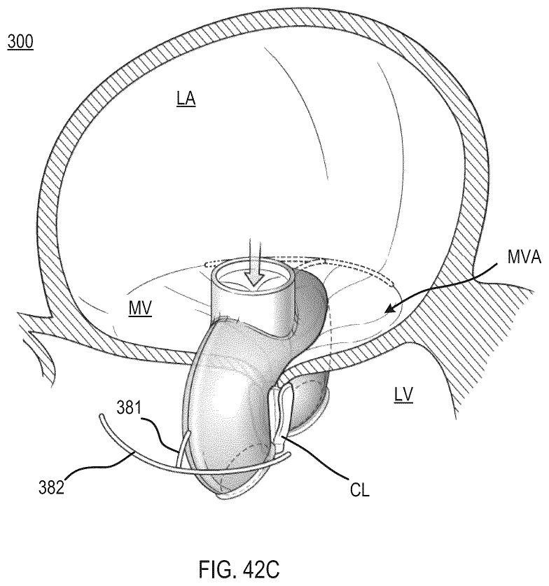

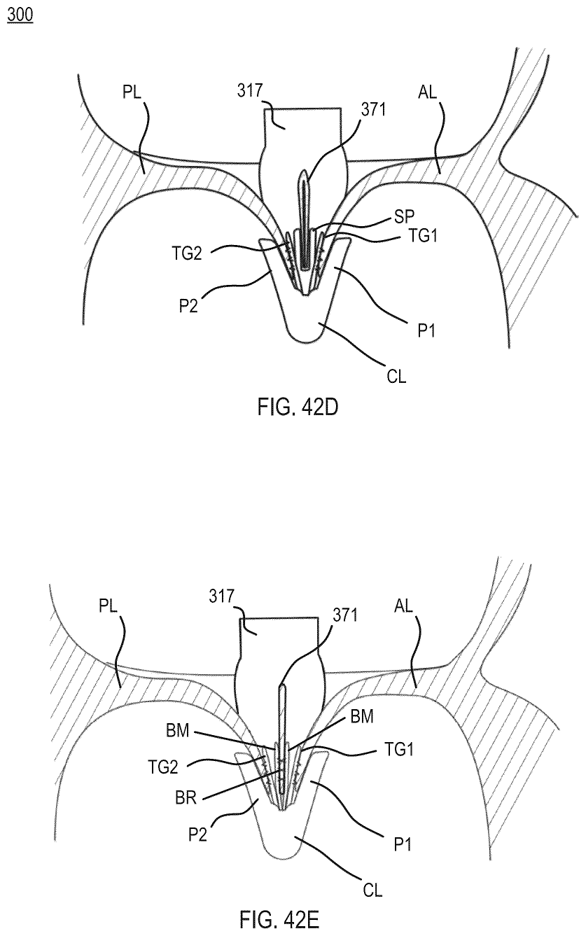

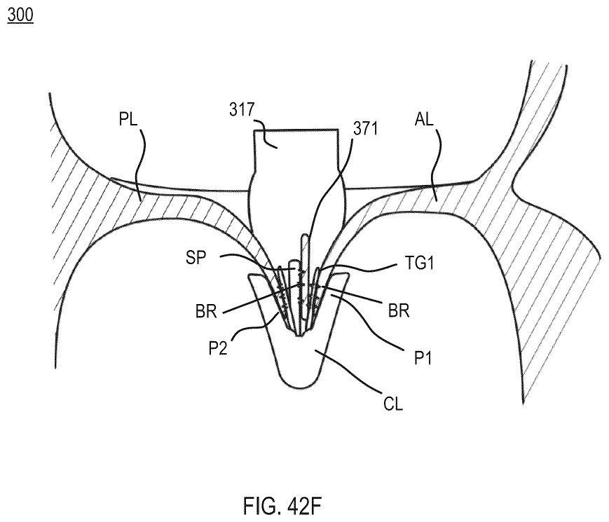

A prosthetic valve according to an embodiment is shown in FIGS. 42A-42C. Prosthetic valve 300 includes a body 310 with an inlet portion 312, transition portion 313, and outlet portion 314, with first limb 316 and second limb 317. Body frame 320 includes elongate, longitudinal struts 321a extending from the inlet 360 to the first outlet 335 and second outlet 337 on the outer sides of the body 320, and a U-shaped elongate strut 321b between first limb 316 and second limb 317, interconnected with a series of hoops or rings 321c. Body 310 further includes an outlet cuff 324 at the outlet end of each limb. Body 310 includes body covering 322 over the entire outer surface of body 310. Body 310 defines a flow passage 330 between inlet 331 and first outlet 335 and second outlet 337, including flow control passage 332, transition passage 333, first limb passage 334, and second limb passage 336.

Prosthetic valve 300 further includes a clip connector 370, which in this embodiment is implemented as a web 371 of material extending between first limb 316 and second limb 317, and which can be captured between the paddles of a clip CL and the native leaflets of the mitral valve MV. Embodiments and uses of various clips CL are described below.

FIG. 42D illustrates a clip CL having a first paddle or clip member P1, a second paddle or clip member P2, and a spacer SP. Anterior leaflet AL is captured between first paddle P1 and a first tissue gripper TG1 movable relative to paddle P1 to allow insertion of anterior leaflet AL free margin therebetween. Posterior leaflet PL is captured between a second paddle P2 and a second tissue gripper TG2 in a similar manner. Independent leaflet capture is achieved by selectively operating first paddle P1 and first tissue gripper TG1 to engage a first (e.g. anterior) leaflet, or second paddle P1 and second tissue gripper TG2 to engage a second (e.g. posterior) leaflet, as is the case with current PASCAL and latest generation MitraClip™ devices. Captured leaflets may be retained between a tissue gripper TG1, TG2 and a respective cooperating paddle P1, P2 even with the paddle in an open position relative to the opposite paddle, or with the paddle spaced away from the spacer SP. As illustrated in FIG. 42D, paddles P1, P2 of clip CL are shown in a fully closed position with captured tissue of anterior leaflet AL and posterior leaflet PL in an approximated spatial relationship.

Web 371 of clip connector 370 of prosthetic valve 300 may be fabricated by multiple plies of textile material (as illustrated) or in a laminate configuration to enhance its structural strength. Spacer SP is configured with an appropriately sized slot to engage web 371 and secure it in a reliable manner and withstand the dynamic load applied to prosthetic valve 300 during the cardiac cycle. Clip CL may be designed in a manner that closing of clip CL may impart an additional web-clamping load across the slot in spacer SP. FIG. 42E illustrates a variant for the coupling of web 371 of clip connector 370 to clip CL. Clip CL is configured with a pair of barbed members BM. Web 371 is of sufficient thickness and structural integrity to be penetrated by a series of barbs BR of barbed members BM to allow secure coupling of prosthetic valve 300 to clip CL. Structural stiffness and spacing of barbed members BM, and orientation of barbs BR allow insertion of web 371 in one direction and resist retraction of in the opposite direction. Alternatively, similar to tissue TG1, TG2, barbed members BM may be movable and operable between an open configuration to receive web 371 and a closed position to secure web 371 therewithin. Such closed position may coincide with a final closed position of clip CL.

FIG. 42F illustrates a further variant for coupling web 371 of clip connector 370 between a spacer SP and a captured leaflet (e.g. anterior leaflet AL). Tissue gripper TG1 is configured with a second series of barbs BR on the opposite side of the barbs BR used to capture anterior leaflet AL. Spacer SP is configured with a similar series of barbs BR. Inserting web 371 between spacer SP barbs BR and tissue gripper TG1 and closing clip CL will securely couple prosthetic valve 300 to clip CL. The insertion of web 371 is facilitated by having paddle PA and tissue gripper TG1 engaged with anterior leaflet AL, but with the latter being selectively positioned in paddle P1 in its open position spaced away from spacer SP.

Prosthetic valve 300 further includes annulus connector 380 (not shown in FIG. 42A for ease of illustration). In this embodiment, annulus connector includes a first arm 381 and second arm 383. First arm 381 is an arcuate, elongate rod or strut coupled to inlet portion 312 of body 310 and extending laterally and downwardly, and terminates at its distal end in a first annulus anchor 382, which is a transverse, arcuate, elongate rod or strut sized and oriented to engage the annulus of the native valve, e.g. mitral valve annulus MVA of mitral valve MV, as shown in FIG. 42C. (Note that FIGS. 42B and 42C illustrate slight different implementations of annulus connector 380—in FIG. 42B, first arm 381 and second arm 383 are coupled to inlet portion 312, whereas in FIG. 42C first arm 381 and second arm 383 are coupled to first limb 316 and second limb 317.) Second arm 383 is a mirror image of first arm 381, and terminates in a second annulus anchor 384, which is a mirror image of first annulus anchor 382. In THE embodiment of FIG. 42B, annulus connector 380 is configured to engage with the upper, atrial side of the mitral valve annulus MVA, but in the embodiment of FIG. 42C, it is instead configured to engage with the lower, ventricle side of the mitral valve annulus MVA, or the prosthetic valve 300 could include two annulus connectors, one on each side of the annulus.

Prosthetic valve further includes a flow control device 360, which in this implementation is a tri-leaflet valve, disposed in flow control passage 322 and coupled to body frame 320 in the inlet portion 312 of body 110. Blood flow through prosthetic valve is shown with arrows, i.e. blood can flow from the left atrium LA, into inlet 331, into flow control passage 332, through flow control device 360, into transition passage 333, into both first limb passage 334 and second limb passage 336, and out of first outlet 335 and second outlet 337 into left ventricle LV. This blood flow would take place during the diastolic portion of the cardiac cycle. During the systolic portion, the flow control device 360 would prevent blood flow in the opposite direction, from the left ventricle LV to the left atrium LA.

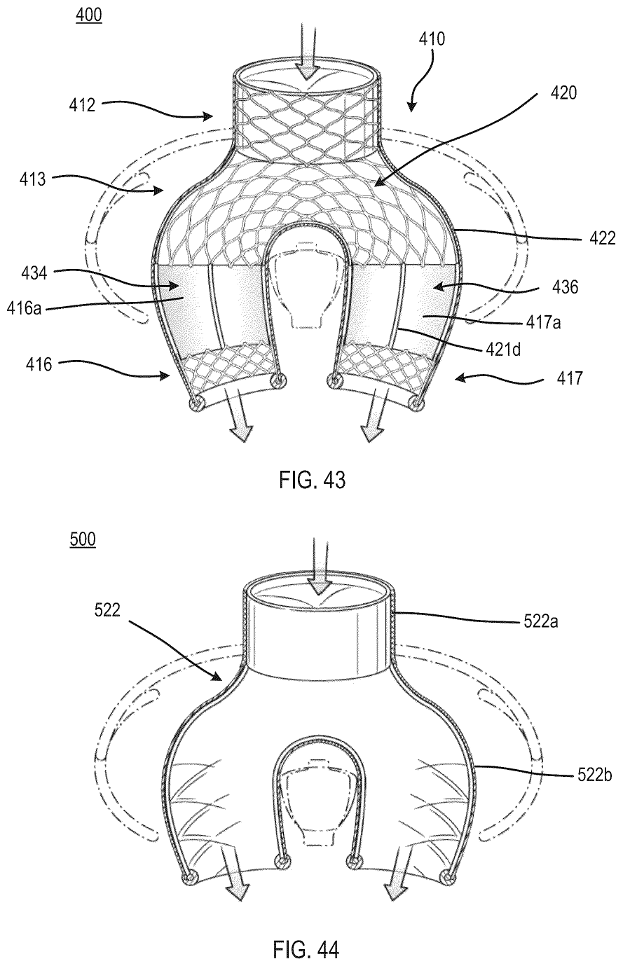

A prosthetic valve according to another embodiment is shown in FIG. 43. Prosthetic valve 400 in FIG. 43 is similar to prosthetic valve 300 in FIGS. 42A to 42C—the following description focuses on differences of interest, and omits details that are common. A clip and annulus connector are shown in phantom for reference. This embodiment has a variation in construction that may reduce native leaflet wear.

Prosthetic valve 400 includes a body frame 420 that is formed with different structures in different portions. In the inlet portion 412, transition portion 413 and parts of first limb 416 and second limb 417, body frame 420 is implemented with a wire mesh construction with diamond-shaped cells, such as by using laser-cut tubing, as is commonly used for the stents or bodies of prosthetic valves. However, in the portion of first limb 416 and second limb 417 that are to be disposed in the flow control portions of the clipped valve, and thus in contact with the edges of the native leaflets, there is less structure to body 410. In particular, the stent-like structure of the limbs have a gap in the leaflet-contact area 416a of the first limb 416 and leaflet contact area 417a of the second limb 417, and the gap is spanned by a small number of wires (or slender rods) 421d that link the stent-like portions. The wires can be preferentially arranged to be adjacent to laterally inside and outside edges of the limbs, so that when the prosthetic valve 400 is disposed in a mitral valve, the wires are adjacent to the clip and to the valves commissures, i.e. are away from the native leaflets, to minimize direct contact with the native leaflets. Additional wires or other supporting structures may be added as need to maintain the shape of the limbs in the leaflet contact areas. The outlet end of each limb may be formed with structure other than a stent frame, e.g. a simple circle or oval of wire.

The entire body frame 420 is covered with a body covering 422, which in this embodiment is fabricated with pericardium tissue. Body covering 422 is affixed to the stent-like portions of the body frame, i.e. above and below the leaflet contact areas of the limbs, but may not be attached to the underlying wires in the leaflet contact area. Thus, the native leaflets' engagement with the body covering 422 in the leaflet contact area imposes less stress and wear on the tissue of the native leaflets because the body covering 422 is backed only by blood in the first limb passage 434 and second limb passage 436.

A prosthetic valve according to another embodiment is shown in FIG. 44. Prosthetic valve 500 in FIG. 44 is similar to prosthetic valve 300 in FIGS. 42A to 42C—the following description focuses on differences of interest, and omits details that are common. A clip and annulus connector are shown in phantom for reference. FIG. 44 illustrates an alternative approach to holding prosthetic valve 500 in correct spatial relationship with the flow control portions FCP, i.e. the spatial relationship is maintained with an annulus connector, and a clip connector is not used. This embodiment has another variation in construction that may reduce native leaflet wear. Whereas typical stent mounted prosthetic valves are covered completely or partially by a fabric such as Dacron, prosthetic valve 500 includes a body covering 522 that has two portions-body covering inlet portion 522a and body covering limb portion 522b—each formed of different materials. Body covering limb portion 522b, which is the portion of body covering 522 that would contact the native leaflets during use, is formed of pericardium or similar biological material. Such biological material is less prone to wearing the native leaflets than the fabric material covering the remainder of prosthetic valve 500.

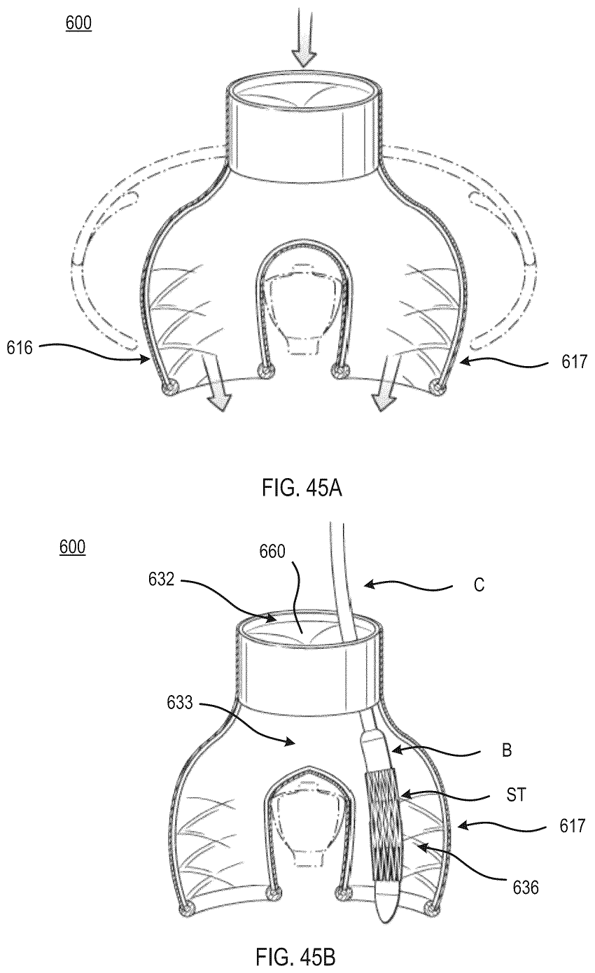

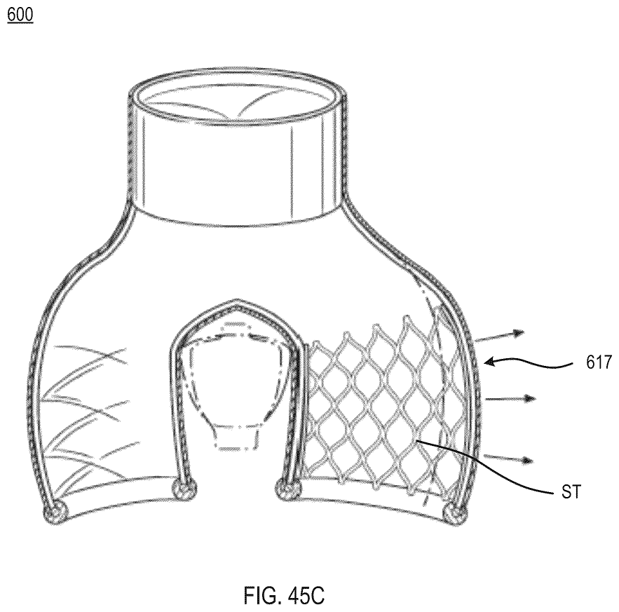

A prosthetic valve according to another embodiment is shown in FIGS. 45A to 45C. Prosthetic valve 600 in FIGS. 45A to 45C is similar to prosthetic valve 300 in FIGS. 42A to 42C—the following description focuses on differences of interest, and omits details that are common. A clip and annulus connector are shown in phantom for reference. This embodiment is used to illustrate a procedure that can be used to address leakage between a limb of the prosthetic valve and the native leaflets.

To be effective in preventing mitral regurgitation, the native leaflets should sealingly engage the limbs of the prosthetic valve. It is well known that as the heart deteriorates in heart failure, the native leaflets can become more distracted, and regurgitation can increase. It is envisioned that native leaflet distraction could become sufficiently large that the native leaflets no longer sealingly engage the limbs of the prosthetic valve. This potential issue can be addressed by a procedure in which one or more of first limb 616 and second limb 617 may be expanded to a larger perimeter after prosthetic valve 600 has been delivered. Such a procedure can be performed in conjunction with the procedure in which prosthetic valve 600 is delivered and deployed, e.g. by evaluating the sealing of the native leaflets to first limb 616 and second limb 617, such as by measuring the presence and severity of regurgitation, and the using the procedure to address any such regurgitation. Alternatively, the procedure can be performed separately, for example well after the initial procedure to deliver and deploy prosthetic valve 600 has been performed and deterioration of the heart causes the onset of, or increase in, regurgitation.

The procedure to reshape or increase the perimeter of first limb 616 and/or second limb 617 can be accomplished in several ways. First, as shown in FIG. 45B, a catheter C having an expandable balloon B on which is disposed a balloon-expandable stent ST (e.g. constructed of stainless steel or cobalt chrome) can be delivered to the native valve and into second limb passage 636 of second limb 617 (via flow control passage 632, flow control device 660, and transition passage 633). Balloon B can then be inflated, expanding stent ST into engagement with—and then expanding—the second limb portion of body frame 620. The resulting condition of prosthetic valve 600 is shown in FIG. 45C, with stent ST in place in second limb 617. The dashed line if FIG. 45C shows the original size of second limb 617, the arrows indicate the expansion by stent ST, and the solid line illustrates the new, expanded size of second limb 617.

Another approach to expanding the perimeter of, for example, second limb 617 that can result in the condition shown in FIG. 45C, is to use a stent ST that is self-expanding (e.g. one formed from shape memory material such as Nitinol), and deliver it to second limb 617 with a catheter (not shown) with a delivery lumen from which stent ST can be discharged into position. A benefit of using a self-expanding stent is that, as is well known, such stents can be retrieved (e.g. via the delivery catheter before deployment is complete, or via a retrieval catheter if already deployed) if the deliver is unsatisfactory or the stent fails.

A third approach to expanding the perimeter of, for example, second limb 617 that can result in the condition shown in FIG. 45C, is to omit the stent ST and use the balloon B on catheter C directly to further expand the perimeter of the portion of body frame 620 in second limb 617 from the perimeter with which it was initially delivered and deployed, e.g. if that portion of body frame 120 is constructed of expandable material such as stainless steel or cobalt chrome (rather than from a shape memory material).

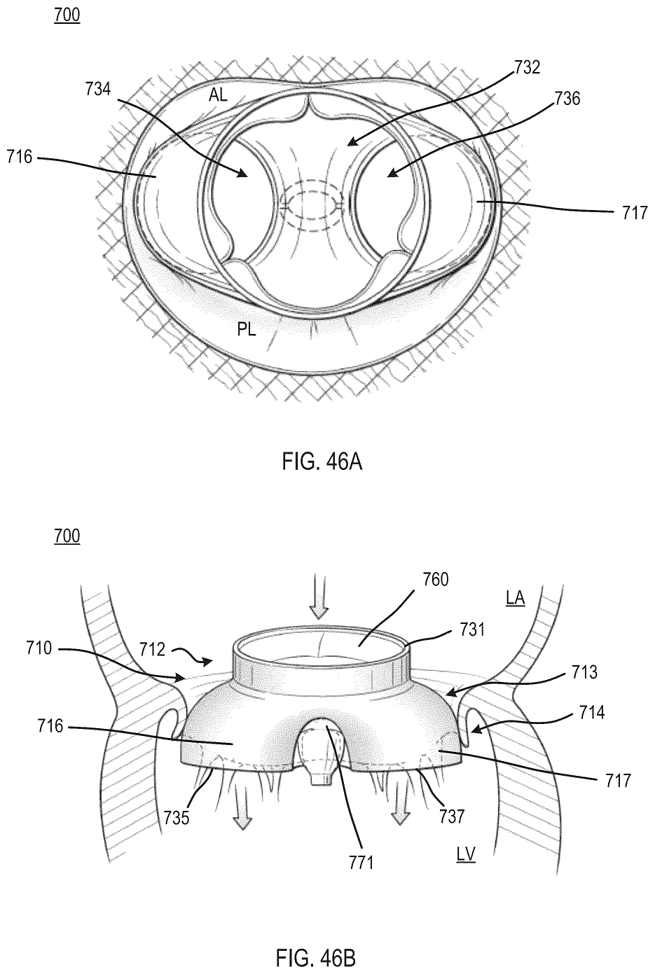

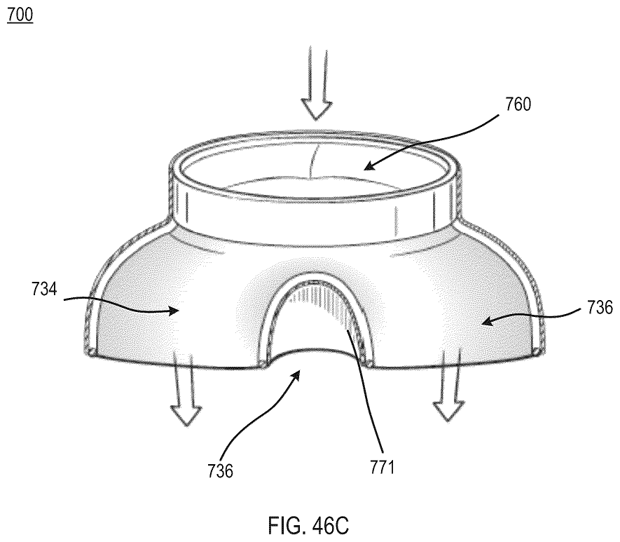

A prosthetic valve according to another embodiment is shown in FIGS. 46A to 46C. Prosthetic valve 700 in FIGS. 46A to 46C is similar to prosthetic valve 300 in FIGS. 42A to 42C—the following description focuses on differences of interest, and omits details that are common. This embodiment is used to illustrate that prosthetic valve 700 can have a relatively short axial height (especially within the left atrium) and a considerably larger inlet diameter flow control passage.

Prosthetic valve 700 has a body 710 with an inlet portion 712, transition portion 713, and outlet portion 714 (with first limb 716 and second limb 717). Body 710 defines a flow passage that includes a flow control passage 732, a transition passage 733, a first limb passage 734, and a second limb passage 736, and extends between an inlet 731 and a first outlet 735 and a second outlet 737. A flow control device 760 is disposed in flow control passage 732. As can be seen in FIGS. 46B and 46C, flow control device 760 has a relatively short axial height (i.e. in along its central, longitudinal axis). The entire body is also has a relatively short axial height, between inlet 731 and first outlet 735 and second outlet 735. Thus, when prosthetic valve 700 is disposed in a native valve, such as the mitral valve between left atrium LA and left ventricle LV, as shown in FIG. 46B, inlet 731 is disposed in left atrium LA but leaves ample clearance from the walls of the atrium to allow good blood flow into flow control device 750. First outlet 735 and second outlet 737 are disposed in left ventricle LV, but do not project far into the ventricle, and thus minimize contact with portions of the native valve apparatus or the ventricle wall. As shown in FIGS. 46A to 46C, flow control device 760 also has a large diameter relative to the overall size of prosthetic valve 700, as do first outlet 735 and second outlet 737 (and the flow passage between inlet 731 and the outlets), thus providing a large flow area for blood to pass through prosthetic valve 700 from left atrium LA to left ventricle LV during diastole, as indicated by the arrows in FIGS. 46B and 46C.

Similar to prosthetic valve 300, prosthetic valve 700 includes a clip connector 770 which is configured from a structural web 771 extending from and spanning between first limb 716 and second limb 717 of valve 700. Clip connector 770 is couplable to clip CL in a variety of ways as previously described in FIGS. 42D to 42F. Once coupled with clip CL, web 771 of clip connector 770 is engaged between opposing paddles or clip members of clip CL and also between the captured portions of opposed and approximated native leaflets (e.g. anterior leaflet AL and posterior leaflet PL in mitral valve MV).

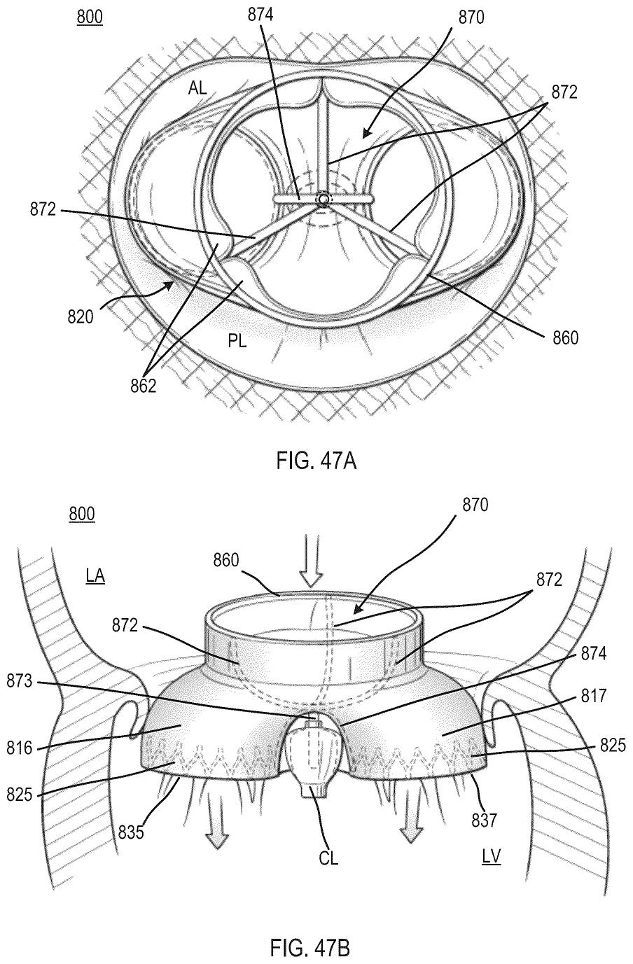

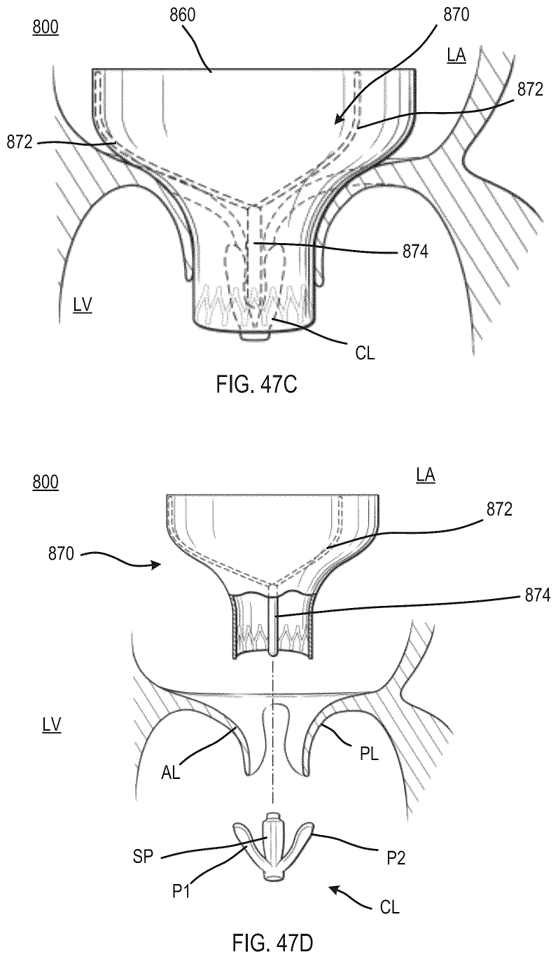

A prosthetic valve according to another embodiment is shown in FIGS. 47A to 47D. Prosthetic valve 800 in FIGS. 47A to 47D is similar to prosthetic valve 700 in FIGS. 46A to 46C—the following description focuses on differences of interest, and omits details that are common. This embodiment is used to illustrate structures for coupling prosthetic valve 800 to clip CL.

Prosthetic valve 800 has a clip connector 870 that transfers fluid dynamic loads imposed on prosthetic valve 800 to clip CL via an axial clip post 873. Axial clip post in turn is connected to body frame 820 by two paths: via three radial valve struts 872 coupled between axial clip post 873 and the upper rim of the frame of flow control device 860 (which may be coupled to, or a portion of, body frame 820); and via a U-shaped crotch strut 874 coupled between axial clip post 873 and the portion of body frame 820 between first limb 816 and second limb 817. Body frame 820 includes outlet portions 825 (which may be short sections of stent structures) at the outlet ends of first limb 816 and second limb 817, to maintain first outlet 835 and second outlet 837 open. Crotch strut 874 can be coupled to outlet portions 825. Axial clip post 873 is coupled to clip CL via any suitable mechanical joint, such tongue-and-groove, a barbed fitting, a snap fit, etc. As such, prosthetic valve 800 may be coupled to clip CL: i) after clip CL has been previously and fully deployed (i.e. both leaflets of a target native valve have been captured by clip CL); ii) after clip CL has been partially deployed with only one of the native leaflets captured between a central spacer and a first clip member (such as between spacer SP and paddle P1 of clip CL shown in FIGS. 42D to 42F), and prior to capturing a second native leaflet between the central spacer and a second clip member (such as second paddle P2 shown in FIG. 42D to 42F; or iii) prior to leaflet capture by clip CL (i.e. prosthetic valve 800 and clip CL forming a device assembly prior to delivery to the patient's target heart valve). A releasable mechanical joint may also be used, thereby allowing prosthetic valve 800 to be decoupled from clip CL and replaced by a different size or configuration of prosthetic valve if a surgical intervention warrants such replacement.

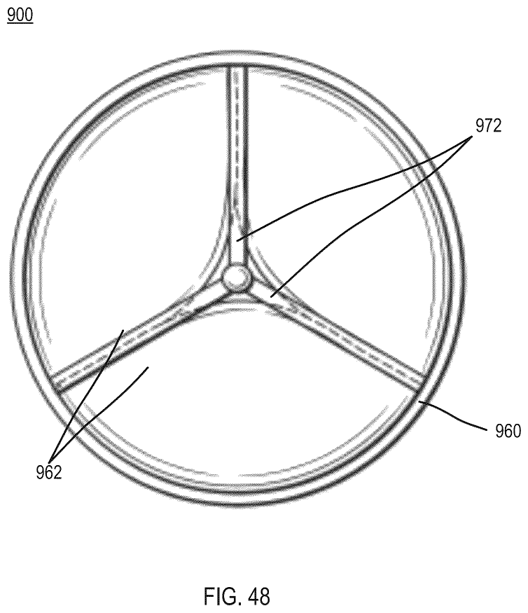

Radial valve struts 872 are configured and arranged to be disposed below the coaptation line of the leaflets 862 of flow control device 860 as best seen in FIGS. 47A (leaflets 862 shown open, during diastole) and 47B (leaflets 862 are shown coapted, during and radial valve struts 872 are shown in phantom). In an alternative embodiment, shown in FIG. 48, a prosthetic valve 900 includes radial valve struts 972 that are configured and arranged to be disposed above the coaptation line of the leaflets 962 of flow control device 960. In both embodiments, radial valve struts 872 and 972 can be securely coupled to the frame of the flow control device, and do not interfere with the operation of the leaflets of the flow control device-thus, these designs facilitate the use of already-developed prosthetic valves for the flow control device, rather than requiring re-engineering of their design.

Prosthetic valve 800 is shown in an end view disposed in native mitral valve MV in an delivered position and in an exploded view, respectively, in FIGS. 47C and 47D. Clip CL is shown in FIG. 47D with its paddles P1, P2 open, and the relationship of native leaflets AL and PL and the clip connector 870 with clip CL is clearly seen. Spacer SP is of a suitable size and volume to advantageously allow configuration of a mechanical joint, or other suitable interface, to appropriately engage a clip connector 870 of prosthetic valve 800. The latter can be achieved with either or both of paddles P1, P2 in their open spaced apart position, or with paddles P1, P2 in a closed position and proximate to spacer SP.

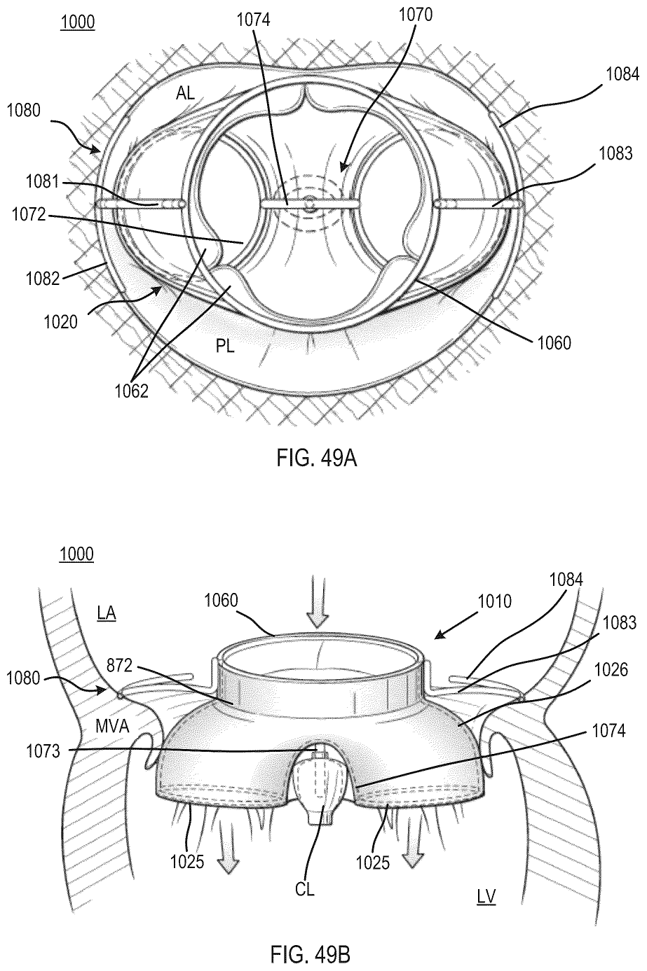

A prosthetic valve according to another embodiment is shown in FIGS. 49A to 49B. Prosthetic valve 1000 in FIGS. 49A and 49B is similar to prosthetic valve 300 in FIGS. 42A to 42C—the following description focuses on differences of interest, and omits details that are common. This embodiment illustrates an alternative design for an annulus connector.

As shown in FIGS. 49A and 49B, prosthetic valve 1000 includes a body frame 1020 that integrates with a clip connector 1070 and an annulus connector 1080. In contrast to clip connector 870 of prosthetic valve 800 in FIGS. 47A to 47D, the load path for clip connector 1070 is only through crotch strut 1074. Outlet portions 1025 of body frame 1020 are wire hoops or rings, and each is coupled on its laterally inner side to a lower end of crotch strut 1074 and on their laterally outer side to a body frame side strut 1026 running axially along a laterally outer side of body 1010. Each body frame side strut 1026 is coupled at its upper end to the frame of flow control device 1060 and/or to annulus connector 1080.

Annulus connector 1080 includes first arm 1081 and second arm 1083, each extending from the frame of flow control device 1060 and/or upper end of a corresponding body frame side strut 1026, and having at their distal ends first annulus anchor 1082 and second annulus anchor 1084, respectively. In this embodiment, annulus connector 1080 engages the atrium side of mitral valve annulus MVA. However, alternatively, or in addition, annulus connector could include arms that extend through the commissures of the mitral valve and have annulus anchors disposed to engage the ventricle side of mitral valve annulus MVA. First annulus anchor 1082 and/or second annulus anchor 1084 may include tissue piercing members, such as barbs, for enhancing securement to heart tissue.

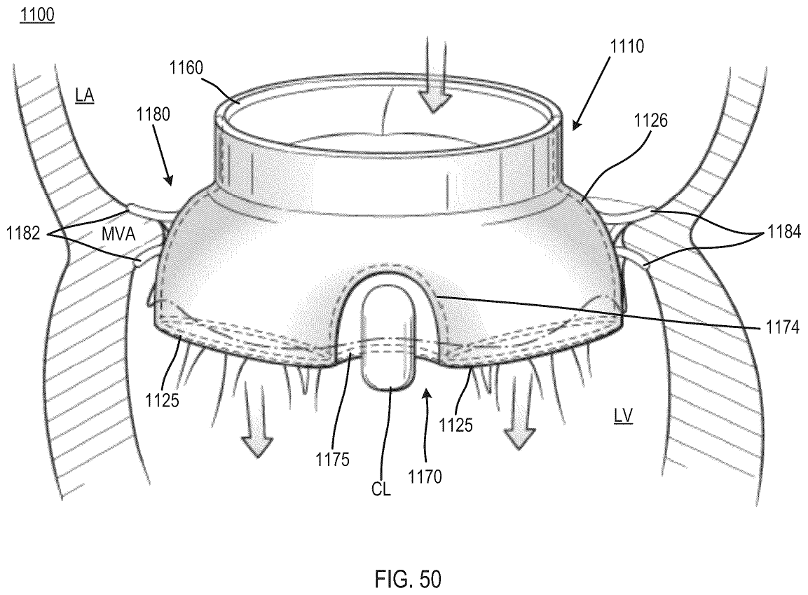

A prosthetic valve according to another embodiment is shown in FIG. 50. Prosthetic valve 1100 in FIG. 50 is similar to prosthetic valve 1000 in FIGS. 49A and 49B, but includes an annulus connector 1180 that engages both the atrium and ventricle sides of mitral valve annulus MVA.

As shown in FIG. 50, prosthetic valve 1100 includes a body frame that includes outlet portions 1125, each coupled on its laterally inner side to a lower end of crotch strut 1174 and on their laterally outer side to a body frame side strut 1126 running axially along a laterally outer side of body 1110. Each body frame side strut 1126 is coupled at its upper end to the frame of flow control device 1160. Annulus connector 1180 includes two first annulus anchors 1182 and two second annulus anchors 1184 extending from a respective body frame side strut 1126. One first annulus anchor 1182 engages the atrium side of mitral valve annulus MVA and the other first annulus anchor 1182 engages the ventricle side of mitral valve annulus MVA. Similarly, one second annulus anchor 1184 engages the atrium side of mitral valve annulus MVA and the other second annulus anchor 1184 engages the ventricle side of mitral valve annulus MVA.

Clip connector 1170 includes a transverse strut 1175, coupled at its ends to the two body frame outlet portions 1125, and coupled at its center to clip CL. Unlike some of the previous embodiments, transverse strut 1175 can be disposed on the ventricle side of clip CL, and even below the level of the captured native leaflet free margin within clip CL.

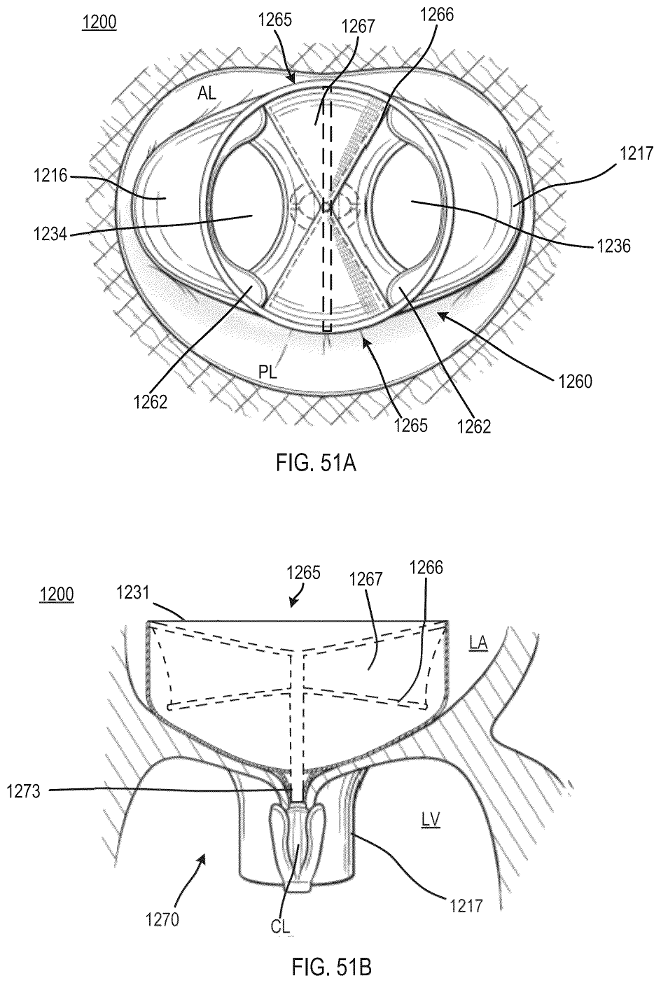

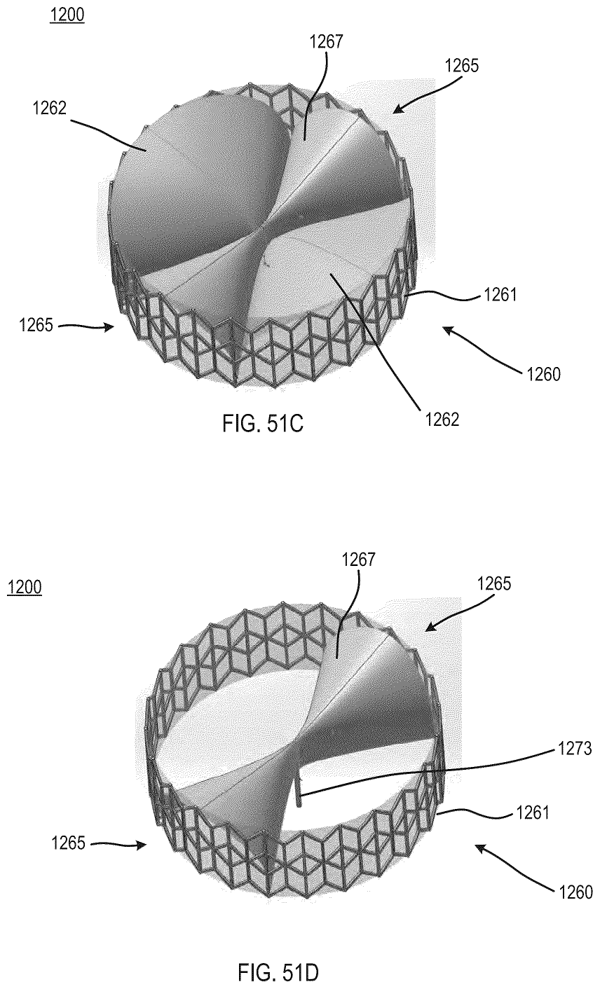

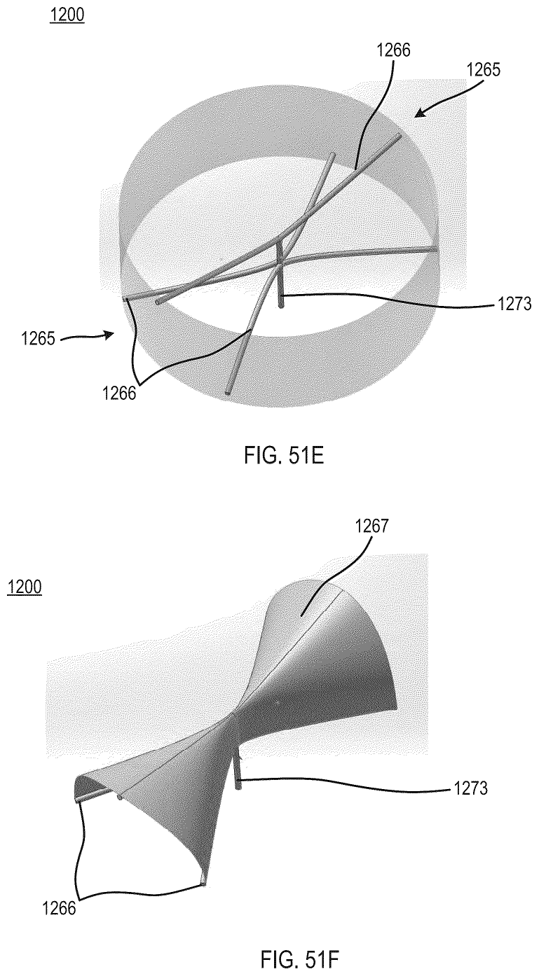

A prosthetic valve according to another embodiment is shown in FIGS. 51A and 51B in a top view and a partial cross-sectional end view, respectively. Prosthetic valve 1200 includes a non-standard flow control device 1260 that can provide better blood flow through prosthetic valve 1200. Flow control device 1260 can be used with any of the prosthetic valve embodiments described above, e.g. prosthetic valves 300, 400, 500, 600, 700, 800, 900, 1000, and 1100, instead of a standard tri-leaflet design.

As shown in FIGS. 51A and 51B, and in more detail in a perspective views in FIGS. 51C and 51D, flow control device 1260 includes a stent frame 1261 supporting two conventional leaflets 1262, each subtending one third of the circumference of the flow control device 1260. However, instead of being adjacent to each other, and joined at a commissure, the leaflets 1262 are spaced apart, disposed diametrically opposite each other, and aligned with first limb 1216 and second limb 1217, and correspondingly with first limb passage 1234 and second limb passage 1236. Rather than coapting against each other, leaflets 1262 coapt against static half-cusps 1265, each subtending one sixth of the circumference of the flow control device 1260 and disposed between leaflets 1262. Flow control device 1265 is shown in FIG. 51C in the configuration it assumes during systole, i.e. with the tissue leaflets 1262 coapted against the static half-cusps 1265. Flow control device 1260 is shown in FIG. 51D with the tissue leaflets omitted for clarity of illustration of the static half-cusps 1265.

As also shown in more detail in FIGS. 51D to 51F, each static half-cusp 1265 includes a static cusp frame 1266 and a static cusp membrane 1267 supported on static cusp frame 1266. Both the leaflets 1262 and the static cusp membrane 1267 may be formed from tissue such as pericardium. Static cusp frame 1266 may be formed of the same material as the main frame of flow control device 1260, e.g. stainless steel, cobalt chrome, or Nitinol. As shown in FIGS. 51B and 51D to 51F, static cusp frames 1266 may be coupled to axial clip post 1273 of clip connector 1270. Variations in the construction of the static half-cusp assembly are possible, including draping or encapsulating frame 1266 with a suitable biopolymer membrane, for example silicone poly (urethane urea) formulation. Alternatively, the volume delimited by the static cusp frame 1266, static cusp membrane 1267 and stent frame 1261 may include a collapsible open-cell foam polycarbonate urethane draped by a pericardium or biopolymer membrane. Alternatively, the static half cusps may be constructed to include a biopolymer, biocompatible, or bioengineered material capable of maintaining its shape and geometry in use, and suitable to resist calcification, withstand stresses and strains of the cardiac cycle, and that is non-thrombogenic. Such materials are also suitable for the movable cusps in prosthetic valves 300, 400, 500, 600, 700, 800, 900, 1000, and 1100, instead of the more commonly used animal pericardium. One example of a prosthetic valve using such biopolymer material is the Tria Valve produced Foldax Inc.

In operation of flow control device 1260, during diastole leaflets 1262 are open, collapsed against the periphery of flow control device, i.e. as shown FIG. 51A. Blood can flow from the left atrium LA into inlet 1231, through the apertures between leaflets 1262 and static half-cusps 1265, and into first limb passage 1234 and second limb passage 1236. As is apparent from FIG. 51A, the alignment of leaflets 1262 with the limb passages provides a smooth, relatively straight flow path. During systole, leaflets 1262 coapt and seal against static cusp membranes 1267, blocking retrograde blood flow or regurgitation, similar to the coaptation of leaflets in a tri-leaflet valve. In configurations of prosthetic valve 1200 having limb passages 1234, 1236 that are not diametrically opposite as illustrated in FIG. 51A, alignment of leaflets 1262 can be tailored to align with limb passages by varying the amount that the static half-cusps 1265 each subtend the circumference of the flow control device 1260. For example, in an embodiment having a first limb 1216 and a second limb 1217 angularly oriented 160 degrees apart relative to clip CL, a first static half cusp 1265 can be configured to subtend one-ninth of the circumference and a second half cusp 1265 configured to subtend two-ninths of the circumference such that the resulting alignment of leaflets 1262 is in register with the limb passages 1234, 1236.

The prosthetic valve embodiments described above include a single flow control device to control the flow through two (or more) flow control portions of a clipped native valve, by incorporating a bifurcated flow control passage with two (or more) limb passages extending through two (or more) limbs, each preferably sealingly engaging the native leaflets in a respective flow control portions. In the following prosthetic valve embodiments, a separate flow control device is used to control the flow through each flow control portion of a clipped native valve. Thus, for a clipped native valve with two flow control portions through which it is desired to control flow with a prosthetic valve (rather than relying only on the function of the clipped native leaflets founding the flow control portion), a prosthetic valve includes two flow control devices. For a clipped native valve with a single flow control portion, or with multiple flow control portions but for which it is necessary or desirable to address regurgitation through only one of the flow control portions, the prosthetic valve includes a single flow control device. Other structures and functions described for the prosthetic valve embodiments above are also applicable to the embodiments described below, and additional structures or functions are included in the embodiments described below, as will be made clear from the following description. In general, the same reference numbering scheme is used for the preceding and following embodiments, for ease of reference, and unless otherwise apparent from the detailed description below, any structure in the following embodiments that corresponds to structure in the embodiments above can include all of the same details of design and implementation, and all of the same options and alternatives, as described above.

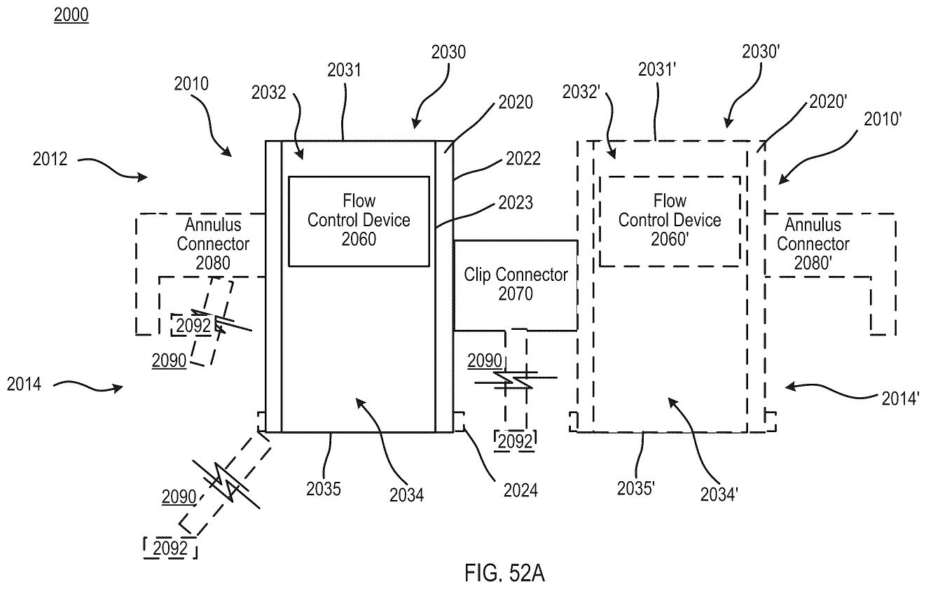

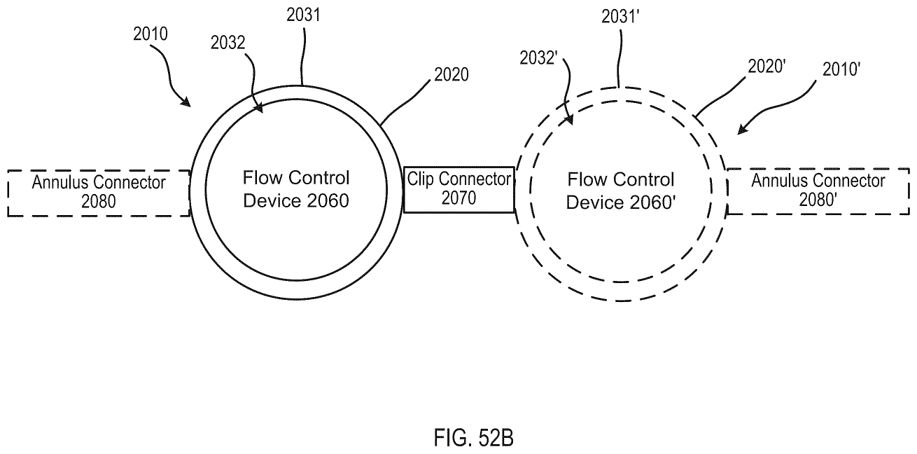

An embodiment of a prosthetic valve 2000 is illustrated schematically in a side view and top view, respectively, in FIGS. 52A and 52B. Prosthetic valve 2000 includes a body 2010 with an inlet portion 2012 and an outlet portion 2014. Body 2010 defines a flow passage 2030 therethrough that includes a flow control passage 2032 in the inlet portion 2012 and an outlet passage 2034 in the outlet portion 2014.

The portions of the flow passage 2030 are in fluid communication with each other and can conduct fluid, e.g. blood, from an inlet 2031 at the entrance to the flow control passage, through the flow control passage 2032 and through the outlet passage 2034 out of an outlet 2035 at the lower end of body 2010.

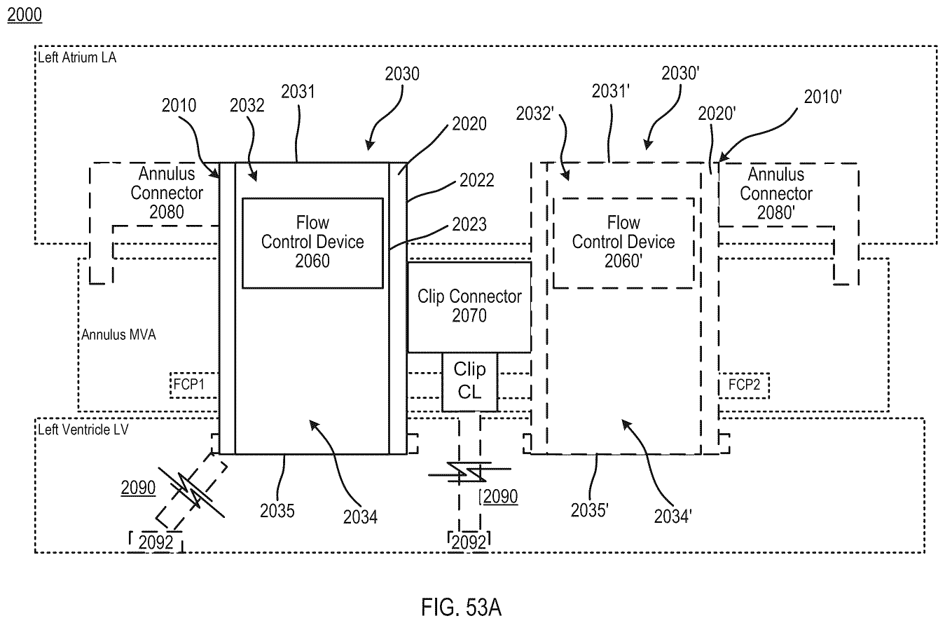

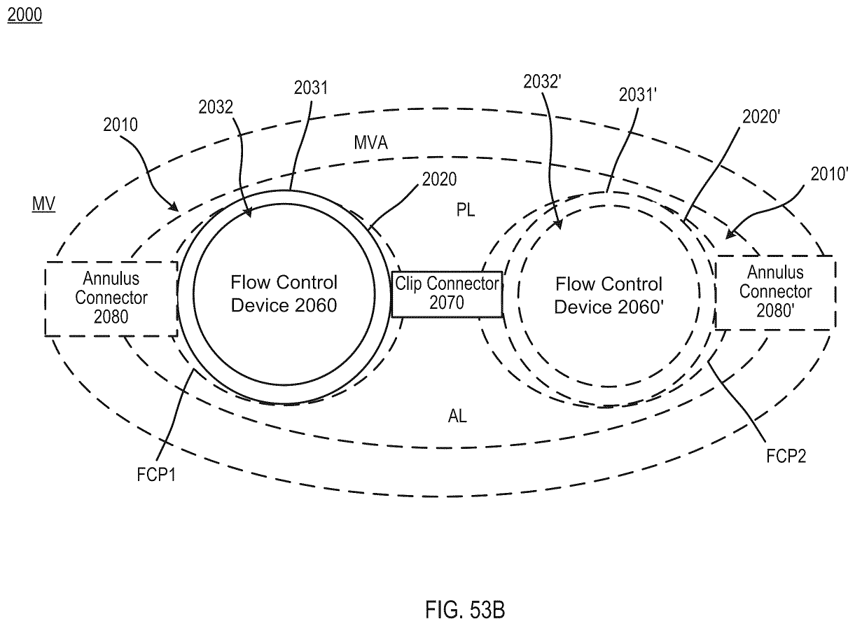

Flow through the flow passage 2030, and in particular through flow control passage 2032, is controlled by flow control device 2060. Flow control device 2060 can be constructed, and function, similar to any of the flow control devices described above for other embodiments. As shown schematically in FIGS. 52A to 53B, flow control device 2060 may be cylindrical, with a circular cross section. Flow control device 2060 may be mounted to inlet portion 2012 of body 2010 and disposed so that all flow through flow control passage 2032 must pass through flow control device 2060. Flow control device 2060 is configured to permit fluid to flow therethrough in the direction from the inlet 2031 to the outlet 2035, but to prevent fluid to flow in the opposite direction.

It is well known that tissue valves can fail, and it is also known that this problem can be solved by delivering another tissue-based stent valve inside a failed valve. Thus, it is contemplated that if flow control device 2060 fails, a new tri-leaflet valve can be placed inside flow control device 2060.

Prosthetic valve 2000 also includes a clip connector 2070 that is part of, or coupled to, body 2010, and is configured to engage with a clip such as those described above, and thereby to retain prosthetic valve 2000 in operative relationship with a native heart valve to which the clip is attached. In particular, clip connector 2070 is configured to carry fluid dynamic load applied to prosthetic valve 2000 during the cardiac cycle of the heart to the clip CL and thence to the native leaflets, annulus, and surrounding heart tissue to resist displacement of the prosthetic valve. The largest loads to be carried tend to be during systole, and the displacement to be resisted is towards the atrium of the heart.

Clip connector can be implemented in a variety of configurations, including those described above, as well as additional variations describe in more detail below. As describe above, clip CL can be any of the commercially available designs, or can be customized or modified specifically for use with prosthetic valve 2000. For example, as described in more detail below for specific embodiments, clip CL can have a spacer (similar to the spacer of the PASCAL clip) disposed between the paddles of the clip, and the spacer can be configured to fill or occlude a portion of the space between the native leaflets of a clipped native valve in a clipping procedure, thus reducing the size of, or filling a portion of, the native valve orifice area. The spacer can be configured and sized to increase a resulting flow control portion (e.g. adjacent to a commissure between the native leaflets) relative to clipping the same native valve with a clip not having a spacer, and whereby the paddles are proximally disposed to each other.

As shown schematically in FIGS. 52A to 53B, prosthetic valve 2000 may include a second body 2010′ and associated flow control device 2060′, which can also be coupled to the clip connector 2070, and may also have an optional annulus connector 2080′ (or be coupled to the same annulus connector 2080). Body 2010′ may be identical in structure and function to body 2010, including a flow passage 2030′ with inlet 2031′, flow control passage 2032′, outlet passage 2034′, and outlet 2035′. Body 2010′ may have a body frame 2020′, etc. A prosthetic valve 2000 with both body 2010 and 2010′ may be used to control blood flow in a clipped native valve having two flow control portions needing regurgitation reduction-body 2010 can be disposed in a first flow control portion FCP1 and body 2010′ can be disposed in a second flow control portion FCP2, as shown in FIGS. 53A and 53B.

Prosthetic valve 2000 is shown disposed in a native heart valve in a side view and top view, respectively, in FIGS. 53A and 53B. Note that for convenience of illustration the native heart valve is illustrated as a mitral valve MV. Note also that prosthetic valve 2000 is illustrated with the optional second body 2010′ disposed in one of the two flow control portions of clipped mitral valve MV. Mitral valve MV is shown with the anterior leaflet AL and posterior leaflet PL joined by a clip CL in an edge-to-edge approximation, similar to the mitral valve MV shown in FIG. 37B. Thus, mitral valve MV has two flow control portions-FCP1 and FCP2-defined between the clip, the leaflets, and the commissures of the mitral valve MV. As discussed above with reference to FIGS. 37A to 38F, there are many possible arrangements of clips on either the mitral valve or tricuspid valve, creating one, two, or three flow control portions-prosthetic valve 2000 may be used with any of those clipped valve configurations to address regurgitation in one or two of the flow control portions.

As shown in FIGS. 53A and 53B, prosthetic valve 2000 can be disposed in mitral valve MV with inlets 2031 and 2031′ disposed in the left atrium LA and outlets 2035 and 2035′ disposed in the left ventricle LV. Body 2010 is shown disposed in flow control portion FCP1, and body 2010′ is shown disposed in flow control portion FCP2. Clip connector 2070 is engaged with clip CL. Optional annulus connectors 2080 and 2080′ can be engaged with mitral valve annulus MVA. Similarly, optional heart tissue tether(s) 2090 can be engaged with heart tissue, e.g. in the left ventricle LV. When prosthetic valve 2000 is disposed in mitral valve MV, it is operable to reduce or eliminate regurgitation through flow control portions FCP1 and/or FCP2, i.e. to prevent retrograde flow of blood from the left ventricle to the left atrium during systole, but to allow blood to flow freely through prosthetic valve 2000 from left atrium LA to left ventricle LV during diastole.

The height of inlet portion 2012 of body 2000, may be of any suitable distance, though it is desirable that it not be so large that the inflow of blood into inlet 2031 is impeded, i.e. sufficient room is left above and around inlet 2031 inside the atrium of the heart for blood to freely enter.

Each of body 2010 and 2010′ may be configured so that outer surfaces thereof engage in a substantially sealing relationship with the anterior leaflet AL and the posterior leaflet PL and thereby to reduce or prevent the flow of blood therebetween, during at least a portion of the pumping cycle of the heart. In some embodiments, each of the first body 2010 and the second body 2010′ may be sized (e.g. perimeter) and configured (e.g. cross-sectional shape is circular, elliptical, ovoid, etc.) to substantially fill, or over-fill (stretch) the corresponding flow control portion, maintaining the edges of the leaflets in sealing relationship with the outer surfaces of the first body 2010 and the second body 2010′ throughout the cardiac cycle, thus preventing flow between the outlet portions and the leaflets from the atrium to the ventricle during diastole and from the ventricle to the atrium during systole. In this configuration, substantially all blood flow from the atrium to the ventricle during diastole is therefore carried through the prosthetic valve (and thus through the flow control devices 2060, 2060′), and blood flow from the ventricle to the atrium during systole (regurgitation) is substantially prevented (by the flow control devices 2060 and 2060′). This configuration offers several benefits. First, the native leaflets would move little or not at all during the cardiac cycle, which should reduce wear resulting from repetitive contact between the leaflets and the outer surfaces of the bodies of the prosthetic valve (there is little momentum on the native leaflet during impact with the outlet portions). The native leaflets are pliable and will tend to fill any irregular shapes or defects in closure. Second, complete valve sealing, i.e. prevention of regurgitation, should be assured. Finally, the heart tends to deteriorate over time in patients needing valve repair or replacement. For such patients, regurgitation should not occur again as the prosthetic valve assumes virtually full responsibility for the function of the native valve, and the residual valve tissue will be able to fill any gaps that may occur as the heart dilates (or alternatively any gaps that may occur as the valve leaflets retract with disease progression). These benefits are particularly applicable for a native valve to which an edge-to-edge clip has been applied. After the clip is applied the total opening size of the valve is limited to the area of the resultant flow control portion(s), which is a smaller area than that of the original opening of the native valve. Thus, the surface or orifice area that must be occluded by a valve is reduced and the load on the prosthetic valve is reduced. In many instances the clip can securely hold the load created by cardiac contraction, highest during systole.

In another configuration, the bodies 2010, 2010′ could be sized to be smaller than the flow control portions, allowing a gap to be formed during diastole and permitting some of the blood flowing from the atrium to the ventricle to flow through the gap (in addition to the blood flowing through the flow passage and the flow control device). The bodies 2010, 2010′ are preferably sized so that during systole the leaflets can sealingly engage the bodies' outer surface and prevent retrograde flow between the limbs and the leaflets.

The bodies 2010, 2010′ of prosthetic valve 2000 are shown schematically in FIGS. 52B and 53B as being circular in cross section, whereas the flow control portions of the native valve that result from leaflet clipping may be oval or slit like, as shown in FIG. 53B for ease of illustration. However, shaping the bodies with a corresponding cross-section could better follow the shape of the flow control portions and fill the leak space. In some embodiments, the cross-sectional shape of the bodies, at least in the leaflet-contacting areas, could be more elliptical, or with a teardrop (more V-shaped) shape, with the narrower portion oriented toward the commissures. Although the bodies are shown schematically in FIGS. 52B and 53B as being approximately linear, or symmetrically arranged about a center line through the clip, as can be seen in FIG. 36A, there is a natural curve to the coaptation line of the native mitral valve leaflets. When looking down on the mitral valve with the anterior leaflet above, there is a upward curve to the line of closure. To conform to this anatomy, in some embodiments the bodies of the prosthetic valve could be arranged to follow the curve of the coaptation line (i.e. curve formed by the leaflet free margins of opposed mitral or tricuspid leaflets during systole).

Bodies 2010, 2010′ are shown schematically in FIGS. 52B and 53B as being straight and being parallel with each other. However, in some embodiments with two bodies, the bodies may be straight but may be non-parallel, and may be angled towards or away from each other. In other embodiments they may not be straight, but may instead be arcuate.

Bodies 2010, 2010′ are shown schematically in FIGS. 52A to 53B as being generally tubular in shape. However, in some embodiments it may be useful for the bodies to flare outwardly (larger perimeter) towards their respective outlets, as this may encourage more blood to enter the flow passage 2030 during systole and urge closed the leaflets of the flow control devices 160, 160′. Thus, the outlet ends of the bodies 2010, 2010′ could have a trumpet bell shape, for example.

Although bodies 2010, 2010′ are shown schematically in FIGS. 52B and 53B as having ends (i.e. at outlets 2035, 2035′) that are flat, i.e. linear and orthogonal to a central vertical axis of prosthetic valve 2000, in other embodiments the ends of the bodies 2010, 2010′ can be of any other configuration, including angled and/or arcuate, provided that they can be reliably positioned in the native valve so that preferably the entirety of the outlets are below the locations where the native leaflets are sealingly engaged with the bodies 2010, 2010′. The outlets may also have an outflow perimeter that is not planar but rather a scalloped perimeter. For instance, the portion of the outflow perimeter that engages the anterior leaflet AL may extend deeper within the ventricle than the corresponding portion that engages the posterior leaflet PL. As such, the upward surge of blood during systole will first come into contact with this deeper extending portion of the outlet and may ensure better systolic filling of the prosthetic valve 2000.

Each of body 2010, 2010′ can be constructed with materials and techniques similar to known prosthetic heart valves such as those discussed above. In the following description, only body 2010 is describe for simplicity, but all discussion is equally applicable to body 2010′. Body 2010 can have a body frame 2020 formed of wires, struts, mesh, braids, or other suitable constructions, in metal (such as cobalt chrome, stainless steel, shape memory metals such as Nitinol, etc.), polymer, or other suitable material. Body frame 2020 can be formed in a single, unitary piece, or it could be constructed by separate pieces that are joined together, e.g. one piece for each of the inlet portion 2012 and a separate piece for outlet portion 2014. In embodiments in which the body frame 2020 is formed in separate pieces, the pieces could be delivered and implanted separately to make delivery easier, and then coupled together in place in the heart valve. As described in more detail below with reference to specific embodiments, body frame 2020 does not necessarily extend to the outlet portion 2014 of body 2010. For example, a stiff graft (such as Dacron, Teflon etc. with or without coatings) could be used with no frame or with minimal frame.

The construction of bodies 2010, 2010′ could vary. In some embodiments, the portion of the body frame 2020 in the outlet portion 2014 can be configured with a stent frame, with the potential for body covering 2022 and/or body lining 2023 to include, or the addition of, padding (formed of materials such as silicone and pericardium). It may be useful to make the outlet portion 2014 more complaint so that the outlet portion moves with each heartbeat and reduces the wear when leaflet tissues contact the device. Thus, outlet portion 2014 could be constructed similar to the arrangements described above for pseudo-valves, such as the embodiments shown in FIG. 5D, 7A-B, 15A-E, or 18A-B. In such embodiments, the outlet portion can be constructed with a frame that allows the superimposed biologically compatible covering (such as pericardium) to move back and forth with each cardiac cycle. In other embodiments, the outlet portion 2014 could be constructed similar to the occlusion devices shown in FIGS. 13A-B and FIGS. 25A-B, in which the occlusion device is rigid or static, and the native leaflets moves toward the limbs to seal against leak and prevent wear.

In some embodiments, the outlet portion 2014 of prosthetic valve 2000 can be configured to have its shapes be adjustable to improve the seal between the outlet portion and the native leaflets. For example, oval shaped balloons or oval shaped stents could be introduced to shape the body portion limbs after the prosthetic valve 2000 has been placed in the native valve. Such an approach could also be useful if the body covering 2022 and/or body lining 2023 on (or in) an outlet portion 2014 wears out. A new body lining 2023 could be applied from inside the body portion 2014, delivered through the flow passage 2030 on a stent or a frame. This approach would be particularly useful if the body portion 2014 is constructed with a segment in which there is little or no frame material.

Flow control device 2060 is coupled to, and supported by, body frame 2020 in inlet portion 2012, or may optionally form some or all of the inlet portion of the body frame 2020.

Body frame 2020 can be covered on the outside with a body covering 2022 and/or on the inside with a body lining 2023, each of which may be formed from any suitable material that is biocompatible, sufficiently impermeable to fluids such as blood to form the flow passage 2030 and maintain fluid within (or outside of) flow passage 2030, and atraumatic to native valve tissue (leaflets, chordae, heart chamber walls, etc.) that contact the body 2020. Suitable materials can include animal pericardium and synthetic materials such as Dacron (the latter material may be more suitable for covering areas of the body 2020 that do not contact heart tissue as it can be somewhat abrasive).

Body covering 2022 and/or body lining 2023 may cover or line the entirety of body 2020, or may be discontinuous, and cover only portions body 2020. Each may also be attached continuously to each area of body frame 2020 that it covers or lines, but may also be attached around the periphery or margins of selected areas on the body 2020, but not attached within those areas. This construction can allow blood to pass between, for example, struts in the body frame 2020 and expand/balloon out the body covering 2022 and/or body lining 2023 so that it gently contacts the native valve leaflets. The native leaflets would contact against material of body covering 2022 and/or body lining 2023 (for example pericardium) that is backed by blood within flow passage 2030 rather than against a solid portion of body frame 2020. Body frame 2020 could be formed with struts that are widely spaced in the region of contact to ensure the body covering 2022 and/or body lining 2023 will be expanded by blood. This could reduce considerably wear on native leaflet tissues.

As shown in FIG. 52A and FIG. 53A, body 2010 can also include an outlet cuff 2024 at the outflow end of outlet portion 2014 that includes padding material to reduce the risk of injury to cardiac tissue that may contact those portions of the body 120 during the cardiac cycle. Such padding material could be any useful biocompatible material. Silicone, polyurethane, bio-polymers or bio-elastomers, Dacron, PTFE (Teflon) fabrics (often used in rolled or folded form in the sewing cuff of prosthetic valve sewing rings) are suitable options that are commonly used in valve structures. Such padding or damage reducing materials could be added to any part of prosthetic valve 2000.

Although clip CL may be a commercially-available edge-to-edge leaflet clip such as the MitraClip™ or PASCAL, and prosthetic valve 2000 being configured to engaged with such a clip after it has been used to clip the native leaflets, in some embodiments the clip CL may be configured differently than such commercially-available clips (for example any of the clips described above with reference to FIGS. 42D to 42F), and/or may be included as part of a system with prosthetic valve device 2000 and configured to be delivered sequentially or concurrently with prosthetic valve 2000 as part of a total valve repair/replacement procedure. As described above, prosthetic valve 2000 is configured to be anchored to the clip CL, which in turn is coupled to the tissue of the anterior leaflet AL and posterior leaflet PL, and prosthetic valve 2000 will carry a considerable fluid dynamic load during systole that must in turn be carried by the clip and the leaflets. Thus, an enlarged clip anchor or clip structure having wider paddles or multiple paddles may be useful. For example, the clip could be composed of two or three paddles (instead of the single paddle of some of the clip devices) for engagement with each of the one or more leaflets with which the clip is engaged, to increase the area of the leaflet(s) engaged by the clip(s). This allows the dynamic load to be distributed more evenly over a larger leaflet area (and over a greater number of underlying chordae tendineae that are attached to the engaged leaflet portions).

Rather than relying on the clip connector (and thus clip CL, native valve leaflets, or other native valve tissue) to carry all of the fluid dynamic loads imposed on the prosthetic valve, in some embodiments those loads can be carried in part by other structures without putting the clip or the native leaflets in the load path. Thus, in some embodiments prosthetic valve can include an optional annulus connector 2080 and/or an optional heart tissue tether 2090.

As shown in FIGS. 52A to 53B, optional annulus connector 2080 may be part of, or coupled to, body 2010, and is configured to engage with an annulus (and/or other nearby tissue, including the atrial wall, the native leaflets, and/or the chordae tendineae) of a native heart valve to enhance the stability of the prosthetic valve 2000 when placed in the native heart valve, e.g. to inhibit lateral rocking of the prosthetic valve relative to the plane of the native valve, and or displacement away from the annulus towards the atrium (during systole) or the ventricle (during diastole). Annulus connector 2080 may be implement similarly to annulus connectors described above with reference to various embodiments of selective occlusion devices and prosthetic valves. Annulus connector 2080 may be configured with non-tissue penetrating members or with tissue penetrating members. Optional body 2010′ may also have an annulus connector 2080′, or may share the same annulus connector 2080 with body 2010.

As shown in FIGS. 52A to 53B, one or more optional heart tissue tethers 2090 may be coupled to bodies 2010, 2010′, clip connector 2070, clip CL, and/or annulus connectors 2080. 2080′. For ease of illustration, not all options are shown in all of the figures. Heart tissue tethers 2090 and their heart tissue anchors 2092 may be implemented in the same manner and heart tissue tethers 190 and heart tissue anchors 192 described above for prosthetic valve 100 and other embodiments above.

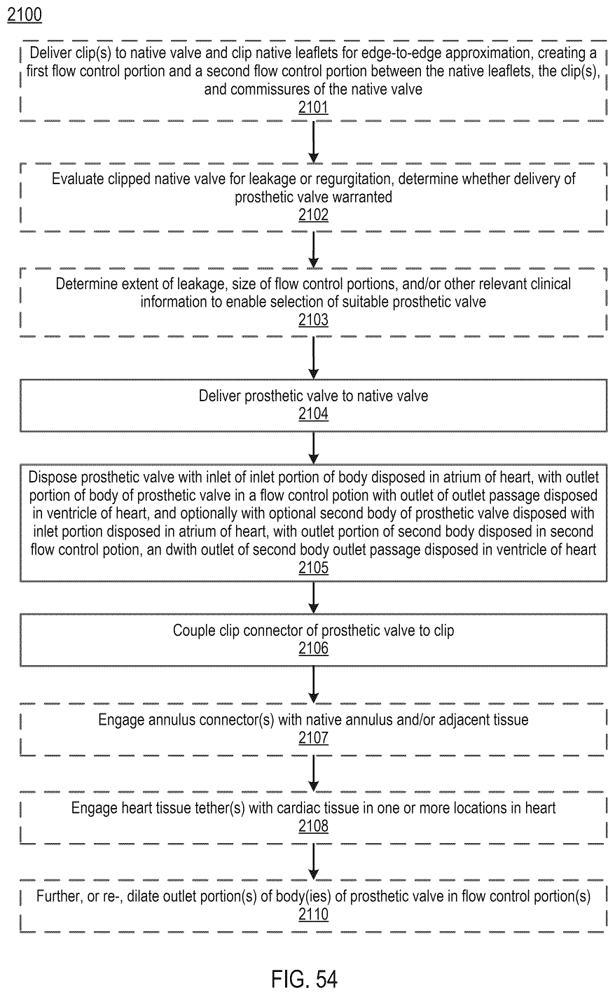

Prosthetic valve 2000 may be delivered to and positioned in the native valve, and secured to the clip CL by the clip connector, to the annulus (and/or nearby tissue) by the annulus connectors, and or to other heart tissue by the heart tissue tether(s) in various methods and sequences. The delivery, positioning, and/or securement may also be performed as part of an integrated procedure with an edge-to-edge approximation procedure with clip(s) CL, sequentially after an edge-to-edge approximation during the same interventional process, or as a separate procedure on a patient who has previously been subjected to an edge-to-edge approximation procedure. Some options are described with reference to the method 2100 shown in the flow chart in FIG. 54. At 2101, one or more clips CL may optionally be delivered to the native valve, and used to clip the native leaflet for edge-to-edge approximation. As described above, this procedure can create two flow control portions, each bounded by the native leaflets, the (or a) clip, and a commissure of the native valve. As noted above, 2101 may have been previously performed in a separate procedure, or may be performed as part of the same procedure as the subsequent portions of method 2100. At 2102, the clipped native valve can be evaluated for leakage or regurgitation, and a determination made as to whether any such leakage or regurgitation was sufficiently severe to warrant usage of prosthetic valve 2000. At 2103, the extent of the leakage or regurgitation, the size of the flow control portions, and/or other relevant clinical information could be determined (e.g. through imaging) to enable selection of a suitable prosthetic valve (e.g. size of the outlet portion(s) 2014, 2014′). At 2104, the prosthetic valve 2000 is delivered to the native valve, such as by known endovascular techniques, using a delivery catheter. At 2105, the prosthetic valve 2000 is disposed in the native valve with the inlet 2031 of flow passage 2030 disposed in the atrium of the heart, with the outlet portion 2014 of body 2010 of prosthetic valve 2000 disposed in the first flow control portion FCP1, with the outlet 2035 of the outlet passage 2034 disposed in the ventricle of the heart. Optionally, for embodiments of prosthetic valve 2000 that include a second body 2010′, prosthetic valve 2000′ may be disposed with the inlet 2031′ of flow passage 2030′ disposed in the atrium of the heart, with the outlet portion 2014′ of body 2010′ disposed in the second flow control portion FCP2, with the outlet 2035′ of the outlet passage 2034′ disposed in the ventricle of the heart. At 2106, clip connector 2170 is coupled to clip(s) CL that are clipped to the native leaflets. In integrated procedures, clip connector 2170 may be coupled to clip(s) CL before clip(s) CL are clipped to the native leaflets for the edge-to-edge approximation.

Optionally, at 2107 annulus connector(s) 2180 may be engaged with the native annulus (on the ventricle side and/or atrial side), and/or adjacent tissue. Although in the flow chart of FIG. 54, 2107 is shown as being after 2106, in some embodiments the annulus connector(s) 2180, 2180′ may be engaged with the native annulus first, i.e., with the prosthetic valve in position in the native valve, and then the clip connector 2170 may be coupled to clip(s) CL. Also optionally, at 2108, one or more heart tissue tether(s) 2190 may be engaged with cardiac tissue in one or more locations in the heart. Further optionally, at the completion of the method 2100, or in a subsequent procedure, if some blood regurgitation is identified, and determined to arise from insufficient seal between the native leaflets in the flow control portion(s) of the native valve and the outlet portion(s) 2014, 2014′ of bodie(s) 2010, 2010′, then at 2110, one or both of the outlet portion(s) 2014, 2014′ may be further, or re-, dilated to reshape or increase the perimeter of the outlet portion(s) and improve the seal with the native leaflets, as described in more detail above.

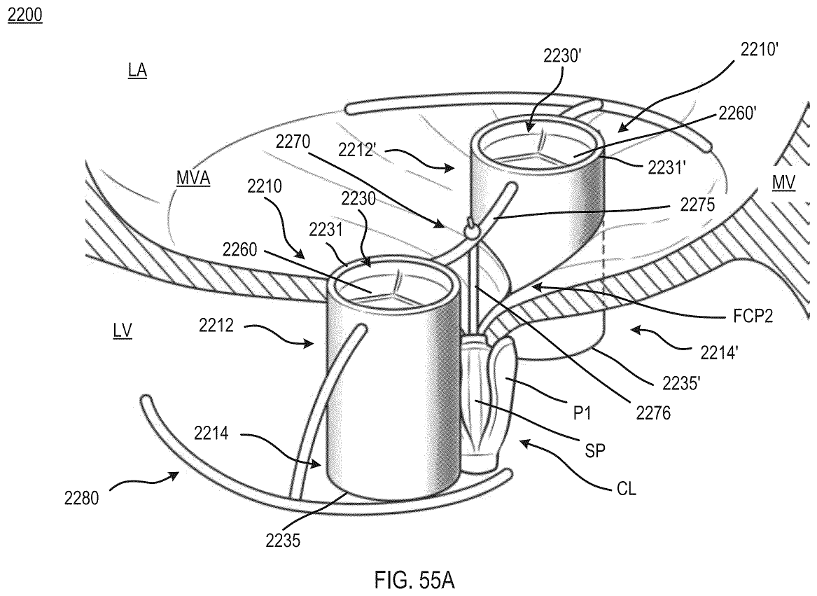

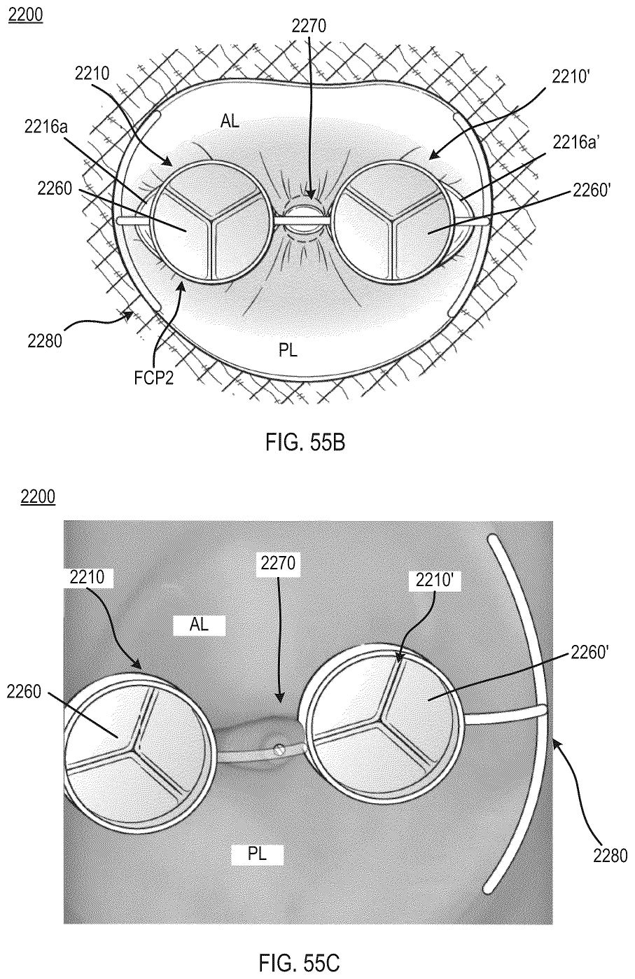

A prosthetic valve according to another embodiment is shown in FIGS. 55A to 55C, shown disposed in a centrally-clipped mitral valve MV. Prosthetic valve 2200 in FIGS. 55A to 55C includes two bodies, 2210 and 2210′, which are shown disposed in two flow control portions, FCP1 and FCP2, of mitral valve MV.

Prosthetic valve 2200 has a first body 2210 with an inlet portion 2212 and outlet portion 2214, and a second body 2210′ with an inlet portion 2212′ and outlet portion 2214′. Body 2210 defines a flow passage 2230 that extends between an inlet 2231 (shown disposed in left atrium LA) and an outlet 2235 (shown disposed in a left ventricle LV), and has a flow control device 2260 disposed therein. Similarly, body 2210′ defines a flow passage 2230′ that extends between an inlet 2231′ (shown disposed in left atrium LA) and an outlet 2235′ (shown disposed in a left ventricle LV), and has a flow control device 2260′ disposed therein. As noted above, prosthetic valve 2200 is disposed in a centrally-clipped mitral valve, with one body 2210, 2210′ disposed in each of flow control portions FCP1 and FCP2. Prosthetic valve 2200 is coupled to clip CL by clip connector 2270, which in this embodiment includes a transverse strut 2275 coupled between body 2210 and 2210′, and a tension member (e.g., suture) 2276 coupled between transverse strut 2275 and spacer SP of clip CL. Prosthetic valve 2200 also includes an annulus connector 2280, coupled to both bodies 2210 and 2210′, and configured similarly to the annulus connectors of several embodiments described above, in this instance engaged with the ventricle side of mitral valve annulus MVA.

FIG. 55B illustrates a variation on the portion of prosthetic valve 2200. The outlet portions of the bodies 2210, 2210′ include leaflet contact areas 2216a, 2216a′ that are non-circular in cross-section, extending laterally towards the commissures of the native mitral valve, which helps to close the roughly triangular portion of the flow control passages FCP1, FCP2 that may not otherwise be filled by the bodies 2210, 2210′. These portions of leaflet contact areas 2216a, 2216a′ may be formed by “padding material” such as Dacron or pericardium to produce the desired shape on the outside of body frame 2220, 2220′.

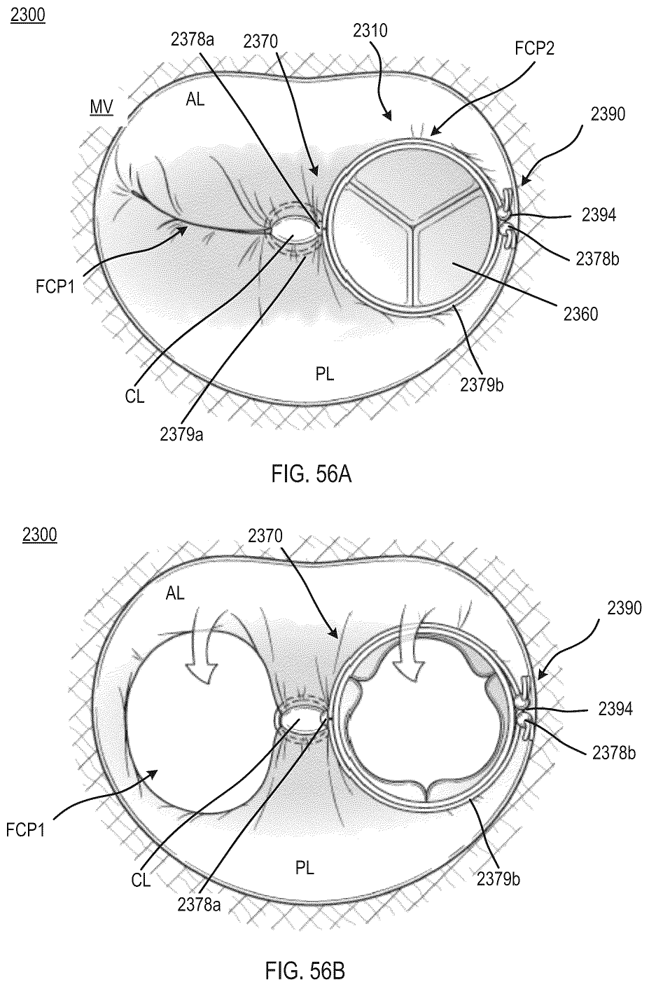

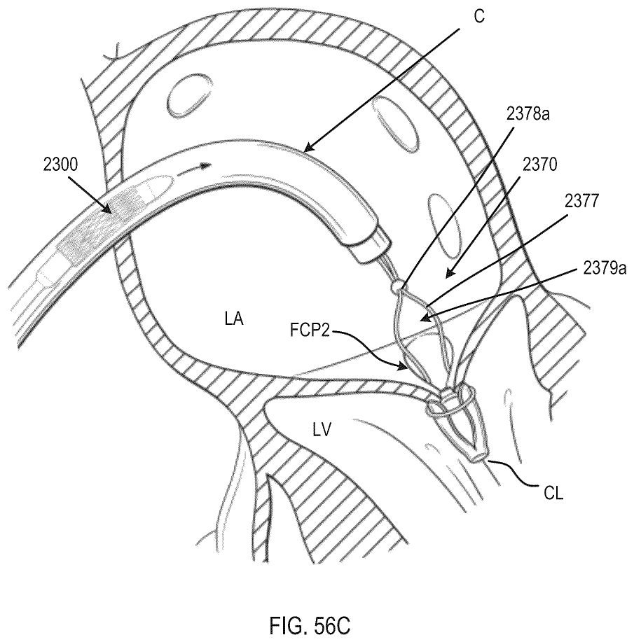

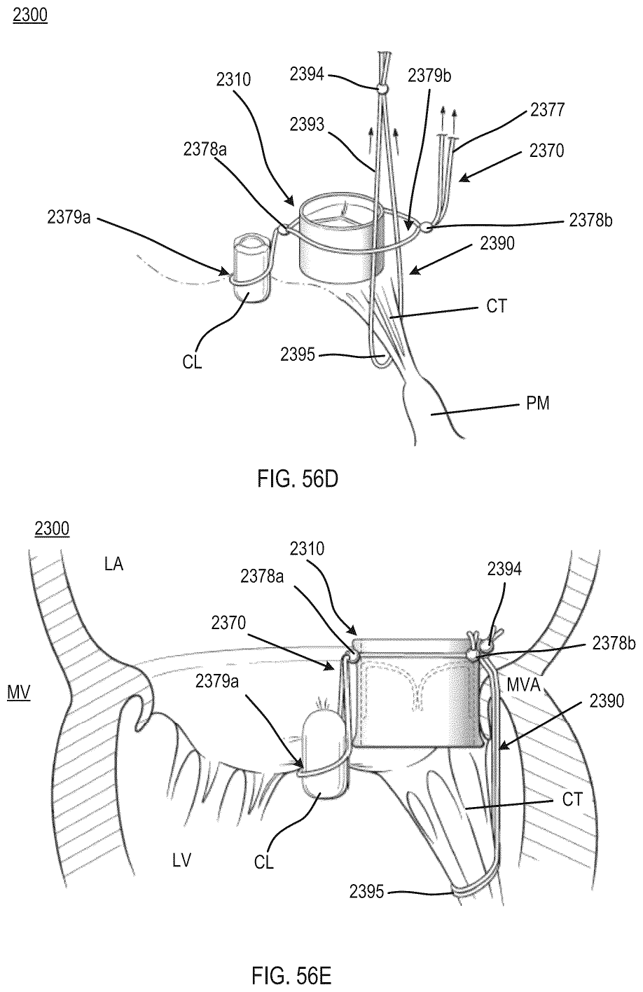

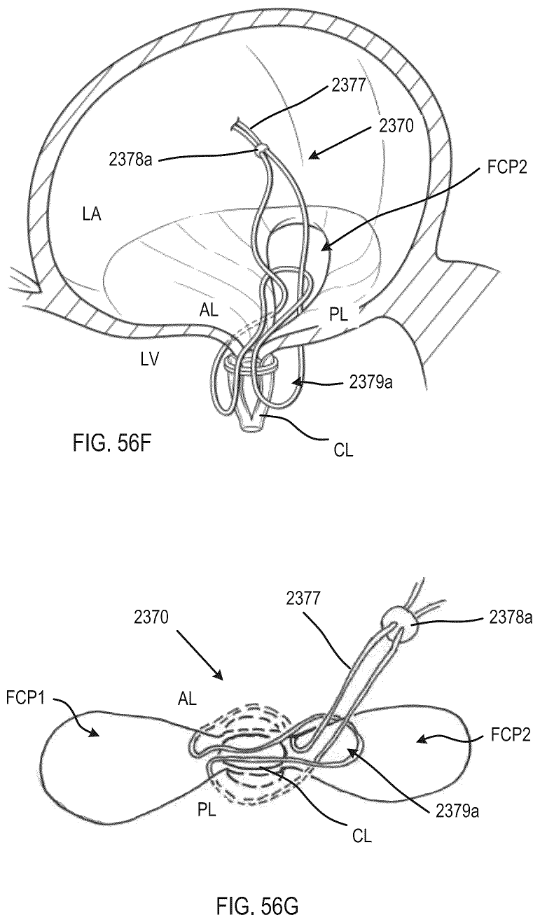

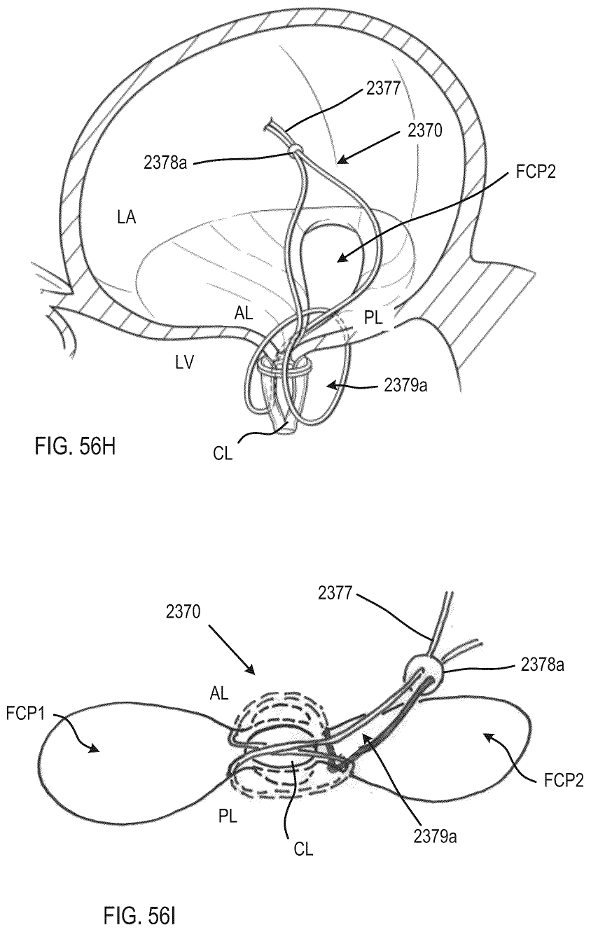

A prosthetic valve according to another embodiment is shown in FIGS. 56A to 56I, shown disposed in a centrally-clipped mitral valve MV. Prosthetic valve 2300 in FIGS. 56A, 56B, 56D, and 56E includes a single body, 2310, which is shown disposed in one of the two flow control portions, FCP1 and FCP2, of mitral valve MV. Such a prosthetic valve and procedure may be useful when only one flow control portion of a centrally-clipped mitral valve (or of clipped tricuspid valve) has unacceptable levels of regurgitation that requires treatment.

Body 2310 of prosthetic valve can be implemented in accordance with any of the options and features disclosed above. The differentiating aspects of this embodiment are the mechanisms for securing prosthetic valve 2300 into operative relationship with the mitral valve MV, using a combination of a suture-based clip connector 2370 and a suture-based heart tissue tether 2390.

Clip connector 2370 is implemented as an elongate suture 2377 with two suture crimps 2378a, 2378b slidably disposed on suture 2377. The free ends of suture 2377 are adjacent, forming a bight between them. A distal (closer to the bight) suture crimp 2378a forms with the bight a distal suture loop 2379a (best seen in FIGS. 56C to 56E). The size (perimeter) of distal loop 2379a is adjustable by sliding the distal suture crimp 2378a toward the bight (preferably the suture crimps are configured to be slidable in one direction, and to resist sliding in the other direction, so that a suture loop can be tightened around a structure, and not release). A proximal (closer to the free ends of suture 2377) suture crimp 2378b forms with the distal suture crimp 2378a a proximal suture loop 2379b. and operative to form two loops in suture 2377 and selectively shorten the length of each loop. As explained in more detail below, clip connector 2370 is configured so that distal suture loop 2379a can be disposed around clip CL and tightened by sliding distal suture crimp 2378a distally (thus securing suture 2377 to clip CL) and proximal suture loop 2379b can be disposed around body 2310 of prosthetic valve 2300 and tightened by sliding proximal suture crimp 2378b distally (thus securing body 2310 to clip CL via suture 2377).

As shown in FIG. 56C, the distal end of a delivery catheter C can be inserted into the left atrium LA (using any suitable technique, e.g. transseptal delivery), and suture 2377 can be delivered out of the delivery lumen of catheter C. Suture 2377 can be delivered in looped form, i.e. by delivering the bight end from catheter C while free ends remain external to the patient's body (e.g. at the leg, for a transfemoral delivery), and the bight end can be manipulated and maneuvered using conventional techniques. Thus, distal suture loop 2379a can be inserted through flow control portion FCP2, into left ventricle LV, then disposed over the ventricle end of clip CL, and the free ends of suture 2377 can be pulled proximally to urge the distal end of distal suture loop 2379a upwardly against the upper (atrial) end of clip CL. Distal suture crimp 2378a can then be slid distally over suture 2377 to tight distal suture loop 2379a. Alternatively, a free end of suture 2377 can be delivered from catheter C, and manipulated and maneuvered until it is in the configuration shown in FIG. 56C, and the free end externalized from the patient so that distal suture crimp can then be applied to the two free ends of the suture 2377 outside the body, and pushed down suture 2377, through catheter C, and into the position shown in FIG. 56C before being slid further down suture 2377 to tighten distal suture loop 2379a.

The same catheter C can then deliver prosthetic valve 2300, as shown in FIG. 56C, into proximal suture loop 2379b (not shown in FIG. 56C). Then, as shown in FIGS. 56D and 56E, body 2310 of prosthetic valve 2300 can be disposed in flow control portion FCP2, with the proximal loop 2379b of suture 2377 disposed around the body 2310 of prosthetic valve 2300. Proximal suture crimp 2378b can be slid distally along suture 2377 to secure proximal suture loop around body 2310, and the free ends of suture 2377 can be clipped off close to proximal suture crimp 2378b-compare FIG. 56D to 56E.

Two alternative techniques for disposing distal suture loop 2379a around clip CL are shown in FIGS. 56F to 56I, contrasted to the technique shown in FIG. 56C. In the technique shown in FIGS. 56F and 56G, distal suture loop 2379a is inserted through flow control portion FCP2, into left ventricle LV, then passed upwardly through the other flow control portion FCP1 into left atrium LA. The free end of suture 2377 can then be passed through distal suture loop 2379a (e.g. external to the patient), and pulled proximally, tightening the bight of suture 2377 around clip CL and the approximated edges of the anterior leaflet AL and posterior leaflet PL. Distal suture crimp 2378a can then be slide distally to tighten distal suture loop 2379a around the clip CL and leaflet tissue. Alternatively, as with the option shown in FIG. 56C, instead of delivering the bight of suture 2377 through catheter C, a free end of suture 2377 can be delivered into the atrium, around the clip, and externalized, establishing the configuration shown in FIGS. 56F and 56G.

Another technique is shown in FIGS. 56H and 56I. In this technique, a free end of suture 2377 is delivered (e.g. by catheter C) into the left atrium LA, through flow control portion FCP1 into left ventricle LV, around the posterior leaflet PL side of clip CL, out of flow control portion FCP2 into the left atrium LA, over clip CL, back through flow control portion FCP1 into left ventricle LV, round the anterior leaflet AL side of clip CL, back out of flow control portion FCP2 into left atrium LA, between suture 2377 and posterior leaflet PL, and back out of left atrium LA, then externalized from the patient. Tension can be applied to the free ends of suture 2377 to tighten the knot around clip CL and the clipped portions of the native leaflets. Distal suture crimp 2378a can then be applied, and pushed down suture 2377 into left atrium LA, forming distal suture loop 2379a.

As noted above, prosthetic valve 2300 includes a heart tissue tether 2390. Since prosthetic valve 2300 is offset laterally from clip CL, the fluid dynamic forces imposed on prosthetic valve 2300 during the cardiac cycle (pushing it strongly toward the left atrium LA during systole and less strongly towards the left ventricle LV during diastole) can impose a rocking force on prosthetic valve 2300, i.e. rotating about clip CL. The upwardly-directed rocking force (created during systole) can be countered by heart tissue tether 2390. Heart tissue tether 2090 is also implemented with a suture 2393 and a suture crimp 2394. As best seen in FIGS. 56C and 56E, a suture loop 2395 of suture 2393 can be disposed around sub-annular tissue, in this instance chordae tendineae of one of the native leaflets extending between papillary muscle PM (the one closest to the flow control portion, or in this instance the posteromedial papillary muscle, which is closes to the illustrated flow control portion FCP2) and the native leaflet. Suture 2393 can be passed through flow control portion FCP2, between valve body 2310 and the mitral valve annulus MVA, e.g. near or in the valve commissure. Suture 2393 can be secured against valve body 2310 by proximal suture loop 2379b, then suture crimp 2394 can be slid distally along suture 2393 to draw valve body 2310 down (towards left ventricle LV and papillary muscle PM). The downwardly directed tension force on valve body 2310 from suture 2393 acts counter to the rocking force produced by blood pressure during systole, thus reducing or eliminating rocking of prosthetic valve 2300 about clip CL.

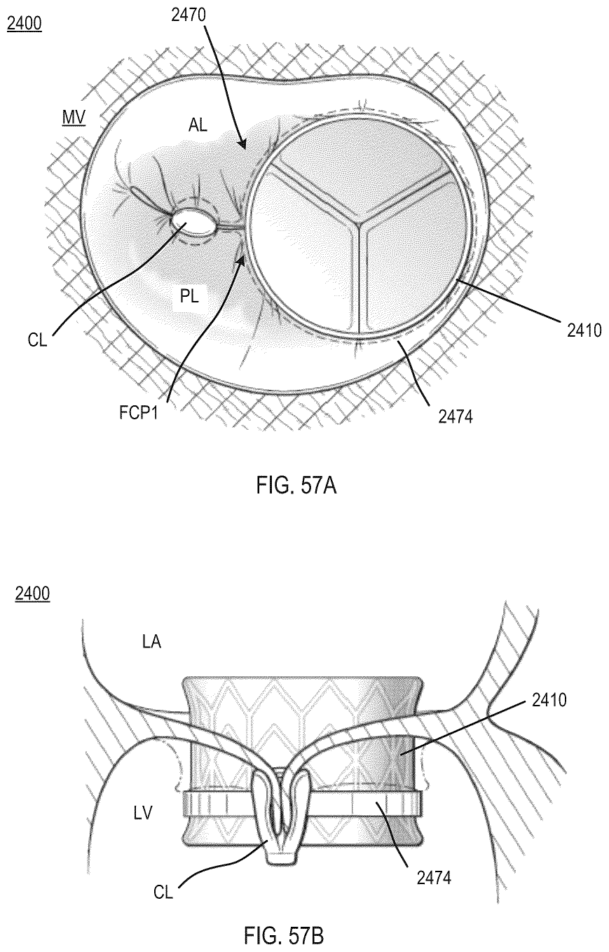

A prosthetic valve according to another embodiment is shown in FIGS. 57A and 57B. Prosthetic valve 2400 is shown disposed in a mitral valve MV in which a clip CL has been applied to anterior leaflet AL and posterior leaflet PL in an eccentric position, i.e. not centered. In this instance, the clip CL has been applied to the A1 and PI cusps. Thus, there is a single large flow control portion FCP1 (or there may be a very small flow control portion (not identified in the figures) between the clip and the nearer commissure). The differentiating aspect of this embodiment is the mechanism for securing prosthetic valve 2400 into operative relationship with the mitral valve MV, using a hoop coupled to clip CL.

Clip connector 2470 is implemented as a clip connector ring or hoop 2474 that is coupled to clip CL. Hoop 2474 may be collapsed or compressed into a constrained configuration rendering it suitable for catheter delivery. Clip connector hoop 2474 can be formed from self-expanding material (such as Nitinol) and can be coupled to clip CL outside the patient's body, and delivered together with clip, such as through a catheter, and disposed on the ventricle side of the anterior leaflet AL and posterior leaflet PL. The clip can be engaged with the leaflets, and the clip connector hoop 2474 can subsequently be released from the delivery catheter. Clip connector hoop 2474 can then self-expand and elastically resume a unconstrained, expanded configuration (as illustrated in FIG. 57B), so that it is disposed below (on the left ventricle LV side of) the native leaflets, concentric with flow control portion FCP1. Prosthetic valve 2400 can then be delivered (e.g. by the same delivery catheter as was used to deliver the clip CL and clip connector hoop 2474) into the left atrium LA, with body 2410 disposed in flow control portion FCP1 and clip connector hoop 2474, and body 2410 can be expanded (or allowed to self-expand) into secure engagement with clip connector hoop 2474, in the configuration shown in FIGS. 57A and 57B.

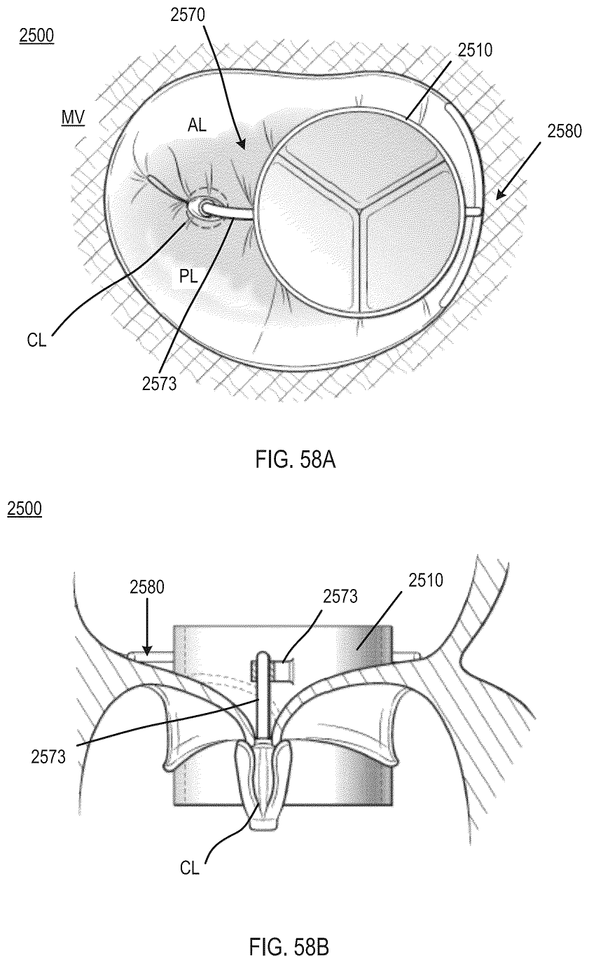

A prosthetic valve according to another embodiment is shown in FIGS. 58A and 58B. Prosthetic valve 2500 is also shown disposed in an eccentrically-clipped mitral valve MV. The differentiating aspect of this embodiment is the mechanism for securing prosthetic valve 2500 into operative relationship with the mitral valve MV, using a strut extending from the clip CL.

Clip connector 2570 is shown with two slight variations in these figures. In FIG. 58A, clip connector 2570 includes a vertically-oriented, U-shaped clip post 2573 extending laterally from the frame of body 2510. The free end of clip post 2573 can be coupled to clip CL with any of the mechanical coupling options describe above. For example, terminal end of clip post 2573 may be inserted into a suitable configured opening 2574 in spacer of clip CL.

In FIG. 58A, clip connector 2570 includes an axial clip post 2573 that extends vertically from clip CL, and is engaged by a strut 2575 that extends laterally from the frame of body 2510.

As shown in FIGS. 58A and 58B, prosthetic valve 2500 includes an annulus connector 2580, similar to that of many of the embodiments described above.

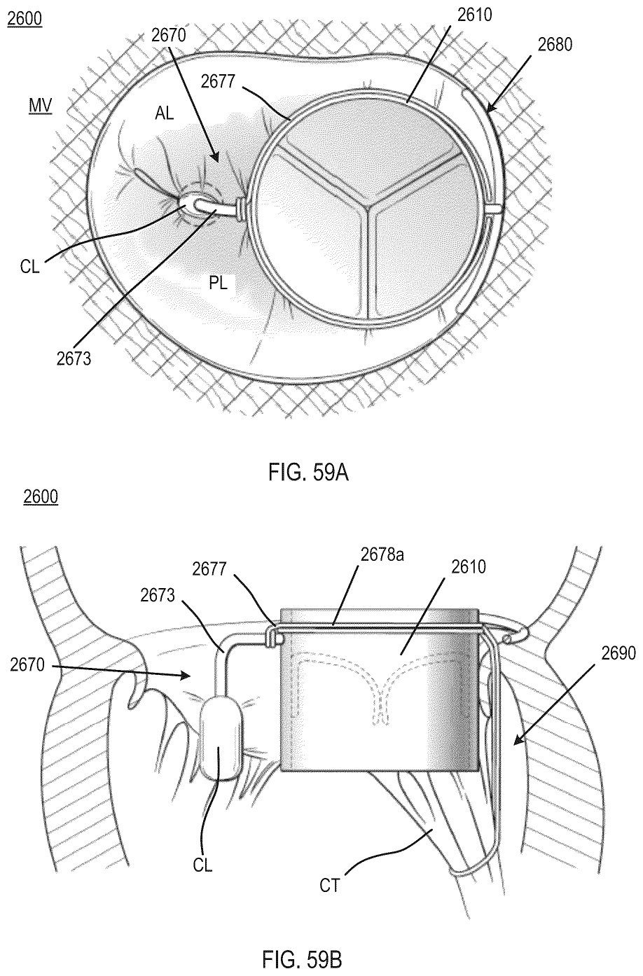

A prosthetic valve according to another embodiment is shown in FIGS. 59A and 59B. Prosthetic valve 2600 is also shown disposed in an eccentrically-clipped mitral valve MV. The differentiating aspect of this embodiment is the mechanism for securing prosthetic valve 2600 into operative relationship with the mitral valve MV, essentially combining features of prosthetic valve 2300 (FIGS. 56A to 56I) and prosthetic valve 2500 (FIGS. 58A and 58B).

Clip connector 2670 includes an L-shaped axial post 2673 extending from clip CL (similar to axial post 2573 of prosthetic valve 2500), and a distal suture loop 2678a that is coupled to post 2673 and secured around body 2610 (similar to suture 2377 of prosthetic valve 2300).

As shown in FIGS. 59A and 59B, prosthetic valve 2600 includes an annulus connector 2680, similar to that of many of the embodiments described above.

Similar to prosthetic valve 2300, prosthetic valve 2600 also includes heart tissue tether 2690 disposable around chordae tendineae CT.

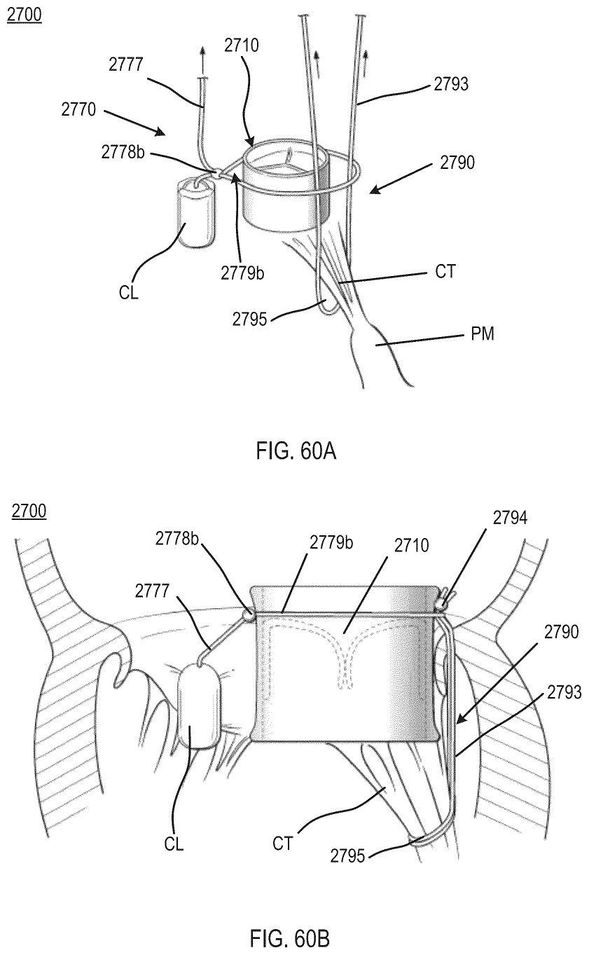

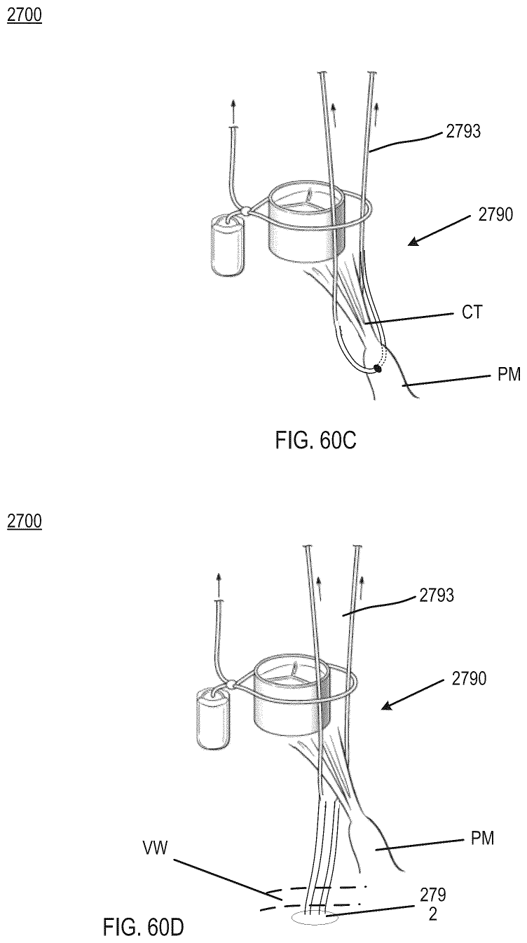

A prosthetic valve according to another embodiment is shown in FIGS. 60A to 60D. Prosthetic valve 2700 is also shown disposed in an eccentrically-clipped mitral valve MV. This embodiment is very similar to prosthetic valve 2300 (FIGS. 56A to 56I), but has a slightly different coupling mechanism for the clip connector 2770.

Similar to clip connector 2370 of prosthetic valve 2300, clip connector 2770 includes elongate suture 2777, but with just one suture crimp, proximal suture crimp 2778b, which forms with the bight of suture 2777 a proximal suture loop 2779b, which is configured to be disposed around body 2710 of prosthetic valve 2700 and tightened by sliding proximal suture crimp 2778b distally (thus securing body 2710 to clip CL via suture 2777). In this embodiment suture 2777 has a single free end, and the other end is fixed to the atrium side of clip CL.

Similar to prosthetic valve 2300, prosthetic valve 2700 also includes heart tissue tether 2790, with a suture 2793, a suture loop 2795 disposable around chordae tendineae CT, and a suture crimp 2794. Two variations on heart tissue tether 2790 are shown in FIGS. 60C and 60D. In the variation in FIG. 60C, heart tissue tether 2790 engages with papillary muscle PM, rather than chordae tendineae CT. Suture 2793 passes through papillary muscle PM (e.g. by piercing papillary muscle PM with a needle coupled to suture 2793 and drawing suture 2793 through). Alternatively, an anchor (screw, hook, ring, etc.—not shown) can be coupled to papillary muscle PM and suture 2793 can be coupled to, or passed through, the anchor. In the variation shown in FIG. 60D, heart tissue tether 2790 includes a tissue anchor 2792, shown schematically as a button or pledget, that can be disposed on an outer (epicardial) side of a wall of the ventricle, VW, e.g. at the ventricle's apex, and the suture 2793 can be secured to the anchor 2792.

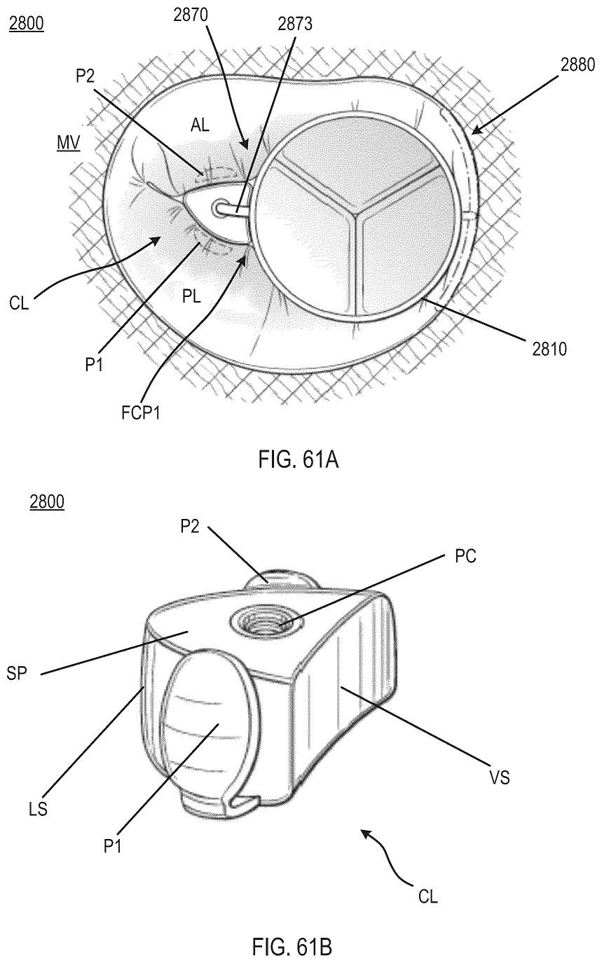

A prosthetic valve according to another embodiment is shown in FIGS. 61A and 61B. Prosthetic valve 2800 is also shown disposed in an eccentrically-clipped mitral valve MV. The differentiating aspect of this embodiment is a clip that combines clipping, spacing, and occluding functions, enabling a larger flow control portion and thus a larger flow control device, with effective sealing against paravalvular leakage.

As shown in FIG. 61A, body 2810 of prosthetic valve 2800 is disposed in a flow control passage FCP1 created by clipping the posterior leaflet PL and anterior leaflet AL eccentrically (i.e. not centrally, in this instance by clipping the A1 and P1 cusps). Prosthetic valve 2800 is similar to other prosthetic valves disclosed above, such as prosthetic valve 2500 shown in FIGS. 58A and 58B, and similarly includes as part of clip connector 2870 an axial clip post 2873 similar to post 2573 of prosthetic valve 2500.

The native leaflets are clipped with clip CL, shown in more detail in FIG. 61B. Clip CL includes a spacer SP, first paddle P1, second paddle P2, and a post connector PC to which axial clip post 2873 can be secured by any suitable mechanism (as described above in more detail). As shown in FIG. 61A, anterior leaflet is secured to clip CL between paddle P2 and spacer SP, and posterior leaflet PL is secured to clip CL between paddle P1 and spacer SP. As is apparent from FIG. 61A, spacer SP has a significant width between paddles P1 and P2, such that when the native leaflets are secured to clip CL, their coapting edges are separated, rather than being close together as is the case with clips such as the MitraClip™. This spaced clipping creates a larger (longer perimeter, greater flow area) flow control portion FCP1 than if the edges of the leaflets AL, PL were clipped directly together. In turn, this enables placement of a larger diameter prosthetic valve body 2810, with a larger flow area. The edges of leaflets AL and PL can sealingly engage the V-shaped (from a top view) leaflet surface LS of spacer SP, and the side of valve body 2810 can sealingly engage valve surface VS of clip CL. Spacer SP essentially fills the triangular space between the leaflets AL, PL, the commissure (anterolateral commissure ALC), and the prosthetic valve 2800, thus also functioning as an occluder. The clipped margins of the anterior leaflet AL and posterior leaflet PL are maintained in a fixed spatial relationship relative to each other throughout the cardiac cycle. There is no blood flow through the occluder, and in between the clipped leaflet margins during any phase of the cardiac cycle. Thus, paravalvular leakage, or regurgitation, of blood between the atrium and ventricle is reduced or eliminated.

Prosthetic valve 2800 also includes an annulus connector 2880, which in this embodiment is disposed below the native annulus, resisting upwardly (towards the atrium) directed fluid dynamic forces, e.g. during systole. The differentiating aspect of this embodiment is the clip with spacer-occluder structure and function. The mechanism for securing prosthetic valve 2600 into operative relationship with the mitral valve MV, essentially combining features of prosthetic valve 2300 (FIGS. 56A to 56I) and prosthetic valve 2500 (FIGS. 58A and 58B) . . .

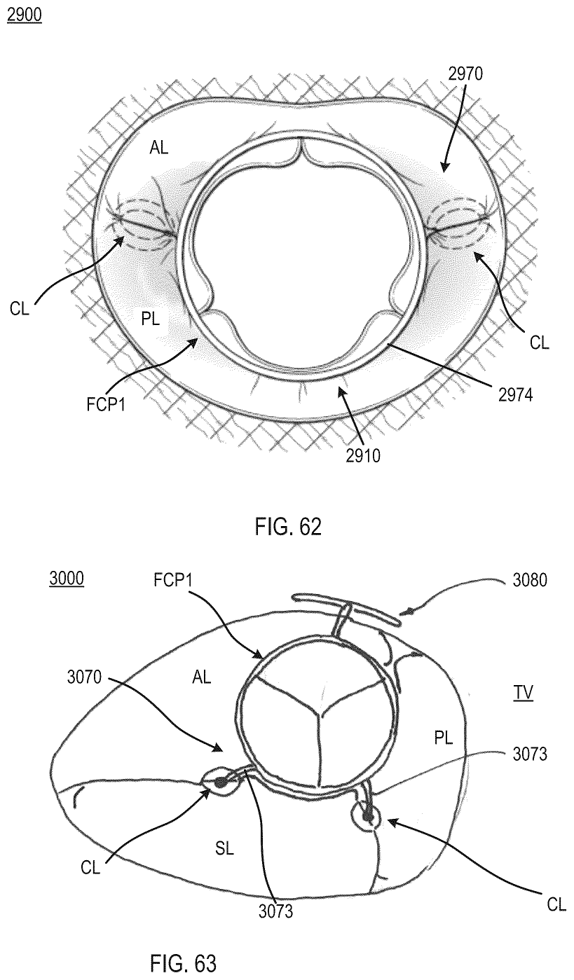

A prosthetic valve according to another embodiment is shown in FIG. 62. Prosthetic valve 2900 is shown disposed in a mitral valve that has been clipped with two spaced clips CL, creating therebetween a single, large flow control portion FCP1. Body 2910 of prosthetic valve 2900 can be secured to at least one of the clips CL, and preferably to both of the clips CL, using any of the structures and techniques described above for other embodiments. For example, as shown in FIG. 62, prosthetic valve 2900 includes a clip connector 2970 that includes a hoop 2974, similar to the hoop 2474 described above for prosthetic valve 2400 (FIGS. 57A and 57B). Hoop 2974 is preferably coupled to both clips CL thereby preventing rocking of the prosthetic valve 2900. Alternatively, clip connector 2970 could be implemented with a suture loop, such as described above for prosthetic valves 2300 (FIGS. 56A-56I), 2600 (FIGS. 59A and 59B), or 2700 (FIGS. 60A and 60B). Securing body 2910 to appropriately sized hoop 2974 in this manner prevents over-stressing or over-tensioning of the free margin lengths of anterior leaflet AL and posterior leaflet PL that are delimited between the spaced apart clips CL.

As described above, the many embodiments of prosthetic valves described above can be used to address regurgitation in mitral valves or tricuspid valves. FIGS. 63 to 66 illustrate some exemplary applications to tricuspid valves.

As shown in FIG. 63, a tricuspid valve TV has been clipped with two clips CL in a triple orifice technique (as described above with reference to FIGS. 38E and 38F, which creates three two control portions, FCP1 (the largest) and FCP2 and FCP3 (smaller)). FIG. 63 illustrates a prosthetic valve 3000 disposed in flow control portion FCP1. In FIG. 63, the heart is in systole, such that all leaflets (including the leaflets in the prosthetic valve) are in the closed position. Prosthetic valve 3000 is shown with an annulus connector 3080 engaged with an atrium side of the tricuspid valve annulus, but in other variations the (or another) annulus connector 3080 could engage the ventricle side of the tricuspid valve annulus, and/or the prosthetic valve 3000 could include a heart tissue tether or any of the other mechanisms described above in addition to clip connectors to secure the prosthetic valve in position relative to the native valve. In addition, prosthetic valve 3000 includes a clip connector 3070, which is illustrated with axial clip posts 3073 connected to the two clips CL. However, in other variations any of the clip connector embodiments described above could be used to secure prosthetic valve 3000 to clips CL.

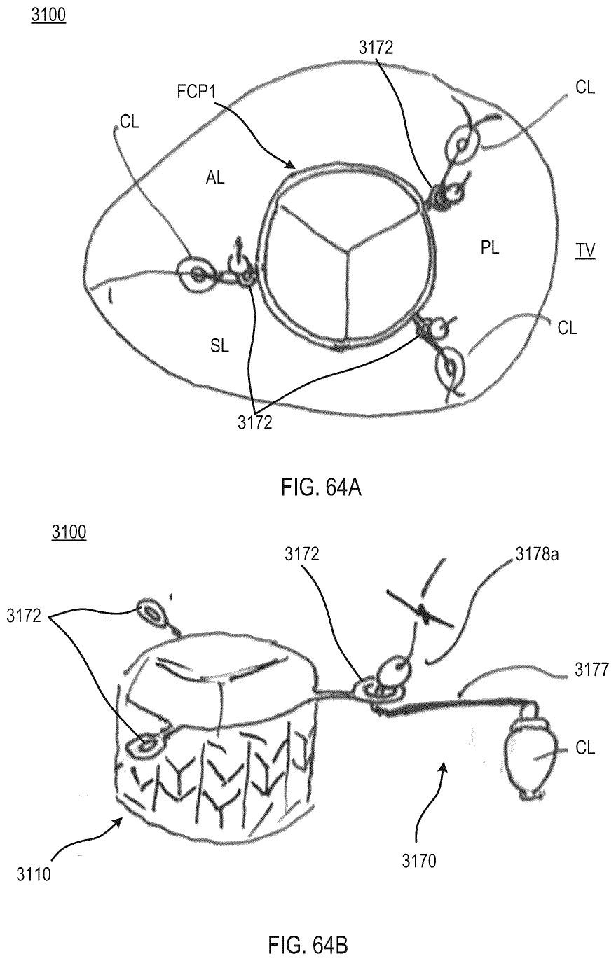

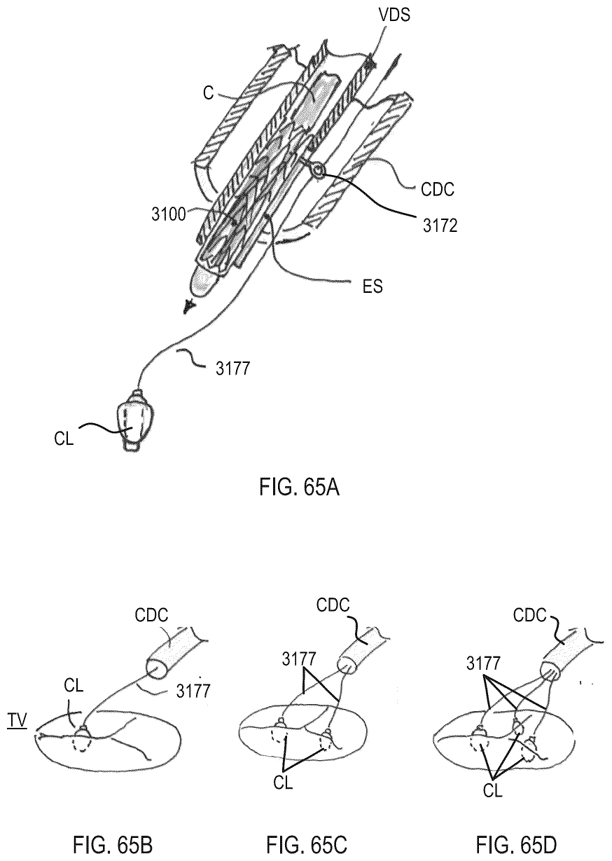

As shown in FIG. 64A, a tricuspid valve TV has been clipped with three clips CL in a modified triple orifice technique that produces a larger, more central flow control portion FCP1. FIG. 64 illustrates a prosthetic valve 3100 disposed in flow control portion FCP1. In FIG. 64, the heart is in systole, such that all leaflets (including the leaflets in the prosthetic valve) are in the closed position. Prosthetic valve 3100 is shown with a clip connector 3170 that includes three eyelets 3172 that project radially from body 3110, which can be engaged with sutures 3177, each of extends from a respective clip CL and which has a length from clip CL to respective eyelet 3172 by a distal suture crimp 3178a (most easily seen in FIG. 64B). Suture 3177 can conveniently be the guide wire over which each clip CL is delivered to tricuspid valve TV. The delivery process for the clips CL and the prosthetic valve 3100 is illustrated in FIGS. 65A to 65D.

As shown in FIG. 65A, a delivery system for clips CL and prosthetic valve 3100 includes a catheter C supporting prosthetic valve 3100 for delivery through valve delivery sheath VDS. Valve delivery sheath VDS includes eyelet slots ES through which eyelets 3172 can project radially. Valve delivery sheath VDS is disposed in a lumen of clip delivery cannula CDC, through which clips CL can be delivered. Each clip CL has a delivery guidewire to which it is coupled, which in this embodiment is suture 3177 of clip connector 3170. The sutures 3177 are threaded through eyelets 3172, and clips CL are disposed at the distal end of sutures 3177, distal to eyelets 3172. Each of the three clips CL can be delivered to tricuspid valve in sequence, as shown in FIGS. 65B to 65D, each clipping an adjacent pair of leaflets (as shown in FIGS. 65B to 65D, by way of example only, the first clip CL clips anterior leaflet AL to septal leaflet SL, the second clip CL clips septal leaflet SL to posterior leaflet PL, and the third clip CL clips anterior leaflet AL to posterior leaflet AL), resulting in the clipped tricuspid valve shown in FIG. 64A. Prosthetic valve 3100 can then be delivered from valve delivery sheath VDS out of clip delivery cannula CDC, and positioned in flow control portion FCP1. The proximal end of each of suture 3177 can then be tensioned (e.g. from outside of the patient's body) and distal suture crimps 3178a pushed over sutures 3177 and against eyelets 3172, thus securing prosthetic valve 3100 to clips CL. Suture 3177 can then be clipped or cut close to distal suture crimps 3178a, and the delivery system withdrawn from the patient.

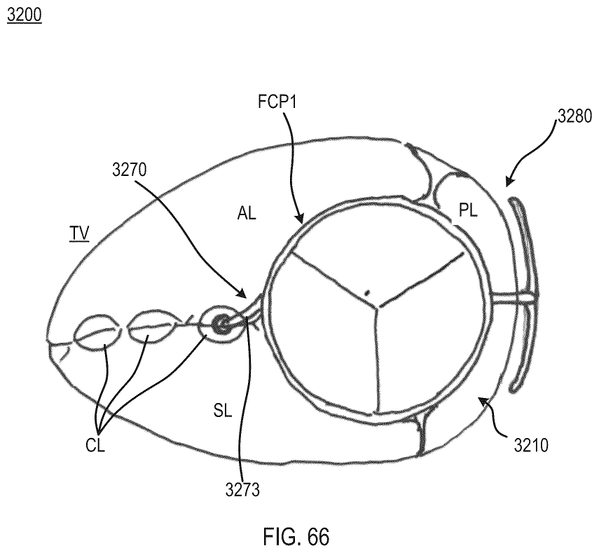

As shown in FIG. 66, a tricuspid valve TV has been clipped with three clips CL in a “bicuspidization” clipping technique (as described above with reference to FIGS. 38C and 38D), which creates a single control portion FCP1. FIG. 66 illustrates a prosthetic valve 3200 disposed in flow control portion FCP1. In FIG. 66, the heart is in systole, such that all leaflets (including the leaflets in the prosthetic valve) are in the closed position. Prosthetic valve 3000 is shown with an annulus connector 3280 engaged with an atrium side of the tricuspid valve annulus, but in other variations the (or another) annulus connector 3280 could engage the ventricle side of the tricuspid valve annulus, and/or the prosthetic valve 3200 could include a heart tissue tether or any of the other mechanisms described above in addition to clip connectors to secure the prosthetic valve in position relative to the native valve. In addition, prosthetic valve 3200 includes a clip connector 3270, which is illustrated with an axial clip post 3273 connected to the clip CL closest to valve body 3210. However, in other variations any of the clip connector embodiments described above could be used to secure prosthetic valve 3200 to one or more of the clips CL.

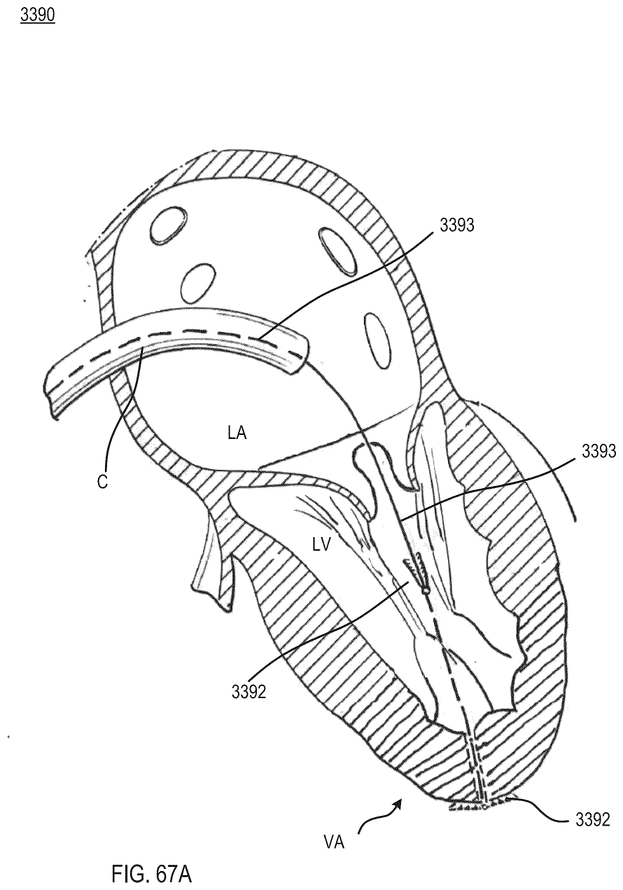

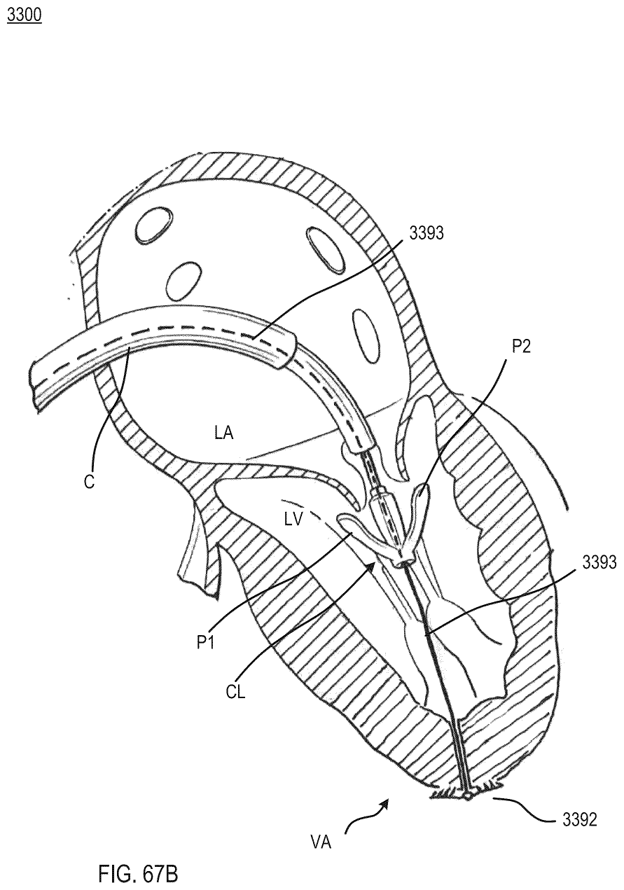

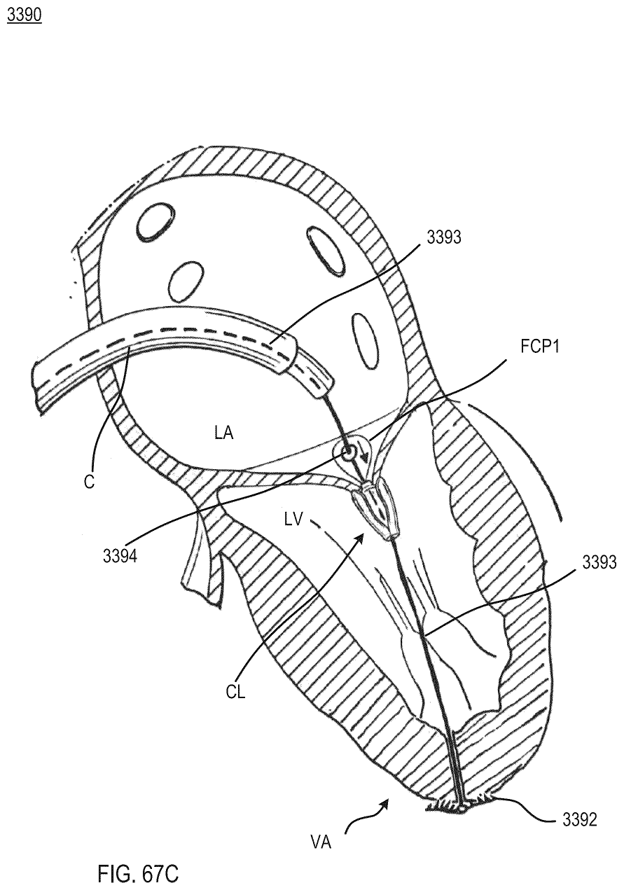

As discussed above, any of the prosthetic valves embodiments described herein can include a heart tissue tether that can be between the prosthetic valve and heart tissue, such as on the ventricle side of the native atrioventricular valve, which can provide a tension force that opposes the fluid dynamic forces imposed on the prosthetic valve during systole that would tend to displace the prosthetic valve towards the atrium and/or rock the prosthetic valve with respect to the plane of the native valve. As noted in the description of prosthetic valves 100 and 2000, such heart tissue tethers can be coupled to the clip connector and/or clip (among other options). FIGS. 67A to 67C illustrate a heart tissue tether 3390 that can be coupled between a clip CL and the ventricular apex VA of the heart, and a method for delivering the heart tissue tether 3390 and clip CL to the heart. As shown in FIG. 67A, heart tissue tether 3390 can be delivered into the left ventricle LV by a catheter C. Heart tissue tether 3390 includes a tether anchor 3392 and a suture 3393, which can serve as a guidewire during delivery of heart tissue tether 3390. In FIG. 67A, tether anchor 3392 is shown in two positions-a first position near the middle of left ventricle LV during delivery, in a delivery (closed or collapsed) configuration, disposed at the distal end of suture 3393, and a second position disposed on the epicardial surface of the ventricle apex VA, in an deployed (expanded) configuration, after passing through a puncture though ventricle apex VA, disposed at the distal end of suture 3393 (shown in dashed line for the delivered position).

In FIG. 67B, clip CL is shown in left ventricle LV after being delivered from catheter C, riding over suture 3393, which passes through a lumen in clip C, and functions as a guidewire for delivery of clip CL. Suture 3393 is not under tension, thus allowing full manipulation, positioning, and orientation of clip CL by its delivery catheter, including closing of paddles P1 and P2 to engage the native leaflets.

In FIG. 67C, clip C is shown fully deployed, i.e. having clipped together the native leaflets. The free end of suture 3393 can be tensioned, and suture crimp 3394 pushed distally over suture 3393, against clip CL, and then secured to suture 3393 to fix the length of suture 3393 between ventricle apex VA and clip CL, and to provide desired tension on clip CL. At this point in the procedure, suture 3393 can be clipped or cut proximal to suture crimp 3394, and the remainder of suture 3393 withdrawn. Any of the prosthetic valves described above can then be delivered to the native valve (e.g. through catheter C) and secured to clip CL with a suitable clip connector. Heart tissue tether 3390 then serves to oppose fluid dynamic forces imposed on the prosthetic valve.

Additional embodiments of selective occlusion devices are described below. In these embodiments, a separate occluder (whether a static occluder or a movable membrane occluder (or pseudo valve) or a prosthetic valve occluder) is used to control the flow through each flow control portion of a clipped native valve. Thus, for a clipped native valve with two flow control portions through which it is desired to control flow with a selective occlusion device (rather than relying only on the function of the clipped native leaflets founding the flow control portion), a selective occlusion device includes two flow occluders. For a clipped native valve with a single flow control portion, or with multiple flow control portions but for which it is necessary or desirable to address regurgitation through only one of the flow control portions, the selective occlusion device includes a single occluder. Other structures and functions described for the selective occlusion device embodiments, and in some instances the prosthetic valve occluder embodiments, above are also applicable to the embodiments described below, and additional structures or functions are included in the embodiments described below, as will be made clear from the following description. In general, the same reference numbering scheme is used for the preceding and following embodiments, for ease of reference, and unless otherwise apparent from the detailed description below, any structure in the following embodiments that corresponds to structure in the embodiments above can include all of the same details of design and implementation, and all of the same options and alternatives, as described above.

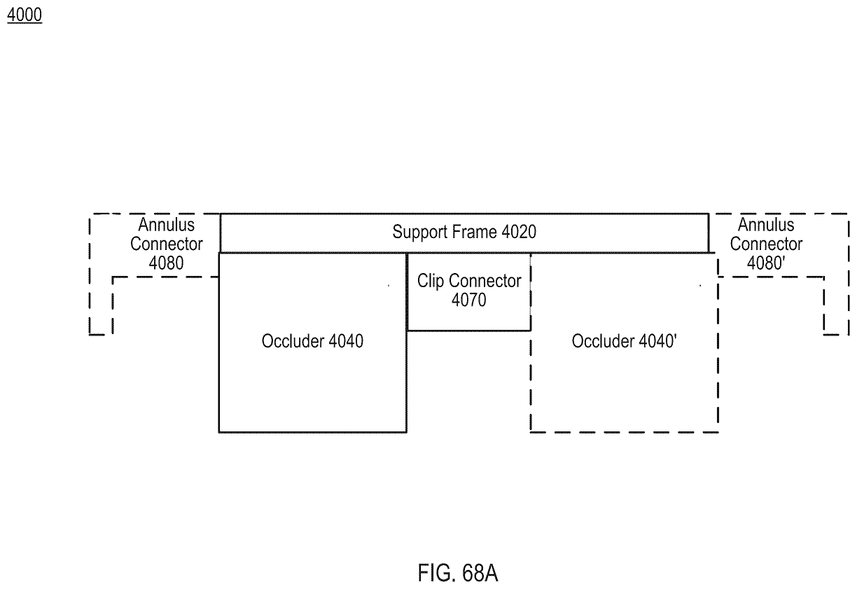

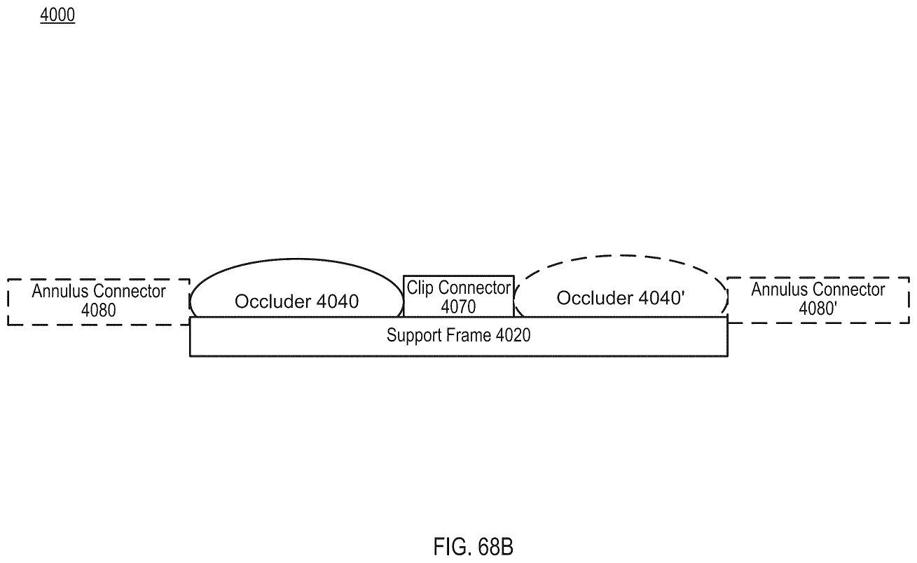

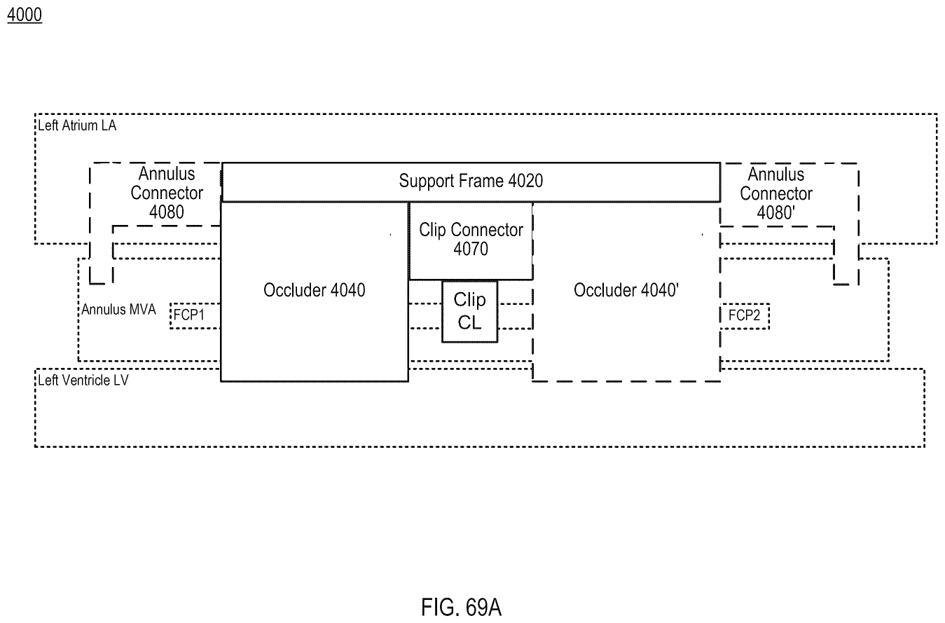

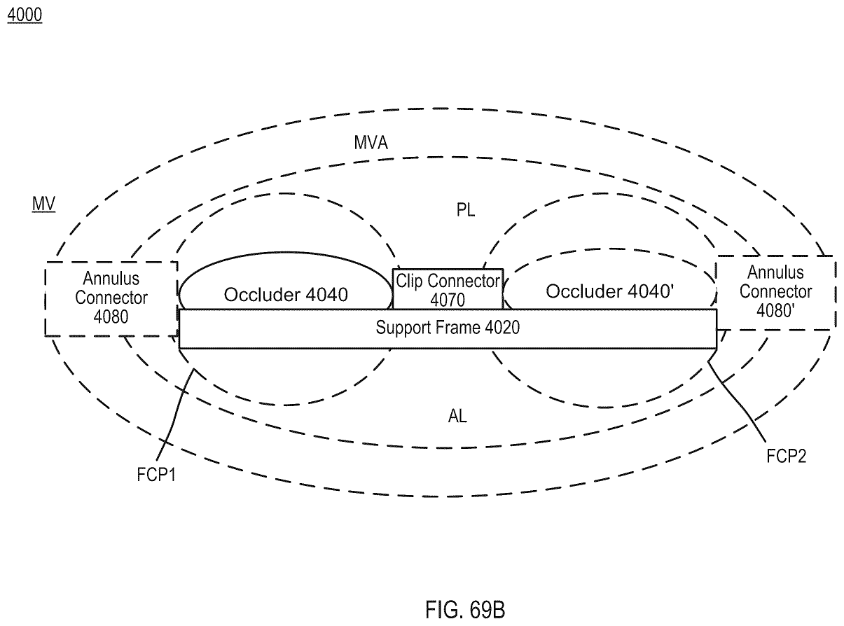

An embodiment of a selective occlusion device (or “SOD”) 4000 is illustrated schematically in a side view and top view, respectively, in FIGS. 68A and 68B. SOD 4000 includes a support frame 4020 and an occluder 4040. Occluder 4040 can be constructed, and function, similar to any of the occluders described above for other embodiments. As shown schematically in FIGS. 68A to 69B, occluder 4040 may be configured and appropriately sized to at least occupy the area of regurgitation between native leaflets during systole in a clipped atrioventricular valve. As described above in connection with other SOD embodiments, occluder 4040 may be implemented in some embodiments as a static structure, i.e., it need not flex inwardly or outwardly to engage and disengage the native leaflets of the mitral valve MV or tricuspid valve TV during the systole and diastole portions of the heart cycle. Instead, such static occluders may retain their shape and be sized and located in the native valve such that the native leaflets engage the occluder 4040 during systole and disengage the occluder 4040 during diastole. In other embodiments, occluder 4040 may be implemented with one or more flexible membranes, which act as a pseudo-valve by moving in coordination with the leaflets of the native valve, as described above in more detail. In other embodiments, occluder 4040 may be implemented as a prosthetic valve occluder.

SOD 4000 also includes a clip connector 4070 that may be part of, or coupled to, support frame 4020 and/or occluder 4040, and is configured to engage with a clip such as those described above, and thereby to retain SOD 4000 in operative relationship with a native heart valve to which the clip is attached. In particular, clip connector 4070 is configured to carry fluid dynamic load applied to SOD 4000 during the cardiac cycle of the heart to the clip CL and thence to the native leaflets, annulus, and surrounding heart tissue to resist displacement of the SOD. The largest loads to be carried tend to be during systole, and the displacement to be resisted is towards the atrium of the heart.

Clip connector can be implemented in a variety of configurations, including those described above, as well as additional variations described in more detail below. As described above, clip CL can be any of the commercially available designs, or can be customized or modified specifically for use with SOD 4000.

As shown schematically in FIGS. 68A to 69B, SOD 4000 may include an optional annulus connector 4080, which is configured to be coupled to occluder 4040 and an annulus of a native valve. Further, SOD 4000 may include a second occluder 4040′, which can also be coupled to the support frame 4020 and, optionally, clip connector 4070, and may also have an optional annulus connector 4080′ (or be coupled to the same annulus connector 4080). An SOD 4000 with both occluder 4040 and 4040′ may be used to control blood flow in a clipped native valve having two flow control portions needing regurgitation reduction-occluder 4040 can be disposed in a first flow control portion FCP1 and occluder 4040′ can be disposed in a second flow control portion FCP2, as shown in FIGS. 69A and 69B.

SOD 4000 is shown disposed in a native heart valve in a side view and top view, respectively, in FIGS. 69A and 69B. Note that for convenience of illustration the native heart valve is illustrated as a mitral valve MV. Note also that SOD 4000 is illustrated with the optional second occluder 4040′ disposed in one of the two flow control portions of clipped mitral valve MV. Mitral valve MV is shown with the anterior leaflet AL and posterior leaflet PL joined by a clip CL in an edge-to-edge approximation, similar to the mitral valve MV shown in FIG. 37B. Thus, mitral valve MV has two flow control portions-FCP1 and FCP2—each portion defined between the clip, the leaflets, and one of the commissures of the mitral valve MV. As discussed above with reference to FIGS. 37A to 38F, there are many possible arrangements of clips on either the mitral valve or tricuspid valve, creating one, two, or three flow control portions-SOD 4000 may be used with any of those clipped valve configurations to address regurgitation in one or two of the flow control portions.

As shown in FIGS. 69A and 69B, SOD 4000 can be disposed in mitral valve MV with a portion disposed in the left atrium LA and a portion disposed in the left ventricle LV. Clip connector 4070 is shown engaged with clip CL. Optional annulus connectors 4080 and 4080′ can be engaged with mitral valve annulus MVA. When SOD 4000 is disposed in mitral valve MV, it is operable to reduce or eliminate regurgitation through flow control portions FCP1 and/or FCP2, i.e., to prevent retrograde flow of blood from the left ventricle to the left atrium during systole, but to allow blood to flow freely from left atrium LA to left ventricle LV during diastole.

Each of occluders 4040 and 4040′ may be configured so that outer surfaces thereof engage in a substantially sealing relationship with the anterior leaflet AL and/or the posterior leaflet PL during systole and thereby to reduce or prevent undesired retrograde flow (regurgitation) of blood therebetween from the left ventricle LV to the left atrium LA.

The occluders 4040, 4040′ of SOD 4000 are shown schematically in FIGS. 68B and 69B as being oval in cross section, roughly corresponding to the shape of the flow control portions of the native valve that result from leaflet clipping, e.g., oval or slit like, as shown in FIG. 69B for ease of illustration. In some embodiments, the cross-sectional shape of the occluders could be more elliptical, or with a teardrop (more V-shaped) shape, with the narrower portion oriented toward the commissures, or other suitable shape that occupies at least the area of regurgitation between the native leaflets during systole in a clipped mitral valve.

Each of occluder 4040, 4040′ can be constructed with materials and techniques similar to the other occluders discussed above.

Although clip CL may be a commercially-available edge-to-edge leaflet clip such as the MitraClip™ or PASCAL, and SOD 4000 being configured to engage with such a clip after it has been used to clip the native leaflets, in some embodiments the clip CL may be configured differently than such commercially-available clips (for example any of the clips described above with reference to FIGS. 42D to 42F), and/or may be included as part of a system with SOD 4000 and configured to be delivered sequentially or concurrently with SOD 4000 as part of a total valve repair/replacement procedure. As described herein, SOD 4000 is configured to be anchored to one or more clips CL, which in turn is/are coupled to the tissue of the anterior leaflet AL and/or posterior leaflet PL, and SOD 4000 will carry a considerable fluid dynamic load during systole that must in turn be carried by the clip(s) and the leaflets, and optionally, the annulus connector 4080 if provided. Thus, an enlarged clip anchor or clip structure having wider paddles or multiple paddles may be useful. For example, the clip could be composed of two or three paddles (instead of the single paddle of some of the clip devices) for engagement with each of the one or more leaflets with which the clip is engaged, to increase the area of the leaflet(s) engaged by the clip(s). This allows the dynamic load to be distributed more evenly over a larger leaflet area (and over a greater number of underlying chordae tendineae that are attached to the engaged leaflet portions).

Rather than relying on the clip connector (and thus clip(s) CL, native valve leaflets, or other native valve tissue) to carry all of the fluid dynamic loads imposed on the SOD, in some embodiments those loads can be carried in part by other structures without putting the clip(s) CL or the native leaflets in the load path. Thus, in some embodiments SOD 4000 can include an optional annulus connector 4080 and/or an optional heart tissue tether (not shown but similar to the embodiments of prosthetic valves described above). Optionally, annulus connector 4080 can be configured to cooperate with the clip CL to carry some of the dynamic load (especially if fixedly connected with the Annulus MVA or with subvalvular heart tissue located below the valve annulus on the ventricle side). Additionally, the annulus connector 4080 may serve to maintain the SOD in a fixed spatial relationship relative to the flow axis of the native valve during the different phases of the cardiac cycle.

As shown in FIGS. 68A to 69B, optional annulus connector 4080 may be part of, or coupled to, support frame 4020, and is configured to engage with an annulus (and/or other nearby tissue, including the atrial wall, the native leaflets, and/or the chordae tendineae) of a native heart valve to enhance the stability of the SOD 4000 when placed in the native heart valve, e.g. to inhibit lateral rocking or tilting of the SOD relative to the plane of the native valve, and or displacement away from the annulus towards the atrium (during systole) or the ventricle (during diastole). Annulus connector 4080 may be implemented similarly to annulus connectors described above with reference to various embodiments of selective occlusion devices and prosthetic valves. Annulus connector 4080 may be configured with non-tissue penetrating members or with tissue penetrating members. Optional occluder 4040′ may also have an annulus connector 4080′, or may share the same annulus connector 4080 with occluder 4040. SOD 4000 may include a third annulus connector (not shown for this embodiment, but shown in another embodiment described below).

While two flow control regions FCP1 and FCP2 are shown in FIGS. 69A and 69B, it is noted that in some embodiments, a clip CL may be placed such that a single flow control region is formed. For such embodiments, a single occluder may be used. For example, 70 shows a SOD 4100, including a single occluder 4140 coupled to clip CL. SOD 4100 may be placed into a native valve (for illustration, a mitral valve), and anterior leaflet AL and posterior leaflet PL may be approximated with single eccentric clip CL, resulting in single flow control portion FCP1. Single occluder 4140, may be configured to be a pseudo-valve, having a flexible membrane 4144 supported on support frame 4120 at frame attachment portion 4144c with any of the techniques described above for other occlusion devices. Flexible membrane 4144 has first leaflet engaging portion 4144a configured to selectively engage with anterior leaflet AL and second leaflet engaging portion 4144b configured to selectively engage with posterior leaflet PL. Support frame 4120 has clip connector 4170, shown as a simple post, at one end that engages clip CL. Other end of support frame has annulus connector 4180 extending from it to engage mitral valve annulus MVA (similar to any embodiments above with annulus connectors).

FIG. 71 shows an SOD 4200 which also includes a single occluder 4240. SOD 4200 may be similar to SOD 4100, except that single occluder 4240, is a static occluder, which may be similar to occluders shown in embodiments of FIG. 12A to 14C. Single occluders 4140 and 4240 are well suited to treat eccentric regurgitation, as for example, between the anterior leaflet AL and posterior leaflet PL adjacent to the posteromedial commissure PMC (i.e., between A3 and P3). A small leakage or regurgitation may exist between the anterior AL and posterior PL, between the clip and anterolateral commissure AL, with such leakage considered acceptable to warrant no further treatment. Similar to SOD 4100, SOD 4200 is coupled to clip CL via clip connector 4270 which may be similar or the same as clip connector 4170. Further, SOD 4200 includes support frame 4220 supporting occluder 4240 and extending from clip connector 4270 to an annulus anchor 4281 disposed on both sides of the annulus of the native valve.

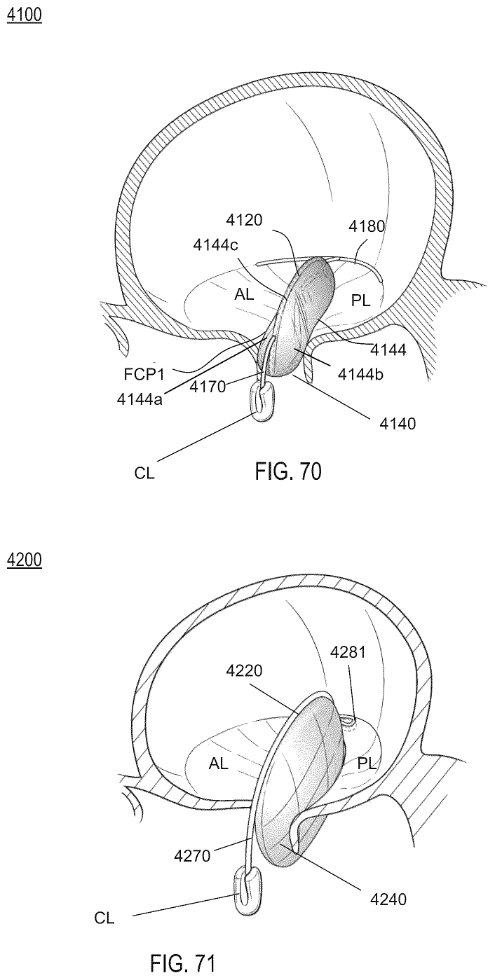

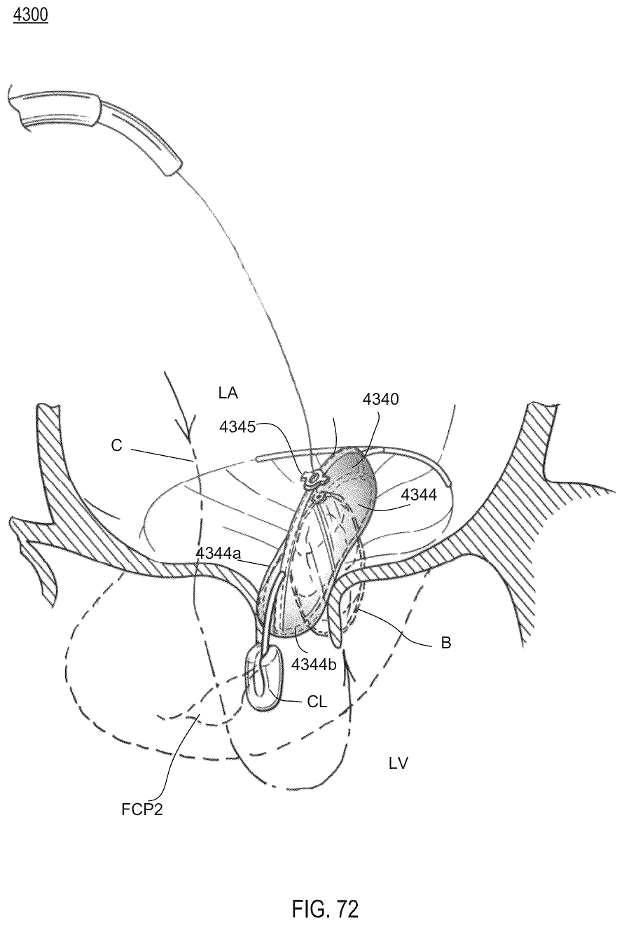

FIG. 72 illustrates a device and a method for repairing or salvaging an SOD 4300 previously implanted in mitral valve MV with an eccentric clip CL (similar to SOD 4100 in FIG. 70). SOD 4300 may have deteriorated over time and may no longer be functioning as well as desired. SOD 4300 has occluder 4340 with movable membrane 4344 (like SOD 4100), and movable membrane 4344 may have lost some mobility, compromising seal against native leaflets. Function of SOD 4300 can be restored by percutaneously delivering an inflatable balloon B to the interior of occluder 4340, i.e., to ventricle side of movable membrane 4344. Balloon B can be delivered via catheter C, which can be introduced into left atrium LA via, for example, transseptal delivery originating in femoral vein, through inferior vena cava IVC and right atrium RA. Balloon B can be delivered through mitral valve MV into left ventricle LV, for example by delivering through flow control portion FCP2 on the other side of clip CL from occluder 4340. Once disposed inside occluder 4340, balloon B can be inflated. The inflated balloon B can prevent first leaflet engaging portion 4344a and second leaflet engaging portion 4344b from being spaced too close to each other, and correspondingly too far away from the corresponding native leaflets. In some embodiments, balloon B can be inflated to a size at which it functions as a static occluder. Alternatively, balloon B can be delivered through orifice (i.e., weep hole) 4345 of occluder 4340. Orifice 4345 provides fluid communication between the LA and LV and is appropriately sized and configured to allow delivery of balloon B therethrough during the salvage procedure. Although illustrated with an eccentric clip CL, the device and method related to embodiment 4300 may also apply to embodiments with a centrally disposed clip having one occluder or even two occluders coupled to the central clip.

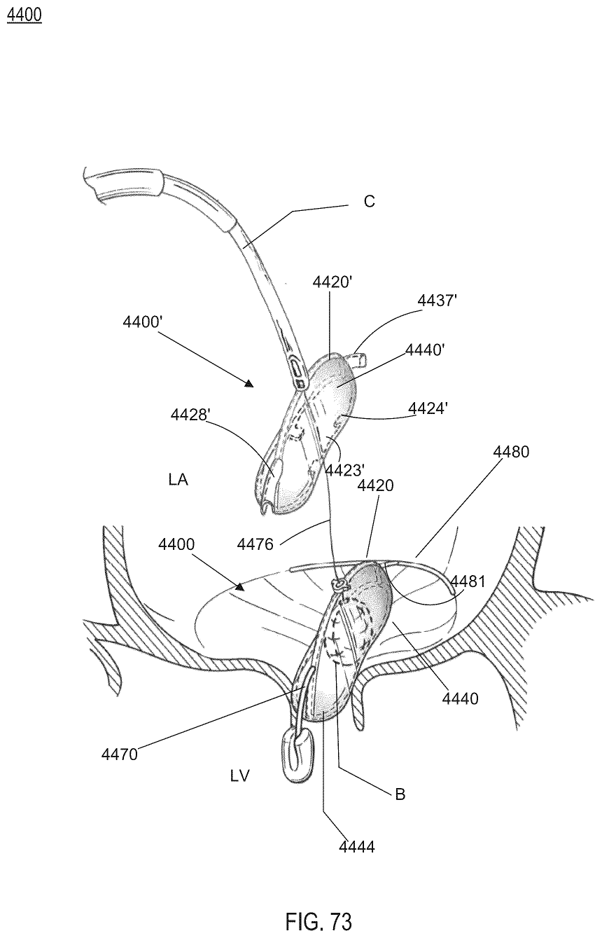

FIG. 73 illustrates another embodiment of a device and a method for repairing or salvaging previously implanted SOD 4400 (similar to the embodiment of the device and the method as shown in FIG. 72). In this embodiment, a second, repair SOD 4400′, can be delivered to native mitral valve MV and can be disposed over previously implanted SOD 4400. Repair SOD 4400′ is similar to SOD 4400 but has a support frame 4420′ that is different from the support frame 4420 of SOD 4400. Support frame 4420′ can include an annulus connector arm clip 4437′ for engaging with arm 4481 of the annulus connector 4480 of SOD 4400, a lower, peripheral membrane support member 4423′, and a portion 4428′ that can engage with clip connector 4470 of SOD 4400. The function of SOD 4400 can be restored by a percutaneous procedure. First, a small anchoring balloon B can be delivered to the left ventricle LV side of movable membrane 4444, inflated, urged in the direction of left atrium LA and secured to SOD 4400. A tension member 4476 can extend from anchoring balloon B through support frame 4420 into left atrium LA. Repair SOD 4400′ can be delivered using catheter C and tension member 4476 and placed onto SOD 4400. In an example embodiment, catheter C may be configured to push onto SOD 4400′ to place it over SOD 4400, as it slides down tension member 4476. Peripheral membrane support member 4423′ is urged through first flow control portion FCP1, between occluder 4440 and native leaflets AL, PL, annulus connector arm clip 4437′ can be engaged with connector arm 4481, and portion 4428′ can be engaged with clip connector 4470. Tension member 4476 can be crimped at the upper (atrial) side of SOD 4400′, completing the securement of SOD 4400′ to SOD 4400, and thus to native mitral valve MV. Optionally, peripheral membrane support member 4423′ of occluder 4440′ may be configured with a series of retaining tangs 4424′ to engage with the terminal, open end of occluder 4440 to aid in the securement of SOD 4400′. Occluder 4440′ replaces the function of deteriorated occluder 4440.

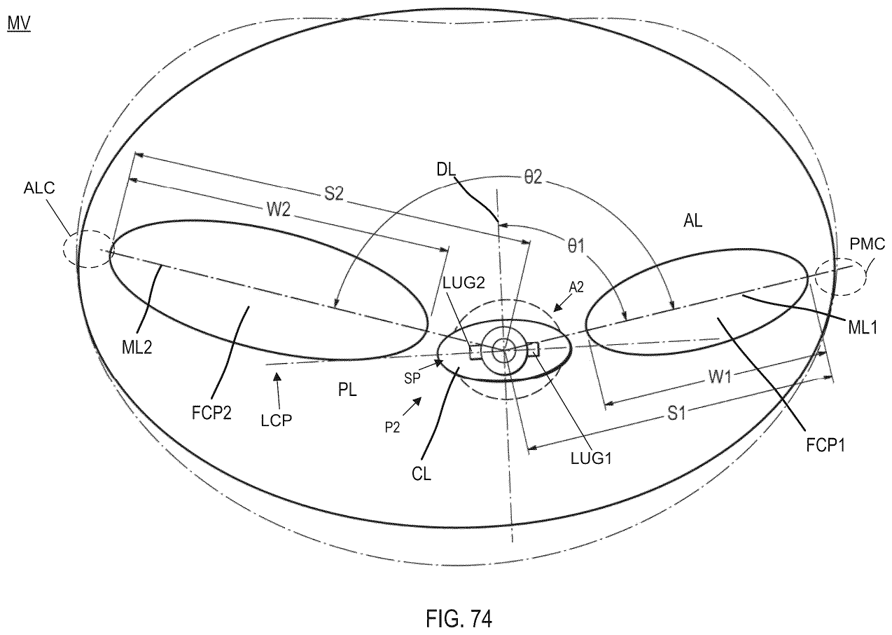

As discussed in detail above with reference to FIGS. 37B, FIG. 74 shows a native mitral valve MV in which a single clip CL (clip CL is indicated by a dashed line and is located below anterior leaflet AL and posterior leaflet PL) has been disposed centrally to approximate the edges of the anterior leaflet AL and posterior leaflet PL at their respective A2 and P2 segments creates two flow control portions-FCP1, which is bounded by the anterior leaflet AL, posterior leaflet PL, clip CL, and posteromedial commissure PMC, and FCP2, which is bounded by the anterior leaflet AL, posterior leaflet PL, clip CL, and anterolateral commissure ALC. FIG. 74 schematically illustrates the clipped valve MV during systole, and shown that the native leaflets are not coapted, i.e., there is a residual flow area between the leaflets, and thus the valve MV is subject to regurgitation of blood during systole. As shown in FIG. 74, in such a clipped mitral valve MV, the shape of the space between the native leaflets AL, PL is oval or approximately elliptical in shape, with respective mid-lines ML1 and ML2 that are angularly offset relative to each other and to the clip CL. The approximated leaflets are captured relative to a leaflet clipping or coaptation plane LCP extending longitudinally through clip CL. It may be desirable to configure an SOD to conform to the geometry of the spaces between the leaflets of the clipped valve. With reference to FIG. 74, the clipped valve MV has a first flow control portion FCP1 that has a space between the leaflets during systole with an offset S1 (the lateral distance from the central axis of clip CL to the radially outer end of the space, which may be the commissure of the native valve on that side of the clip CL and a width W1 (the extent of the space along its midline ML1, i.e., from the commissure to the point where the leaflets contact each other by being clipped with clip CL). Similarly, the space between the leaflets AL, PL in the second flow control portion FCP2 has an offset S2 and a width W2 along midline ML2. The midlines ML1, ML2 of the two spaces are angularly separated by an angle θ₂. The midlines ML1, ML2 also have an angular relationship to a reference datum line DL through the center of clip CL, perpendicular to the coaptation plane LCP of the clipped leaflet margins. As shown in FIG. 74, this angular relationship can be defined by an angle θ₁ between midline ML1 of the space in the first flow control portion FCP1 and datum line DL of clip CL, as well as angle θ₂-θ₁ between midline ML2 of the space in the second flow control portion FCP2 and datum line DL of clip CL.

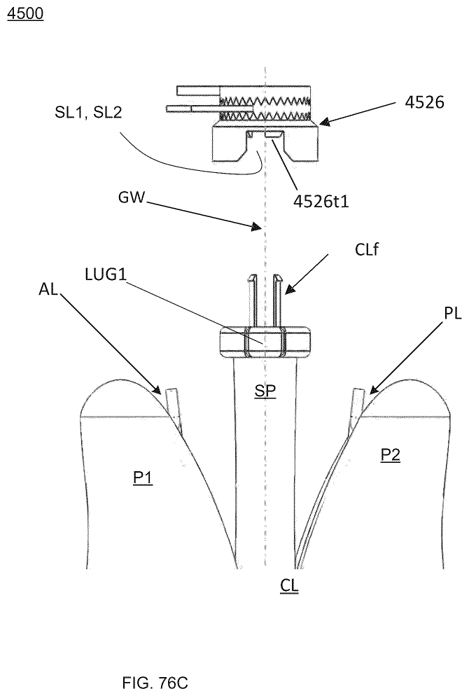

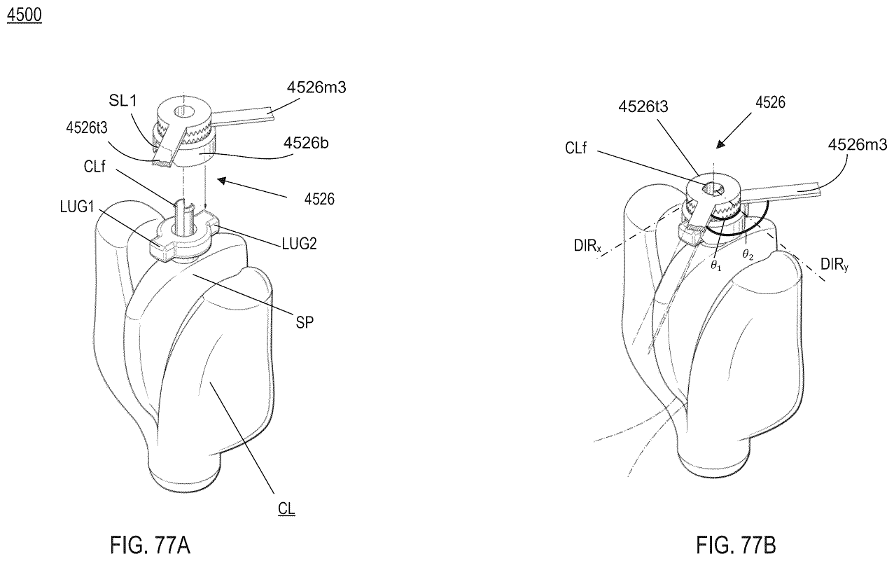

FIG. 74 also shows spacer SP attached between the paddles of clip CL and having lugs LUG1 and LUG2. Spacer SP and lugs LUG1 and LUG2 provide a reference frame for proper positioning of SOD 4500, and for attaching SOD 4500 to clip CL as further described below in relation to FIGS. 77A-77B.

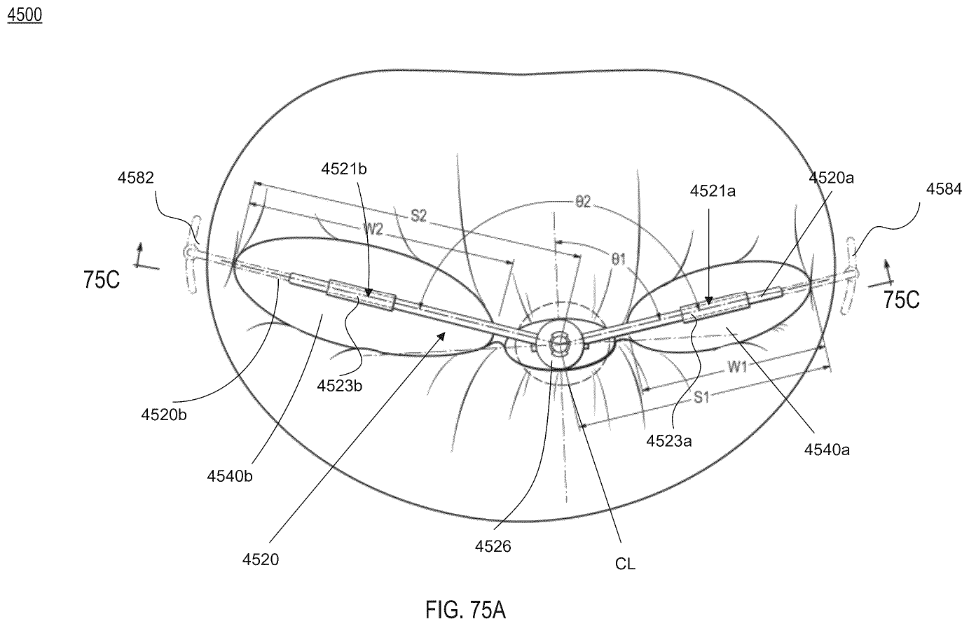

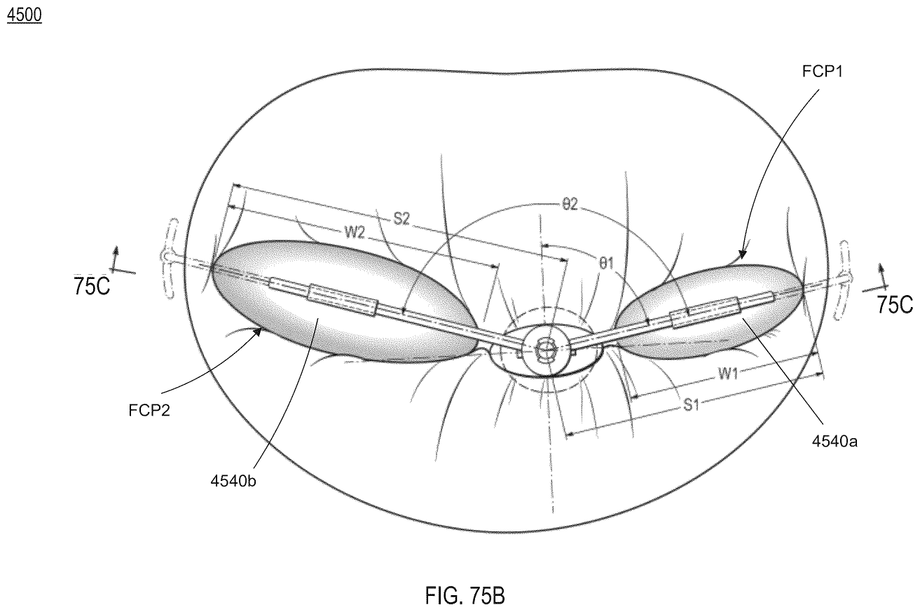

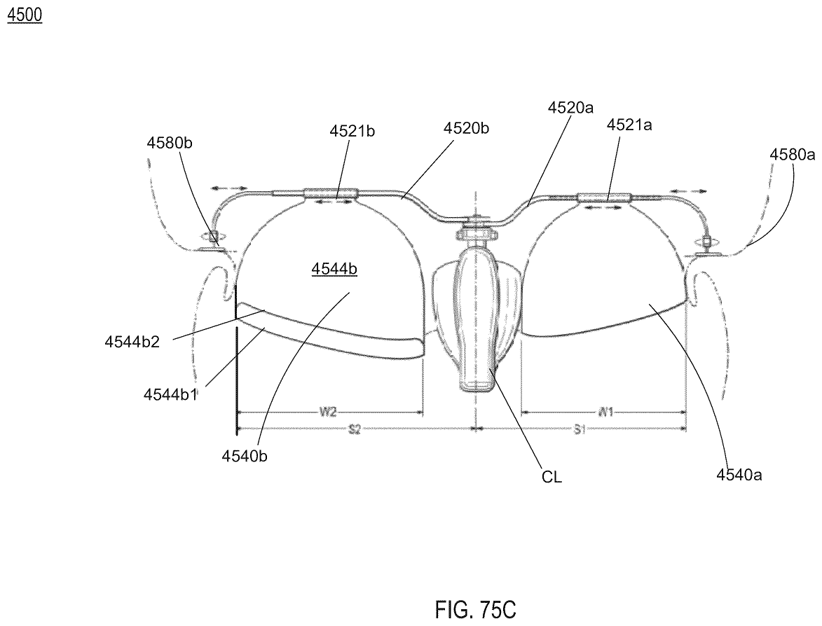

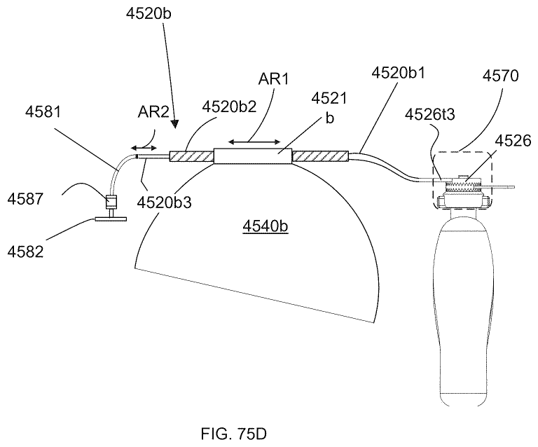

As shown in FIGS. 75A to 75D, SOD 4500 can be selectively configured to conform to the geometry of the clipped mitral valve MV shown in FIG. 74. FIGS. 75A-75B shows a top view of SOD 4500 (i.e., the view from the left atrium), while FIG. 75C shows a cross-sectional view of SOD 4500 along a cut plane 75C, as shown in FIGS. 75A-75B. Further, FIG. 75D shows a left side of the cross-sectional view of SOD 4500.

SOD 4500 includes a support frame 4520 that includes a first occluder arm 4520a supporting first occluder 4540a on an occluder connecting portion 4521a and a second occluder arm 4520b supporting second occluder 4540b on an occluder connection portion 4521b. The occluder arms are coupled together at occluder arm pivot 4526 for adjustable relative angular position.

As shown in FIG. 75A, first occluder 4540a may be selectively positioned along first occluder arm 4520a by moving (e.g., sliding) connecting portion 4521a along first occluder arm 4520a. Similarly, second occluder 4540b may be selectively positioned along second occluder arm 4520b by moving connecting portion 4521b along second occluder arm 4520b. In various implementations, each arm (e.g., arm 4520a and 4520b) is coupled at a radially outer end to an annulus of a native valve using a respective annulus connector 4580a and 4580b, as shown, for example in FIGS. 75A, 75C, and 75D. As shown in FIG. 75D, arm 4520b (and/or 4520a) may include several segments. For instance, arm 4520b may include segment 4520b1, connecting arm 4520b to occluder arm pivot 4526, segment 4520b2 along which connecting portion 4521b is configured to slide, and segment 4520b3, connecting segment 4520b2 with annulus connector 4580b. In an example implementation, segment 4520b1 may have a curvature (as shown in FIG. 75D) to properly position occluder 4540b within FCP2. Further, segment 4520b2 may be selected to be substantially straight, or having sufficiently gradual curvature such that connecting portion 4521b is allowed to slide along segment 4520b2, as indicated by an arrow AR1. In some implementations, segment 4520b3 is configured to move (e.g., slide) relative to segment 4520b2, as indicated by an arrow AR2. For example, segment 4520b2 may contain a channel, such that when segment 4520b3 is inserted into that channel, it can slide in and out of segment 4520b2. For example, the channel of segment 4520b2 may be a groove, and segment 4520b3 may slide inside that groove. For example, segment 4520b3 may be spring loaded and slide in and out of segment 4520b2 based on external loads (e.g., based on forces imposed on occluder 4540b). Thus, a length of segment 4520b3 may be self-adjusting. In another example embodiment, segments 4520b2 and 4520b3 form a telescopic joint. The sliding motion of segment 4520b3 allows for adjusting a radial position of annulus connector 4580. Annulus connector 4580 includes arm 4581/4583 and corresponding annulus anchor 4582/4584, which is pivotally connected to the respective arm by annulus anchor connector 4587 for adjustable angular position to most suitably engage with the curvature of the MVA, depending on the angles θ₂ and θ₁ set between occluders 4540a and 4540b (or to accommodate pivoting forces).

SOD 4500 is shown installed in native valve, such as a mitral valve. FIGS. 75A and 75B show SOD 4500 during systole. FIG. 75B shows the same view as FIG. 75A, but with membranes of occluders 4540a and 4540b shaded to further indicate that occluders 4540a and 4540b occlude the spaces between the leaflets AL, PL in flow control portions FCP1 and FCP2. Note that in FIG. 75C, occluder 4540b includes flexible membrane 4544b which has a first leaflet engaging portion 4544b1 to engage the anterior leaflet AL and a second leaflet engaging portion 4544b2 to engage the posterior leaflet PL. The first leaflet engaging portion or first free margin 4544b1 extends deeper within the LV than the second leaflet engaging portion or second free margin 4544b2. The upward surge of blood during systole may first come into contact with this deeper extending portion and may ensure better systolic filling of occluder 4540.

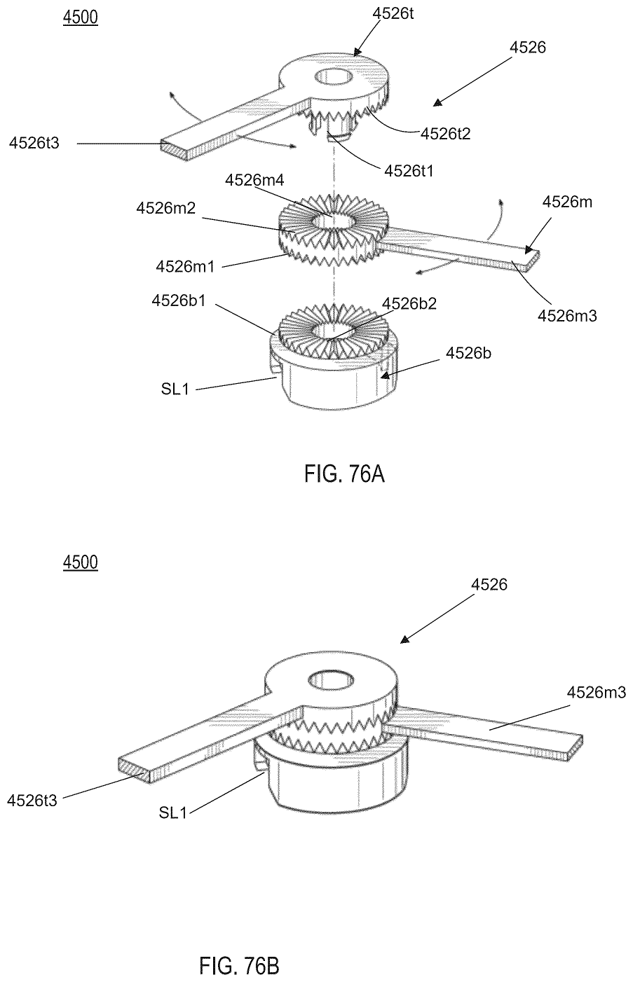

As shown in FIG. 75A, 4520b and 4520a are positioned at an angle θ₂ relative to each other. Angle θ₂ may be selected by configuring occluder arm pivot 4526, as shown in more detail in FIGS. 76A-C and FIGS. 77A-B. FIG. 76A shows an exploded view of occluder arm pivot 4526. In the example embodiment, occluder arm pivot 4526 includes a top portion 4526t, a middle portion 4526m, and a bottom portion 4526b. Top portion 4526t includes an arm 4526t3, and middle portion 4526m includes an arm 4526m3. In some implementations, arm 4526t3 may be connected to segment 4520b1 of connecting arm 4520b, as shown in FIG. 75D. Alternatively, 4520t3 may be a portion of segment 4520b1. Similarly, arm 4526m3 may be connected to segment 4520a1 of connecting arm 4520a. Alternatively, 4520m3 may be a portion of segment 4520a1.

In an example embodiment, occluder arm pivot 4526 may be part of clip connector 4570, as shown for example in FIG. 75D. In an example implementation, a bottom portion of occluder arm pivot 4526 is configured to engage with lugs LUG1 and LUG2 on clip CL via, for example, a post/socket interface between the lugs and occluder arm pivot 4526 (e.g., a slot in occluder arm pivot 4526 may be selectively engaged with the lugs, which define the reference angular positions for arms 4526t3 and 4526m3 of occluder arm pivot 4526.

As shown in FIG. 76A, bottom portion 4526b includes teeth 4526b1 configured to match teeth 4526m1 of middle portion 4526m, such that teeth 4526m1 can be inserted between teeth 4526b1, thus matching middle portion 4526m to bottom portion 4526b. In an example embodiment, middle portion 4526m can match bottom portion 4526b at a set of angles. The number of angles at which middle portion 4526m matches bottom portion 4526b is equal to the number of teeth available for middle portion 4526m, thus, if middle portion 4526m has, for example, 12 teeth, middle portion 4526m may match bottom portion 4526b at angles 0, 30, 60, 90, 120, 150, 180, 210, 240, 270, 300, 330, having an angular increment of 30 degrees. In an example embodiment, number of teeth for 4526m1 may be large (e.g., 36, 72 teeth, and the like) to have a small angular increment. Further, as shown in FIG. 76A, top portion 4526t includes teeth 4526t2 configured to match teeth 4526m2 of middle portion 4526m, such that teeth 4526t2 can be inserted between teeth 4526m2, thus matching top portion 4526t to middle portion 4526m. In an example implementation, number of teeth 4526t2 (and teeth 4526m2) may be the same as number of teeth 4526m1. Alternatively, the number of teeth 4526t2 may be different from the number of teeth 4526m1.

FIG. 76A also shows that portions 4526t, 4526m, and 4526b of occluder arm pivot 4526 may be held together by a coupler element 4526t1 configured to pass through a hole 4526m4 and connect to a socket 4526b2. In an example implementation, coupler element 4526t1 is configured to click-in-place when inserted into socket 4526b2, thereby securing coupler element 4526t3 to socket 4526b2. It should be appreciated, that portions 4526t, 4526m, and 4526b may be coupled via any other suitable connection (e.g., bolts, screws, clips, and the like). FIG. 76B shows an example occluder arm pivot 4526 with portions 4526t, 4526m, and 4526b tightly coupled to secure the desired angle θ₂ between occluder portions 4540a and 4540b.

FIG. 76C shows a side view of SOD 4500. Clip CL is shown to capture anterior and posterior leaflets and is further shown to include fingers CLf and lugs LUG1, LUG2 on a spacer to which occluder arm pivot 4526 can be coupled. As shown in FIG. 76C, occluder arm pivot 4526 is guided by a guidewire to clip CL, and sockets SL1, SL2 of occluder arm pivot 4526 are configured to engage with lugs LUG1, LUG2 of clip CL.

In various embodiments, SOD 4500 may be configured to match the geometry of a clipped valve (FIG. 74), and after matching the clipped valve, SOD 4500 may be delivered to the clipped valve. In an example embodiment, a method for configuring SOD 4500 to match the clipped valve includes several steps. At step 1, clip CL with spacer SP having lugs LUG1 and LUG2 may be installed to clip the leaflets of the natural valve. At step 2, an imaging may be performed (e.g., ultrasound imaging, imaging with a catheter camera, and the like) to determine angular position of lugs LUG1 and LUG2, as shown in FIG. 74, relative to flow control portions FCP1 and FCP2. At step 3, the method may include determining a angle θ₂ between MV orifices through vertex at clip center (as shown in FIG. 74), and angle θ₁, as shown in FIG. 74. The angles θ₂ and θ₁ can be determined via a suitable imaging technique (e.g., ultrasound imaging, via a camera of a catheter, and the like). At step 4, the method may include determine offset distances S1 and S2 from the clip centerline of the resulting clipped orifices, as shown in FIG. 74. Distances S1 and S2 are also determined with a suitable imaging technique. At step 5, the method may include extracorporeally, pre-setting occluder sections at an angular position of θ₂ (engaging 4526t to 4526m), as well as pre-setting angle θ₁ relative to datum line DL (engaging assembly of 4526t, 4526m to 4526b in the correct orientation), as shown in FIG. 74. At step 6, the method may include extracorporeally, pre-setting offset for each occluders 4540a and 4540b S1 and S2 distances away from clip CL. Once the angles θ₂ and θ₁ and offsets S1 and S2 are configured, resulting in a patient-specific occluder configuration, at step 7, the method includes collapsing occluder 4500, that is being in the patient-specific occluder configuration, into a delivery catheter. At step 8, the collapsed occluder 4500 is delivered to the left atrium, and at step 9, occluder 4500 is coupled to clip CL in correct patient-specific occluder configuration with respect to clip CL by engaging a socket SL1, SL2 of occluder arm pivot 4526 with lugs LUG1 and LUG2 on spacer SP.

Alternatively, occluder can be offered in a range of classified pre-determined sizes and configurations to be selected according to imaging data (e.g., echo data) and prior to delivery and engagement with previously deployed clip.

FIG. 77A-77B show an example method of placing occluder arm pivot 4526 of SOD 4500 over a spacer SP having lugs LUG1 and LUG2. In the example embodiment, bottom portion 4526b includes sockets SL1 and SL2 (more clearly shown in FIG. 76C) configured to attach to LUG1 and LUG2. Arms 4526t3 and 4526m3, as shown in FIG. 77B, are then configured to be positioned such they form an angle θ₂ between each other, and angle θ₁ is formed between arm 4526t3 and direction DIRy (DIRy is perpendicular to direction DIRx, and DIRx is parallel to a line connecting LUG1 and LUG2). Note, that angles θ₁ and θ₂ as shown in FIG. 77B are the same as angles θ₁ and θ₂ shown in FIGS. 74, and 75A-75B. Occluder arm pivot 4526 is securely connected to spacer SP (and clip CL) through upstanding flexible fingers CLf. Fingers CLf are elastically displaced inwardly when inserted through the center hole of occluder arm pivot 4526, and return to their non-displaced free state to axially retain SOD 4500 to clip CL through an axial abutment face, as illustrated in FIG. 77B.

Clip connector 4570 having occluder arm pivot 4526 may be one possible way for attaching a SOD to a clip. It should be appreciated that various other designs of clip connectors may be used. When capturing native leaflets, the deployed clip CL may not necessary be placed such that its longitudinal axis is perpendicular to the MV plane or mitral valve annulus MVA. The SOD, especially when configured with annulus connectors, is preferably positioned with an SOD axis (longitudinal axis passing through a clip connector of the SOD) perpendicular to the MV plane. In an example embodiment, a SOD may be pivotably engaged to clip CL to advantageously allow the suitable orientation of SOD relative to the MV plane, regardless of the orientation of the clip relative to the MV plane. Additionally, the SOD can be configured with a height adjustment dH, as shown in 78B. Such height adjustment may allow the SOD to be variably positioned in height relative to the clip (and to the plane of the MV), to set the occluder portions at a desired depth within the left ventricle LV with the aim of optimizing the contact zone between the SOD and the free margins of the native leaflets.

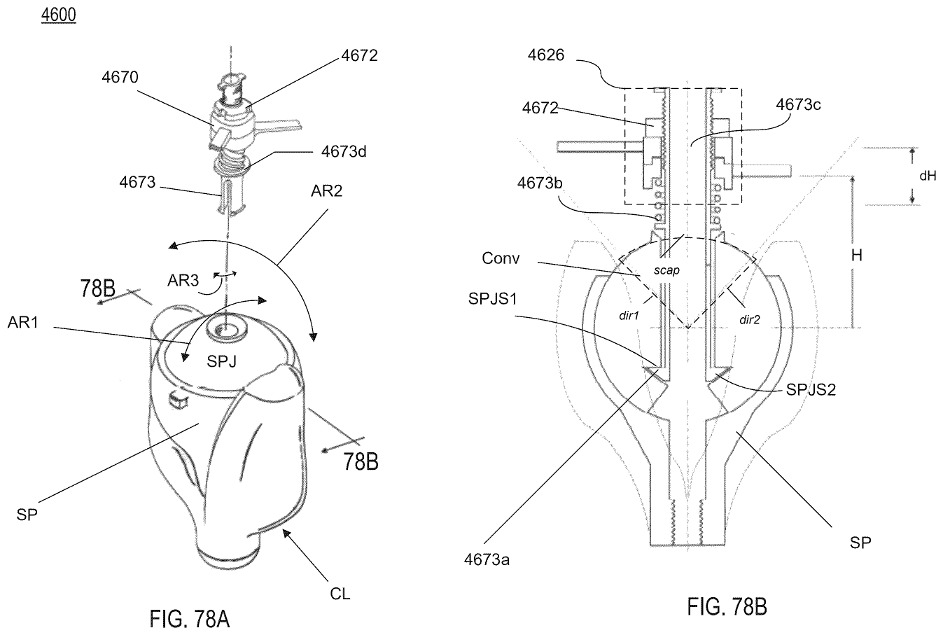

An example clip connector 4670 for an SOD 4600 is shown in FIGS. 78A-78B. FIG. 78A shows a perspective view of clip connector 4670 and clip CL, while FIG. 78B shows a cross-sectional view along a cut plane 78B-78B (shown in FIG. 78A) of clip connector 4670 coupled to clip CL. Clip connector 4670 may be configured to engage clip CL at a spherical joint element SPJ which can rotate as indicated by arrows AR1, AR2, and AR3, such that a proper position for occluders can be selected. Thus, for example, the or each occluder (not shown in FIGS. 78A and 78B) of SOD 4600, may be angled or tilted laterally relative to the vertical axis of clip CL, along the direction indicated by arrow AR1. Alternatively, or in addition, the or each occluder of SOD 4600, may be angled or tilted longitudinally relative to the vertical axis of clip CL, along the direction indicated by arrow AR2, disposing one occluder higher or lower relative to the clip, and one occluder higher in the atrium than the other occluder. In the example embodiment, SPJ may be rotated (i.e., relative to clip spacer SP) within a spherical cap scap defined by directions dir1 and dir2, as shown in FIG. 78B.

In the example embodiment, clip connector 4670 includes an axial clip post 4673 configured to be inserted into spherical joint element SPJ, as further shown in FIG. 78B. Axial clip post 4673 may have hook elements 4673a configured to hook with sockets SPJS1 and SPJS2. Further, axial clip post 4673 of clip connector 4670 may include a spring 4673b placed between occluder arm pivot 4626 and a lip element 4673d (as shown in FIG. 78A). Occluder arm pivot 4626 may be configured to move down (or up) a stem element 4673c by compressing (or releasing) spring 4673b. The vertical movement of occluder arm pivot 4626 by an incremental height dH may be controlled by an adjustment nut 4672, so that adjustments to position H may be made either prior to delivering SOD 4600 to clip CL or when attaching SOD 4600 to clip CL. The adjustment to H may be determined based on suitable imaging of a native valve clipped with clip CL. Thus, the height of the occluder arms and therefore occluders (not shown in FIGS. 78A and 78B) may be adjusted relative to the clip CL along its vertical axis, and thus higher or lower in flow control portion of the native valve.

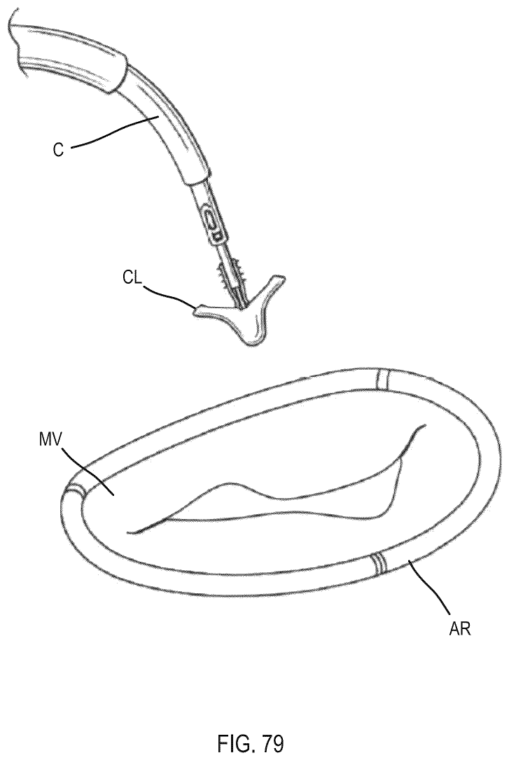

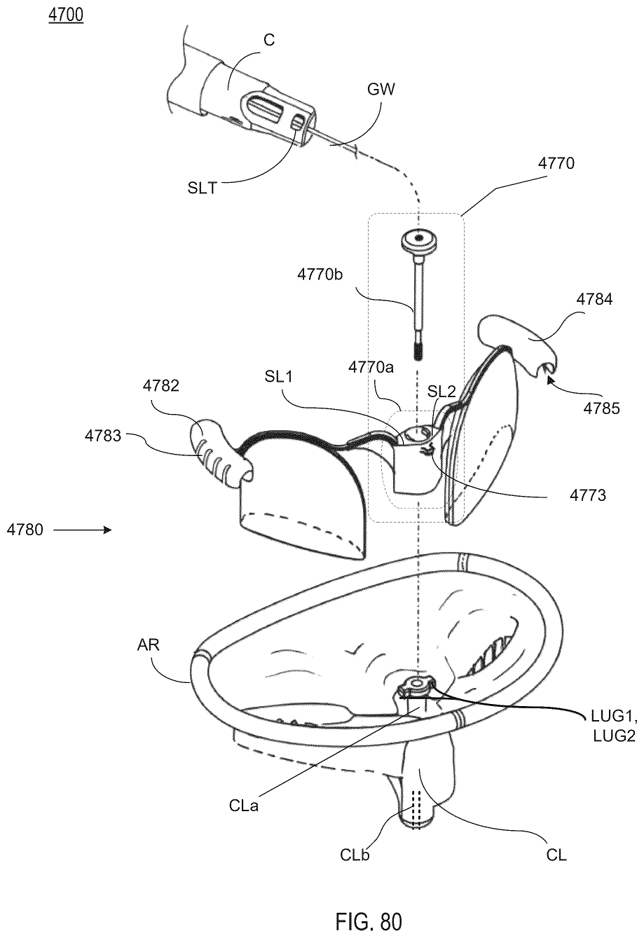

Another approach to addressing regurgitation of valves, such as mitral valves, is annuloplasty, a procedure in which an annuloplasty ring AR is implanted in the mitral valve, and reduces the perimeter of the native valve annulus, thus bringing the native leaflets closer together an allowing them to coapt properly. Annuloplasty rings AR can be implanted surgically, or percutaneously (such as the IRIS mitral annuloplasty ring produced by Boston Scientific or the Cardioband mitral system produced by Edwards Lifesciences). In some instances, treatment of a regurgitant mitral valve by an annuloplasty procedure may not reduce the regurgitation sufficiently, and it may be desirable to further treat the valve with an SOD in accordance with the current disclosure, e.g., as described with reference to the embodiments shown in FIGS. 1A to 4B (except that the valve has already been treated with an annuloplasty ring). In some instances, a regurgitant valve may be treated with both an annuloplasty procedure and a clipping procedure (in either order) and may still benefit from further treatment with an SOD, e.g. as described with reference to any of the many embodiments above directed to treating a clipped valve with an SOD. It is therefore contemplated that in some embodiments one or more clips CL may be delivered to a mitral valve MV that has already been treated with an annuloplasty ring AR and in the same or a related procedure an SOD may be delivered to the mitral valve MV, as shown in FIGS. 79 and 80. As shown in FIG. 80, SOD 4700 may be similar to other embodiments of SODs described above, but annulus connector 4780 can have annulus anchors 4782 and 4784 that are configured to securely engage with annuloplasty ring AR. For example, annulus anchor 4782 may include a number of spring-loaded tabs or detents 4783 configured to clamp onto cross-section of engaged annuloplasty ring AR. Alternatively, annulus anchor 4784 may include a number of barbs 4785 to securely engage with annuloplasty ring AR. Further, as shown in FIG. 80, SOD 4700 includes a clip connector 4770 having a first element 4770a for coupling with clip CL, and a second connector pin 4770b (also referred to as a tie bolt 4770b) for attaching first element 4770a to clip CL (e.g., second connector pin 4770b may be a bolt configured to screw into a receiving socket of clip CL).

As shown in FIG. 80, clip CL includes lugs LUG1, LUG2, and first element 4770a is configured to include sockets SL1 and SL2, into which lugs may be inserted when first element 4770a is placed onto a receiving part CLa of clip CL. In an example embodiment, SOD 4700 is delivered via a catheter C guided by a guidewire GW. Catheter C also may be used to deliver and place clip CL for approximating anterior and posterior leaflets of a mitral valve. In some cases, when clip CL includes lugs LUG1, LUG2, catheter C includes slots SLT as shown in FIG. 80 for coupling to lugs of clip CL. Such coupling allows for clip CL to resist a torque when unthreading guidewire or threading connector pin 4770b.

With reference to both FIGS. 79 and 80, a procedure for repairing mitral valve MV may include several steps as follows. At step 1, a clip delivery catheter C is used to deliver clip CL over guidewire GW to left atrium LA, and clip CL captures the native leaflets of mitral valve MV. In some cases, the native leaflets may have previously been repaired, e.g., mitral valve MV may have undergone an annuloplasty repair. The clip delivery catheter C is configured to engage, via slots in clip delivery catheter C, with lugs LUG1, LUG2 of clip CL when delivering and placing clip CL. At step 2, slots of clip delivery catheter C are disengaged from the lugs of clip CL, and the catheter is retrieved from left atrium LA, while keeping the guidewire GW attached to the deployed clip CL. At step 3, a SOD delivery catheter (SOD delivery catheter may be similar in form and/or in function as clip delivery catheter C) is advanced over the guidewire GW, and lugs of SOD 4700 (e.g., lug 4773 is shown in FIG. 80) are engaged with slots SLT of the SOD delivery catheter, thus allowing orienting SOD 4700 relative to a clip CL. At step 4, SOD 4700 is connected to clip CL via a suitable coupling (e.g., via first element 4770a and connector pin 4770b). The coupling is configured to engage with the exposed lugs of clip CL (e.g., slots SL1 and SL2 of first element 4770a are configured to engage lugs LUG1, LUG2 of clip CL). Further, annulus connectors 4782 and 4784 are configured to engage with the previously implanted annuloplasty ring AR. At step 5, while SOD delivery catheter is still engaged with SOD 4700, the guidewire may be retrieved from within SOD delivery catheter (e.g., the guidewire may have a threaded end, and may be rotated and unscrewed from a threaded socket CLb located within clip CL. At step 6, a tie bolt delivery catheter (not shown) may be placed and advanced within the lumen of the SOD delivery catheter, for delivering tie bolt 4770b to an assembly of SOD 4700 and clip CL to secure tie bolt 4770b to clip CL. In an example embodiment, tie bolt 4770b has a threaded end (with a pitch and diameter of the thread being the same as the pitch and the diameter of the threaded end of the guidewire) configured to engage with the threaded socket CLb. At step 7, SOD delivery catheter is configured to disengage from SOD 4700 (e.g., lugs 4773 of SOD 4700 are configured to be disengaged from slots of the SOD delivery catheter), and the SOD delivery catheter is retrieved from the left atrium LA. It should be appreciated, that method described above may be applied to mitral valves MV which do not include mitral valve annuloplasty ring AR.

An alternative treatment procedure is contemplated for treatment of a valve that has already been clipped, by percutaneously delivering an annuloplasty ring and then delivering an SOD. For example, SOD 4700 may be coupled to clip CL via lasso-type means as described with reference to the embodiments shown in FIGS. 10A-10B, and 56C-56I.

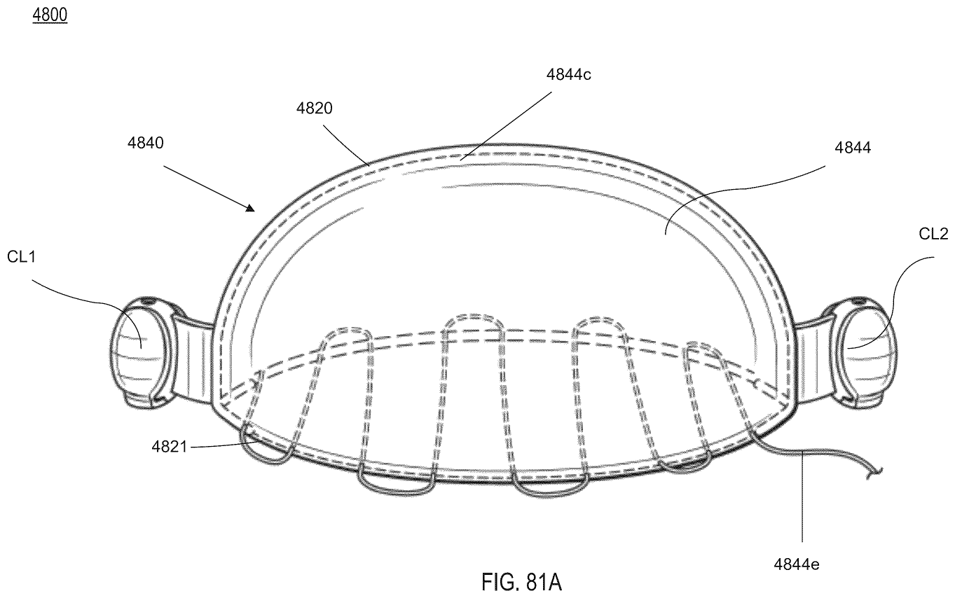

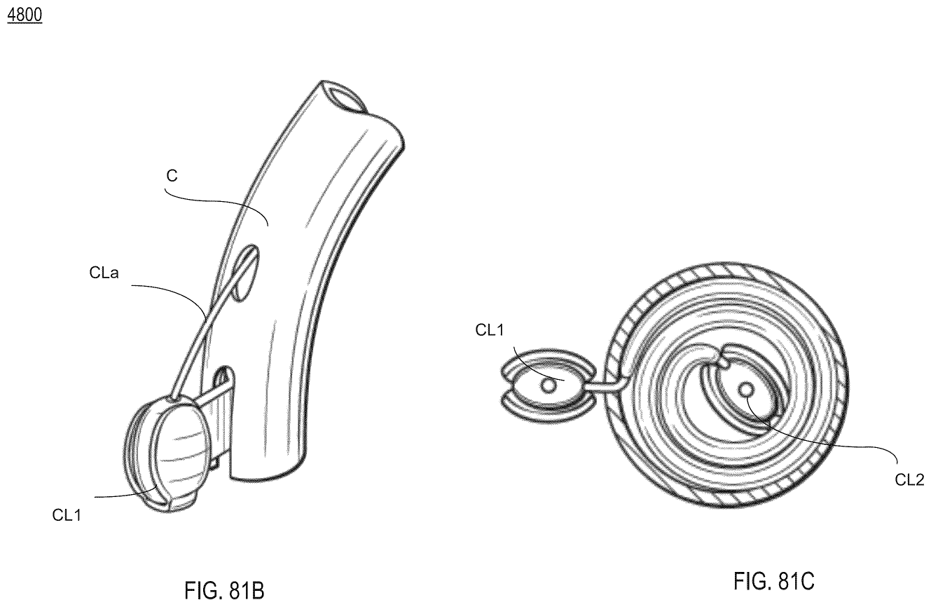

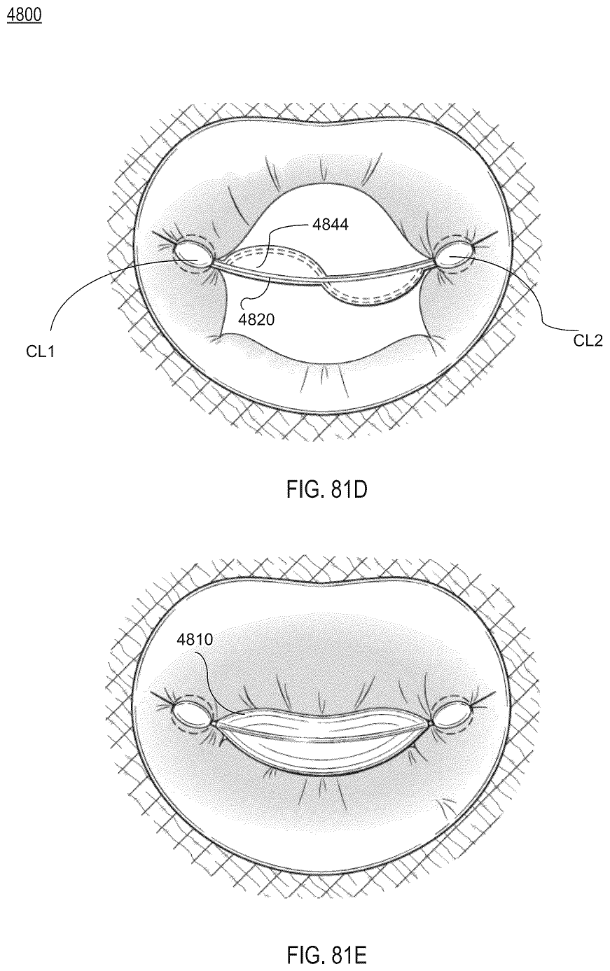

In some embodiments, a SOD can be integrated with leaflet clips CL so that in a single procedure both the clips and SOD can be delivered to, and implanted in, the native valve. One such embodiment in shown in FIGS. 81A-E. SOD 4800 includes an occluder 4840 having a flexible membrane element 4844 supported on support frame 4820 at frame attachment portion 4844c. Support frame 4820 includes integrated clips CL1 and CL2, when the two clips are used to clip a native valve, as shown, for example, in FIG. 37D. SOD 4800 is configured to be placed in a single flow control portion located between clips CL1, CL2. Support frame 4820 optionally includes a membrane support member 4821 around lower periphery of SOD flexible membrane 4844 to bias them towards an open position, i.e., to coapt with native leaflets during systole. In delivery configuration, SOD 4800 can be disposed in lumen of delivery catheter, rolled into a coil, with one of clips CL2 inside lumen, and other clip CL1 disposed (or disposable during procedure) on outside of catheter, as shown in FIGS. 81B and 81C. In this embodiment, SOD delivery catheter C is configured to deliver clip CL1 to approximate leaflets of a native valve at a first location. Subsequently, SOD 4800 may be released from SOD delivery catheter C, so that it uncoils into a configuration that is suitable for deploying SOD 4800 (e.g., SOD 4800 is coiled in SOD delivery catheter C, and is biased towards the deployed configuration, when it is uncoiled). After SOD 4800 is uncoiled, clip CL2 is configured to be delivered and engaged with the native valve at a second location to approximate the leaflets of the native valve at that location.

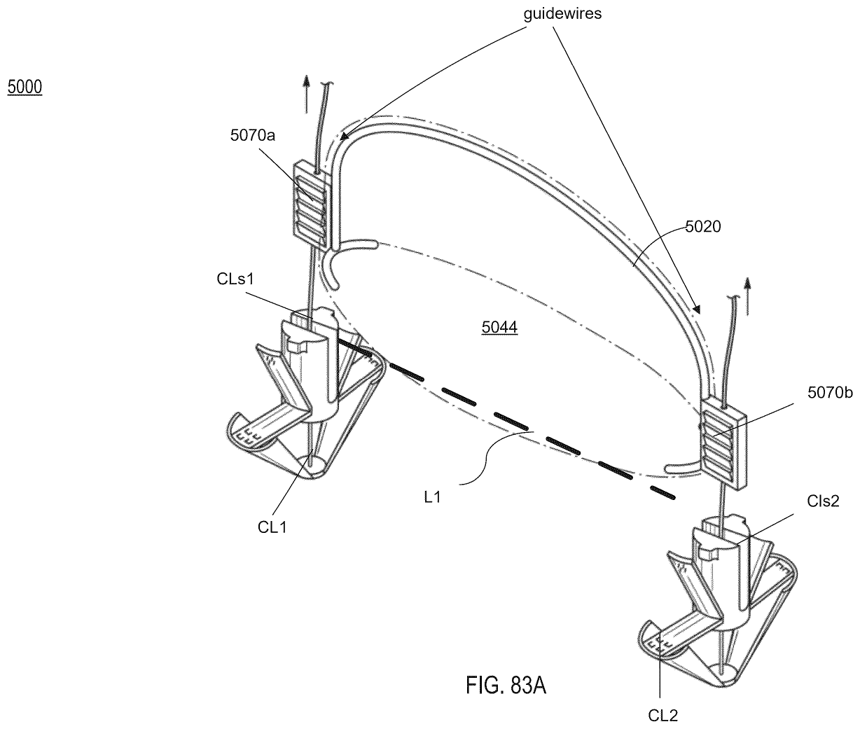

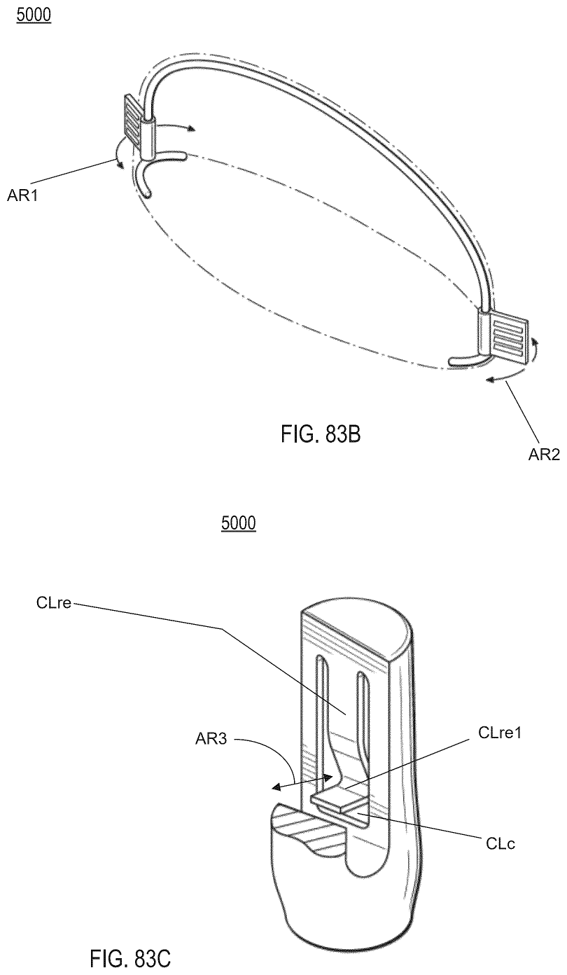

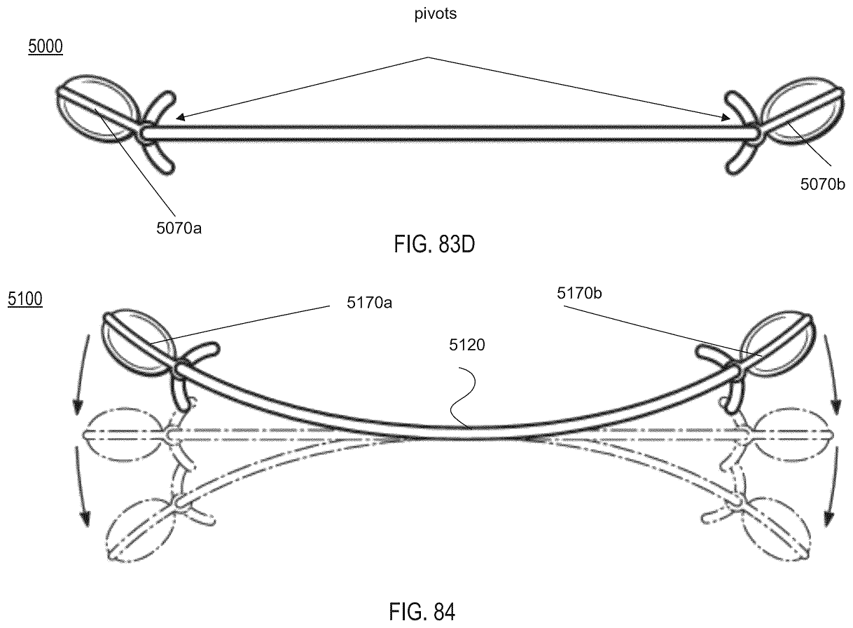

In an alternative embodiment, instead of clip CL1 and CL2, clip connectors may be used, as further discussed in relation to FIGS. 83A-83C, when SOD 4800 is deployed after deploying clips CL1 and CL2.

FIG. 81B shows a clip actuator wire CLa extending from clip CL1 into the lumen of the delivery catheter C. Optionally, to avoid undesirable flow forces on SOD during process of deployment from blood flow during heart cycle, a removable suture 4844e (as shown in FIG. 81A) can be used to keep the lower edge of flexible membrane 4844 of occluder 4840 secured together. Once both clips are secured to the leaflets of a native valve, thereby securing SOD 4800 to the native valve, removable suture 4844e can be withdrawn from membrane 4844, as shown in FIG. 81A. Implanted SOD 4800 is shown in FIGS. 81D (diastole) and 81E (systole). FIG. 81D shows that support frame 4820 of SOD is pre-curved, and/or is flexible, to match natural coaptation line of native valve when viewed en face, between commissures. Length of lower edge of occluder membrane 4844 is suitably sized so as to encompass the regurgitant flow area during systole, in a diseased mitral valve. The lower edge is sufficiently long to define the sealing perimeter 4810 during systole (shown in FIG. 81E) and ensure appropriate coaptation with native leaflets along full length of natural coaptation line. This length is longer than the straight-line distance between clips CL and consequently, the collapsed occluder membrane 4844 will assume a serpentine shape during diastole, as shown in FIG. 81D).

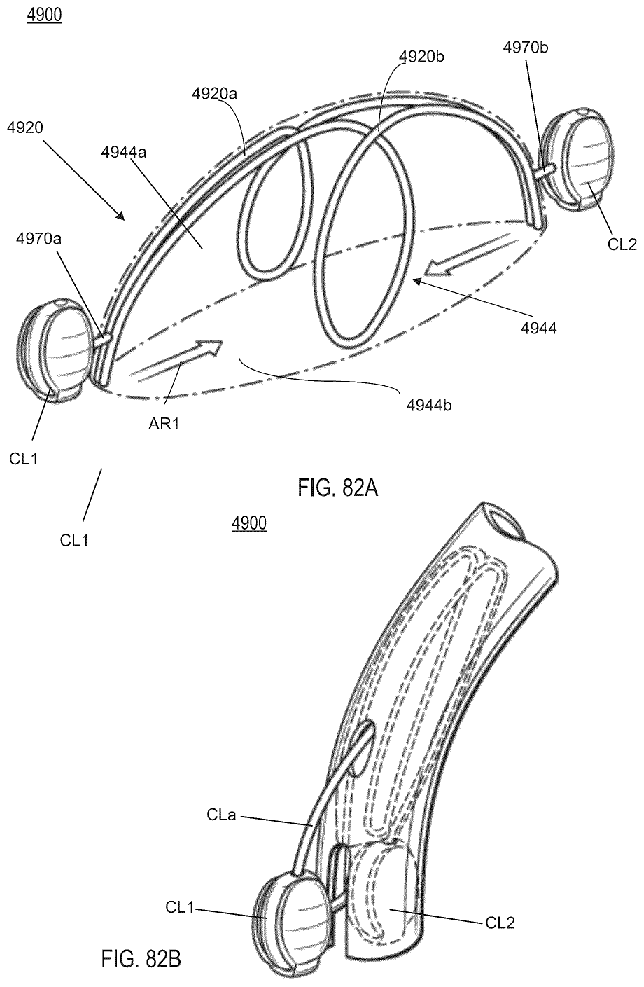

In an alternative embodiment, as shown in FIGS. 82A-B, SOD 4900 includes support frame 4920 which has a different configuration than support frame 4820 of SOD 4800. For example, support frame 4920 can be compressed laterally, i.e., by bringing clip ends together laterally, as shown by arrows AR1, rather than coiling as with support frame of SOD 4800. As shown in FIGS. 82A-B, support frame 4920 may have a two-wire design (e.g., support frame 4920 is formed from wire 4920a and 4920b). In some cases, each wire 4920a and 4920b may include one or more loops for providing a lateral stability to flexible membrane 4944. Further, loops of wires 4920a and 4920b may help bias membranes to open when SOD 4900 is positioned within a follow control portion of a native valve. Further, the loops may promote (i.e., help) an elastic collapsibility of support frame 4920 within a lumen of a delivery catheter (e.g., the loops may reduce strain on support frame 4920 and lower the likelihood of plastic deformation to support frame 4920). When SOD 4900 is delivered to the flow control portion, and is in unconstrained state, the loops are configured to be pre-shaped to keep first leaflet engaging portion 4944a and a second leaflet engaging portion 4944b of membrane 4944 spread apart at mid-span location between clips CL1, CL2. This opening between membranes is required to pressurize the inside of the SOD at end of diastole, and thereby ensure proper filling of SOD (and spreading apart of entire lower edge to rapidly attain the sealing perimeter in systole). Compared to having a support member along the entire lower edge of flexible membrane 4944 (i.e., the membrane free margin), the loops offer more collapsible configuration suitable for catheter delivery. Lastly, the loops are resilient to pressure loading and will collapse toward one another in diastole much easier than if membrane 4944 had a support member along lower edge. Variants with multiple loops in frame member are also possible to set the degree of desired collapsibility or lateral stability of membrane.

As previously described, in relation to FIGS. 81A-81C, in some embodiments in which an SOD is to be engaged with two clips, it may be desirable to deliver clips CL1, CL2 separately from the SOD. In some cases, the clips may be delivered as a part of a single procedure, e.g., with a delivery catheter that is used for delivering clips CL1, CL2 and SOD 5000, rather than integrating the clips as in the embodiments described with reference to FIGS. 81A-82B. As shown in FIGS. 83A-D, SOD 5000 is configured to be coupled with clips CL1 and CL2 via tabs 5070a and 5070b. For example, clips CL1 and CL2 include respective slot CLs1 and CLs2, for coupling with respective tabs 5070a and 5070b attached to support frame 5020. SOD 5000 can be delivered over sutures (or guidewires, as shown in FIG. 83A) over which clips CL1 and CL2 may be delivered. The guidewires are configured to guide tabs 5070a and 5070b into slots CLs1 and CLs2 of respective clips CL1 and CL2. Tabs 5070a and 5070b can be pivotally mounted to support frame 5020, as shown in FIG. 83B with arrows AR1 and AR2, to accommodate relative angle between clip slots and line L1 connecting clips CL1 and CL2.

FIG. 83C shows an example implementation of a slot (e.g., slot CLs1). Slot CLs1 includes a receiving element CLre configured to bend into a cavity CLc, when receiving tab 5070a, and latching to one of the protrusions of tab 5070a, thus bending out of cavity CLc, when one of the protrusions is placed in a part CLre1 of receiving element CLre.

While FIG. 83D shows a top view of SOD 5000 with pivots for tabs 5070a and 5070b, FIG. 84 shows an alternative embodiment of an SOD 5100 that has tabs 5170a and 5170b fixed relative to support frame 5120. Support frame 5120 is flexible and can take a shape that correlates to angles of clip slots. The relative angle between clip slots is dependent on the clipping location along the native leaflet and may be difficult to predict ahead of the procedure. As such, it is advantageous to have an SOD with tabs that are movably connected.

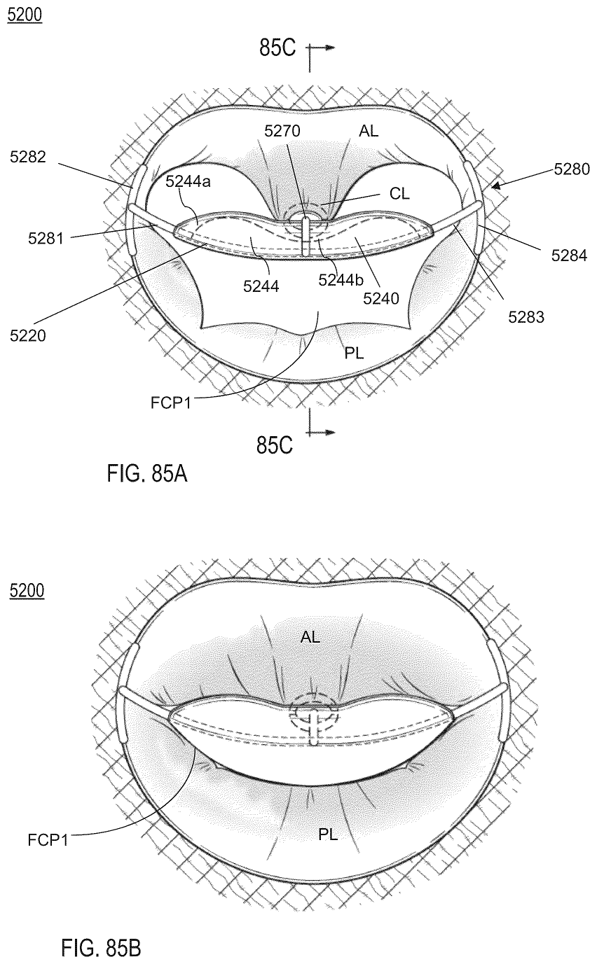

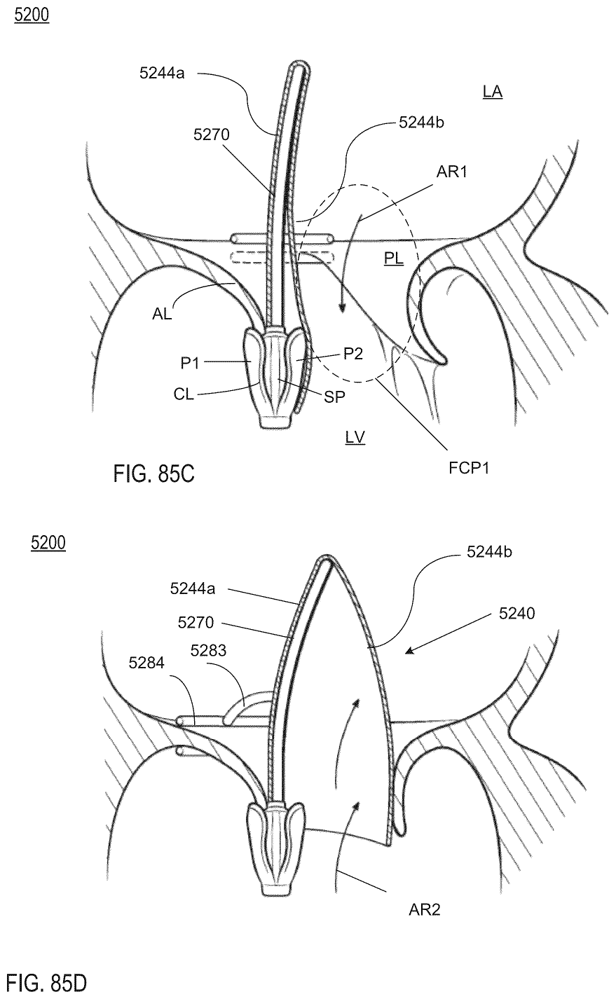

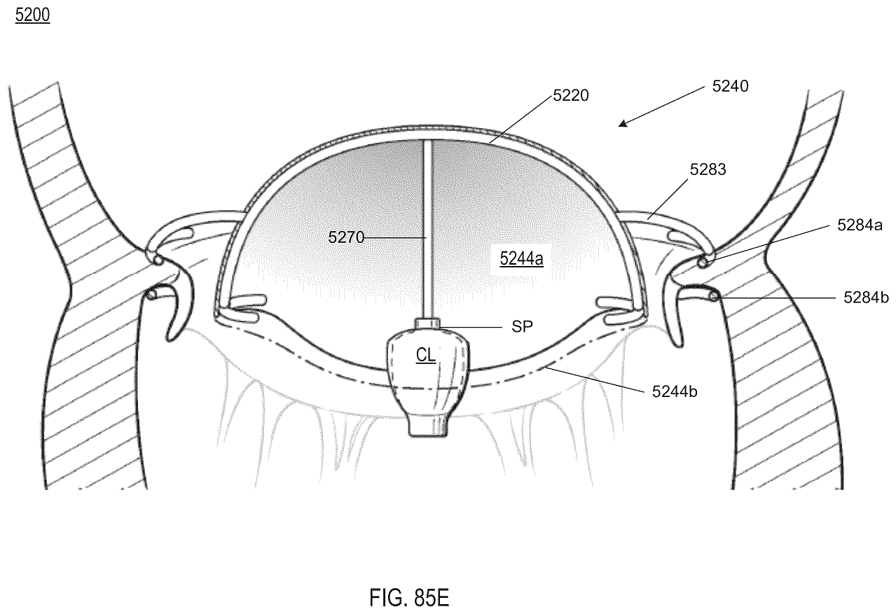

In the SOD embodiments described above, the clip CL is used to approximate the two native leaflets, in one or more locations along the leaflet margins or coaptation line, and the SOD is secured to the native valve via the clip CL, and optionally an annulus connector (with one or more annulus anchors). However, in other embodiments, it may be desirable to couple the SOD to the native valve in part by one or more clips, each of which is secured only to a single leaflet. One such embodiment is shown in FIGS. 85A-E. SOD 5200 includes occluder 5240, frame 5220, clip connector 5270 attached to clip CL, which, in turn, is attached to a single anterior leaflet AL. Further, occluder 5240 includes an annulus connector 5280 with two arms 5281 and 5283, anchors 5282 and 5284. Occluder 5240 has one membrane 5244 than can lie flat against or adjacent to clip CL and the native leaflet to which it is clipped, providing greater flow area during diastole. While in various other embodiments, a support frame of an SOD is coupled to the clip via a clip connecter, in this embodiment, clip connector 5270 is coupled to a first leaflet engaging or first movable membrane portion 5244a, while a second leaflet engaging or second movable membrane portion 5244b is not coupled to clip CL. Unlike some of the previously described embodiments, clip CL only captures one native leaflet (e.g., anterior leaflet AL)

As shown in FIG. 85A, a single flow control portion FCP1 of the clipped valve is a larger flow area than two flow control portions that would have been formed if a central clip CL was used for approximating anterior and posterior leaflets AL, PL. FIG. 85A shows SOD 5200 during diastole and FIG. 85B shows SOD 5200 during systole. Note that SOD 5200 occludes flow control portion FCP1 during the systole. FIG. 85C shows a cross-sectional area of SOD 5200 coupled to clip CL along a plane 85C (plane 85C is shown in FIG. 85A). As shown in FIG. 85C-D, a part of first movable membrane portion 5244a is coupled to a clip connector 5270, which is coupled to clip CL, preferably through clip spacer SP. As shown in FIGS. 85C-D, Clip CL is configured to engage anterior leaflet AL. When anterior leaflet AL is captured between clip spacer SP and paddle P1 of clip CL, first membrane portion 5244a and captured portion of leaflet AL are retained in an approximated relationship to each other (and preferably in direct contact with one another), at this clipped location, throughout the cardiac cycle. Further, SOD 5200 is engaged to annulus of a native valve via annulus connector anchor 5284 and annulus connector arm 5283. FIG. 85C shows SOD 5200 during diastole (e.g., a flow of blood through FCP1 from left atrium LA to left ventricle LV is indicated by an arrow AR1), while FIG. 85D shows SOD 5200 during systole (e.g., the flow blood is occluded by second movable membrane portion 5244b in sealing engagement with posterior leaflet PL, as indicated by an arrow AR2).

In this embodiment, as shown in FIG. 85A, SOD 5200 is preferably configured with two annulus anchors 5282 and 5284, one adjacent each commissure, each annulus anchor including a member (e.g., 5284a) above the MV annulus, and an opposing member (e.g. 5824b) below, so as to trap therebetween annulus tissue (as shown in FIG. 85E), as with other embodiments described above. In this figure, membrane 5244a is shaded and membrane 5244b is shown in phantom line.

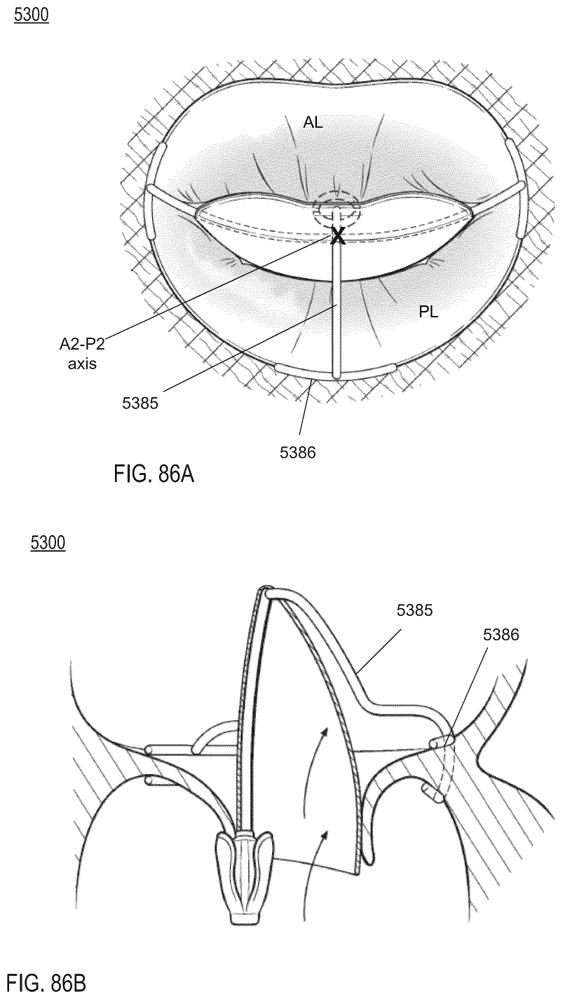

In another embodiment, as shown in FIGS. 86A-B, SOD 5300 includes a third annulus connector including a third arm 5385 and third annulus anchor 5386 to provide greater stability in the A-P direction through the anterior and posterior leaflets of a native valve, in a manner to keep the apex of SOD 5300 (labelled “X” in FIG. 86A) from tilting or rocking toward the anterior or posterior annulus of the mitral valve. FIG. 86A shows SOD 5300 during diastole, while FIG. 86B shows SOD 5300 during systole, similar to FIGS. 85C-D.

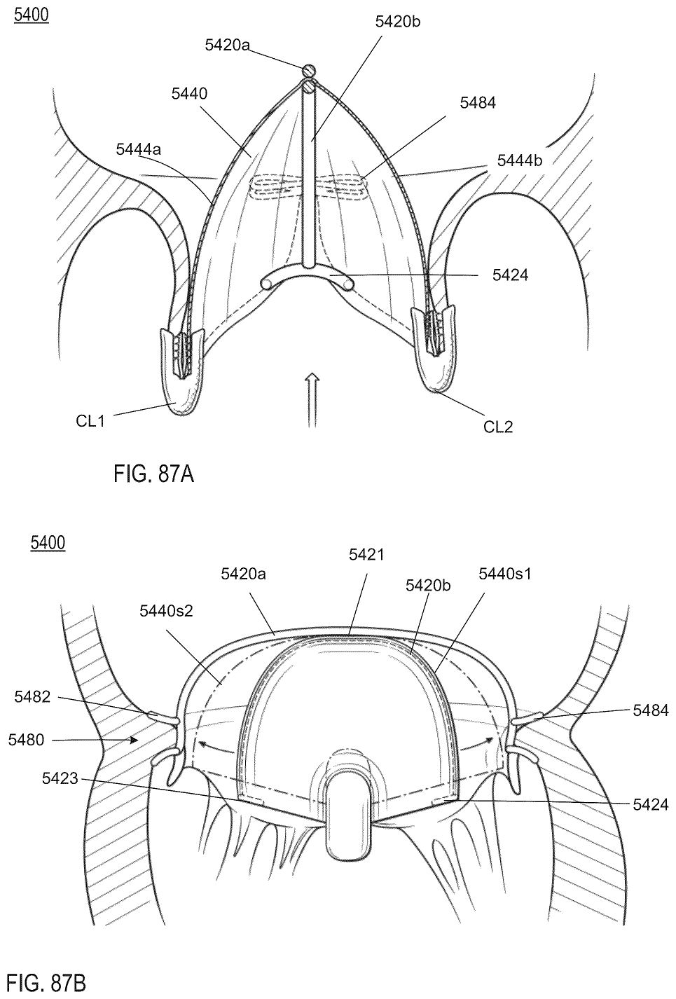

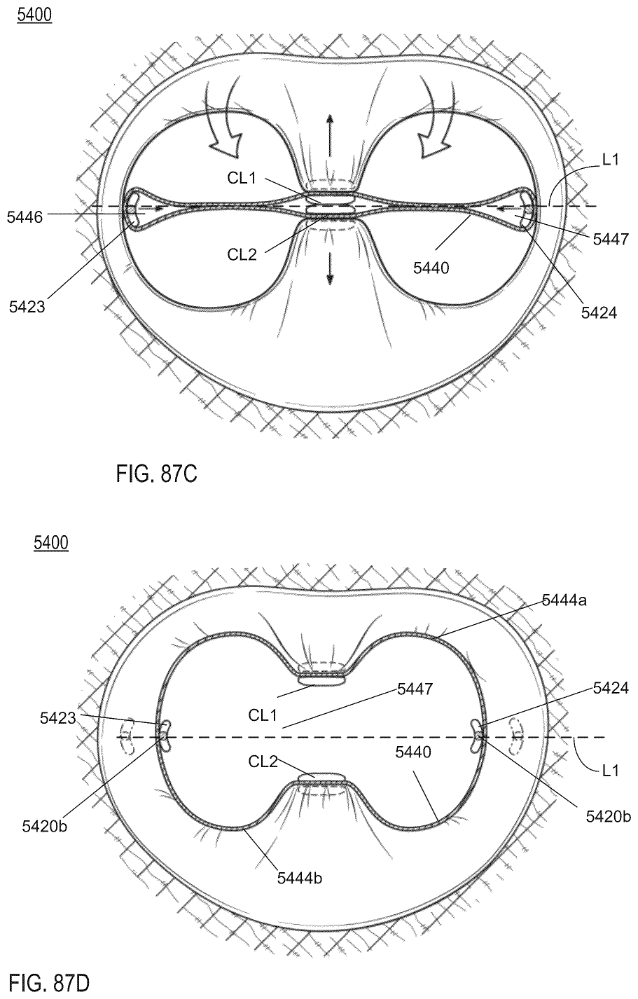

In some embodiments, an SOD can be connected to a native valve by securing the support frame of the SOD to the native annulus of the native valve by an annulus connector and securing only the occluder of the SOD to each of two separate clips, each clip connected to a single native leaflet. Thus, in such an embodiment, there is no direct, hard connection of the SOD frame to any one of the clips. One such embodiment in shown in FIGS. 87A-D. SOD 5400 includes a flexible membrane occluder 5440 engaged with clips CL1 and CL2. As shown in FIG. 87A, clips CL1 and CL2 are joining edges of native leaflets and occluder membrane simultaneously, and move towards and away from each other during diastole and systole, respectively. The embodiment of FIGS. 87A-D provides a large flow orifice area during diastole, as shown in FIG. 87C. Further, stability for SOD 5400 may be provided by annulus connector 5480. SOD 5400 and clips CL1 and CL2 are delivered and engaged with leaflets of a native valve during the same surgical procedure. FIG. 87B shows a side view of SOD 5400, with occluder 5440 taking a smaller shape 5440s1 during systole (during systole a flow control portion decreases along line L1, as shown in FIG. 87C), and a larger shape 5440s2 during diastole (during diastole the flow control portion increases along line L1). The flexible frame 5420b's terminal ends flex inward in systole (FIG. 87D) due to the pressurizing of SOD inner cavity, and clips CL1 and CL2 move slightly downward into the LV as occluder 5440 transitions between a first collapsed configuration, with flexible membrane portions 5444a and 5444b in close proximity to one another and to line L1 (FIG. 87C), and a second pressurized, blood-filled configuration (FIG. 87D) with flexible membrane portions 5444a and 5444b in spaced-apart relationship to one another and spaced away from line L1.

Further, a relatively rigid outer support frame 5420a extending between and terminating in annulus anchors 5482 and 5484 is used to provide stability for SOD 5400. Flexible support frame 5420b is coupled to outer support frame 5420a via a joint element 5421. Joint element 5421 does not move between diastole and systole. FIG. 87C shows a top view of SOD 5400 during diastole, and FIG. 87D shows a top view of SOD 5400 during systole. As shown, occluder 5440 is coupled to clip CL1 and CL2 and anchored to the native annulus at annulus anchors 5482 and 5484 (not shown in FIGS. 87C and 87D). Terminal ends of flexible member 5420b are configured with transverse members 5423 and 5424 sized to maintain membranes 5444a and 5444b apart from one another, in diastole, resulting in membrane openings 5446 and 5447 therebetween. Openings 5446 and 5447 provide fluid communication to the interior of occluder 5440 from the LV such that, at the start of systole, the rapid rise in systolic pressure will urge membranes 5444a and 5444b to move apart and permit filling of occluder 5440 with blood. As shown in FIG. 87D, transverse members 5423, 5424 move towards each other during the transition from diastole to systole (the positions in systole shown in solid lines and corresponding to occluder shape 5440s1, and in diastole in dashed lines and corresponding to occluder shape 5440s2) due to inward flexing of the terminal ends of flexible support frame 5420b due to the pressurizing of the interior of occluder 5440 by blood during systole.

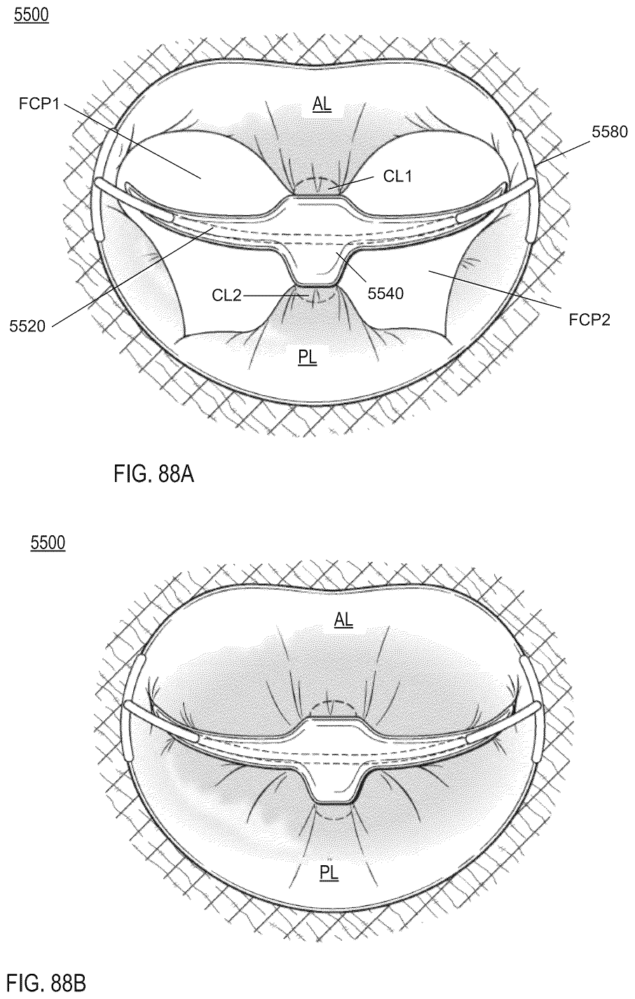

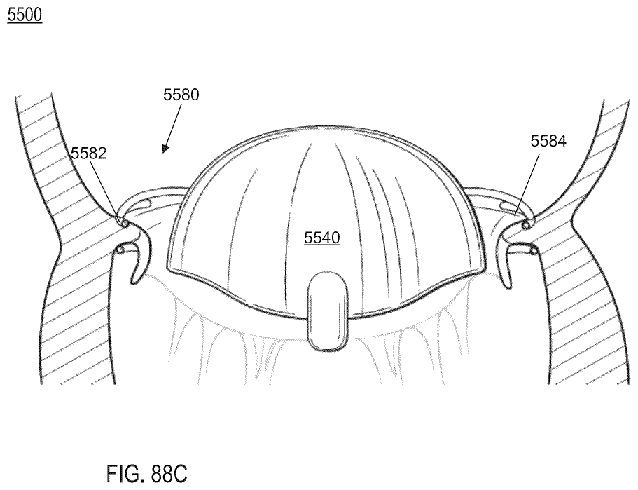

In an alternative to SOD 5400, a static occluder can be used instead of a flexible membrane occluder. Such an alternative embodiment is shown in FIGS. 88A-C. SOD 5500 includes a static occluder 5540, supported on frame 5520 that includes annulus connector 5580; occluder has clip engaging portions that can be engaged by clips to secure leaflets AL (at A2) and PL (at P2) to occluder; flow control portions FCP1, FCP2 formed between clips, occluder, leaflets, and commissures. As shown in FIGS. 88A-B, occluder 5540 may be shaped to correspond to the shape of the native valve opening (as redefined by being clipped to the central portion of occluder 5540) to optimize the coaptation of the native leaflets with occluder 5540. The shape of occluder 5540 is configured and sized to occupy at least the regurgitant area between the clipped native leaflets, in systole, so as to restore competency to the native valve, as shown in FIG. 88B. In this embodiment, the captured leaflets are held in a spaced apart fixed relationship during the cardiac cycle (i.e., occluder serves as a wider spacer, unlike MitraClip and to a lesser extent Pascal, and embodiment of FIG. 12A, where leaflets are closely approximated). Spacing apart of captured leaflet margins produces a larger orifice area in diastole as shown in FIG. 88A. FIG. 88C shows a side view of static occluder 5540. Similar to other occluders (e.g., flexible occluders), occluder 5540 is configured to connect with annulus via annulus connector 5580 which includes two annulus connectors 5582 and 5584, one adjacent each commissure, each annulus connector including a member above the MV annulus, and an opposing member below, so as to trap annulus tissue therebetween.

In another embodiment, a static occluder can be implemented as an inflatable structure. As shown in FIG. 89, SOD 5600 includes occluder 5640, which is implemented as a fluid-filed (or fluid-fillable) bladder. Occluder 5640 includes in an internal structure frame 5620 that provides a load path or structural connection between clips CL1, CL2 and annulus connector 5680, to supplement, or replace, the bladder structure as a load path. The bladder can be filled with a suitable fluid (e.g., saline or other physiologically-compatible liquid, or CO₂ or other physiologically-compatible gas) via a fluid port 5650. Bladder may be reconfigurable between a delivery configuration, in which the bladder is devoid of fluid, or partially filled, to occupy less volume in the delivery device, and a deployed configuration, in which the bladder is filled with more fluid than the delivery configuration, thus occupying a larger volume to provide the occlusion function of occluder 5640. The deployed volume may be adjusted during the treatment procedure to achieve the desired degree of occlusion between the native leaflets, and thus the desired degree of reduction in regurgitation of the valve. This volume adjustment is represented by the difference between the dashed perimeter line and the solid perimeter line in FIG. 89. The deployed volume can also be adjusted in a later surgical procedure to resolve residual mitral regurgitation that may develop with disease progression. The bladder affords a degree of compliance to the bladder leaflet-contacting surfaces. As such, the leaflet-contact surfaces are reconfigurable or deformable in use under the load imparted by native leaflets during coaptation in systole.

It is contemplated that the position of a clip may be adjusted after an initial clipping procedure, or during the course of a clipping procedure, if the initial placement does not sufficiently reduce the degree of regurgitation, and that an SOD may be delivered to the valve in connection with the clip repositioning. For example, a mitral valve MV may be initially clipped with a single clip CL in an off-center position, e.g., closer to the commissure on the side of the valve in which regurgitation is greater, such as shown in FIG. 37C. If the regurgitation is still excessive, the clip CL may be repositioned closer to, or at, the center of the leaflets, such as shown in FIG. 37B. In connection with the repositioning of the clip (e.g., as part of the same procedure, as described in more detail above for various embodiments), an SOD may be delivered to and implanted in the valve MV, with an occluder disposed in each of the flow control portions FCP1, FCP2 produced by the clipping procedure.

In an example embodiment, a method for deploying an SOD and a clip includes the following steps. At step 1, a clip CL is placed towards a location of maximum observed mitral regurgitation (e.g., an eccentric location in mitral valve MV). At step 2, an echo assessment (or any other suitable imaging) of clipped mitral valve MV is made while keeping delivery catheter engaged to clip CL. At step 3, the method includes determining if mitral regurgitation MR is not sufficiently low, as indicated by the assessment made at step 2 (e.g., MR grade>1 according to current guidelines). If MR grade>1 (step 3, yes), at step 4, the method includes disengaging clip CL from its first position (e.g., eccentric position) and repositioning clip CL to a second position (e.g., more centrally to approximate and capture leaflets at the A2-P2 location). Alternatively, if the MR grade is less than or equal to one (step 3, no), clip CL may be left in place. At step 5, an SOD is delivered to the left atrium LA and is coupled to clip CL using any suitable technique as described above.

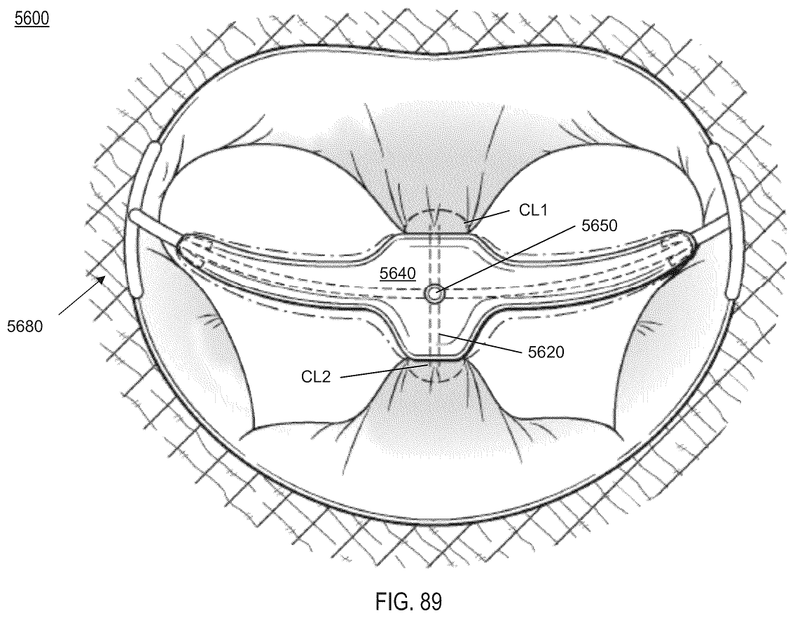

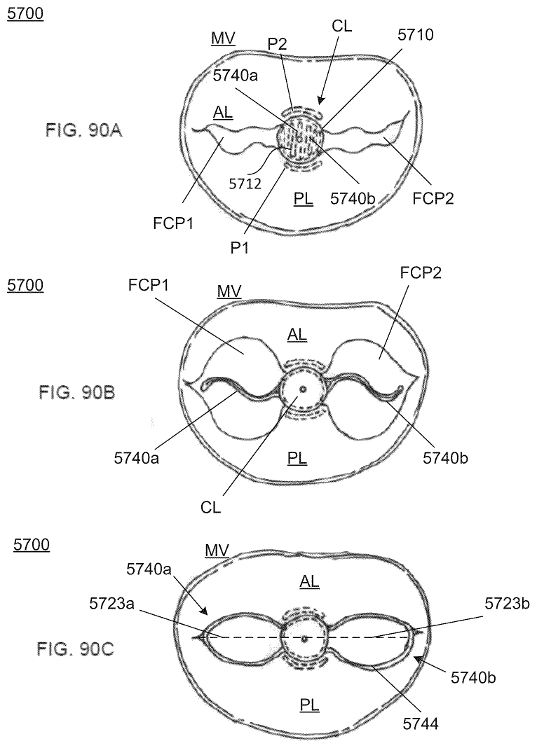

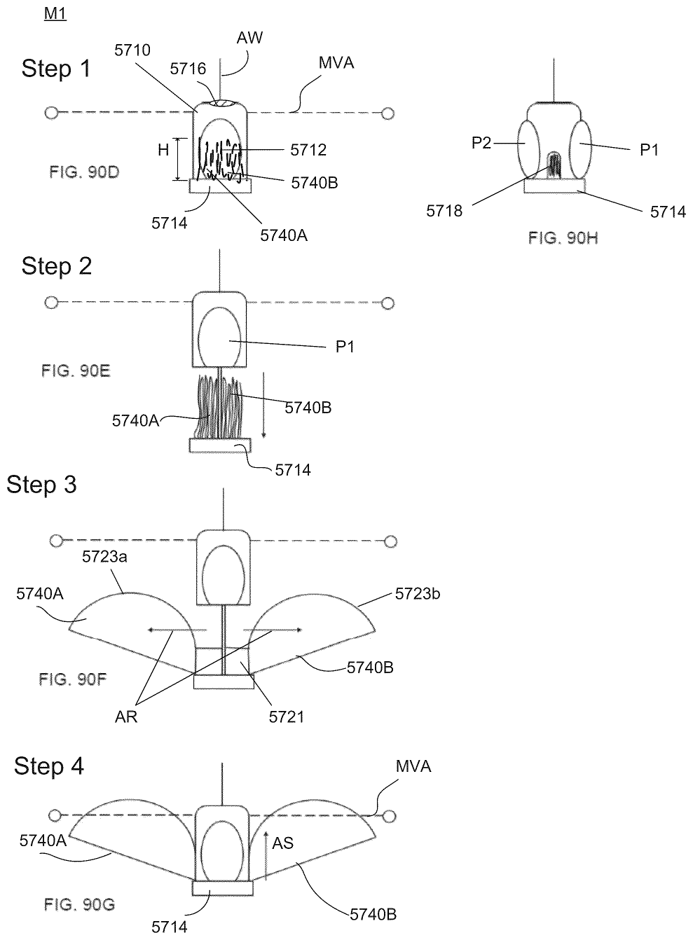

In some embodiments, an SOD can be configured to be integrated with a clip, and to contain the occluders in a stored or delivery configuration, so that the occluders may be optionally deployed if the clipping of the native valve does not sufficiently mitigate the regurgitation of the native valve. One such embodiment is shown in FIGS. 90A-90H. SOD 5700 includes a body 5710 that incorporates a clip CL (in this embodiment, having two paddles-P1 and P2-on opposite sides of the body 5710), and occluders 5740a and 5740a. Body 5710 includes an occluder storage cavity 5712 in which occluders 5740a and 5740b may be stored (e.g., in a storage or delivery configuration) during the delivery of SOD 5700. As with other embodiments described above, clip CL is configured to connect the anterior leaflet AL and posterior leaflet PL of a native valve, such as the mitral valve shown in FIGS. 90A-G, in this embodiment by capturing each native leaflet between paddle P1, P2 of clip CL and body 5710 (which is similar to a spacer included in other clip embodiments disclosed above).

FIG. 90A shows native mitral valve MV after SOD 5700 has been delivered and the anterior leaflet AL and posterior leaflet PL clipped to body 5710 between paddles P2, P1, respectively. As shown in FIG. 90A, occluders 5740a and 5740b are stowed within occluder storage cavity 5712. Mitral valve MV is shown in systole, and the native leaflets AL, PL are not coapting against each other in the two flow control portions FCP1, FCP2 produced by the clipping procedure—the valve is thus still suffering from regurgitation. This residual regurgitation can be addressed by disposing occluders 5740a and 5740b in flow control portions FCP1, FCP2, respectively. FIG. 90B shows the mitral valve MV, during diastole, with occluders 5740a and 5740b deployed into the corresponding flow control portions FCP1 and FCP2. In this embodiment, occluders 5740a and 5740b are configured as pseudo-valves, e.g., with collapsible, flexible movable membranes 5744, which are configured to collapse during diastole (as shown in FIG. 90B, allowing blood to flow through flow control portions FCP1, FCP2) and to expand and to coapt with native leaflets AL, PL during systole (as shown in FIG. 90C, to prevent regurgitant blood flow from the left ventricle to the left atrium). Occluders 5740a, 5740b may be implemented similar to other embodiments of pseudo-valve occluders disclosed above, and may include support frame, with an occluder connecting portion 5721 and membrane support members (e.g., arcuate wire frames) 5723a, 5723b, as best seen in FIG. 90C (dashed lines), and FIG. 90F.

FIGS. 90D-90H illustrate a method M1 by which SOD 5700 can be deployed in a native valve (e.g., mitral valve MV as shown in FIGS. 90A-C). FIG. 90D shows step 1 of process M1, in which SOD 5700 is delivered to mitral valve MV so that is disposed substantially below the plane of the mitral valve annulus MVA. An elongated actuator wire AW (e.g., a guidewire or any other suitable element such as a thin cable) passes through an opening 5716 located at a top portion of body 5710 (as shown in FIG. 90D) and through occluder storage cavity 5712, and engaging with an occluder storage closure 5714. Occluder storage closure 5714 is movable relative to body 5710 between a first position (FIG. 90D) in which it closes occluder storage cavity 5712 and a second position (FIG. 90E) in which it is spaced from body 5710. Occluder storage closure 5714 is also configured to support occluders 5740A and 5740B via occluder connection portion 5721, as shown in FIG. 90F. At step 2 of process M1, elongated actuator wire AW is configured to be operable to move occluder storage closure 5714 away from body 5710, allowing occluders 5740a, 5740b to be released.

FIG. 90F shows step 3 of process M1, with occluders 5740A and 5740B self-deploying, such as by self-expansion of membrane support members 5723a, 5723b, as indicated by arrows AR. At step 4, as shown in FIG. 90G, actuator wire AW is operated to draw occluder storage closure 5714 back into engagement with body 5710, as indicated by arrow AS, thereby moving occluding elements 5740A and 5740B into operative locations between leaflets AL and PL, in flow control portions FCP1, FCP2, respectively. As best seen in FIG. 90H (left side view of FIG. 90D), body 5710 also includes slots 5718 (one on each side) through which occluder connection portion 5721 can extend when occluders 5740A, 5740B are in their deployed configuration. (Note that in FIG. 90H, occluders 5740A, 5740B are shown in their stored configuration.)

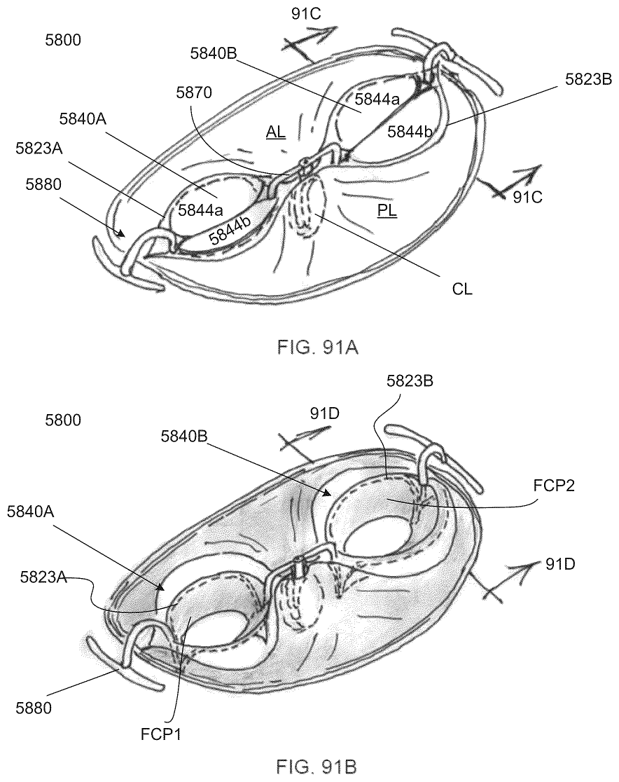

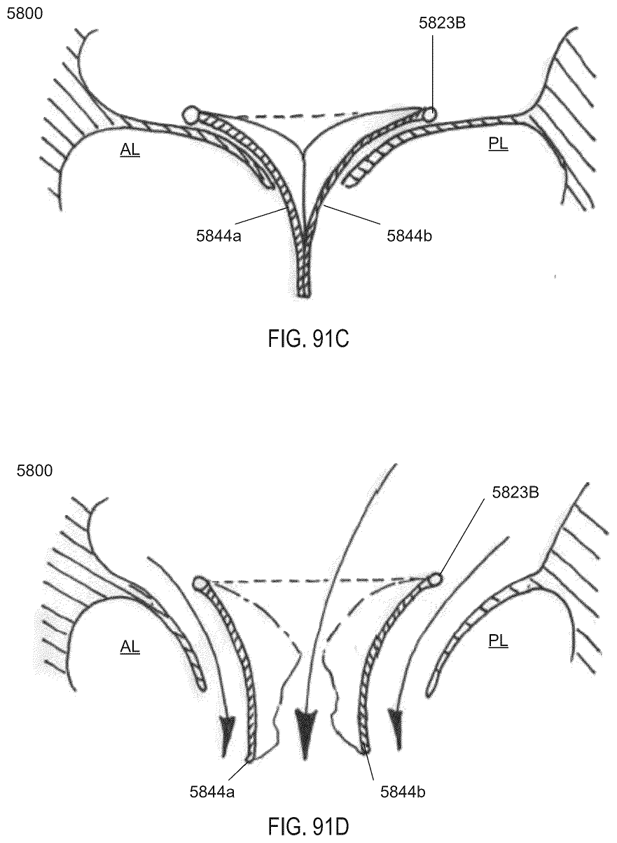

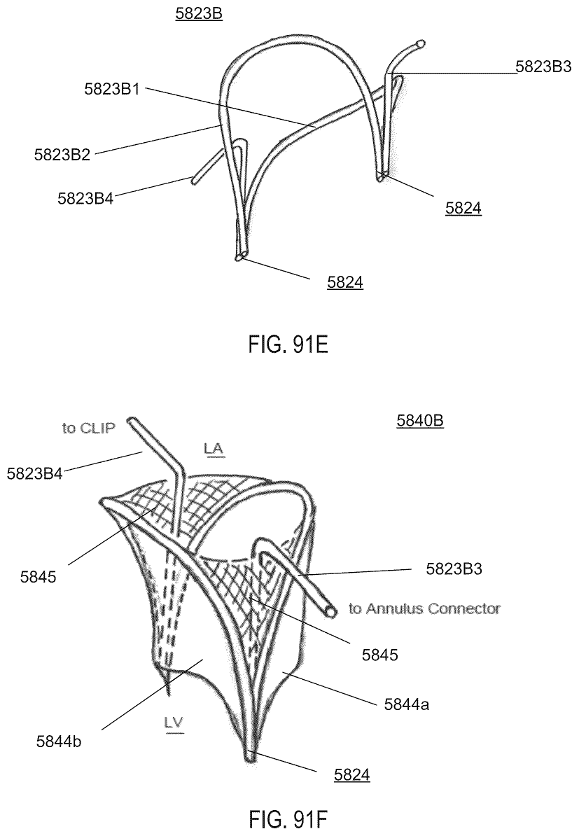

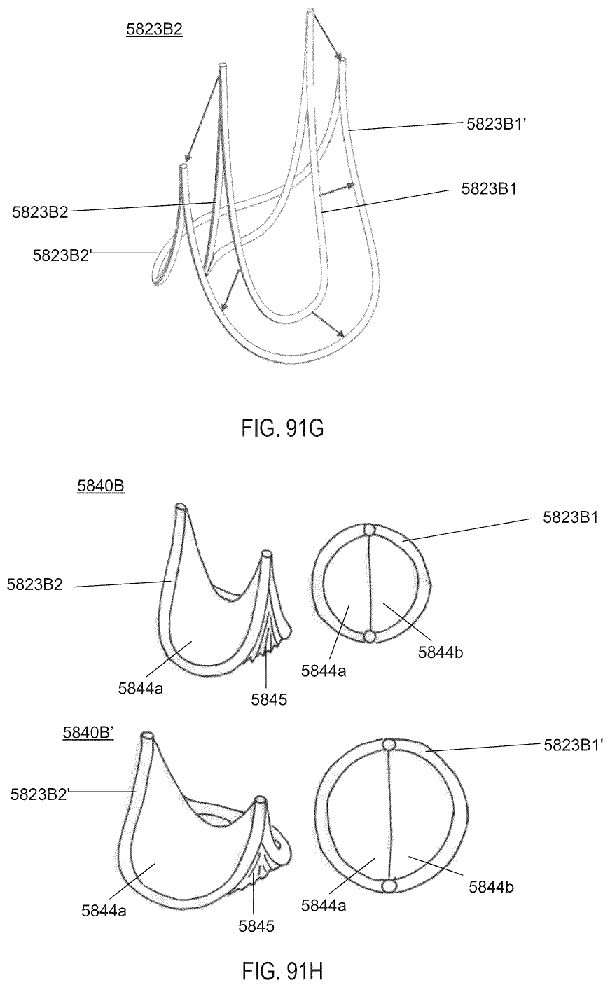

In some embodiments, an SOD can include occluders that function both to movably coapt with the native leaflets (as with the pseudo-valve embodiments described above) and to permit blood to flow in one direction (e.g., from atrium to ventricle) internally (similar to the prosthetic valve embodiments described above). One such embodiment is shown in FIGS. 91A-91H. In this embodiment, SOD 5800 includes occluders 5840A and 5840B supported on a support frame, which in turn is coupleable to a clip CL via a clip connector 5870. SOD 5800 also includes annulus connector 5880 engageable with the annulus of the native valve. Each of occluders 5840A and 5840B includes membrane support frames 5823A and 5823B, respectively. FIGS. 91E and 91F illustrate membrane support frame 5823B in more detail (membrane support frame 5823A is essentially identical). Membrane support frame 5823B includes two U-shaped support frame members 5823B1 and 5823B2, joined together at the open ends of the U-shapes to form commissural posts 5824, and two L-shaped connector frame members 5823B3 and 5823B4, which are connected at their first ends to the commissural posts 5824 of the support frame members 5823B1 and 5823B2, and at their second ends to annulus connector 5880 and clip connector 5870, respectively. As shown in FIG. 91F, support frame members 5823B1 and 5823B2 support first and second leaflet engaging portions 5844a, 5844b, respectively, which together essentially function as movable prosthetic leaflets or membranes of a bicuspid valve. That is, first and second leaflet engaging portions 5844a, 5844b (i.e., first and second movable membrane portions) can coapt with each other at their lower ends, to prevent blood flow therebetween, and can be spaced apart (as shown in FIG. 91F) to permit blood flow therethrough. These prosthetic leaflets may be constructed from animal tissues, such as pericardium, or from bioengineered polymers, as described above with reference to other embodiments. As shown in FIG. 91F, occluder 5840B also includes commissure membranes 5845 coupled between the lower portions of U-shaped support frame members 5823B1 and 5823B2 and extending between the U-shaped support frame members from the lower portions to the level of the commissural posts 5824. As such, the commissure membranes prevent blood from passing between the frame members in systole, thus defining collectively with first and second leaflet engaging portions 5844a, 5844b a flow passage through which blood can selectively flow through occluder 5840B in diastole. During systole, as shown in FIGS. 91A and 91C, the first and second leaflet engaging portions 5844a, 5844b of each occluder 5840A and 5840B are configured to coapt and cooperate with the native leaflets AL and PL, respectively, and are also configured to simultaneously coapt with each other to prevent or reduce regurgitant flow from the LV to the LA. In a sense, the prosthetic leaflet engaging portion 5844a, 5844b act as extensions to the native leaflets that, due to disease, have become retracted or have been drawn apart and consequently unable to adequately coapt with each other in systole. During diastole (as shown in FIGS. 91B and 91D), the leaflet portions 5844a and 5844b are spaced apart from each other and from native leaflets AL and PL, respectively, to allow flow from the LA to the LV (as indicated by arrows in FIG. 91D). To the extent necessary, the native leaflets also engage with commissure membranes 5845 during systole to seal off or prevent retrograde flow from the LV to the LA. As is apparent from FIGS. 91A and 91C, during systole the occluders 5840A and 5840B coapt with the native leaflets, preventing the retrograde flow of blood between the occluders and the native leaflets, and the leaflet engaging portions 5844a, 5844b also coapt against each other, preventing the retrograde flow of blood through occluders 5840A, 5840B-SOD 5800 therefore prevents retrograde flow through the flow control portions FCP1, FCP2 resulting from clipping of the native valve. Correspondingly, during diastole (FIGS. 91B and 91D), blood can flow freely between the occluders 5840A, 5840B and the native leaflets, as well as between the spaced leaflet engaging portions 5844a, 5844b. As such, occluders 5840A and 5840B cooperate with native leaflets AL and PL to function as a one-way valve.

As shown in FIG. 91G, in some embodiments support frame member 5823B may be varied in size. For example, support frame member 5823B may be expanded (e.g., by a balloon or other known technique) as indicated by the arrows in FIG. 91G from a smaller diameter configuration (shown in FIG. 91G with frame members 5823B1 and 5823B2, but omitting 5823B3 and 5823B4) to a larger diameter configuration (with frame members identified as 5823B1′ and 5823B2′). Correspondingly, occluder 5840B is shown in FIG. 91H in a smaller diameter configuration (top portion of FIG. 91H) and a larger diameter configuration (lower portion of FIG. 91H), in bottom perspective and bottom views, with the first and second leaflet engaging portions 5844a, 5844b coapted together. Note that commissure membranes 5845 expand during expansion of frame members 5823B1, 5823B2, and thus needs to be elastic, or expandable from a crimped or collapsed membrane state to an expanded membrane state, to accommodate the size change. In some use cases, the occluder 5840B may be expanded to a desired size during the initial delivery procedure, e.g., to optimally coapt with the native leaflets, or may be expanded in a subsequent procedure for instance, in a procedure where a previously implanted patient presents with recurrent mitral regurgitation due to disease progression. Although shown as circular in bottom view in FIG. 91H, the periphery of frame 5283B2 may be elliptical, oval, etc.

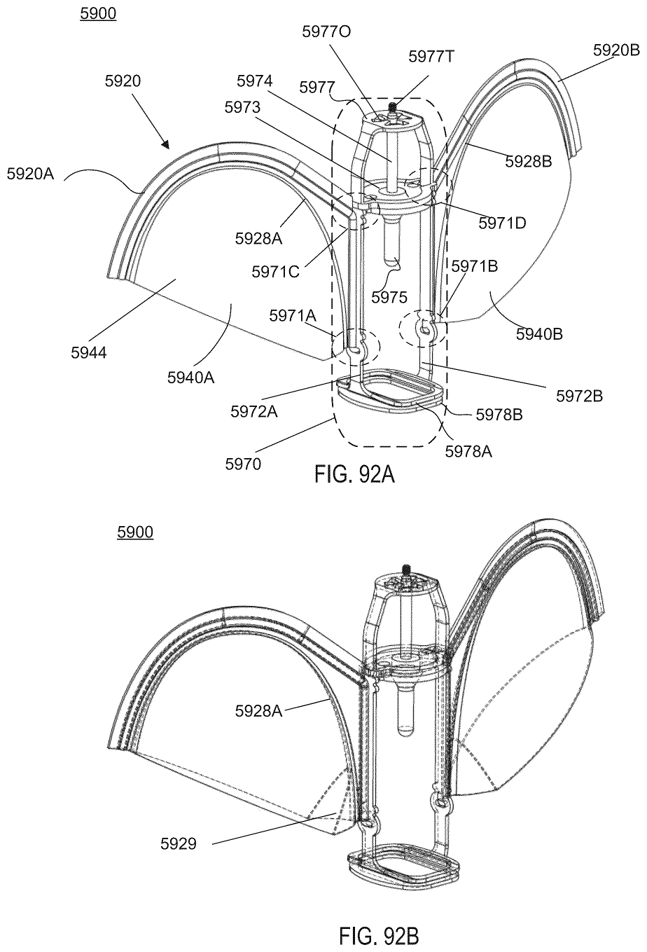

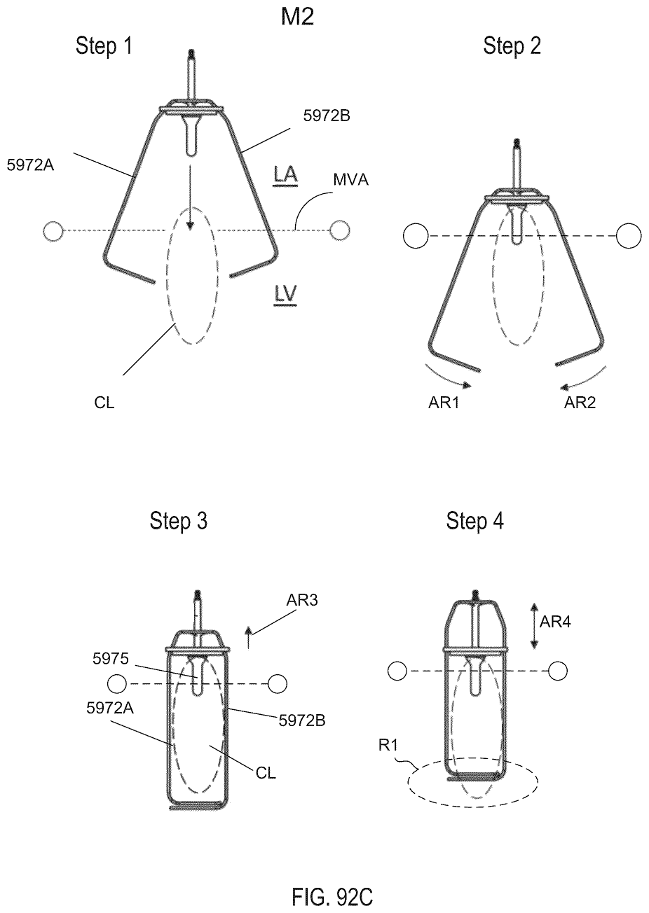

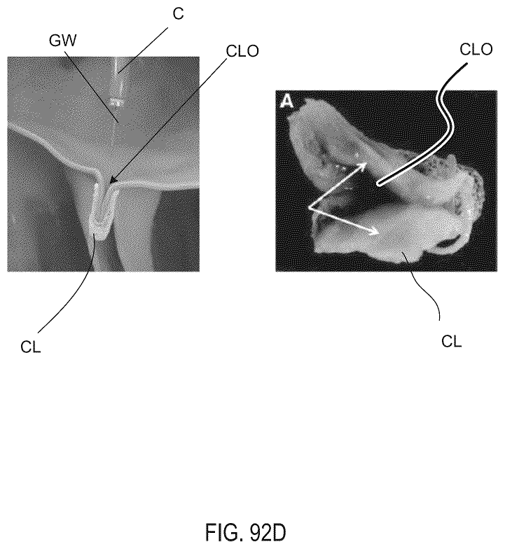

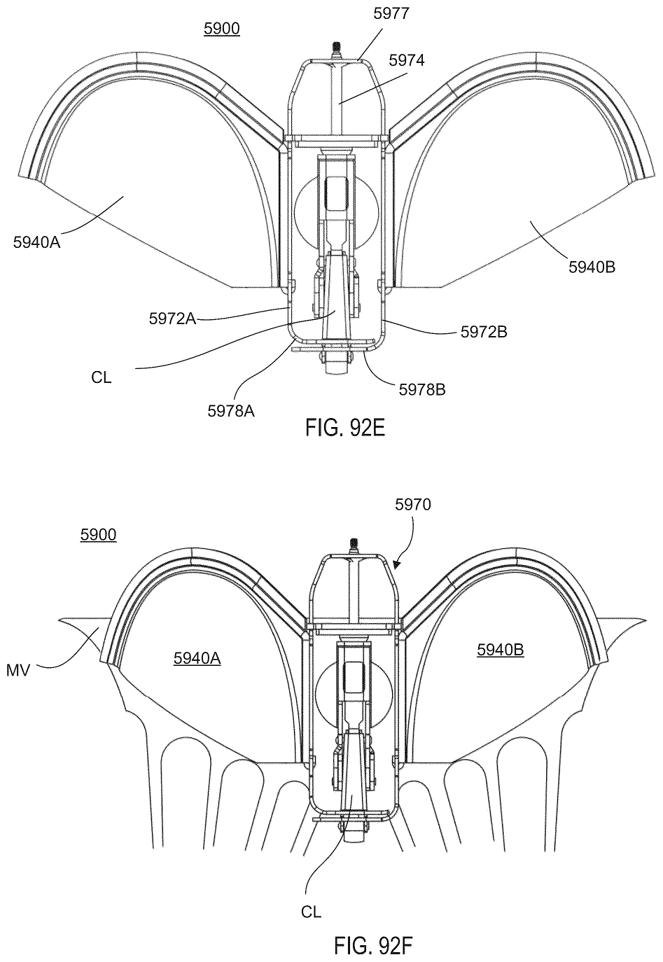

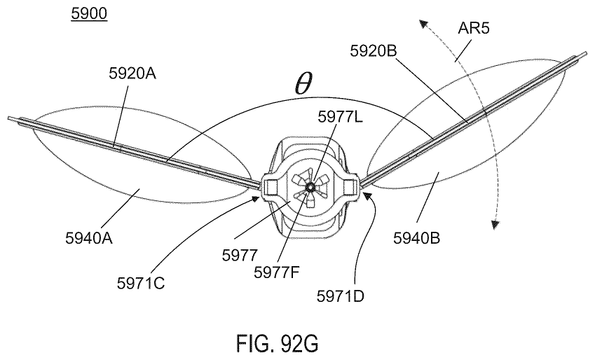

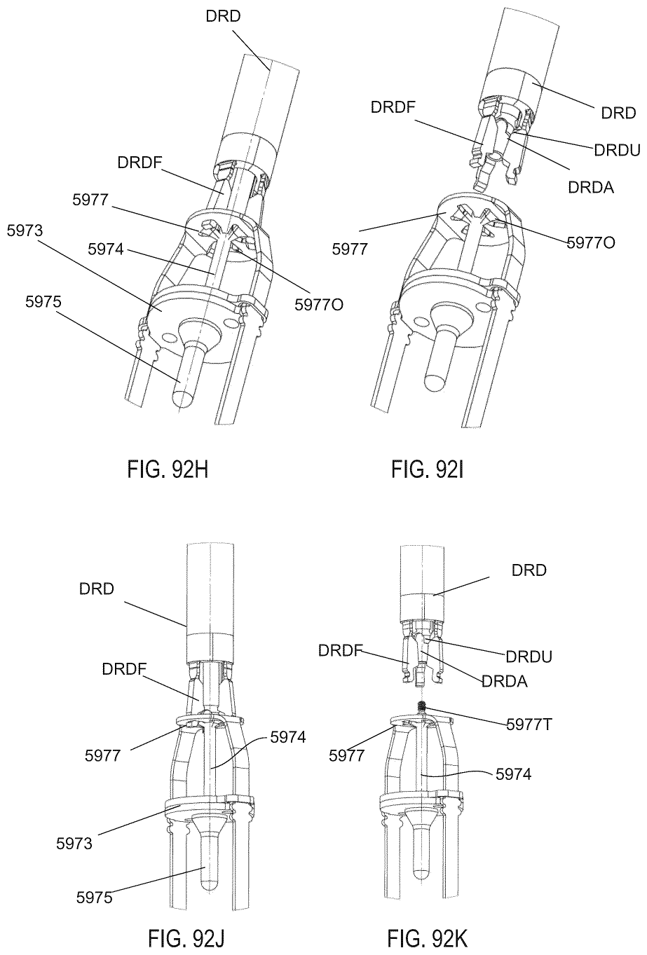

In some embodiments disclosed above, an SOD can be secured to a previously-delivered clip (e.g., during a procedure separate from, and later than, the clipping procedure), such as by a suture loop. In other embodiments, the SOD can incorporate a frame structure that can directly engage with the clip. The frame structure can be deployed in the same procedure that initially deploys the clip, as an adjunct to the clip, or in a separate later procedure where the patient implanted with a previously deployed clip presents with recurrent mitral regurgitation. Such an embodiment in shown in FIGS. 92A to 92I. SOD 5900 includes a clip connector 5970 and occluders 5940A and 5940B that are coupled to, and supported on, clip connector 5970 by support frame 5920.

SOD 5900 is configured to be attached to an existing clip CL via clip connector 5970. Clip connector 5970 can be actuated (such as by a delivery and/or retrieval catheter, as described in more detail below) to selectively engage with, or disengage from, a clip CL by capturing (or releasing) clip CL between two relatively movable components of clip connector 5970. In this embodiment, clip connector 5970 includes clip connector arms 5972A and 5972B, and hoop members 5978A and 5978B, which are joined together at a first, upper end by a coupling element 5977 and the lower ends of which are laterally displaceable towards and away from each other to selectively capture (or release) clip CL. The clip connector arms 5972A and 5972B can be actuated, i.e., caused to move towards or away from each other, by axial movement of an actuating member 5973 relative to coupling element 5977, which axial movement is produced by axial movement of drive member 5974. Drive member 5974 can selectively engage with an actuator DRDA of a delivery/retrieval device DRD (e.g., threadably engage with actuator DRDA at thread portion 5977T), as described in more detail below with reference to FIGS. 92H to 92K. Clip connector 5970 also includes an axial clip post 5975 extending downwardly from actuating member 5973, which can be inserted into a central portion of clip CL, as illustrated and described in more detail below. It should be noted that members 5973, 5974, and axial clip post 5975 form an integral assembly. There is no relative movement between these elements. A coupling member 5977 is configured to slide/translate relative to member 5974 to set the relative position of arm 5972A with respect to arm 5972B.

Clip support frame 5920 is coupled to clip connector arms 5972A and 5972B by coupling joints 5971A, 5971B, 5971C, and 5971D. More specifically occluding element 5940A is supported on an arcuate frame element 5920A coupled to coupling joints 5971A and 5971C, and occluding element 5940B is supported on an arcuate frame element 5920B coupled to coupling joints 5971B and 5971D. Further, coupling joints 5971A-5971D may be implemented as hinges, and thus may allow frame elements 5920A and 5920B to pivot relative to clip connector 5970, as shown and described in more detail with reference to FIG. 92G.

As with other embodiments disclosed above, occluding elements 5940A and 5940B are configured as pseudo-valves, i.e., they include flexible membranes 5944 and to be placed into respective flow control portions FCP1 and FCP2 formed between leaflets of the native valve by clipping of the native valve, to prevent regurgitation of blood during systole. Occluding elements 5940A and 5940B are configured to inflate or have opposed portions of flexible membrane move away from each other during the systole (i.e., to an occluder open configuration), thereby coapting with the native leaflets and occluding the flow control portions. Occluders 5940A and 5940B may optionally include internal frame elements or stitched seams 5928A and 5928B to define the inner cavity of occluder 5940A, 5940B in a streamlined manner to promote adequate washing of the internal membrane surfaces between diastole and systole. In order to bias the occluders towards their open configuration and to aid in filling with blood during systole, occluders 5940A, 5940B may include spring elements 5929 as shown in FIG. 92B. Spring elements 5929 maintain a relative spacing or gap between the opposed portions of flexible membrane, over a segment of the entire membrane open free margin length, so as to in diastole allow the inside of the occluder to be instantaneously exposed to the changing pressure of the LV over the cardiac cycle, and as such entrain the occluder membranes to move from the relatively-collapsed diastolic configuration to the spaced-apart systolic configuration.

FIG. 92C shows an example method M2 for delivering SOD 5900 into a native valve (e.g., mitral valve) and for attaching SOD 5900 to a clip CL by capturing clip CL between clip connector arms 5972A and 5972B. In FIG. 92C, occluders 5940A and 5940B are not shown. During the steps of the method M2, the occluders preferably assume a compact, collapsed configuration held closely against the frame members 5972A and 5972B. This minimizes the dynamic forces exerted on the SOD from the cardiac cycle during deployment, and facilitates positioning of the occluders into respective flow control portions FCP1 and FCP2 during the coupling of clip connector 5970 to clip CL. At step 1 of method M2, SOD 5900 may be delivered to clip CL in an open configuration (i.e., with the actuating member 5973 in an upper position (i.e., proximal to coupling 5977) so that the lower ends of clip connector arms 5972A and 5972B are spaced from each other), such that the lower ends of clip connector arms 5972A and 5972B are disposed below the plane of the native valve annulus, passing to either side of clip CL (i.e., with each arm passing through a respective flow control portion FCP1, FCP2). As shown in step 2, SOD 5900 is disposed so that axial clip post 5975 is engaged with (i.e., disposed within a central portion of) clip CL, and the lower ends of clip connector arms 5972A and 5972B are disposed below clip CL. Driver member 5974 is actuated (e.g., by a delivery catheter, not shown in FIG. 92C), i.e., translated axially so that it urges actuating member 5974 axially downwardly, urging the lower ends of clip connector arms 5972A and 5972B towards each other to close below a bottom portion of clip CL, as indicated by arrows AR1 and AR2. At step 3 of method M2, clip connector arms 5972A and 5972B are shown closed under clip CL with hoop members 5978A, 5978B below the bottom of clip CL. At step 4 of method M2, further actuation of drive member 5974 produces further axial movement of actuating member 5974, drawing the hoop members 5978A, 5978B and axial clip post 5975 towards each other, thereby securely gripping clip CL within the hoop members and the connector arms. The range of relative translation movement between coupling element 5977 and drive member 5974 (as indicated by arrow AR4) allows clip connector 5970 to assume a variety of desired configurations during the deployment of SOD 5900. In the fully open configuration (Step 1 and 2), coupling element 5977 and actuation member 5973 are in closest proximity. In the closed, intermediate configuration (Step 3), coupling element 5977 and actuation member 5973 are spaced apart an intermediate distance AR3, but clip CL is not yet fully captured. In the fully closed, clip-captured configuration (Step 4), coupling element 5977 and actuation member 5973 are in the maximum spaced-apart spatial relationship. Clip connector arms 5972A and 5972B are configured with corresponding terminal hoop members 5978A and 5978B, respectively, appropriately sized to encircle and engage distal end of deployed clip CL over region R1. As such, in Step 4, the distal end of clip CL is retained within the closed perimeter of the hoop members thereby advantageously providing a means of reacting the upward moment force on the occluders in systole (i.e., moment that tends to rotate the occluder about the top of the clip CL and displace same into the LA). Conversely, in diastole, the hoop members provide a means of reacting the downward moment force on the occluders in diastole (i.e., rotate the occluders about the top of the clip CL and displace same into the LV). Optionally (not shown), a textile mesh or flexible textile band may be draped across at least one of the open perimeters of hoop members 5978A or 5978B to limit the amount of insertion of the clip CL distal end within the hoop members. This tends to enhance secure capture of clip CL.

FIG. 92D illustrates the recess, socket, or opening CLO in the central portion of clip CL into which axial clip post 5975 may be engaged and secured. Clip post 5975 is disposed between clip-captured portions of leaflets AL and PL.

FIG. 92E shows SOD 5900, as previously shown in FIG. 92A-92D, attached to clip CL, with clip CL fully captured by clip connector 5970 in the fully closed configuration. Further FIG. 92F, shows SOD 5900 attached to clip CL and disposed within a native mitral valve MV.

FIG. 92G shows a top view of SOD 5900 with occluders 5940A and 5940B and respective such frame elements 5920A and 5920B positioned at an angle θ relative to each other. By virtue of the pivoting coupling joints 5971A through 5971D (only upper joints 5791C and 5971D visible in this view), angle θ may vary (as indicated by arrow AR5) to accommodate the particular anatomy of a patient's clipped valve. Angle θ may be selectively preset in the SOD prior to delivery in the LA, based on the patient's specific anatomy as informed by preoperative or perioperative imaging. Alternatively, with the occluders freely able to pivot about clip connector arms 5972A and 5972B, the SOD may be self-orienting and capable to assume the desirable angle θ once the occluders 5940A, 5940B are deployed in the respective flow control portions FCP1, FCP2.

FIGS. 92H-92K show a delivery/retrieval device DRD configured to selectively engage and disengage with coupling element 5977 of SOD 5900. Delivery/retrieval device DRD includes fingers DRDF that are configured to releasably engage with respective openings 59770 of coupling element 5977. For example, FIG. 92H shows delivery/retrieval device DRD engaged with coupling element 5977 by having fingers DRDF coupled with openings 59770. Fingers DRDF are biased towards each other, to a released configuration and can be forced away from each other, towards an engaged configuration, by axial movement of an actuator DRDA that is disposed between fingers DRDF. Fingers DRDF can be inserted into respective openings 59770 when in their released configuration, and once fully inserted can be driven by actuator DRDA into their engaged configuration, in which a distal tip of each finger DRDF is secured to a distal face of coupling element 5977 adjacent to the edge of each opening 59770, thus securing engaging delivery/retrieval device DRD with SOD 5900, as shown in FIGS. 92H and 92J. Actuator DRDA can also be rotated (while DRD retains clip connector 5970 through fingers DRDF engaged with coupling element 5977), so that it may be threadably engaged with (and disengaged from) drive member 5974, (e.g., via thread portion 5977T) Actuator DRDA can then be operated by translating axially within delivery device DRD, and actuating drive member 5974 (i.e., to move relative to coupling element 5977) to operate clip connector 5970 to capture (or release) clip CL as described above in reference to FIG. 92C. Coupling element 5977 is configured with three cantilevered, bent elastic fingers 5977F that are frictionally engaged with drive member 5974 to provide locking engagement therebetween throughout the translation range of motion AR4 between said components (FIG. 92C). As member 5974 translates through element 5977 from clip connector 5970 open configuration (FIG. 92C Step 1) to closed configuration (FIG. 92C Step 4), the bent fingers 5977F remain in contact with member 5974 during said translation. In the event that clip connector 5970 needs to be returned to the open configuration of Step 1 from the closed configuration of Step 4, a coaxial tube member DRDU is slidingly translated over DRDA (while DRDA is in threaded engagement with 5977T) in a manner that three fingers on terminal end of DRDU come into contact and subsequently displace fingers 5977F out of engagement with member 5974 thereby allowing relative movement therebetween. Once open configuration of Step 1 is restored, DRDU is withdrawn from contact with fingers 5977F and the latter deflect elastically back into contact with member 5974 thus restoring locked engagement therebetween. This arrangement advantageously allows clip connector 5970 (and SOD 5900) to be disengaged from clip CL in the event that procedure needs to be aborted, or SOD 5900 needs to be retrieved. Interspaced between three fingers 5977F and three guiding or locating surfaces 5977L that serve to locate and guide coupling element 5977 relative to member 5974 when fingers 5977F are depressed or displaces out of frictional engagement with member 5974.

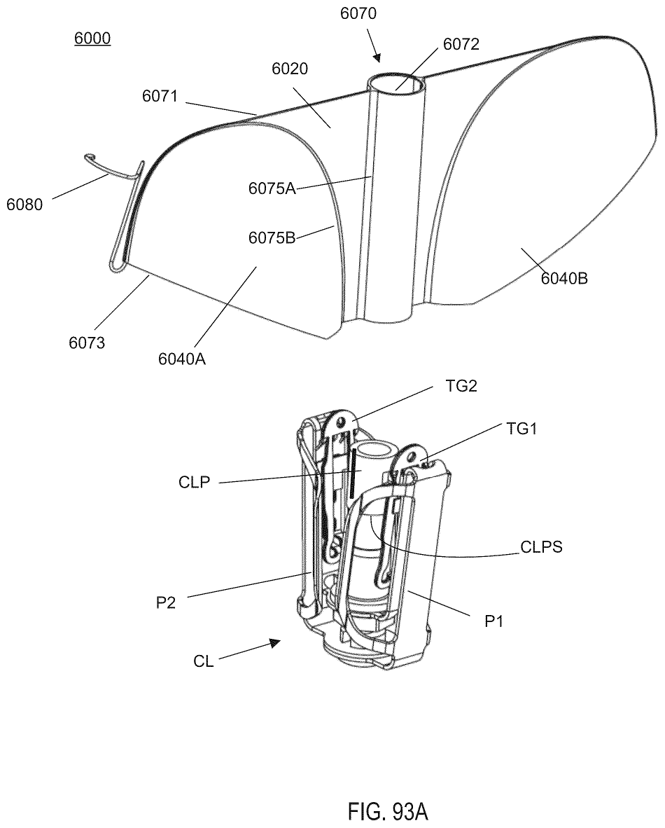

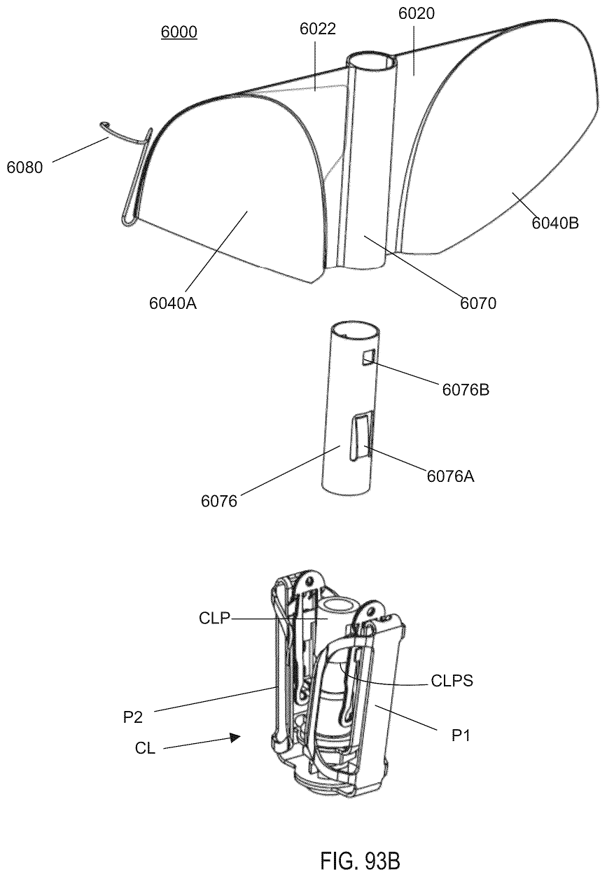

In some embodiments, an SOD can be configured with a minimum of structure and for direct engagement with a clip spacer disposed between leaflet capturing paddles of the clip. One such embodiment is shown in FIGS. 93A and 93B (which is an exploded view of FIG. 93A). SOD 6000 includes occluders 6040A and 6040B, a support frame 6020, a clip connector 6070, and an annulus connector 6080. In this embodiment, clip connector 6070 includes a connecter tube 6076 that is configured to slidably receive and securely engage with a post CLP of clip CL. Connector tube 6076 can include a latching finger 6076A, biased radially inwardly, which can be slide axially downwardly over the top of post CLP, and then snap radially inwardly under a shoulder CLPS on post CLP, axially securing connector tube 6076 (and thus SOD 6000) on clip CL. Support frame 6020 can be implemented as a sheet-like membrane or webbing that joins occluders 6040A and 6040B to clip connector 6070, and may optionally include a structural frame or wire, such as 6022 shown in FIG. 93B. For example, clip connector 6070 may be configured from a sheet of pericardium that is folded along the topmost surface 6071 of SOD 6000. Opening 6072 is provided in pericardium sheet to allow delivery catheter to engage with connector tube 6076 at slots 6076B, when delivering and deploying SOD 6000 into engagement with previously deployed clip (or together with clip CL when SOD 6000 is coupled to clip CL prior to delivery in the LA). Connector tube 6076 is permanently and securely connected to pericardium sheet via a series of securing sutures or by gluing (not shown). A structural stitch or seam 6075A is provided to approximate the two opposing surfaces of the folded pericardium sheet, tautly against connector tube 6076. An additional structural stitch or seam 6075B is provided to delimit the volume of the pseudo-valve occluders 6040A and 6040B by approximating the two opposing surfaces of the folded pericardium sheet along this arcuate stitch or seam 6075B. A flat web of pericardium results, spanning between seams 6075A and 6075B. Folded pericardium sheet terminates in two opposed movable membrane surfaces at open end 6073 of occluders 6040A and 6040B. Frame 6022 (and consequently occluder 6040A) is pivotably connected to connector tube 6076 since frame 6022 may be enclosed within or secured to connector tube 6076 by seam 6075A. Other portions of frame 6022 may be enclosed within or secured by arcuate seam or stitch 6075B. In a similar manner to previously described occluders 5940A and 5940B, occluders 6040A, 6040B may be positioned at an angle θ relative to each other.

SOD 6000 may be delivered to the patient's heart valve concurrently with clip CL (e.g., may be attached to clip CL before delivery) or separately. A first native leaflets (e.g., AL) is captured between paddle P1 and leaflet grasping member TG1, and a second native leaflet (e.g., PL) is captured between paddle P2 and leaflet grasping member TG2. First and second native leaflets may be captured independently of one another, as with current MitraClip G4 or PASCAL clips. Clip post CLP functions as a spacer, as described in connection with other embodiments described above. Optionally, leaflet grasping members TG1 and TG2 may be configured with a series of barbs or tissue gripping elements on the surface oriented toward the clip post CLP. As such, the barbs will engage with the pericardium sheet covering the connector tube 6076, when the SOD 6000 is coupled to clip CL and the clip CL is in the closed, leaflet-captured configuration. This may advantageously serve to enhance the retention or securement of SOD relative to clip CL, in addition to the function already provided by latching fingers 6076A engaged with clip shoulder CLPS. Unlike previously described SOD 5900 which relies on hoop members 5978A, 5978B coupled to distal end of clip CL to react moment forces from the cardiac cycle, SOD 6000 achieves a similar function by having the support frame 6020 span structurally between occluders 6040A and 6040B, and through the clip CL where it is captured between the opposing paddles P1, P2 of clip CL and clip post CLP.

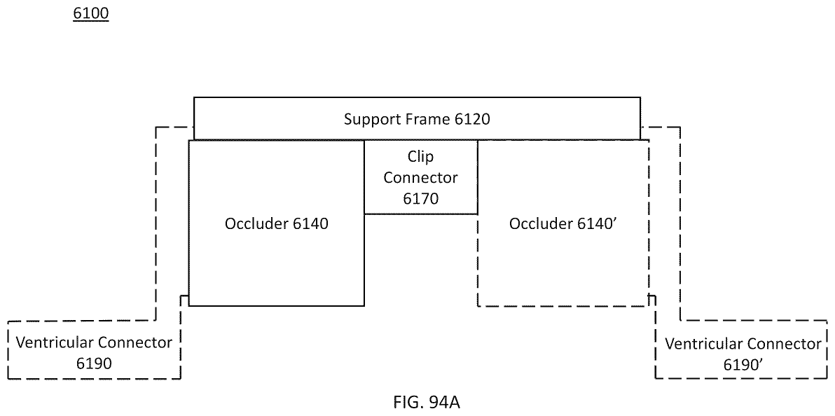

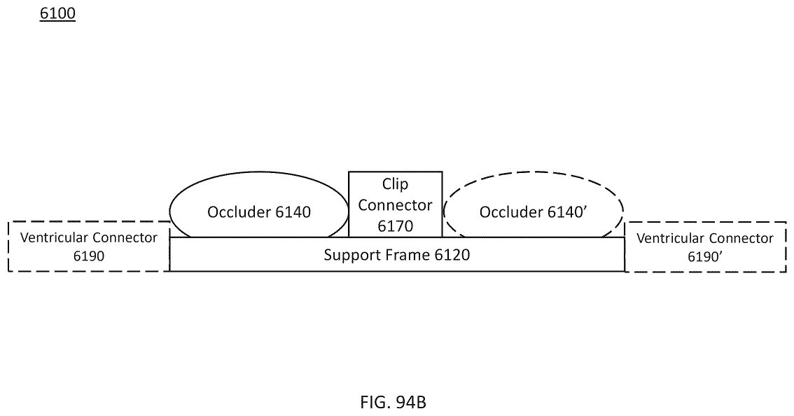

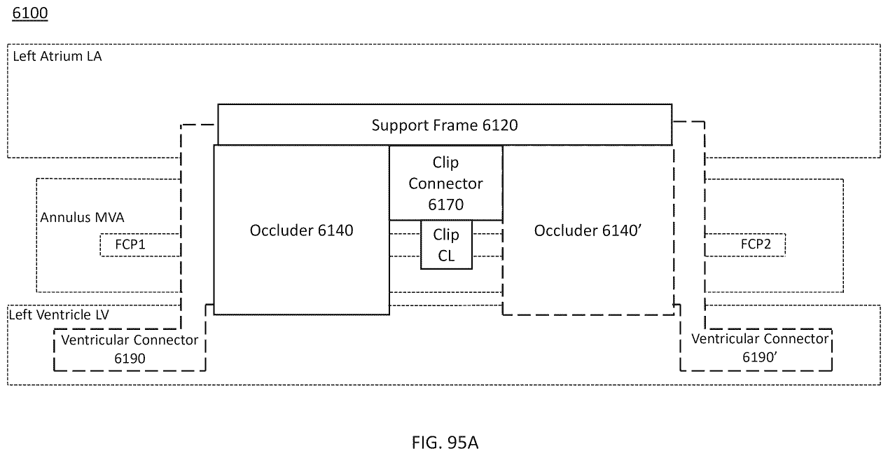

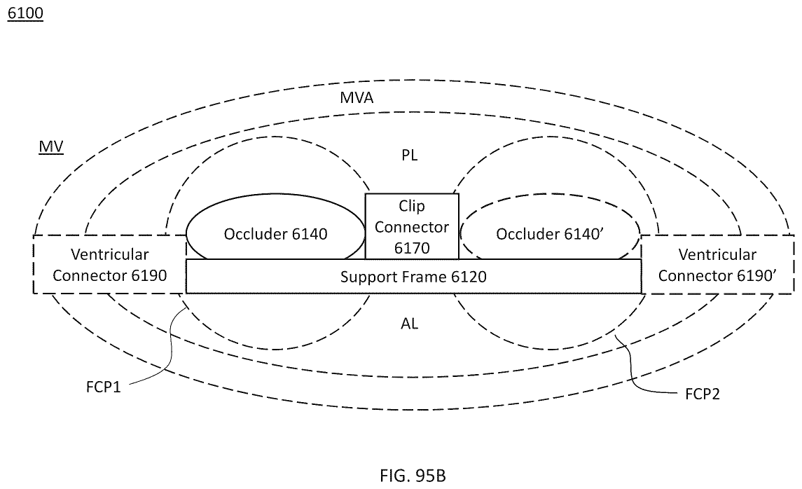

An embodiment of a selective occlusion device (or “SOD”) 6100 is illustrated schematically in a side view and top view, respectively, in FIGS. 94A and 94B. SOD 6100 includes a support frame 6120 and an occluder 6140. Occluder 6140 can be constructed, and function, similar to any of the occluders described above for other embodiments. As shown schematically in FIGS. 94A to 95B, occluder 6140 may be configured and appropriately sized to at least occupy the area of regurgitation between native leaflets during systole in a clipped atrioventricular valve. As described above in connection with other SOD embodiments, occluder 6140 may be implemented in some embodiments as a static structure, i.e., it need not flex inwardly (collapse) or outwardly (expand) to engage and disengage the native leaflets of the mitral valve MV, or the tricuspid valve TV, during the diastole and systole portions of the heart cycle. Instead, such static occluders may retain their shape and be sized and located in the native valve such that the native leaflets engage the occluder 6140 during systole and disengage the occluder 6140 during diastole. In other embodiments, as described above, as well as additional variations described in more detail below, occluder 6140 may be implemented with one or more flexible membranes, which act as a pseudo-valve by moving in coordination with the leaflets of the native valve, or occluder 6140 may be a prosthetic valve.

SOD 6100 also includes a clip connector 6170 that may be part of, or coupled to, support frame 6120 and/or occluder 6140, and is configured to engage with a clip CL such as those described above, and thereby to retain SOD 6100 in operative relationship with a native heart valve to which the clip CL is attached. In particular, clip connector 6170 is configured to carry fluid dynamic load applied to SOD 6100 during the cardiac cycle of the heart to the clip CL and thence to the native leaflets, annulus, and surrounding heart tissue to resist displacement of the SOD. The largest loads to be carried tend to be during systole, and the displacement to be resisted is towards the atrium of the heart.

Clip connector 6170 can be implemented in a variety of configurations, including those described above, as well as additional variations described in more detail below. As described above, clip CL can be any of the commercially available designs, or can be customized or modified specifically for use with SOD 6100.

As shown schematically in FIGS. 94A to 95B, SOD 6100 may include an optional ventricular connector 6190, which is configured to be coupled to occluder 6140 and a ventricular tissue of a native valve. Further, SOD 6100 may include a second occluder 6140′, which can also be coupled to the support frame 6120 and, optionally, clip connector 6170, and may also have an optional ventricular connector 6190′ (or be coupled to the same ventricular connector 6190). An SOD 6100 with both occluder 6140 and 6140′ may be used to control blood flow in a clipped native valve having two flow control portions needing regurgitation reduction-occluder 6140 can be disposed in a first flow control portion FCP1 and occluder 6140′ can be disposed in a second flow control portion FCP2, as shown in FIGS. 95A and 95B.

SOD 6100 is shown disposed in a native heart valve in a side view and top view, respectively, in FIGS. 95A and 95B. Note that for convenience of illustration the native heart valve is illustrated as a mitral valve MV. Note also that SOD 6100 is illustrated with the optional second occluder 6140′ disposed in one of the two flow control portions of clipped mitral valve MV. Mitral valve MV is shown with the anterior leaflet AL and posterior leaflet PL joined by a clip CL in an edge-to-edge approximation, similar to the mitral valve MV shown in FIG. 37B. Thus, mitral valve MV has two flow control portions-FCP1 and FCP2—each portion defined between the clip CL, the leaflets, and one of the commissures of the mitral valve MV. As discussed above with reference to FIGS. 37A to 38F, there are many possible arrangements of clips on either the mitral valve or tricuspid valve, creating one, two, or three flow control portions-SOD 6100 may be used with any of those clipped valve configurations to address regurgitation in one or two of the flow control portions.

As shown in FIGS. 95A and 95B, SOD 6100 can be disposed in mitral valve MV with a portion disposed in the left atrium LA and a portion disposed in the left ventricle LV. Clip connector 6170 is shown engaged with clip CL. Optional ventricular connectors 6190 and 6190′ can be engaged with a ventricular tissue structure located generally below the plane of the atrioventricular valve within a ventricle of the heart, such as a ventricular wall, a papillary muscle head, one or more chordae tendineae, or an apex of the heart. When SOD 6100 is disposed in mitral valve MV, it is operable to reduce or eliminate regurgitation through flow control portions FCP1 and/or FCP2, i.e., to prevent retrograde flow of blood from the left ventricle to the left atrium during systole, but to allow blood to flow freely through FCP1 and/or FCP2, from left atrium LA to left ventricle LV during diastole. More specifically, in diastole, the blood will flow in the space between the native leaflets and the SOD 6100 for static structure or movable membrane occluders, or through the SOD 6100 for prosthetic valve occluders.

Each of occluders 6140 and 6140′ may be configured so that outer surfaces thereof engage in a substantially sealing relationship with the anterior leaflet AL and/or the posterior leaflet PL during systole and thereby to reduce or prevent undesired retrograde flow (regurgitation) of blood therebetween from the left ventricle LV to the left atrium LA.

The occluders 6140, 6140′ of SOD 6100 are shown schematically in FIGS. 94B and 95B as being oval in cross section, roughly corresponding to the shape of the flow control portions of the native valve in diastole that result from leaflet clipping, e.g., oval as shown in FIG. 95B for ease of illustration. In some embodiments, the cross-sectional shape of the occluders could be more elliptical, or with a teardrop (more V-shaped) shape, with the narrower portion oriented toward the commissures, or other suitable shape that occupies at least the area of regurgitation between the native leaflets during systole in a clipped mitral valve. In movable membrane occluders, the shape may be irregular as to also fill cleft openings between native leaflets or scallops.

Each of occluder 6140, 6140′ can be constructed with materials and

techniques similar to the other occluders discussed above, and/or as described in more detail below.

Although clip CL may be a commercially-available edge-to-edge leaflet clip such as the MitraClip™ or PASCAL, and SOD 6100 being configured to engaged with such a clip after it has been used to clip the native leaflets, in some embodiments the clip CL may be configured differently than such commercially-available clips (for example any of the clips described above with reference to FIGS. 42D to 42F), and/or may be included as part of a system with SOD 6100 and configured to be delivered sequentially or concurrently with SOD 6100 as part of a total valve repair/replacement procedure. As described above, SOD 6100 is configured to be anchored to one or more clips CL, which in turn is/are coupled to the tissue of the anterior leaflet AL and/or posterior leaflet PL, and SOD 6100 will carry a considerable fluid dynamic load during systole that must in turn be carried by the clip(s) and the leaflets, and optionally, the ventricular connector 6190 and/or annulus connector from previous embodiments, if provided. Thus, an enlarged clip anchor or clip structure having wider paddles or multiple paddles may be useful. For example, the clip could be composed of two or three paddles (instead of the single paddle of some of the clip devices) for engagement with each of the one or more leaflets with which the clip is engaged, to increase the area of the leaflet(s) engaged by the clip(s). This allows the dynamic load to be distributed more evenly over a larger leaflet area (and over a greater number of underlying chordae tendineae that are attached to the engaged leaflet portions).

Rather than relying on the clip connector (and thus clip(s) CL, native valve leaflets, or other native valve tissue) to carry all of the fluid dynamic loads imposed on the SOD, in some embodiments those loads can be carried in part by other structures without putting the clip(s) CL or the native leaflets in the load path (or at least reducing the loads on the latter). Thus, in some embodiments SOD 6100 can include an optional ventricular connector 6190 and/or an optional annulus connector (not shown in FIGS. 94A-95B but similar to the embodiments of SOD described above). Optionally, ventricular connector 6190 can be configured to cooperate with the clip CL to carry some of the dynamic load exerted on the occluder 6140 (especially if fixedly connected with subvalvular heart tissue located below the valve annulus on the ventricle side). Additionally, the ventricular connector 6190 may serve to position or orient the SOD in an advantageous spatial relationship relative to the flow axis of the native valve in order to promote and maintain sealing engagement between the native leaflets and the occluder 6140 during systole. Additionally, the spatial relationship assumed by the SOD may vary during the different phases of the cardiac cycle, or may vary as a function of the specific patient anatomy, or may vary over time as a function of disease progression and its effect on the native leaflets.

As shown in FIGS. 94A to 95B, optional ventricular connector 6190 may be part of, or coupled to, support frame 6120, and is configured to engage with ventricular tissue of a native heart valve below the plane of the atrioventricular valve (including the ventricular wall, the papillary muscle, one or more chordae tendineae). Together with the clip CL, the ventricular connector 6190 serves to react the dynamic load exerted on the SOD by the cardiac cycle and, as such, reduces the stresses on the clip (and native leaflets connected thereto) that would otherwise result if the ventricular connector 6190 was excluded. Additionally, the ventricular connector 6190 assists in preventing the displacement of the SOD towards the atrium (during systole) or the ventricle (during diastole). Additionally, especially in the context of an SOD with a movable membrane occluder, the ventricular connector 6190 can allow the SOD to self-align according to the dynamic loads encountered during the cardiac cycle, and thereby assume a position or orientation relative to the native valve that maintains sealing engagement of the occluder with the native cusps during systole (while providing minimal flow obstruction and pressure gradient during diastole).

The ventricular connectors described in the embodiments that follow may also be included in some of the previous embodiments described above. As well, the previously described annulus connectors may be included, to the extent necessary, in some of the embodiments described below.

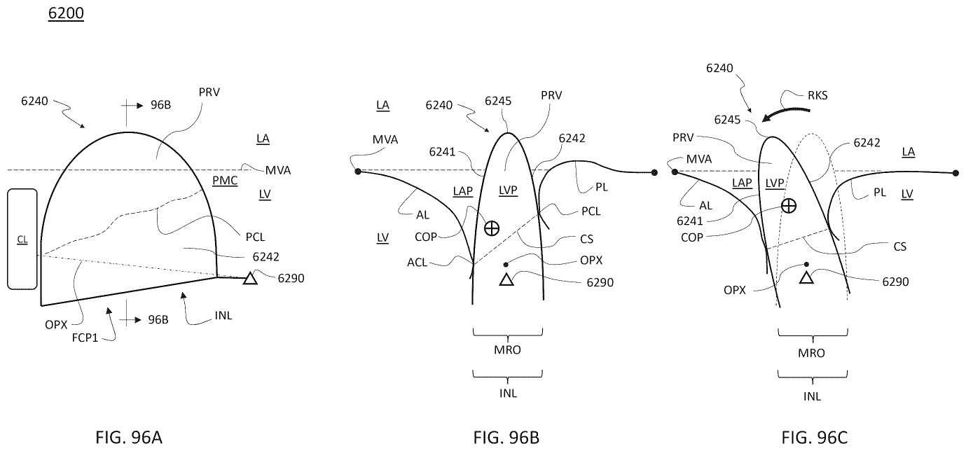

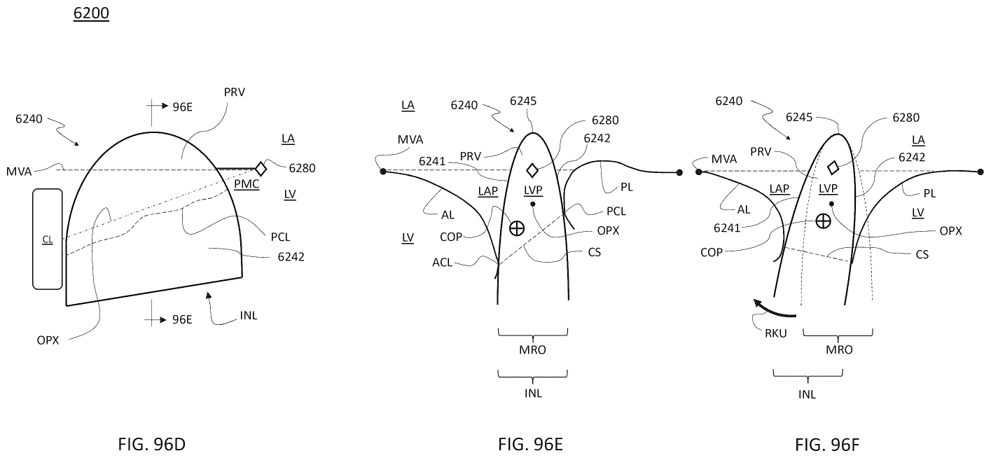

As discussed above, an SOD can be secured in place in a native atrioventricular valve in part with an annulus connector and/or a ventricular connector. The performance of an SOD can be affected by the type of connector used, and by the position of the occluder relative to the native valve annulus established by the connector. Considerations for type of connector and position of the occluder are described below for an SOD 6200 with references to FIGS. 96A to 96F.

FIG. 96A schematically illustrates, in a side elevation view, SOD 6200 having a movable membrane occluder 6240. SOD 6200 is disposed in a flow control portion FCP1 between a centrally-placed clip CL and a commissure PMC of a mitral valve MV. Occluder 6240 is coupled to clip CL (which is secured to native leaflets AL and PL) and is also engaged with a ventricular tissue, for example the wall of the left ventricle LV, through a ventricular connector 6290. As such, an axis extending between these two tissue engagement points of SOD 6200 is defined. More specifically, as will be described in greater detail below, occluder 6240 may, in use, advantageously pivot about an occluder pivoting axis 6291 extending between clip CL and ventricular connector 6290.

Referring to FIGS. 96B and 96C, the spatial relationship of SOD 6200 to the mitral valve, in systole, is schematically illustrated in cross-sectional views of occluder 6240 along a cut plane 96B-96B in FIG. 96A (i.e., through apex 6245). Ventricular connector 6290 is illustrated in end view. Closed end of occluder 6240, including apex 6245, extends above the mitral valve annulus MVA into the left atrium LA. Occluder open end or inlet INL is located within the left ventricle LV, below MVA, and as such is exposed to pressures of the left ventricle LVP and is open to receive and stop the blood flow that would otherwise enter the LA through the regurgitant orifice MRO of the mitral valve.

During systole, the internal volume of occluder 6240 fills with blood and the internal surface of occluder 6240 is exposed to ventricular pressure LVP. Anterior leaflet AL is engaged with movable membrane 6241 (defining anterior coaptation line ACL therebetween) and posterior leaflet PL is engaged with movable membrane 6242 (defining posterior coaptation line PCL therebetween). This provides the desired sealing engagement between SOD 6200 and mitral valve MV that reduces the undesirable leakage or regurgitation in systole.

As illustrated in FIG. 96A, proximal to clip CL, the native leaflets extend deeper into the LV and are also captured as such within the clip. Proximal to the commissure PMC, the native leaflets are shorter and extend less within the LV. This results in an angled posterior coaptation line PCL between posterior leaflet PL and membrane 6242 (shown) and similarly for the anterior coaptation line ACL between anterior leaflet AL and membrane 6241 (not shown).

Coaptation lines PCL and ACL join to form a continuous coaptation perimeter around occluder 6240. The resulting surface bounded by this coaptation perimeter defines a coaptation surface CS through the pressurized occluder in systole. In FIG. 96B, surface CS is depicted as an oblique line since it is sectioned in this cutaway view. As will be described in greater detail below, the resulting volume bounded by the occluder membranes above the coaptation surface CS is the pressurized volume PRV that energizes the occluder 6240 to function in use.

In reference to FIG. 96B, above the coaptation lines ACL and PCL, the outside surface of occluder 6240 is exposed to atrial pressure LAP, while the inside surface is exposed to higher ventricular pressure LVP. This results in a net differential pressure (LVP-LAP) across movable membrane 6241 and 6242, thereby urging the membranes to spread apart from one another and also move toward their respective native cusps AL and PL. In systole, this ensures an effective seal between native leaflets and occluder 6240.

It is preferable to have a closed portion of occluder 6240 extending above mitral valve annulus MVA into left atrium LA. This ensures that at least a portion of occluding membranes 6241, 6242 remain beyond the reach of coaptation with native leaflets, and are therefore always exposed to the differential pressure (LVP-LAP). The higher that apex 6245 extends into left atrium LA, the greater the driving force that energizes occluder 6240 to open (i.e., urging movable membranes apart), since the occluder surface area across which the differential pressure acts will also increase with such extension. Below the coaptation lines ACL, PCL (i.e., below coaptation surface CS deeper into LV), there is no differential pressure across membranes 6241, 6242, respectively, since both sides of each membrane are exposed to the same ventricular pressure LVP.

Variation in positioning of SOD within a given mitral valve, the diseased state of the native valve (i.e., having retracted, prolapsing or flail leaflets), or the variations in specific patient anatomies, may result in coaptation lines ACL and PCL being at different depths below the MVA, on opposing sides of occluder 6240. For example, as illustrated in FIG. 96B, coaptation line ACL is relatively deep within the LV and engages occluder 6240 closer to inlet INL than coaptation line PCL. Consequently, the length of membrane 6241 exposed to the differential pressure (LVP-LAP), as well as the membrane surface area corresponding thereto, is greater than the opposing length of membrane 6242, and its corresponding membrane surface, exposed to this same differential pressure. This results in the center of pressure COP of pressurized volume PRV being eccentrically offset toward membrane 6241 since it relatively has a larger membrane surface exposed to said differential pressure (FIG. 96B). This also generates a net force on the occluder directed towards anterior leaflet AL. Since occluder 6240 is anchored through ventricular connector 6290, in a location below coaptation surface CS, and also below center of pressure COP, a resulting moment along the A-P direction, RKS, acts to tilt or rotate occluder 6240 in a counter clockwise direction, about an occluder pivoting axis OPX, in order to centralize the COP and equalize/stabilize the forces exerted on the occluder from its pressurization. As illustrated in FIG. 96C, apex 6245 tilts towards the left, from its initial position of FIG. 96B shown in dashed outline, as it rotates about occluder pivoting axis OPX. The COP is now centralized.

In securing SOD 6200 with ventricular connector 6290 in proximity to occluder inlet INL, the resulting occluder pivoting axis OPX (i.e., located below the COP and above INL; between COP and INL) offers advantages in device operability. As illustrated in FIGS. 96B-96C, occluder inlet INL remains generally aligned and in register with the regurgitant orifice MRO in the mitral valve, despite the tilting or rotation of occluder 6240 about occluder pivoting axis OPX that may result during use. Consequently, the sealing engagement between the occluder surfaces and native leaflets, in systole, is ensured.

FIGS. 96D-96F illustrate the same SOD 6200 as described in FIGS. 96A-96C with the exception that the ventricular connector 6290 is replaced with an annulus connector 6280, as described in some of the previous embodiments. The occluder pivoting axis OPX extends between clip CL and annulus connector 6280, and is distal to the occluder inlet INL (i.e., located above both the COP and also the occluder inlet INL).

As illustrated FIG. 96E, the same anatomic conditions as those illustrated in FIG. 96B, the center of pressure COP is eccentrically offset toward membrane 6241, and also generates a net force on the occluder directed towards anterior leaflet AL. Since occluder 6240 is now anchored through annular connector 6280, in a location above coaptation surface CS, and also above center of pressure COP, a resulting moment along the P-A direction, RKU, acts to tilt or rotate occluder 6240 in a clockwise direction, about occluder pivoting axis OPX. As illustrated in FIG. 96F, in contrast to apex 6245 tilting towards the left in FIG. 96C, it is the occluder inlet INL that shifts towards the left, from its initial position of FIG. 96E shown in dashed outline, as it rotates about occluder pivoting axis OPX. The COP is now centralized.

As illustrated in FIGS. 96E-96F, occluder inlet INL is displaced away from regurgitant orifice MRO in the mitral valve, due to the rotation of occluder 6240 about pivoting axis 6281. In use, this may result in a compromise to the sealing engagement between the occluder surfaces and native leaflets, in systole.

To prevent unwanted disengagement and mitral regurgitation, SOD embodiments with annulus connectors and having movable membrane occluders may benefit from being structurally reinforced to avoid the device rotation described above and to maintain sealing engagement in systole. This may be achieved in a number of ways including increasing the mechanical stiffness of the occluder support frame members or optionally also adding a ventricular connector to such embodiments. Such structural remedies may make SOD collapsibility more challenging if delivery is intended through a transcatheter approach.

In comparison, SOD embodiments with ventricular connectors (i.e., located below the plane of the MVA and in proximity to the occluder inlet), the frame members can be of reduced stiffness to intentionally allow rotation of the occluder about its pivoting axis (as per FIG. 96C) since the occluder inlet remains advantageously aligned with the regurgitant orifice, and the sealing engagement between occluder membranes and native leaflets is ensured. As such, device collapsibility is easier to achieve.

Although the device elements and functions described in FIGS. 96A-96C apply in particular to an SOD 6200 having a movable membrane occluder 6240, some or all of the elements described may also be included and provide advantages to SOD embodiments having movable membrane occluders with no communication to circulatory blood flow (i.e., no occluder inlet), static membrane occluders, fixed geometry occluders, or even occluders including a prosthetic valve. More specially, having a SOD with a an occluder pivoting axis OPX (i.e., extending between a ventricular occluder and clip CL) that is located below the occluder-to-native leaflet coaptation line or zone, allows the occluder to find/transition to/occupy a more favorable position or orientation within the flow control portion that improves sealing across the mitral valve during systole.

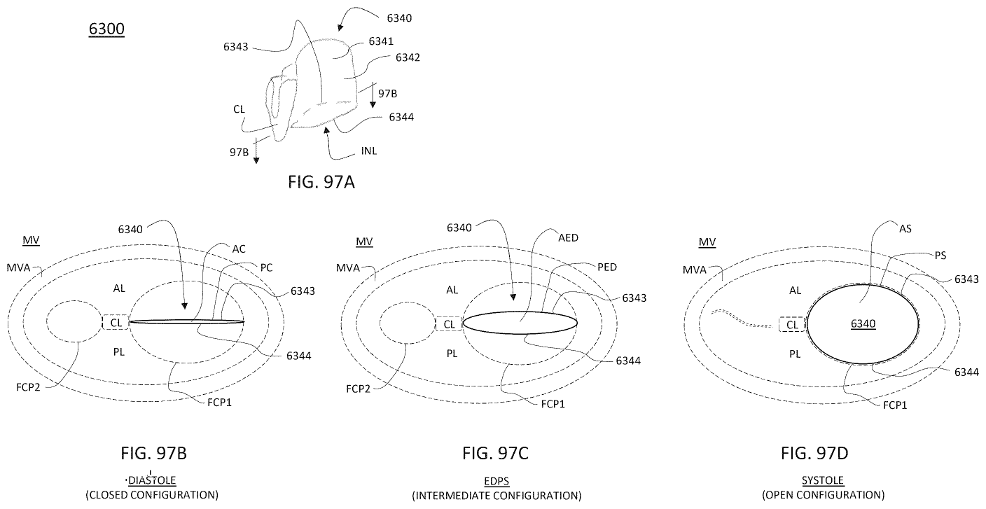

FIGS. 97A-97D illustrate a SOD 6300 having a movable membrane occluder 6340 disposed in a single flow control portion FCP1 of a mitral valve MV. Occluder 6340 includes two cooperating occluder membranes 6341, 6342. In systole, membrane 6341 engages with anterior leaflet AL, and membrane 6342 engages with posterior leaflet PL.

As schematically illustrated in FIGS. 97B-97D, SOD 6300 is capable of assuming three predetermined occluder configurations corresponding to different phases in the cardiac cycle. FIGS. 97B-97D show a top view of a mitral valve (i.e., from the left atrium), with a cross-sectional view through occluder open end INL along cut plane 97B-97B of FIG. 97A.

As illustrated in FIG. 97B, at diastole, occluder 6340 is in a fully-collapsed, or closed configuration. Membranes 6341, 6342 are in close proximity to or preferably in contact with one another, and are disengaged and spaced away from native leaflets AL, PL, respectively. Occluder inlet INL assumes an inlet area in the closed configuration, AC, and corresponding inlet perimeter in the closed configuration, PC, along occluder free margins 6343, 6344. Inlet area AC may approach zero when free margins approach or come into complete contact. This diastolic configuration of occluder 6340 offers minimum obstruction and resistance to the blood flowing through FCP1 from the left atrium into the left ventricle in diastole.

As illustrated in FIG. 97D, at systole, occluder 6340 is in a leaflet sealing, or fully-open configuration. Membranes 6341, 6342 are spaced apart from one another and are in sealing engagement or contact with native leaflets AL, PL, respectively. Occluder inlet INL assumes an inlet area in the fully-open configuration, AS, that is a maximum inlet area, and a corresponding inlet perimeter in the fully-open configuration, PS, along occluder free margins 6343, 6344. This systolic configuration of occluder 6340 provides desired sealing between occluder 6340 and native leaflets AL, PL to prevent leakage or regurgitation across the mitral valve MV in systole.

As illustrated in FIG. 97C, at the end of diastole and just prior to the start of systole, occluder 6340 can advantageously assume a predetermined, membrane-spaced, or intermediate configuration whereby opposing membranes 6341, 6342 are spread apart, at least at one location along the length of free margins 6343, 6344, to create at least an inlet opening area in the intermediate configuration, AED, and a corresponding inlet perimeter in the intermediate configuration, PED. Area AED is greater than area AC and less than area AS. This intermediate configuration of occluder 6340, also referred to herein as the EDPS configuration, advantageously prepares occluder 6340 to receive systolic blood flow (i.e., blood flow from the LV toward the LA) within membrane-spaced occluder inlet INL, through area AED and between membranes 6341, 6342.

As described in greater detail in the embodiments that follow, an SOD such as SOD 6300 advantageously includes a membrane-spacing element (not shown in this embodiment) that preferentially disposes the SOD occluder in the intermediate, or EDPS, configuration at a point in the cardiac cycle when the diastolic dynamic load on the SOD reduces (i.e., when blood flow into LV from LA has substantially stopped), and just prior to start of systole (i.e., when compression of the LV is about to begin)—at this point there is a substantial absence of blood flow between the native leaflets. For brevity in this disclosure, the foregoing will be referred to as the EDPS point in the cardiac cycle.

In the EDPS configuration, the occluder membranes 6341, 6342 are partially spaced from each other, and in closer proximity to their respective native leaflets AL, PL and, as such, disposes the movable membranes to more readily receive blood at the start of systole, and move quickly (earlier in systole) toward their eventual sealing position in the systolic configuration (FIG. 97C). Without the benefits of this intermediate EDPS configuration, the membranes 6341, 6342 would be starting their movement toward the systolic configuration further away from their respective leaflets (i.e., from the diastolic configuration FIG. 97B). Providing the movable membranes 6341, 6342 with a “head start” towards the native leaflets improves the performance of occluder 6340, that is it fills with blood effectively and in a timely manner during systole, and avoids the likelihood of membranes 6341, 6342 arriving late to engage the native leaflets AL, PL with resulting leakage or regurgitation across the mitral valve MV. Since SOD 6300 assumes the EDPS configuration only at the end of diastole, the spread-apart membranes do not cause obstruction during LV filling since that has already occurred. With the start of systole, the spread-apart membranes are further activated into motion, from the EDPS configuration to the systolic configuration, by the fluid inertial forces (i.e., by the dynamic loading) of systolic blood flow into the occluder 6340.

Although it is preferable to have both membranes 6341, 6342 moving relative to one another and towards their respective native leaflets in systole, at least one of the membranes is preferably movable relative to the other opposed membrane during the different phases of the cardiac cycle to benefit from the advantages described in reference to the EDPS configuration.

The advantages described above relating to an SOD capable of assuming an EDPS configuration, may also apply to or be advantageously implemented in some of the previously described embodiments of SOD having flexible, movable membrane occluders (ex., FIGS. 1C, 4A, 18A, 83B, 85A, 87A).

FIGS. 97B-97D schematically illustrate the relative magnitude of the occluder inlet areas delimited by the free margins of the movable occluder membranes 6341, 6342 of SOD 6300 at various stages in the cardiac cycle. The schematic figures are not intended to illustrate the specific geometric shape of the occluder inlet opening, which can assume a variety of different shapes. The latter also applies to the outline defining the flow control portions FCP1 and FCP2 which are representative, but are not intended to be scaled. It is also understood although the occluder inlet area may vary in size between the three SOD configurations, the perimeter may remain essentially the same (i.e., folds along the free margin or contacting margins between opposed membranes are not schematically represented).

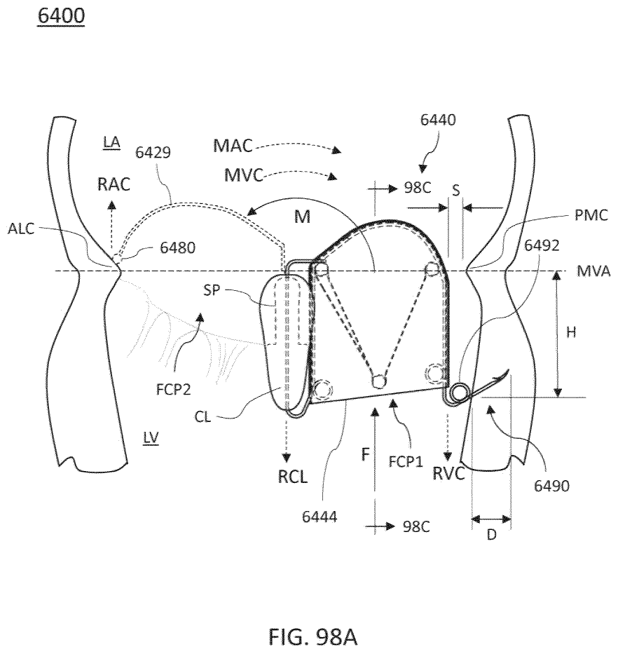

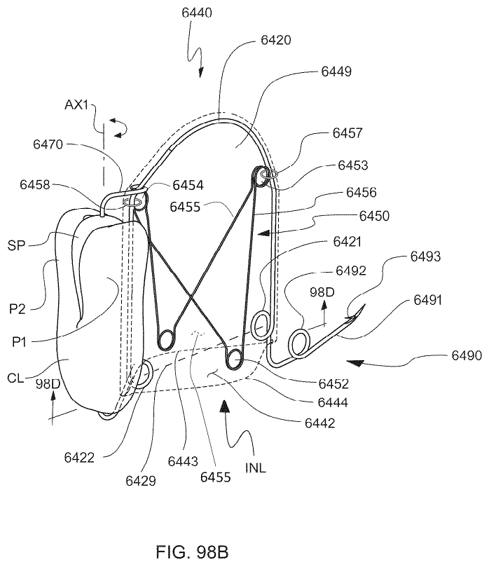

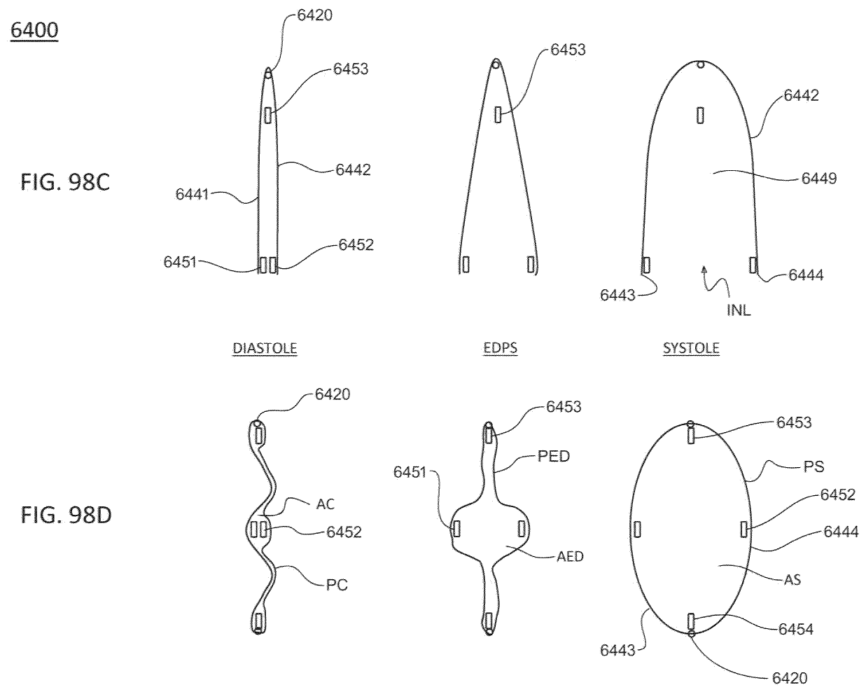

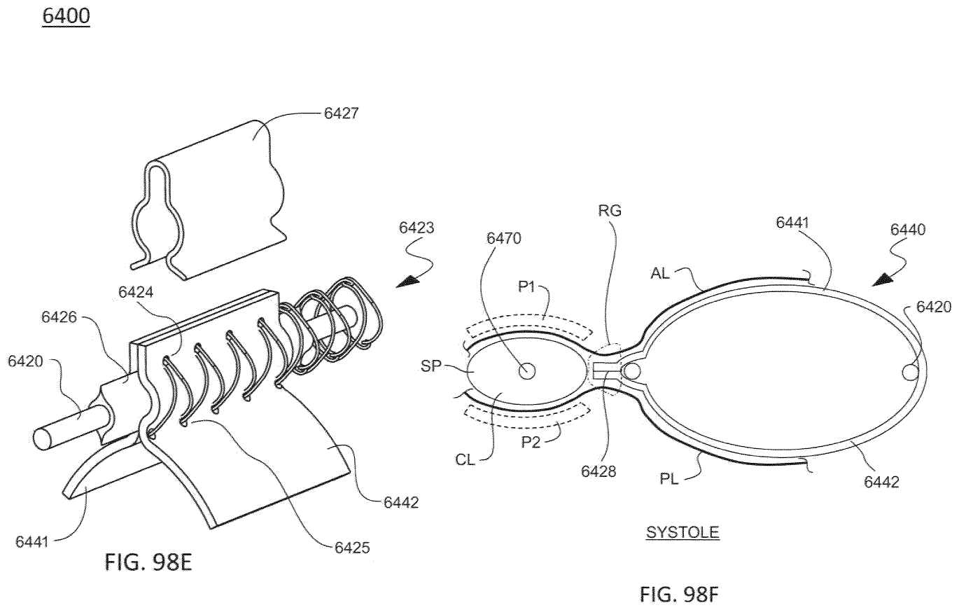

FIGS. 98A-98F illustrate an SOD 6400 having a movable membrane occluder 6440 disposed in a single flow control portion FCP1 of a mitral valve MV. Occluder 6440 includes two flexible, movable membranes 6441, 6442 configured to engage anterior and posterior leaflet AL, PL, respectively, when the SOD assumes the systolic configuration as previously described with reference to FIG. 97D. The occluder membranes are variably spaced apart during the different phases of cardiac cycle, and define therebetween an occluder inner cavity or volume 6449 that is variable in magnitude depending on how much blood enters the occluder open end or inlet INL.

For example, in this specific embodiment, the movable membranes are made from a sheet of thin (for example with a thickness in the range of 0.15-0.20 mm), pliable porcine pericardium. The membranes are secured to an arcuate support frame 6420 which is pivotably engaged to clip CL through clip connector 6470. More specifically, the clip connector is coupled to a spacer SP disposed between paddles P1, P2 of the clip. During use, spacer SP (and clip connector 6470) will also be situated between the native leaflet portions that are captured and retained by clip CP. As such, the pivoting motion of occluder 6440 about occluder-clip pivot axis AX1 (i.e., to align the occluder to the leakage between native leaflets to be sealed), will always only result in desirable contact between pericardium and native leaflets.

SOD 6400 is provided with a ventricular connector 6490, which in this embodiment is implemented as a penetrating tissue-anchoring member or tissue barb 6491, suitable for anchoring to and retaining cardiac tissue. As shown, support frame 6420 extends laterally beyond the occluding portion of occluder 6440, on the opposite side of clip CL, to define the ventricular connector 6490 and terminate in barb 6491. Support frame 6420 is preferably made from a superelastic materials, such as Nitinol, which not only improves the collapsibility of SOD 6400 to facilitate its delivery to the MV through a transeptally-placed cannula, but also possesses the suitable material properties for fashioning the elastic barb elements 6491 that allow secure anchoring of ventricular connector 6490 to the LV. Barb 6491 can be produced on the terminal end of support frame 6420, or can be produced as a separate element that is subsequently joined (i.e., by laser welding) or connected to the support frame (i.e., by mechanical crimping). Once anchored, barb 6491 secures occluder 6440 in a desired orientation about occluder-clip pivot axis AX1 in spatial relation to the regurgitant orifice in the MV. Since the dynamic load imparted on the SOD tends to be greatest in systole (i.e., in a direction from the LV to the LA), the barb is preferably configured with an upwardly angled shape, toward the LA, to better resist loads and secure the retention of the SOD along this direction.

As shown in FIG. 98A, ventricular connector 6490 is engaged with ventricular tissue at a depth H below the plane of the MVA (i.e., below the cusp insertion line of the mitral valve leaflets). Depth H may be in the range of 5-25 mm, and preferably within the range of 10-15 mm. In this embodiment, ventricular connector 6490 is generally aligned with the commissure between the anterior AL and posterior PL leaflet, the posteromedial commissure PMC (as shown), and is positioned above the level of the papillary muscle head (not shown). Barb 6491 is inserted into the wall of the LV between the chordae tendinea that may be present. Since the ventricular connector 6490 is generally at the level of occluder inlet INL, and therefore also below the coaptation lines ACL, PCL between occluder membranes 6441, 6442 and native leaflets AL, PL, respectively, the device operability benefits previously described in reference to SOD 6200 (FIGS. 96B-96C) also apply to SOD 6400.

Ventricular connector 6490 is provided with a tissue-penetration limiting member 6492, such as a loop or eyelet, included between occluder 6440 and barb 6491. As shown, penetration limiting member 6492 may be formed when setting the desired shape of the Nitinol support frame by configuring one looped winding in the portion of the support frame reserved for the ventricular connector. Penetration limiting member 6492 is configured and sized to advantageously control both the depth of penetration D into the ventricle wall, and the predetermined offset distance S of frame member inwardly away from the annulus of the MV (and also from the wall of the LV). Offset distance S may be advantageous when it is desirable to avoid potential interference between small commissural leaflets, that are sometimes present, and the edge of the occluder support frame 6420 at this location.

Penetration limiting member 6492 may be configured with multiple looped windings to increase the structural flexibility of SOD 6400 at this location. Such an arrangement can advantageously allow occluder 6440 to pivot or tilt toward the anterior or posterior annulus (i.e., along the A-P direction, RKS), in use, in order to provide the device functionality previously described in reference to SOD 6200 of FIG. 96C. This multiple-windings arrangement also offers advantages during the contraction of the LV during systole. The displacement of the LV and any deflection of the wall portion containing the embedded barb 6491, can be resiliently absorbed by the looped windings rather than transmitting such deflections directly and rigidly to occluder 6440 which may displace the occluder from sealing engagement with the native leaflets. Alternatively, penetration limiting member 6492 can be implemented by a flexible wire tether connecting the frame 6420 to barb 6491, or a necked down wire cross-section locally over this portion of the frame, or a mechanical pivoting joint connecting the barb 6491 to the support frame 6420.

Additionally, penetration limiting member 6492 may be configured and sized as a coupling interface for engagement with a delivery catheter (not shown) for implanting SOD 6400. An appropriately sized fitting in the catheter distal end can releasably engage the eyelet of penetration limiting member 6492 to allow spatial manipulation of SOD 6400 during implantation, including manipulation to insert barb 6491 into the LV wall. Contemplated methods of implantation will be described in greater detail below with reference to FIGS. 116A-116E.

As shown in FIG. 98A, in systole, the dynamic fluid inertial loads exert a force F on the single occluder 6440 of SOD 6400. Force F induces a moment M about clip CL tending to rotate the occluder in a counter clockwise direction about the clip attachment point (i.e., into the LA). Force F is reacted by the clip CL with a reaction force RCL. However, since the clip is attached to generally movable leaflet tissue, it is difficult to effectively counteract this moment M just at the clip attachment point.

Adding a ventricular connector 6490 reduces the magnitude of the clip reaction force RCL at the clip by introducing a ventricular connector reaction force RVC that shares in reacting the occluder force F. As well, the ventricular connector introduces a ventricular connector counteracting moment MVC, which acts in a clockwise direction and opposite to moment M. As shown, SOD 6400 is secured in position relative to the native mitral valve MV by clip CL (engaged with the native leaflets) and by ventricular connector 6490 (engaged with the wall of the LV).

Alternatively to, or in addition to ventricular connector 6490, an annulus connector 6480 (shown in dotted line) may be included in SOD 6400. In the alternative arrangement, this would introduce a annulus connector reaction force RAC and annulus connection reaction moment MAC, which would serve to also counteract moment M and help react and redistribute occluder force F and moment M acting on clip CL to other heart tissue structures such as the annulus or the ventricle. If added in this embodiment, the annulus connector 6480 (shown in dashed lines) would be positioned by a support frame 6429 (shown in dashed lines) on the opposite side of the clip CL from the side occupied by occluder 6440 (i.e., adjacent to FCP2, proximal to the opposite commissure ALC).

It is understood that including a second occluder 6440′ (not shown) in flow control portion FCP2 will induce an additional force F′ to be reacted by clip CL and, if supplied, reacted also by ventricular connector and/or annulus connector. However, the moment M′ (not shown) about the clip CL resulting from the dynamic load on second occluder 6440′ will counteract, at least in part, the moment M from the first occluder 6440.

With reference to FIGS. 98B, SOD 6400 includes a membrane-spacing element 6450, which is this embodiment is implemented as an elastic, biasing element. As such, occluder 6440 is able to assume, during use, the three occluder configurations during the cardiac cycle (i.e., open systolic configuration, closed diastolic configuration, and EDPS intermediate configuration) as illustrated schematically in FIGS. 98C-98D. SOD 6400 will be have the associated advantages as previously described in reference to embodiment 6300 (FIGS. 97B-97C).

As shown, membrane spacing element 6450 is made from a single wire of very thin (for example, 0.004″ diameter), superelastic (for example, Nitinol) wire, that is shape-set into a pair of adjoined, opposed “V-shaped” sections 6455, 6456. Each V-shaped section has an apex and first and second arms extending from the apex. The upper ends of the first arms are coupled together, and the upper ends of the second arms are coupled together. Four wire-loop attachment portions or loops 6451, 6452, 6453, 6454 are also shape-set and provided in membrane spacing element 6450, serving to attach the element to occluder 6440. Loop 6451 is at the apex of V-shaped section 5455, and loop 6452 is at the apex of V-shaped frame 6456. Loop 6453 couples the first arms of each of the V-shaped sections together, and loop 6454 couples the second arms of each of the V-shaped sections together. To allow it to move freely with the occluder membranes 6441, 6442, membrane spacing element 6450 is preferably pivotably attached to support frame 6420 by a pair of sutures 6457, 6458 placed through loops 6453, 6454, respectively, and tied separately around support frame 6420. This manner of attachment can be further reinforced by having the stitching pattern that secures the pericardium occluder membranes 6441, 6442 to the support frame 6420 also pass through the loop attachment portions. Alternatively, membrane spacing element 6450 can be fixedly attached to support frame 6420, or attached to one or both occluder membranes 6441, 6442 at this same location without connection to the support frame. Membrane spacing element 6450 is minimally attached to occluder membranes 6441, 6442 by a pair of sutures placed through loops 6451, 6452, respectively, proximal to the respective occluder free margins 6443, 6444. Additional discrete securing sutures may be placed through the occluding membrane (ex., membrane 6442) and tied around the adjacent spring V-shaped section (e.g., section 6456) at one or more suitable locations. Alternatively, a continuous running stitch, or capturing seam, can be added along the profile of V-shaped section 6455, 6456 to secure same to the respective occluder membrane 6441, 6442, respectively.

Preferably, membrane spacing element 6450 is disposed on the inside surface of occluder 6440 so as to avoid potential interference or abrasive contact with the native leaflets. Alternatively, membrane spacing element 6450 can be covered with a separate pericardium ply or sheet, or a suitable inert bioprosthetic material to shield it from direct contact with blood or with native leaflet tissue.

In the unconstrained free state, wire loop portions 6451, 6452 assume a spaced apart configuration as shown in FIG. 98B. During diastole, the fluid inertial forces of the blood flowing from LA to LV (i.e., the dynamic load) against occluder membranes 6441, 6442 will collapse occluder 6440 towards its fully closed, diastolic configuration, and also membrane spacing element 6450 (i.e., urging loops 6451, 6452 into close proximity). Membrane spacing element 6450 is configured and sized with the appropriate spring constant and structural stiffness so as to not resist the closing of occluder membranes 6441, 6442 against one another (FIGS. 98C, 98D, left image). At EDPS, under reduced dynamic load, the internal energy that was stored in membrane spacing element 6450 during diastole is converted to displacing and spreading apart occluder free margins 6443, 6444, at least over the portion proximal to loops 6451, 6452 (FIGS. 98C, 98D, center image), resulting in an inlet area AED being produced at occluder inlet INL. The spring constant causes the membrane spacing element 6450 to resume its free unconstrained state as occluder 6440 assumes the intermediate EDPS configuration.

As occluder 6440 transitions between the EDPS configuration and the fully-open, systolic configuration, membrane spacing element 6450 elastically deforms or expands, outwardly from its free state, driven by the fluid inertial forces of the systolic blood flow into occluder 6440 until sealing engagement between the occluder membranes and the native leaflets occurs (FIGS. 98C, 98D, right image). Beyond this point, the pressure differential (LVP-LAP) across the occluder membranes above the coaptation line, in a sealing pressurized occluder 6440, may further elastically deform outwardly membrane spacing element 6450. At end of systole and start of diastole, membrane spacing element 6450 will elastically recoil towards its free state configuration. As such, the internal energy that was stored in the expanded spring state will also help to approximate the occluder membranes 6441, 6442 and consequently help flush the occluder inner cavity 6449 of its blood volume.

Alternatively, membrane spacing element 6450 can be designed to be in its free state when the occluder 6440 assumes the open, systolic configuration. This would make for a more reactive opening of the movable membrane occluder during EDPS (i.e., producing a greater area AED), at the expense of greater resistance to closing of the occluder membranes during diastole (i.e., possibly not necessarily a greater area AC).

Membrane spacing element 6450 as illustrated, may also provide a prolapse-limiting function, especially in SOD configurations provided with excess surface area on the occluder membranes (i.e., excess occluder inlet perimeter PS and area AS capacity for the size of regurgitant orifice MRO that needs to be sealed). Such SOD configurations are advantageous since the excess membrane provides compliance or reserve to continue sealing the regurgitant orifice area as it increases in size with the progression of MV disease. This is an advantageous feature of movable membrane occluders over static structure occluders.

Prolapse in the particular context of SOD embodiments having movable membrane occluders means undesirable movement or displacement of the unsupported occluder free margin (i.e., 6443, 6444) towards the left atrium LA, that may compromise sealing during systole between the movable occluder membrane and the native valve leaflets. As shown in FIG. 98B, membrane free margin 6444 is supported at both terminal ends where it is secured to support frame 6420 and additionally at approximately its mid-span, where it is also attached to loop 6452 of membrane spacing element 6450. Securing of the spring V-shaped section 6456 to support frame 6420 at loops 6453, 6454 offers an effective triangulated structure that restrains the attached free margin from migrating upwards or folding toward the LA during systole. Additionally, this triangulated structure resists buckling or bending of the V-shaped section towards support frame 6420. As such, this constrains the mid-span of free margin 6444 to remain within the LV and also generally within the regurgitant orifice of the MV. As a result, during systole, prolapse is limited and an effective occluder seal with the native leaflets is obtained.

As illustrated in FIG. 98E, in this embodiment movable occluder membranes 6441, 6442 may be attached to support frame 6420 by a zig-zag lock stitch or seam 6423. Subsequent stitches are placed alternately through both membranes, one outwardly above (6424) and one inwardly below (6425) the frame 6420. This type of seam ensures that the opposing membranes 6441, 6442 are set in tight contact against the frame and to one another so as to provide a seal therebetween. This seal maintains the blood within the pressurized occluder cavity 6449 during systole. As well, particularly in systole when cavity 6449 is pressurized, lock stitch seams distribute the dynamic load that the occluder 6440 is exposed to more evenly and over a larger surface of the membrane. In contrast, using discrete sutures could introduce concentrated stresses on the occluder membrane around the puncture site. Other types of seams or stitching patterns are also possible.

As shown, support frame 6420 has a circular cross-sectional area, but other cross-sections are possible to provide the desired structural stiffness in use and appropriate elasticity to allow frame collapsibility required for catheter delivery.

It may be desirable to configure and size the support frame 6420 with a structural stiffness that allows the frame to bend inwardly and elastically with the contraction of the left ventricle LV during systole. This inward bending may advantageously serve to assist in spacing apart the occluder membranes 6441, 6442 during systole.

Optionally, the frame 6420 may be covered with a sheath 6426, which may be made from an inert bioprosthetic material, such as a silicone elastomer, ePTFE, or other polymers or other suitable inert materials. Sheath 6426 may have a tubular cross-section or preferably, as shown, an elongated cross-section extending above and below the frame in between membranes 6441, 6442. The sheath 6426 may serve to enhance the desired sealing between the joined occluder membranes, and may even be provided with some degree of compressibility to enhance that effect. Placing the sheath within the lock stitch arrangement may serve to reinforce seam 6423 while also sealing any potential leakage paths created in the membranes by the punctures of the stitching needle. Optionally, to further reinforce seam 6423 between occluder membranes, one or more crimp members 6427 may be placed around the seam and support frame in order to further compress the pericardial membrane against the support frame. The crimp member(s) 6427 may be spaced apart along the support frame at discrete locations or, alternatively, can be provided along the entire length of support frame 6420 that is engaged with the occluder membranes. Crimp members may be made from Nitinol, cobalt chrome, titanium or other suitable bioprosthetic material including polymeric materials possessing the required structural stiffness.

At some sections of occluder 6440 (not shown), a single sheet or ply of pericardium can be folded over the frame 6420 to create the two opposing occluder membranes 6441, 6442. As such the membrane fringe 6428 is eliminated over these sections. The single sheet of pericardium may be attached to frame in the same manner described above, or with the zig-zag portion of the lock stitch over this section replaced with a straight lock stitch (i.e., where successive stitches are placed inwardly below the frame).

Optionally, sheath 6426 may also be configured to extend inwardly, below the lock stitch seam 6423, to occupy space within the occluder volume 6449 and keep membranes 6441, 6442 slightly spaced apart from one another immediately adjacent to the frame. Such an arrangement may eliminate potential stagnation zones in blood flow adjacent to the frame and where otherwise, without the membrane spacing from provided by the sheath, the membranes would clap shut during diastole. This arrangement may also enhance flushing of the occluder inner cavity 6449.

As shown in FIG. 98B, support frame 6520 may be provided with a pair of loop elements 6422, 6421 located adjacent to inlet INL, one proximal to the clip, the other proximal to the ventricular connector 6490. Loop elements 6422, 6421 may serve to locally reinforce the attachment of the membranes 6441, 6442 to the frame (i.e., securely anchoring the membrane free margins). Discrete retention sutures (not shown) may be placed through both the membranes, through the opening in one of the loop elements, and secured by tying around and against the support frame. Additionally, one or more lower zig-zag lock stitches 6425 may be passed through the openings of the loop elements. As such, the occluder membranes 6441, 6442 are retained and kept from sliding up along the support frame 6420 away from inlet INL.

During a transcatheter delivery of SOD 6400, a releasable traction suture 6429 may be placed through the openings of both loop elements 6421, 6422 and manipulated through a delivery catheter (not shown) to retract the loop elements into proximity to one another, thereby collapsing support frame 6420 and occluder 6440 into a compact configuration.

As shown in FIG. 98F, membrane fringe 6428 can be appropriately sized to fill and seal any residual gap RG that may exist between spacer SP and upstanding section of support frame 6420 adjacent to the clip CL.

Occluder membranes 6441, 6442 made from thin, supple, pliable pericardium offer functional advantages during use. The pericardium membrane can fold on itself along free margin 6443, 6444 to adapt to the size (i.e., leakage area) of the regurgitant orifice MRO. As such, it may effectively seal a regurgitant orifice MRO having a smaller perimeter and area than the maximum open inlet area AS (FIG. 97D) that the occluder 6440 is capable of assuming at its limit. As well, the thin pliable nature of these membranes allows them to adapt to any irregular shape of MRO, even filling in clefts or commissural spaces that may be sources of mitral regurgitation.

As described above, SOD 6400 is provided with occluder membranes made from pericardium material. Other suitably thin, pliable, bioprosthetic materials may also be chosen for the occluder membranes including polymeric or plastic materials. In such embodiments, membrane spacing element 6450 may be embedded within the polymeric membrane during the fabrication process, or glued to the membrane. As well, support frame 6420, or any section thereof, may be selectively embedded within the polymeric membrane. The residual gap RG (FIG. 98F) may be filled by a flap or protrusion of polymeric material disposed outwardly away from support frame toward clip CL.

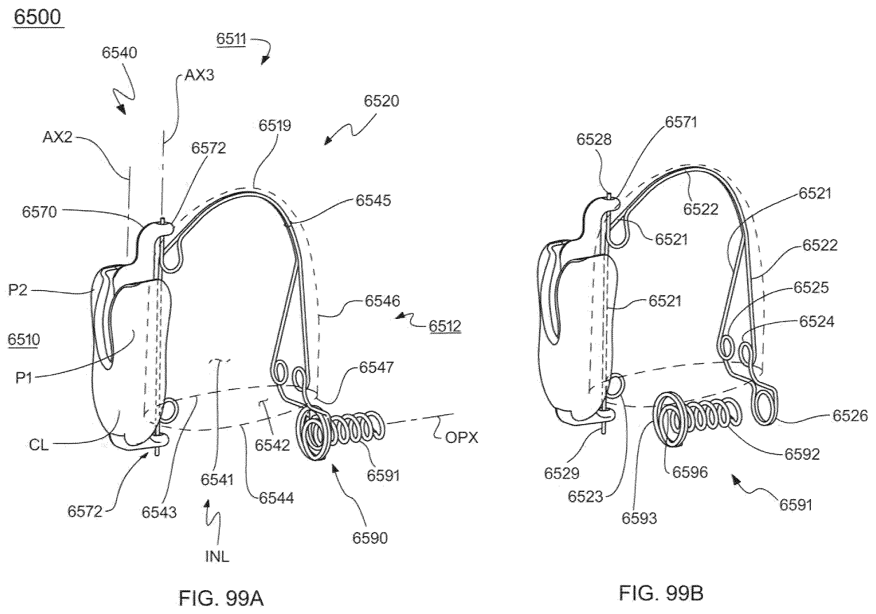

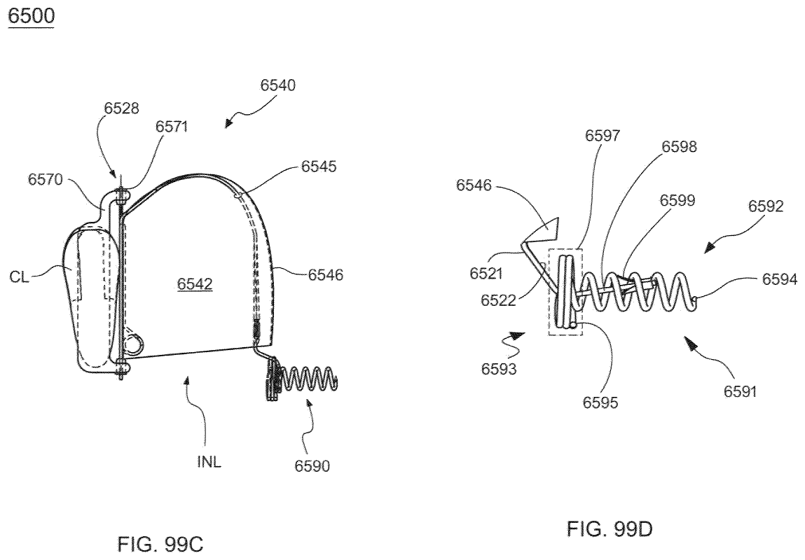

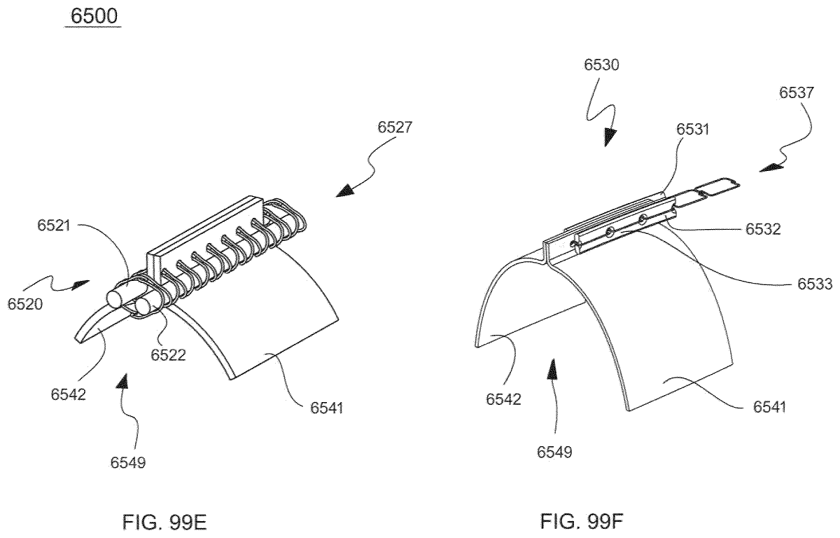

FIGS. 99A-99F illustrate an SOD 6500 which includes a single movable membrane occluder 6540. SOD 6500 may be similar to SOD 6400, except for variations in the ventricular connector 6590 and the configuration of support frame 6520.

Occluder 6540 includes two primary larger movable occluding membranes 6541, 6542 and a one secondary smaller membrane section 6546. Membranes 6541, 6542 are configured and sized to engage with the anterior AL or posterior PL leaflets, respectively. Membrane 6546 in configured and sized to engage with either one of the leaflets AL, PL, or both, and extend into a residual commissural space between leaflets AL and PL that the native leaflets are not able to seal during systole.

The membranes 6541, 6542, 6546 may be made from a single sheet of pericardium secured to the support frame 6520 to define or delimit the three membranes. Occluder 6540 has an inlet INL, a closed end 6519, and an internal cavity or volume 6549 defined by the space that is enclosed by the membranes beyond the inlet INL. The occluder is pivotably engaged to clip connector 6570, which is in turn coupled to clip CL between paddles P1, P2.

A unitary elastic, or preferably superelastic, wire is used to produce support frame 6520 having a frame first and second ends 6528, 6529 that are pivotably connected to the clip connector at a first and second crimped end 6571, 6572 respectively.

As shown in FIG. 99A, clip connector 6570 provides an occluder-clip pivoting axis AX3 that is offset away from the centerline axis AX2 through clip CL. As such, occluder 6540 is positioned more distal to the clip and, in use, closer to the MVA than the previous embodiment 6400.

The closed clip-facing side 6510 of the occluder is supported by a single frame (i.e., only by frame section 6521) with pericardium membranes 6541, 6542 secured thereto by a stitching configuration similar to seam 6423 of embodiment 6400 (FIG. 98E). Frame 6520 is similarly provided with a loop element 6523 to secure the free margins 6543, 6544 adjacent to the occluder inlet INL.

In this embodiment, the closed top side 6511 of the occluder is supported by a double frame (i.e., two parallel frame sections 6521, 6522) with membranes 6541, 6542 secured therebetween with a stitching arrangement described below in greater detail with reference to FIG. 99D. Over most of the arcuate top side 6511 that, in use, will be extending into the LA, the two frame sections 6521, 6522 are disposed on the outer surface of occluder 6540, until both frame section re-enter the occluder inner cavity 6549 through a pair of membrane openings 6545 (i.e., one through each membrane 6541, 6542), beyond which the frame sections diverge downwardly apart from one another.

The closed commissure-facing side 6512 of the occluder, below the membrane openings 6545 and outwardly away from diverging frame sections 6521, 6522, is bounded by occluding membrane 6546. There are no membrane-joining seams over the surface of membrane 6546 as the pericardium sheet used to produce the occluder 6540 is folded at the occluder side 6512. In comparison to previous embodiment SOD 6400, the distal-most limits of occluder 6540 are not bounded by a relatively rigid support frame 6420 (FIG. 98F), but rather a compliant, flexible membrane 6546. As will be described in greater detail below, occluder 6540 is advantageous when sealing against a small commissural leaflet, or sealing a commissural leakage.

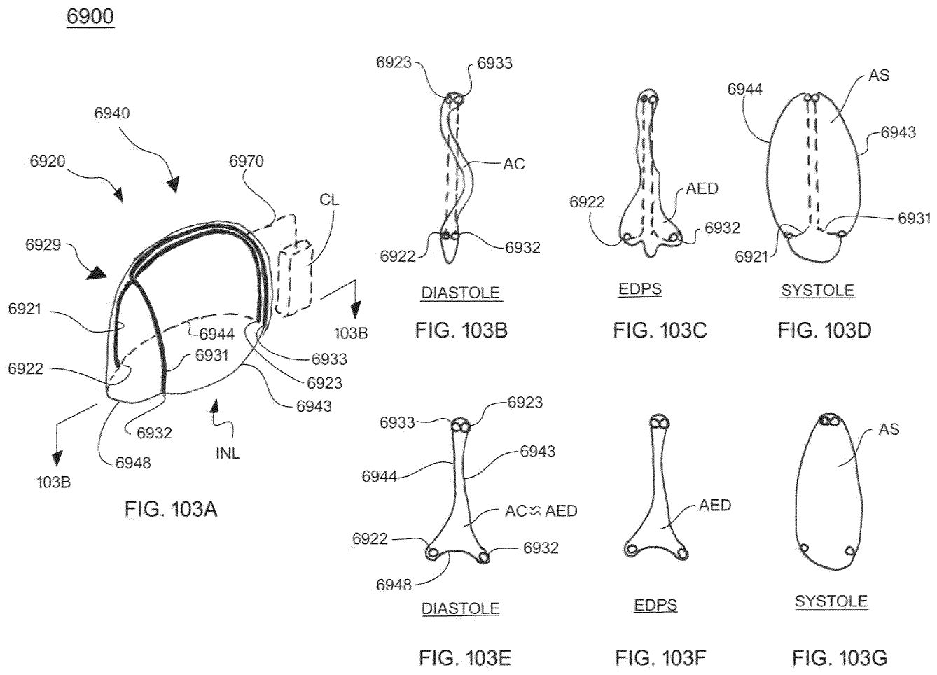

Frame sections 6521, 6522 are configured with loop elements 6525, 6524 adjacent to inlet INL. Membranes 6542, 6546 are minimally attached at loop element 6524, and membranes 6541, 6546 are minimally attached at loop element 6525 in a similar manner as previously described in embodiment 6400. With this minimal membrane attachment, the three membranes will billow outwardly in systole cooperating as one continuous membrane surface. Alternatively, membranes 6542, 6546 may be stitched continuously over the entire diverging length of frame section 6522, and membranes 6541, 6546 stitched continuously over the entire diverging length of frame section 6521. As such, the three distinct membrane sections 6541, 6542, 6546 will be defined with free margins 6543, 6544, 6547, respectively assuming a diastolic configuration similar to what is illustrated in FIG. 103E, and a systolic configuration similar to what is shown in FIG. 103G. The diverging frame sections 6521, 6522 ensure an open inlet INL between the three membrane free margins, during the entire cardiac cycle including diastole, thereby ensuring that blood will flow into occluder inner cavity 6549 and occluder 6540 gets pressurized in systole.

Diverging sections 6521, 6522 converge again below inlet INL and join together at hoop 6526, which serves to engage with a ventricular connector 6590 that includes, in this embodiment, a helically-coiled wire tissue anchor or fastener 6591. Preferably, tissue fastener 6591 is integrally mounted to hoop 6526 but still able to rotate relative to hoop 6526 (i.e., suitable for a catheter delivery procedure wherein both the occluder and the ventricular connector are integrated and delivered simultaneously). Fastener head 6593 of fastener 6591 is configured with a drive portion 6596 adapted to engage a delivery catheter (not shown) that is capable of transmitting torque sufficient to drive tissue fastener 6591 into ventricular tissue.

Tissue fastener 6591 may be made from a single Nitinol wire that is shape-set in a helically wound configuration to form a first, smaller-diameter helical section or shaft 6592, and a second, larger-diameter helical section or head 6593. Other materials are possible for the fastener, such as titanium, or other suitable bioprosthetic materials possessing the required mechanical strength.

Extending from tissue-piercing fastener tip 6594 toward fastener head 6593, helical windings are formed, for example, in a counter clockwise direction at the smaller shaft diameter. Over the drive portion 6596, the small diameter windings transition to the larger diameter head windings, which are also formed in a counter clockwise direction, but advancing instead from head 6593 toward tip 6594.

As such, a locking terminal end 6595 is provided to serve as an anti-rotation feature to keep fastener 6591 engaged with a ventricular tissue. Trying to unscrew the fastener when head section 6595 is in contact with ventricle wall (i.e., CCW rotation), would result in driving the terminal end 6595 deeper into the tissue at a larger diameter tissue entry point than the entry point of tip 6594. As such, attempt to unscrew shaft 6592 would result in further locking the fastener in place at end 6595.

The rotating engagement between hoop 6526 and screw 6591 allows occluder 6540 to pivot relative to the ventricular connector (and the LV) about an occluder pivoting axis OPX. As such, the occluder may tilt or pivot toward the anterior or posterior annulus along the A-P direction (i.e., RKS in FIG. 96C). Since axis OPX is below the inlet INL, SOD 6500 will have the device operability benefits previously described in reference to SOD 6200 (FIGS. 96B-96C).

Relative to barb 6491 of embodiment SOD 6400, the helical windings of fastener 6591 provide a larger bearing surface to react the dynamic loads exerted occluder 6540. As such, the lower contact pressure on the anchored ventricular tissue reduces trauma and risk of connector disengagement. Providing larger diameter windings, or more windings, to the fastener will further reduce tissue trauma.

Shown schematically as a dotted outline, a textile covering 6597 may be included to cover head 6593 and advantageously promote local tissue ingrowth that would encapsulate the fastener head. As such, a more secure attachment of ventricular connector 6590 to the LV is provided. Alternatively, a textile washer or woven fabric insert may be placed between the fastener head and the LV to promote tissue ingrowth. The tissue ingrowth occurs over a relatively short time after implantation (i.e., 10-15 days) and will assist the mechanical fastener 6591 in supporting the dynamic loads exerted on the SOD and reacted at the ventricular tissue.

With reference to FIG. 99D, rather than terminating in hoop 6526, frame sections 6521, 6522 can be joined to form a barb 6598 similar to previously described barb 6491 (FIG. 98B). Alternatively, screw 6591 can be inserted in the ventricular tissue and subsequently barb 6598 inserted into the center of coiled fastener 6591. As such, the barb element 6599 will be engaged with ventricular tissue and/or the helical windings of fastener 6591 to create a more robust anchoring arrangement

The attachment of occluder membranes 6541, 6542 to support frame 6520 is illustrated in FIG. 99E. Over the closed, occluder top portion 6511, the two membranes are inserted and clamped between two parallel frame sections 6521, 6522. A zig-zag lock stitch seam 6527 is preferably used to apply compression against the frame sections thus clamping the membranes therebetween with a more evenly distributed retention force compared to membrane retention with a discrete suture line. In use, the separating loads induced at the frame location from a pressurized occluder cavity 6549 are more uniformly distributed to the clamped membranes compared to membranes that are joined solely by stitching.

FIG. 99F illustrates an alternative membrane-securing configuration to the configuration of FIG. 99E. Frame sections 6531, 6532 of support frame 6530 are produced by laser cutting a Nitinol sheet. The frame cross-section is generally rectangular and is provided with an array of spaced holes or slots 6533 through which to pass thread or suture material. Instead of using a zig-zag lock stitch around the frame sections as shown in FIG. 99E, a straight lock stitch 6537 disposed through holes 6533 is used to secure and clamp the frames sections together, and the occluder membranes 6541, 6542 captured therebetween, under compression.

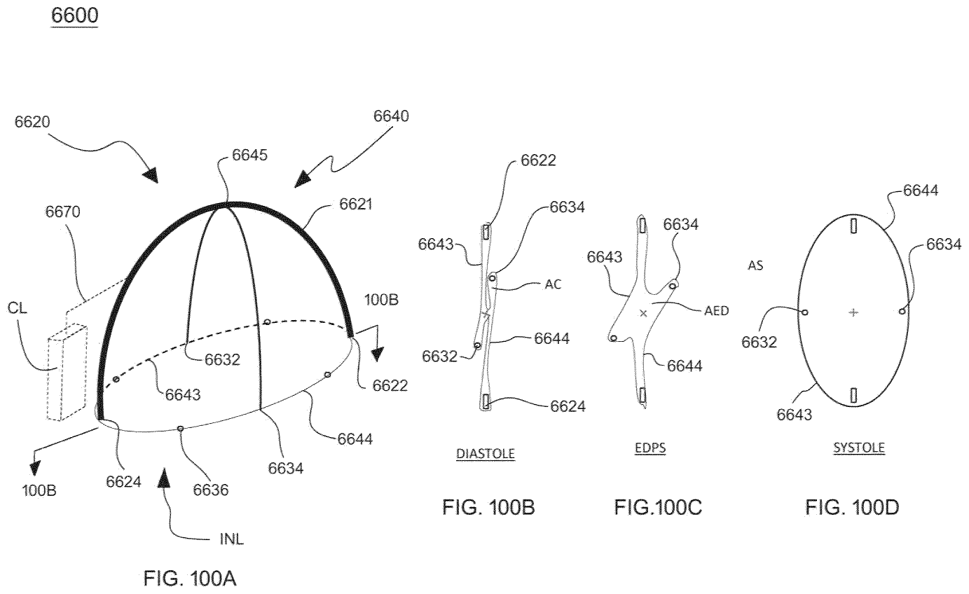

With reference to FIGS. 100A-104D, several embodiments of movable membrane SODs are described, in which the membrane spacing element is implemented by a dynamic frame configuration urging the occluder to assume the EDPS configuration. In these figures, cross-sectional views at the occluder inlet INL will schematically illustrate the position of dynamic frame members and the general shape of the membrane free margin at three occluder configurations: closed, diastolic configuration, intermediate EDPS configuration, and open, systolic configuration.

With a dynamic frame configuration, the movement of frame members from the diastolic configuration (i.e., frame members with stored internal energy) to the EDPS configuration (i.e., frame members in unconstrained free state) is achieved through a release of internal energy that was extracted from the dynamic forces applied on the movable occluding membranes to collapse them. The movement of frame members from the EDPS configuration (i.e., frame members in unconstrained free state) to the systolic configuration (i.e., frame members with stored internal energy) is achieved through the inertial fluid forces being reacted within the occluder internal cavity resulting in movement of frame members beyond their unconstrained free state.

As illustrated in FIG. 100A, SOD 6600 includes occluder 6640, support frame 6620, and a clip connector 6670. Support frame 6620 includes two arcuate frames 6621, 6631 engaged at apex connector 6645. Occluder 6640 includes two movable occluder membranes having membrane free margins 6643, 6644, respectively that define the open perimeter of occluder inlet INL.

Frame 6621 is similar to frame 6420 of embodiment 6400 (FIG. 98B), and has a greater structural stiffness than flexible frame 6631. Membrane free margin 6644 extends from frame end 6622 to frame end 6624 and is also engaged to frame end 6634 generally at its mid-span. Similarly, membrane free margin 6643 extends from frame end 6622 to frame end 6624 and is also engaged to frame end 6632 generally at its mid-span.

In this embodiment, arcuate frame 6631 is pivotably engaged to arcuate frame 6621 through a torsion spring (or other mechanism for providing a torsional bias) included in apex connector 6645. When the torsion spring is in its free non-energized state, frame 6631 assumes an angular orientation relative to frame 6621 corresponding to the EDPS configuration that the occluder will assume to achieve the required membrane spacing at inlet INL, in use, at the EDPS point in the cardiac cycle (FIG. 100C). The resulting occluder inlet area AED advantageously prepares the occluder to receive systolic blood flow and ensures it will fill and be pressurized in systole. In diastole, the dynamic load on the membranes overcomes the torsion spring stiffness at connector 6645, causing frame 6631 to pivot relative to frame 6621 (i.e., counter clockwise) and collapsing membrane free margin 6644, 6643 in proximity to one another. At this point, the occluder assumes the closed diastolic configuration illustrated in FIG. 100B (i.e., with minimum inlet area AC. The internal energy stored in the torsion spring at diastole will be converted to pivoting motion of frame 6631 relative to frame 6621 as the occluder transitions to its EDPS configuration. From EDPS to systole, arcuate frame 6631 will continue to pivot away from frame 6621 (i.e., CW) as the occluder assumes the open, systolic configuration (FIG. 100D). In systole, the internal energy stored in the torsion spring will be released in pivoting motion of frame 6631 back towards frame 6621 (i.e., counter clockwise).

Alternatively, arcuate frame 6631 may be fixedly engaged to frame 6621 at connector 6645, and configured to act as an arcuate spring element itself, whereby the ends of the frame 6631 flex inwardly toward one another during diastole and outwardly away from one another during systole. In this variant, arcuate frame 6631 would be sized in its free, non-energized state to have a spacing between ends 6632, 6634 corresponding to occluder EDPS configuration (i.e., to achieve the required membrane spreading). As such, arcuate frame 6631 would function similar to membrane spacing element 6450 (embodiment 6400;

FIG. 98B) with the frame ends 6632, 6634 operating analogously to loops 6451, 6452 as illustrated in FIGS. 98D. Arcuate frame 6631 would also function as a prolapse-limiting member for occluder 6640.

Alternatively, occluder 6640 may be configured with more than one arcuate spring elements, all fixedly connected at apex 6645, and having the terminal ends thereof spaced along and engaged to the membrane free margins (i.e., for example at the four points 6636 in FIG. 100A) producing an umbrella-like arcuate spring configuration. In this configuration, spring elements would flex inwardly toward one another during diastole and outwardly away from one another during systole. The spring elements would be set in a predetermined, spaced apart relationship corresponding to the EDPS configuration so as to ensure a desirable occluder inlet area AED.

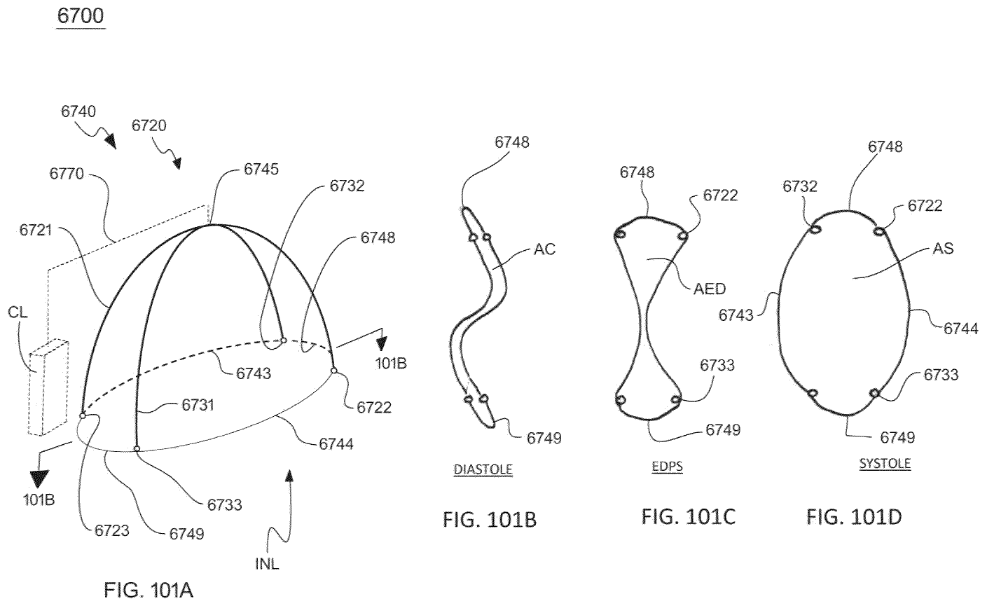

In another embodiment, a membrane spacing element can be implemented with multiple frame members that are pivotably connected. As illustrated in FIG. 101A, SOD 6700 includes occluder 6740, support frame 6720, and a clip connector 6770. Support frame 6720 includes two arcuate frames 6721, 6731 engaged at apex connector 6745 and offset angularly apart by approximately 15-25 degrees. Occluder 6740 includes two larger movable membranes having free margins 6743, 6744, respectively, and two smaller movable membranes having free margins 6748, 6749, respectively. Open perimeter of occluder inlet INL is defined by free margins 6743, 6744, 6748, 6749.

The larger membranes are most suited to engage with the anterior AL or posterior PL leaflets of the MV. Smaller membrane having free margin 6749, adjacent to clip CL, is suitable to fill any residual gap RG (FIG. 98F) that may be present between the clip (or spacer) and the support frame 6720. Smaller membrane having free margin 6748 is suitable for filling commissural leakages or for engaging small commissural leaflets.

In a first preferred configuration, frames 6731, 6721 are made from elastic or superelastic material and are fixedly connected at apex 6745. In use, frame terminal ends 6723, 6733 (and similarly 6722, 6732) are designed to flex toward one another during diastole, and away from one another during systole. In the free, non-constrained state, frames 6731, 6721 are shape-set so that the spacing between terminal ends 6723, 6733 (and similarly 6722, 6732) corresponds to the desired spacing between said terminal ends to achieve the required membrane spacing at EDPS when occluder 6740 will assume the intermediate EDPS configuration (FIG. 101C). The resulting occluder inlet area AED advantageously prepares the occluder to receive systolic blood flow and ensures it will fill and be pressurized in systole.

In diastole, the dynamic load on the membranes is sufficient to elastically bend the frames toward one another (or at least the terminal ends thereof) thereby collapsing the larger opposing membranes against one another and folding the smaller membranes. At this point, the occluder assumes the closed, diastolic configuration illustrated in FIG. 101B (i.e., with minimum inlet area AC). The internal energy stored in bent frames 6721, 6731 during diastole will be converted to relative displacement between the frames as occluder 6740 transitions to its EDPS configuration (FIG. 101C). From EDPS to systole, frames 6721, 6731 will continue to bend or deflect away from one another (i.e., to the extent that the smaller membranes allow) as occluder 6740 assumes the open, systolic configuration (FIG. 101D). In systole, the internal energy stored in the over-bent frames will be released in relative displacement of frames 6721, 6731 back towards one another.

In a variant configuration, frames 6721, 6731 may be pivotably engaged through an apex connector 6745 including a torsion spring element acting between the frames, in a similar arrangement as described in embodiment 6600. In this case, the frames are stiffer than the frames described above and the relative spacing between terminal ends 6723, 6733 (and 6722, 6733) is produced by the pivoting between the rather than bending of the frames.

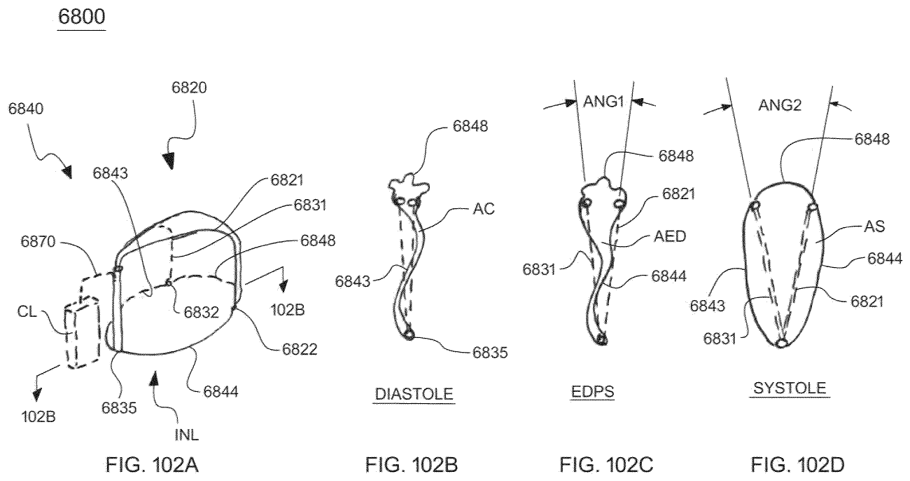

In another embodiment, a membrane spacing element can be implemented with multiple frame members that are pivotably connected near one end. As illustrated in FIG. 102A, SOD 6800 includes occluder 6840, support frame 6820, and a clip connector 6870. Support frame 6820 includes two arcuate frames 6821, 6831 pivotably engaged to a common upstanding post 6835 disposed adjacent to clip CL. Post 6835 may be a tubular member into which frames 6821, 6831 are inserted and retained in pivoting engagement (i.e., able to pivot about axis AX5). Frames 6821,6831 have swinging terminal ends 6822, 6832, respectively.

Occluder 6840 includes two larger movable membranes having free margins 6843, 6844, respectively, suitable for engaging the anterior AL or posterior PL leaflets, and one smaller bellows-type membrane having free margin 6848 suitable for filling leakages at clefts or commissures or leakages that occur proximal to the annulus of mitral valve. Open perimeter of occluder inlet INL is defined by free margins 6843, 6844, 6848.

A torsional spring element (not shown in FIGS. 102A to 102D) may be disposed between frames 6821, 62831 to act in a similar manner to the torsion spring previously described. When the torsion spring is in its free non-energized state, the frame members are angled relative to one another at the predetermined angle ANG1 corresponding to the desired angular relationship between the frames at EDPS to achieve the required membrane spacing at inlet INL, when occluder 6840 will assume the intermediate EDPS configuration (FIG. 102C). The resulting occluder inlet area AED advantageously prepares the occluder to receive systolic blood flow and ensures it will fill and be pressurized in systole. In use, frames 6821, 6831 are designed to pivot toward one another during diastole and come into a generally parallel orientation (FIG. 102B), and away from one another during systole and come to a maximum predetermined angular orientation of ANG 2 (FIG. 102D).

In another embodiment, a membrane spacing element can be implemented with multiple frame members that are movable relative to each other but necessarily connected to each other. As illustrated in FIG. 103A, SOD 6900 includes occluder 6940, support frame 6920, and a clip connector 6970. Support frame 6920 is similar to support frame 6520 of SOD 6500 (FIG. 99B) having two parallel frame sections 6921, 6931 in proximity over the closed top section of occluder 6940, and that subsequently diverge apart towards occluder inlet INL. Unlike support frame 6520, support frame 6920 has the diverging frame sections not joined to one another but free to move at terminal frame ends 6922, 6932 (i.e., adjacent the occluder free margins).

Similar to SOD 6500, occluder 6940 includes two larger movable membranes having free margins 6943, 6944, respectively, suitable for engaging the anterior AL or posterior PL leaflets, and one smaller membrane having free margin 6948 suitable for engaging with small commissural leaflets or sealing leakages that occur proximal to the annulus of mitral valve. Open perimeter of occluder inlet INL is defined by free margins 6943, 6944, 6948.

Frame ends 6922, 6932 flex inwardly toward one another during diastole and outwardly away from one another during systole. This relative displacement may be achieved in a number of ways. For instance, the diverging portions may be configured and sized to elastically and progressively bend along their length, elastically bend at a discrete location along their length or at the junction point 6929, 6939 where they start to diverge from one another, or twist at junction point 6929, 6939 through a torsion effect either locally thereat or over the frame sections that are in parallel alignment to one another. In all variants listed above, frames 6921, 6931 would be configured and sized so that in the free, non-energized state of the support frame 6920, the spacing between frame ends 6922, 6932 corresponds to the desired spacing between the frames at EDPS to achieve the required membrane spacing at inlet INL, when occluder 6840 will assume the intermediate EDPS configuration (FIG. 103C). The resulting occluder inlet area AED advantageously prepares the occluder to receive systolic blood flow and ensures it will fill and be pressurized in systole.

In comparison to FIGS. 103B-103D, the effect of having frames 6921, 6931 statically fixed throughout the cardiac cycle is schematically illustrated in FIGS. 103E-103G. With statically fixed frames, the occluder inlet area AC at diastole (FIG. 103E) is necessarily set to be larger than in a dynamic frame arrangement (FIG. 103B) to ensure that area AED at EDPS is sufficient and available (FIG. 103F). The diastolic configuration in FIG. 103E with a static frame arrangement represents a greater obstruction to flow, from LA into LV, compared to the diastolic configuration in FIG. 103B possible with a dynamic frame arrangement. As well, in such static frame arrangements, the length of the membrane free margins may have to be shortened in order to ensure that inlet area AED remains sufficiently open, since the effect of the dynamic frame creating an opening directly behind its displacement trajectory is not present.

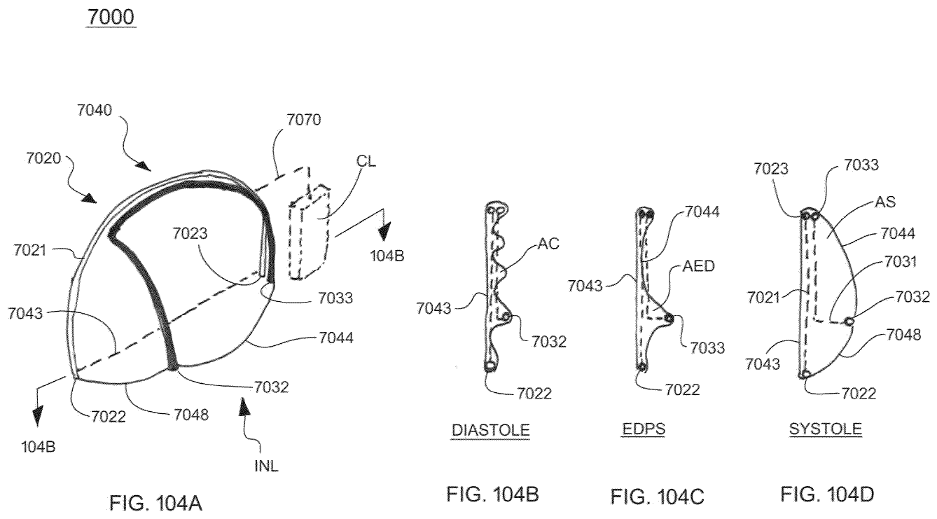

In another embodiment, a membrane spacing element can be implemented with multiple asymmetric frame members. As illustrated in FIG. 104A, SOD 7000 includes occluder 7040, support frame 7020, and a clip connector 7070. Support frame 7020 includes first and second frame members 7021, 7031 with first frame member 7021 extending from the clip CL towards a commissure location (i.e., similar to frame 6420 of SOD 6400; FIG. 98B), and second frame member 7031 extending parallel to first frame member 7021 over the closed top section of occluder 7040 (i.e., similar to support frame 6520 of SOD 6500; FIG. 99B) and subsequently diverging away from first member towards occluder inlet INL (i.e., towards a native leaflet; similar to frame 6931 of SOD 6900; FIG. 103A).

Occluder 7040 includes one larger membrane having free margin 7043 that is suitable for engaging the anterior leaflet AL, and two smaller membranes having free margins 7044, 7048 suitable for engaging with posterior leaflet PL and sealing leakages or clefts that occur between scallops of the posterior leaflet (ex., between P2 and P3). Open perimeter of occluder inlet INL is defined by free margins 7043, 7044, 7048. In this embodiment, the larger membrane is flexible but relatively static to the smaller membranes in order to ensure that the inlet area AED at EDPS is not restricted. The anterior leaflet AL usually does not present any clefts hence engagement with the larger membrane is appropriate.

Frame end 7032 flexes inwardly toward frame end 7021 during diastole and outwardly away from frame end 7021 during systole. This relative displacement may be achieved in a number of ways, as described in SOD 6900 in reference to dynamic frame member 6931. Frames 7021, 7031 are configured and sized so that in the free, non-energized state of the support frame 7020, the spacing between frame end 7032 and relatively static membrane free margin 7043 corresponds to the desired spacing at EDPS as illustrated in FIG. 104C, to achieve the required membrane spacing at inlet INL, when occluder 7040 will assume the intermediate EDPS configuration. The resulting occluder inlet area AED advantageously prepares the occluder to receive systolic blood flow and ensures it will fill and be pressurized in systole.

SODs 6600, 6700, 6800, 6900, 7000 described above are provided with a dynamic frame arrangement to implement the membrane spacing element. If these SODs are instead provided with a similar but static frame arrangement, the spacing between terminal frame ends at the inlet INL, as illustrated in the EDPS configuration (i.e., FIGS. 100C, 101C, 102C, 103C, 104C, 105C) needs to be implemented also in the corresponding diastolic configuration of the occluder (i.e., instead of the more collapsed diastolic configuration possible with a dynamic frame arrangement). This is to ensure that in such static frame arrangements, the movable membrane occluder fills with blood and becomes pressurized during systole.

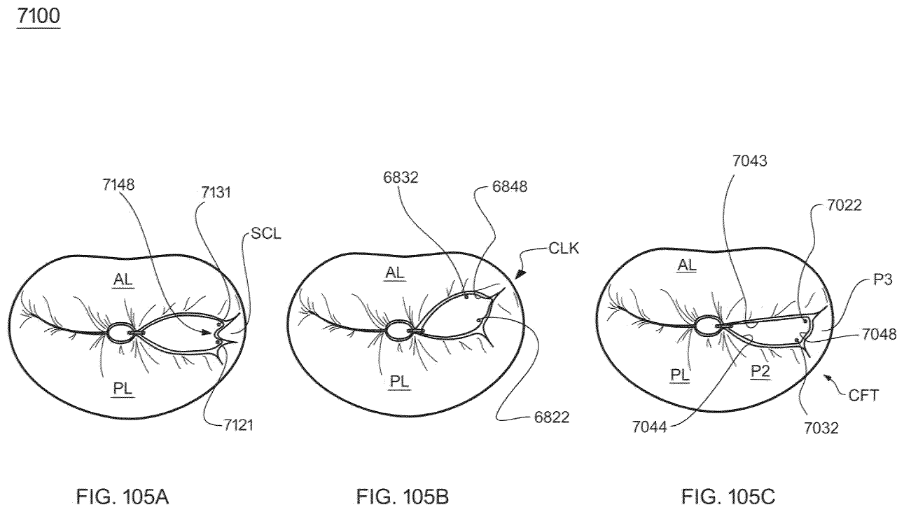

FIGS. 105A-105C illustrate some of the movable membrane SOD embodiments installed within a mitral valve MV, during systole.

FIG. 105A illustrates how the SODs 6700, 6900 are suitable for engaging with a small commissural leaflet SCL. More specifically, is shown how leaflet SCL can engage with membranes 6748, 6948 (i.e., collectively labelled 7148) while extending in between the frame sections 7121, 7131.

FIG. 105B illustrates how the SOD 6800 is suitable for sealing commissural leaks CLK. More specifically, is shown how the excess membrane surface of movable membrane 6848 is advantageous to seal commissure leak CLK at the commissure between the anterior AL and posterior PL leaflets.

FIG. 105C illustrates how the SOD 6900 is suitable for sealing a leakage in a cleft CLK. More specifically, is shown how frame member 7032 may be positioned within the cleft CFT (ex., P2 and P3) and how membranes 7044, 7048 are suitable to engage with scallops P2, P3 of the posterior leaflet PL.

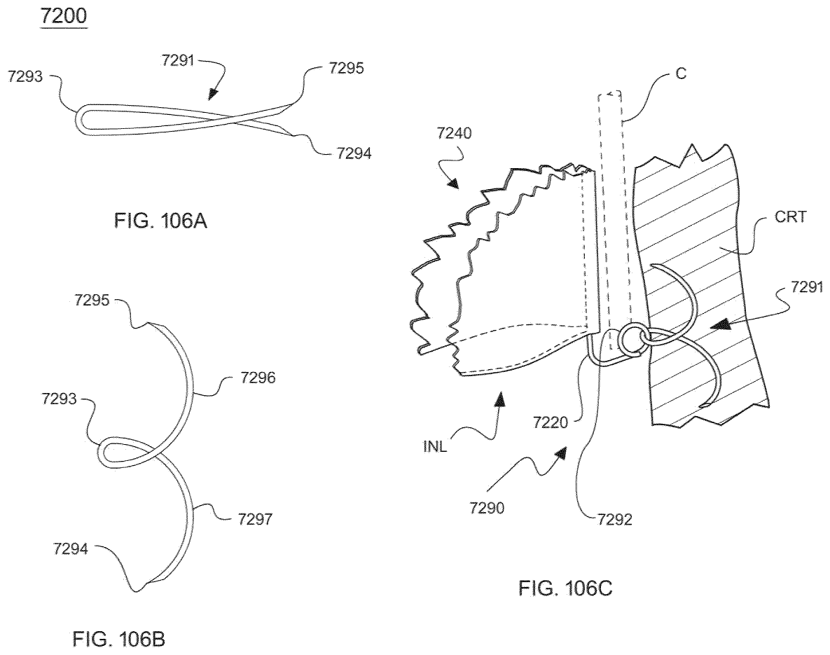

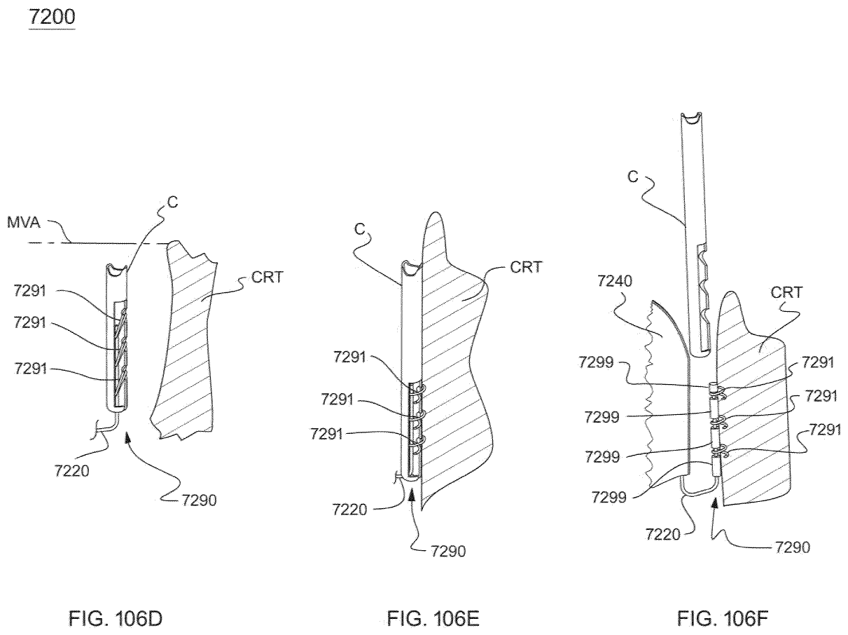

As noted above, a ventricular connector may be implemented in many ways. Another implementation of a ventricular connector is shown in FIGS. 106A-106C for SOD 7200. SOD 7200 includes a ventricular connector 7290, which in this embodiment is implemented as a tissue-anchoring clip 7291.

SOD 7200 includes a movable membrane occluder 7240 similar to occluder 6440 of SOD 6400, with a support frame 7220 that similarly extends laterally beyond the occluding portion of occluder 7240 to terminate in loop member 7292, where it is engaged with the ventricular connector 7290.

Anchoring clip 7291 is made from an elastic metal alloy, or preferably a superelastic material, such as Nitinol, and is shape-set to a free, unconstrained state corresponding to the tissue-grasping configuration (FIG. 106B). As shown in FIG. 106B, the clip has two clip arms 7296, 7297 that diverge and curl away from one another to form opposing hooks, each arm terminating at a straight distal tip 7295, 7294, respectively, configured to pierce cardiac tissue CRT. Clip arms 7296, 7297 are connected at clip loop 7293 that is shaped and sized with a sufficiently open area to allow insertion therethrough of loop member 7292. As shown in FIG. 106C, occluder 7240 and ventricular connector 7290 are pivotably engaged generally adjacent (or below) the occluder inlet INL through the coupling of loop member 7292 and clip anchor 7291. As such, the occluder 7240 can assume a favorable orientation (i.e., tilting along the A-P direction; RKS in FIG. 96C) to provide sealing engagement with the native leaflets during systole.

In FIG. 106A, anchoring clip 7291 is shown in a tissue-piercing delivery configuration as it would be disposed and constrained within the lumen of a delivery catheter C.

In FIG. 106B, anchoring clip 7291 is shown in a tissue-grasping or tissue-retaining, deployed anchoring configuration. Clip is elastically-deformable between the delivery configuration and the anchoring configuration.

With reference to FIGS. 106D-106E, a plurality of anchoring clips 7291 may be required to securely anchor SOD 7200 to the wall of the left ventricle if the dynamic loads exerted on occluder 7240 (i.e., force F; FIG. 98A) are excessive for a single anchoring clip. Ventricular connector 7290 may be configured with an extended frame section 7298 beyond loop member 7292 to dispose a plurality of spaced apart anchoring clips 7291, which may be deployed sequentially or simultaneously with a clip delivery catheter C engaged with extended frame section 7298. As shown in FIG. 106E, delivery catheter C is positioned and oriented at the desired location against the heart tissue. The anchoring clips 7291 are loaded within catheter C in the constrained tissue-piercing configuration and released therefrom through openings formed by retractable gates. Within the lumen of the catheter C, a translating cable or rod member (not shown) is releasably engaged with the terminal end of ventricular connector 7290. When operated, the translating cable retracts frame section 7298 through the delivery catheter C, as shown in FIGS. 106D-106E, thereby piercing the anchoring clips into the cardiac tissue CRT. Further retraction of the frame section 7298 through the catheter C embeds the anchoring clips deeper into the tissue of the ventricle as the clip anchors transition to the fully-deployed, tissue-retaining configuration (FIG. 106F). Ventricular connector 7290 is then released from catheter C by actuating the retractable gates.

Ventricular connector 7290 is shown extending upwards along the wall of left ventricle LV towards the left atrium LA, but it may be secured in other equally suitable orientations, depending on the specific anatomy of the patient. A pivoting joint between frame section 7298 and occluder 7240 may be provided (i.e., at the location of loop 7292) to allow for a range of variable anchoring orientations to the left ventricle LV.\

Anchor clips 7291 are held in a spaced apart relationship by clip spacers 7299. The spacers are covered in an implantable fabric material that promotes a favorable bioreaction with tissue ingrowth that incorporates the spacers into the tissue of the left ventricle wall.

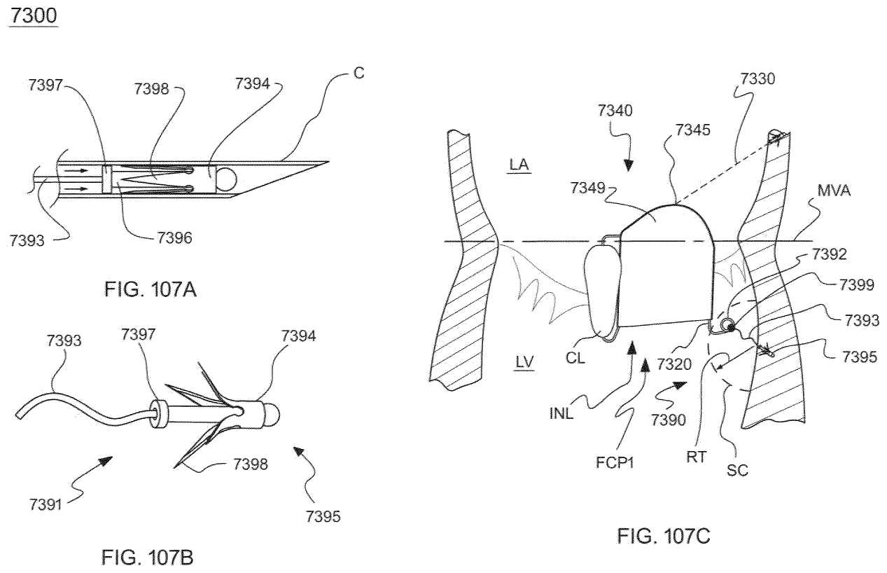

Another implementation of a ventricular connector is shown in FIGS. 107A-107C, in which SOD 7300 includes a ventricular connector 7390 that is implemented as a tethered multi-pronged anchor 7391.

SOD 7300 includes a movable membrane occluder 7340 similar to occluder 6440 of SOD 6400, with a support frame 7320 that similarly extends laterally beyond the occluding portion of occluder 7340 to terminate in loop member 7392, where it is engaged with the ventricular connector 7390.

Anchor 7391 is comprised of a flexible wire element or flexible tether 7393 and an elastically deformable barb element 7395. Tether 7393 is made from a flexible cord or suture material having low flexural stiffness, such as ePTFE or other suitable inert flexible material, and is securely connected at a first wire end to loop member 7392 (i.e., through a knot or other suitable joining element 7399) and at a second wire end to barb element 7395 (i.e., through a crimp member within center body 7396).

In FIG. 107A, anchor 7391 is shown in a constrained, delivery configuration within the lumen of a tissue-piercing catheter C. Anchor 7391 is delivered in the constrained state within the delivery catheter to avoid possible entanglement with chordae and potential injury to leaflets. In FIG. 107B, anchor 7391 is shown in a tissue-grasping or tissue-retaining, deployed anchoring configuration.

Barb element 7395 includes a plunger 7397, a center body 7396, and a number of elastically-deformable, self-expanding spike or prong members 7398. Barb element 7395 is made from an elastic material, or preferably from a superelastic material, such as Nitinol, and is shape-set in its unconstrained, free state at the anchoring configuration shown in FIG. 107B. Prong members 7395 may be produced, for example, from a slotted Nitinol tube that is shape-set with the prongs in a spaced-apart relationship and joined to the center body 7396 (e.g., by laser welding).

In an exemplary installation procedure, the delivery catheter may pierce the left ventricle at the desired location to place anchor 7391, then applying a force within the delivery catheter on plunger 7397 to release the anchor 7391 into the left ventricle while simultaneously retracting the tissue-piercing distal end of the delivery catheter from the left ventricle. At this point, the ventricular connector 7390 is engaged with the ventricular tissue through expanded prongs 7398. Tension applied on the flexible tether 7393 will further secure the expanded prongs within the ventricular tissue. As illustrated in FIG. 107C, with the occluder 7340 coupled to the clip CL and anchor 7391 engaged with ventricular tissue, the length of the flexible tether 7393 defines the radius RT of a spherical cap SC centered at the anchoring point (i.e., a volume within which occluder loop member 7392 may freely move to the extent allowed by the MV anatomy).

As such, given the flexibility of tether 7393, the occluder 7340 is generally free (or less restrained) to self-align within the flow control portion FCP1 and thereby assume a desirable position and/or orientation relative to the native leaflets and regurgitant orifice MRO compared to SOD embodiments that have a more rigid engagement of the ventricular connector with the left ventricle LV.

More specifically, the flexible tether allows the occluder to assume a favorable position (i.e., laterally shifting without necessarily tilting at occluder apex 7345) or a desirable orientation (i.e., tilting of the occluder along the A-P direction; RKS in FIG. 96C) to provide sealing engagement with the native leaflets during systole. This variability in the positioning of occluder 7340 is advantageous as it allows device adaptability relative to a specific anatomy (or disease progression). As well, it offers a degree of device adjustability (i.e., self-alignment of the occluder inlet INL relative to the regurgitant orifice MRO) according to the varying dynamic forces that act on the occluder during the different phases of the cardiac cycle. In systole, with occluder 7340 inner cavity 7349 pressurized above the coaptation line between the native leaflets and the occluder movable membranes, flexible tether 7393 will be under tension in a direction that is favorably suited to ensure sealing engagement between the native leaflets and the occluder membranes. In diastole, the length of the tether 7393 is appropriately determined so that the occluder 7340 stays generally within the plane of the mitral valve with occluder apex 7345 remaining above the mitral valve annulus MVA. Optionally, an atrial connector 7330 may be included to further support or stabilize occluder 7340. Atrial connector 7330 is exemplarily shown as a dashed line in FIG. 107C, extending from occluder apex 7345 to a suitable tissue anchoring location within the left atrium LA (i.e., anchored at an atrial tissue ATR). Atrial connector 7330 may be similar to flexible tether 7393 and anchored to the atrial tissue ATR with a suitable tissue-anchoring member or tissue barb as described in other embodiments.

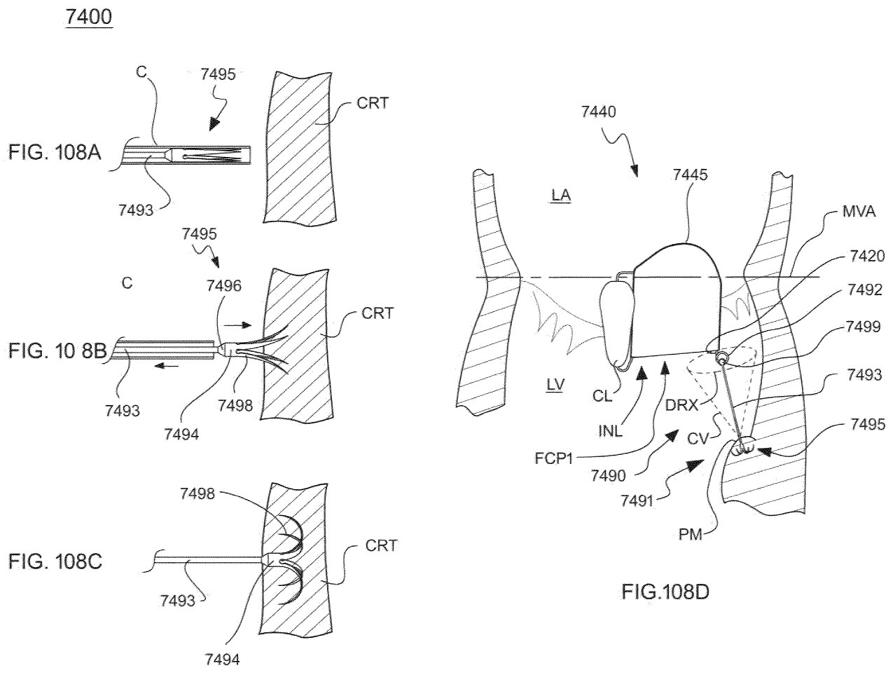

In an alternative embodiment, as shown in FIGS. 108A-108D, SOD 7400 includes a ventricular connector 7490, which in this embodiment is implemented as a papillary muscle anchor 7491.

SOD 7400 includes a movable membrane occluder 7440 similar to occluder 6440 of SOD 6400, with a support frame 7420 that similarly extends laterally beyond the occluding portion of occluder 7440 to terminate in loop member 7492, where it is engaged with the ventricular connector 7490.

Papillary muscle anchor 7491 is similar to multi-pronged anchor 7391 of SOD 7300 except that flexible tether 7393 is replaced with an elastically-flexible cable member 7493 suitable for anchoring deeper within the left ventricle LV, which in this embodiment describes engagement of the anchor 7491 to a papillary muscle PM.

Cable member 7493 may be configured and sized with sufficient flexural stiffness to allow elastic bending, but to prevent structural buckling under the dynamic loading in this application. The cable member may be made from a braided metallic cable material, a thin metallic or polymeric rod or shaft (e.g., Nitinol), and is preferably covered by a PTFE sheath or other suitable inert material to limit the degree of bioreaction and tissue ingrowth over the cable member.

Cable member is securely connected at a first cable end to loop member 7492 (i.e., through a cable crimp element or other suitable joining element 7499) and at a second cable end to tissue-grasping element or tissue-grasper 7495 (i.e., through a flared coupling member 7496).

In FIG. 108A, anchor 7491 is shown in a constrained, delivery configuration within the lumen of a tissue-piercing catheter C, to avoid possible entanglement with chordae and potential injury to leaflets during installation. In FIG. 108B, anchor 7491 is shown in a partially deployed state with the pointed tips of tissue grasper 7495 inserted within the cardiac tissue CRT. In FIG. 108C, anchor 7491 is shown in a tissue-retaining, deployed anchoring configuration.

Tissue grasper 7495 includes a plurality of elastically-deformable, circumferentially-spaced, and outwardly self-expanding tines 7498. Tissue grasper 7495 is made from an elastic material, or preferably from a superelastic material, such as slotted Nitinol tube 7494, with the tines 7498 shape-set in the unconstrained, free state corresponding the outwardly-curled spaced apart anchoring configuration shown in FIG. 108C.

As illustrated in FIG. 108D, anchor 7491 is engaged deeper within the LV than previously described anchor 7391. A flexible tether 7393 as provided by SOD 7300 is not suitable to retain the occluder 7440 within the plane of the mitral valve in such instances since it lacks the required stiffness.

With the occluder 7440 coupled to the clip CL and anchor 7491 engaged with papillary muscle PM, a conical volume CV is bound by the ability of the cable member 7493 to bend about the anchoring point where 7495 in engaged with the papillary muscle PM (i.e., vertex of a cone) and to also sweep around this anchoring point to define the cone CV (i.e., generating line of the cone). The resulting cone angle will depend on the amount of restraint provided by the clip CP on the occluder and the anatomical constraints imposed by the left ventricle.

In use, the cable member 7493 is able to assume an orientation within the conical volume CV, and consequently, the occluder loop 7492 may freely move within a generally elliptical perimeter DRX at the base of the cone CV (i.e., within the directrix of the cone). As such, given the angular mobility of cable 7493, the occluder 7440 is less restrained to self-align within the flow control portion FCP1 and thereby assume a desirable position and/or orientation relative to the native leaflets and regurgitant orifice MRO compared to SOD embodiments that have a more rigid engagement of the ventricular connector with the left ventricle LV.

In systole, cable member 7493 is sufficiently flexible to position the occluder 7440 in sealing engagement with the native leaflets, and sufficiently strong to retain the occluder from displacement into left atrium LA under dynamic loading. In diastole, the cable member is sufficiently stiff not to buckle and thereby maintain the occluder generally within the plane of the mitral valve and the flow control portion FCP1 (i.e., with occluder apex 7445 remaining above the mitral valve annulus MVA).

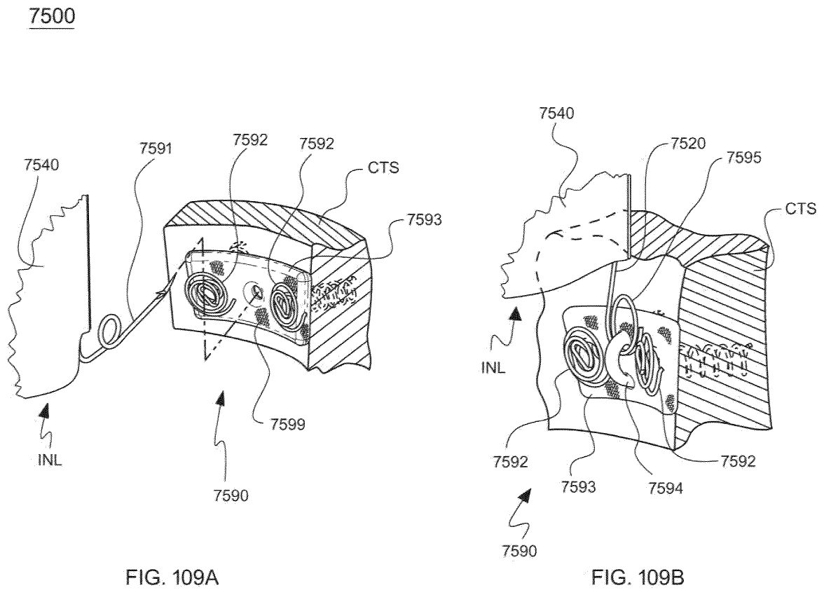

In an alternative embodiment, as shown in FIGS. 109A-109B, SOD 7500 includes a ventricular connector 7590, which in this embodiment is implemented as an anchoring plate or reinforcement pad 7593.

Pad 7593 may be fabricated from an implantable fabric material and may be flexible so that it may be rolled, folded, or compressed into a compact configuration suitable for delivery to the left ventricle by a steerable catheter. Being flexible, it is also compliant to adapt to the surface of the heart where it will be secured. A porosity 7599 in the fabric is also conducive to a favorable bioreaction (i.e., colonization by autologous cells) which advantageously serves to reinforce the mechanical tissue anchoring with biological tissue ingrowth.

Compared to discrete, individually-applied tissue anchors, pad 7593 provides a larger bearing surface over which the dynamic loads exerted on occluder 7540 (i.e., force F;

FIG. 98A) are reacted at the ventricular connector 7590 (i.e., reaction force RVC; FIG. 98A) thus reducing the stresses on the anchored ventricular tissue. This reduces tissue trauma and risk of ventricular connector disengagement. The pad may also include a polymeric or metallic core that more uniformly distributes the dynamic loads reacted at the ventricular connector over the entire contact area provided by the pad, and also improves the resistance of the pad to tissue anchor disengagement or extraction.

As shown in FIG. 109A, pad 7593 may be secured to cardiac tissue by a number of tissue anchors or fasteners 7592, similar to previously described fastener 6591 of SOD 6500, or alternatively, a number of elastically deformable clip anchors (not shown), similar to previously described clip anchors 7291 of SOD 7200, or even other known types of tissue anchors.

Fasteners 7592 may be deployed by a steerable delivery catheter able of transmitting torque to the head of the fastener. The installation procedure may include embedding a first tissue anchor 7592 through the pad 7593 and into the cardiac tissue CTS, and sequentially embedding the rest of the tissue anchors one by one.

With reference to FIG. 109A, pad 7593 is first deployed through a delivery catheter (not shown). Subsequently, occluder 7540 is coupled to pad 7593 (and also anchored to cardiac tissue CTS) by piercing a tissue barb 7591 through the fabric pad 7593 into the underlying cardiac tissue. As shown in FIG. 109A, the pad 7593 may also be provided with an anchoring port or dock 7594 configured to receive and engage the tissue barb 7591 at a predetermined location. Dock 7594 may be provided with a suitable lead in to facilitate engagement with the tissue barb 7591, or with radiopaque markers to help guide the tissue barb 7591 during image-guided delivery, or with a reinforcement collar to better support the local concentrated stress between the barb 7591 and pad 7593.

With reference to FIG. 109B, pad 7593 and occluder 7540 may also be pre-attached or pre-coupled to one another and deployed sequentially through a common delivery catheter. Pad 7593 is first released from the delivery catheter and secured to the cardiac tissue CTS. Occluder 7540 is subsequently released from delivery catheter while coupled to the pad.

In an exemplary configuration shown in FIG. 109B, pad 7593 is configured with a loop or link element 7594 and occluder support frame 7520 is provided with loop element 7595 pivotably coupled to the link element 7594. Alternatively, link element 7594 and loop element 7595 may be coupled together through a separate wire or suture element that can be tied or crimped to secure and pivotably engage the occluder to the pad. All of above occluder-to-pad coupling methods allow the occluder 7540 to pivot about a pivoting axis OPX located generally adjacent to the occluder inlet INL.

As described above in connection with several embodiments, a membrane spacing element may be included in an SOD to establish a desired membrane spacing at inlet INL, to dispose the occluder in an intermediate, EPDS configuration with area AED. In some implementations, the membrane spacing element may be implemented by localized properties or configuration of the flexible membrane, rather than as a separate, discrete structure. As describe in more detail below in connection with the embodiments shown in FIGS. 110A to 114C, options for such implementation of the membrane spacing element include local conditioning of the membrane, localized modification to the membrane, localized flexural stiffness alteration of the membrane, or locally altering or tuning the mechanical properties of the membrane.

One such implementation is shown in FIGS. 110A-110C, for a movable membrane SOD 7600. SOD 7600 includes a support frame 7620 and an occluder 7640 having a membrane-to-frame joint 7649 configured to achieve the required membrane spacing at occluder inlet INL, when the occluder assumes the intermediate EDPS configuration (i.e., with area AED).

Frame 7620 is similar to previously described frame 6420 of SOD 6400. Occluder 7640 includes movable pericardium membranes 7641, 7642 which are joined to one another by a first longitudinal lock stitch seam 7643 extending along and internally adjacent to arcuate frame 7620. The joined membranes 7641, 7642 are then sutured to a backing strip 7647 by second and third lock stitch seams 7646, 7648, respectively. As shown in FIG. 110C, the support frame is captured between the strip and the joined membranes adjacent to seam 7643 (i.e., a type of fell seam).

Pericardium is a thin, pliable biomaterial with a very low flexural modulus. As such, it is very useful as a movable membrane material able to adaptively conform to a regurgitant valve orifice MRO, regardless of its irregular shape. However, because of its low flexural modulus, it may also be challenging to have the membranes assume a predetermined membrane-spaced relationship entirely on their own, without being urged toward said relationship.

Backing strip 7647 has flexural stiffness greater than membranes 7641, 7642 which in this embodiment are made from pericardium. It may be made from a thicker sheet of pericardium (i.e., bovine instead of porcine used for membranes) or fashioned from a suitable bioprosthetic polymer or material having sufficient flexural stiffness.

During diastole, the dynamic load on the occluder membranes causes the backing strip to bend or deflect inwardly as the occluder assumes its collapsed, diastolic configuration (FIG. 110B). At EDPS, the backing strip wants to resume its unconstrained free state and, as such, urges the more flexible membranes to separate from the collapsed, diastolic configuration to the membrane-spaced EDPS configuration. In systole, the backing strip allows the occluder to freely assume the systolic configuration.

Without the joint described above, an occluder having very thin, flexible membranes may fully collapse in diastole (area AC approximately zero) and, consequently, the occluder may remain closed unable to assume the membrane-spaced EDPS configuration which in turn prevents the occluder from filling with systolic blood flow and opening to the systolic configuration.

Optionally, a deformable elastomeric sheath or stiffener 7648 may also be added between the backing strip 7647 and the joined membranes 7642, 7641 to augment the effect of the backing strip as described above. Sheath 7648 may be overmolded on frame 7620 or provided as a separate component in joint 7649. Other types of seam configurations are possible for joint 7649 to achieve the same device operability in use.

Joint 7649 achieves a change in the flexural stiffness of the occluder membrane at a specific predetermined location in the occluder (i.e., adjacent to occluder inlet). Joint 7649 is implemented at least proximally to inlet INL. It may optionally extend all the way to occluder apex 7645, or terminate somewhere below the apex.

Alternatively, the pericardium membranes may be shape-fixed (i.e., as opposed to flat-fixed) over a shaping fixture during the chemical fixation process with the aim of conforming the membranes to the desired EDPS configuration of the occluder.

In an alternative embodiment, as shown in FIGS. 111A-111E, SOD 7700 includes an occluder 7740 which has a different membrane-to-frame joint 7748 than joint 7648 of SOD 7640, and includes an occluder rib stiffener 7744 that is configured with pericardium material to achieve the required membrane spacing at occluder inlet INL, when the occluder assumes the intermediate EDPS configuration (i.e., with area AED). Frame 7720 is similar to previously described frame 6420 of SOD 6400.

Occluder 7740 includes movable pericardium membranes 7741, 7742 which are joined to one another by a pair of parallel lock stitch seams 7743, 7746 extending along and adjacent to arcuate frame 7720. As shown in FIG. 111E, the support frame is captured between the joined membranes and between the parallel seams (i.e., a type of lap seam). In comparison to seam 6423 (FIG. 98E), this type of lap seam 7749 helps to keep the occluder membranes from contacting one another at the location immediately adjacent to the frame support (i.e., except for where the membranes are joined by the lap seam), including at the occluder inlet INL (FIG. 111B). This relative membrane spacing urges the occluder towards its membrane-spaced EDPS configuration (FIG. 111C). Additionally, a polymeric stiffener similar to stiffener 7648 (FIG. 110E) may be incorporated in the space 7748 (i.e., adjacent to frame 7720 and between membranes 7741, 7742), and optionally also inserted within seams 7743, 7746 to help augment the desired membrane spacing.

Occluder rib stiffener 7744 may be configured from selective pleating of the pericardium occluder membranes 7741, 7742 at discrete locations, or the addition of one or more separate pericardium plies attached to a movable membrane 7741, 7742, or to both the membranes, in order to urge occluder 7740 to assume a membrane-spaced inlet INL configuration. As shown, occluder rib stiffener 7744 is implemented in this embodiment as a pair of pericardium stiffening ribs, each one of the ribs starting at occluder inlet INL and extending toward occluder apex 7745. The stiffening ribs are preferably added and attached within occluder cavity 7747.

An arcuate spring element (not shown), preferably of a superelastic material, may be conveniently inserted within the pericardium stiffening ribs to further enhance the function of the stiffening ribs. The arcuate spring element would extend from the free margin of membrane 7742, to the occluder apex 7745, and terminate at the free margin of membrane 7741. The spring element is shaped so that in the unconstrained free state, the spacing between membrane free margins corresponds to the desired spacing when the occluder assumes the EDPS configuration.

The occluder rib stiffener acts to locally increase the flexural stiffness of the otherwise uniformly thin, flexible occluder membrane made from pliable pericardium.

In an alternative embodiment, as shown in FIGS. 112A-112E, SOD 7800 includes an occluder 7840 which is similar to occluder 7740 of SOD, and further includes an occluder inlet stiffener 7846 to achieve the required membrane spacing at occluder inlet INL, when the occluder assumes the intermediate EDPS configuration (i.e., with area AED). SOD 7800 includes a frame 7820 that is similar to previously described frame 7720 of SOD 7700.

Occluder 7840 includes occluder rib stiffener 7844 and movable pericardium membranes 7841, 7842 which are joined to one another and to a frame 7820 by a lap seam 7849. Frame 7820 and lap seam 7849 are similar to frame 7720 and lap seam 7749 described in SOD 7700.

In this embodiment, occluder inlet stiffener 7846 is implemented as an inlet hem 7848, which may be formed by folding the free margins of membranes 7841, 7842 inwardly within the occluder cavity 7847.

Inlet stiffener 7846 helps to keep the occluder membranes 7841, 7842 spaced apart from one another when the dynamic forces on the occluder are reduced at EDPS. This relative membrane spacing urges the occluder towards its membrane-spaced EDPS configuration (FIG. 112C). The inlet rib stiffener acts to locally increase the flexural stiffness of the occluder membranes adjacent the free margins thereof.

Additionally, a closed-perimeter elastic strip or wire 7821 (FIG. 112A) or, alternatively, arcuate spring elements 7822 disposed adjacent to frame 7820 (FIG. 112E) may be incorporated within the inlet hem 7848 and serve to augment the membrane spacing function provided by inlet stiffener 7846.

Wire 7821 and arcuate spring elements 7822 are shaped with an unconstrained free state so that the spacing between membrane free margins corresponds to the desired spacing when the occluder assumes the EDPS configuration.

In reference to FIGS. 113A-113D, a movable membrane SOD 7900 according to another embodiment is described. SOD 7900 includes an occluder 7940 having a membrane spacing element 7945, which in this embodiment is implemented as a stent frame 7946 disposed adjacent to the occluder inlet INL.

Occluder 7940 includes a support frame 7920, which is similar to previously described frame 6420 of SOD 6400, and movable membranes 7741, 7742 which are joined to one another and to frame 7920.

Stent frame is made from an elastic, preferably superelastic alloy (e.g., Nitinol) that is shape-set in an unconstrained, free state configuration corresponding to the predetermined configuration that is desired at EDPS when the occluder assumes the intermediate membrane spaced configuration (FIG. 113C) with occluder inlet area AED. In this example, the stent frame is shape-set into a closed, wavy perimeter.

In diastole, under the effects of the dynamic load on the occluder membranes, the stent frame 7946 will collapse, to a generally sinusoidal or wavy profile as shown in FIG. 113B. Although the stent is shape-set to the EDPS configuration, the stent may be preconditioned to collapse in a predetermined manner (i.e., by selectively varying the flexural stiffness of certain stent struts 7947 or the position and orientation of certain stent struts) so that the occluder membranes come into proximity or contact with one another thereby not obstructing the blood flow from the left atrium LA to the left ventricle LV. For example, in this embodiment, a stent crown 7948 on membrane 7941 may be positioned between two adjacent stent crowns in membrane 7942 when occluder assumes the closed, diastolic configuration.

At EDPS, the internal energy stored in the elastically collapsed stent frame 7946 will be released causing the opposed membranes to separate from one another, as the stent frame resumes its unconstrained free state while the occluder 7940 simultaneously assumes the membrane spaced EDPS configuration.

At the start of systole, blood is able to enter occluder 7940 through open area AED and the fluid inertial forces that are reacted within the occluder cavity 7949 will spread occluder membranes 7941, 7942 further apart enlarging the occluder inlet area to an area AS. In systole, the stent frame 7946 will assume a generally elliptical profile as shown in FIG. 113D (i.e., as the membrane free margins 7943, 7944 react the hoop stresses induced from the pressurized occluder).

The deformation of the stent frame away from its free state configuration is preferably maintained within the elastic material property limits of the stent material. As such, at the end of systole and similarly at the end of diastole, when the dynamic forces on the occluder membranes are reduced, the stent returns to its free state urging the occluder to assume the EDPS configuration.

An occluder configuration as described above, and in similar embodiments of movable membrane SODs, where the volume of the occluder internal cavity 7949 varies considerably between the occluder collapsed diastolic configuration and the open systolic configuration is advantageous in ensuring reliable blood evacuation from the occluder cavity with every heartbeat. This reduces the likelihood of thrombus formation within the occluder cavity and possible injury from a thromboembolic event.

In occluders having pericardium membranes (i.e., as previously described above), stent frame 7946 may be embedded within a membrane fold at an inlet hem (i.e., similar to hem 7848), sutured to the pericardium membrane by an array of sutures.

In occluders having polymeric membranes (i.e., as will be described below) the stent frame may be embedded within the polymeric membrane during the fabrication process, glued to the membrane, or secured thereto by other suitable elements in a manner to not impede the function of the stent frame 7946 as contemplated above.

In this embodiment, the stent frame 7946 attached to the occluder membranes alters the flexural stiffness of the movable membranes at a location adjacent to the occluder inlet INL. This urges the occluder to assume its predetermined EDPS configuration where it is ready to receive blood flow at the start of systole through occluder inlet area AED.

A stent frame 7946 attached to a movable membrane occluder as described above is advantageous to also prevent occluder membrane prolapse (as previously described in reference to SOD 6400).

Alternatively, in lieu of the stent frame 7946, a perimeter-extending stitch using a thin elastic nitinol filament wire may be incorporated or sewn into each of the occluder membranes to serve as a membrane spacing element. For example, a zig-zag stitch disposed perimetrically along the length of the free margins 7943, 7944 may be used.

With reference to FIGS. 114A-114H, another embodiment of a movable membrane SOD 8000 having an occluder 8040 made from a biocompatible polymeric material is described.

As shown in FIG. 114A-114B, occluder 8040 may be produced with uniform, very thin movable membranes 8041, 8042 in a variety of ways including compression molding, injection stretch blow molding and other polymer forming techniques. In the context of movable membrane occluders, polymeric material has a greater flexural modulus than pericardium material.

Occluder 8040 is produced with a predetermined inlet INL shape such that, in the occluder free-state (i.e., when no external forces act on the occluder), the occluder membranes will be spaced apart to one another with an open inlet area corresponding to the area AED desired at EDPS. Membranes 8041, 8042 are configured with a required flexural stiffness (i.e., selecting the appropriate membrane thickness for the flexural modulus of the polymeric material used) such that in diastole, the dynamic fluid forces may overcome the flexural stiffness and collapse the occluder membranes into an approximated configuration to minimize likelihood of mitral stenosis. At this point, the occluder assumes the closed, diastolic configuration (i.e., inlet area AC). At EDPS, when the dynamic forces on the occluder are considerably reduced, the occluder resumes its free state. At this point, the occluder assumes the intermediate EDPS configuration (i.e., inlet open to the predetermined area AED), ready to receive blood within the occluder cavity 8049. In systole, with the occluder cavity exposed to fluid inertial forces sufficient to overcome the flexural stiffness, the membranes move apart and occluder inlet INL is reshaped to an area AS. At this point, the occluder assumes its open, systolic configuration.

In order to tune the operability of the occluder under dynamic loading and to further ensure the occluder functions in the manner described above, additional localized features may be added to the occluder configuration. With reference to FIG. 114C-114H, occluder 8040 may be molded or produced with local thickening relative to the configuration of FIG. 114B. For example, a pair of opposed arcuate rib stiffeners 8043 may be incorporated proximal to inlet INL within cavity 8049. The stiffeners are engaged to both movable membranes 8041, 8042 and are illustrated in their free state in FIG. 114E. In diastole, the stiffeners flex inwardly from the free state (FIG. 114D), and in systole, the stiffeners flex outwardly (FIG. 114F), thereby serving to augment the resiliency of the occluder toward the EDPS configuration. This is particularly advantageous in occluders having ultra-thin membranes (i.e., with very low flexural stiffness) wherein the stiffeners ensure an inlet area of AED is present for the occluder to function. Thin membrane occluders are easier to pack into a small diameter delivery catheter.

Alternatively, or additionally, a pair of stiffening ribs 8044, starting at inlet INL and extending toward occluder apex 8045 may be incorporated. Alternatively, as schematically illustrated in FIG. 114G, stiffening ribs 8047 may be incorporated on the outer surface of the occluder, extending perimetrically along the belly of the movable leaflets 8041, 8042, or the corresponding inner surface of the occluder, or on both inner and outer surfaces. Alternatively, lip or weir 8048 may be incorporated along the entire perimeter of occluder inlet INL. All of the above localized features are in their free state in the occluder configuration corresponding to the EDPS.

As shown in FIG. 114A, SOD 8000 includes a frame support 8020 extending along the closed end of occluder 8040 (i.e., similar to support frame 6420; FIG. 98B). The support frame ensures that the thin membrane occluder does not fold over on itself (i.e., fold at axis AX6) during diastole. Local thickening can also be incorporated over the closed end of the polymeric occluder (i.e., where the movable membranes are joined to one another). By tailoring the material thickness at this location, a minimal support frame 8020 may be needed relative to support frame 6420, or potentially it may be excluded entirely, or eliminated only at some locations, thereby resulting in a more compactable occluder configuration suitable for catheter delivery.

As an alternative to local thickening to the movable membranes described above, embedded fiber reinforcing elements may be introduced in the polymer fabrication process in order to tailor the flexural stiffness of the occluder membranes at strategic locations, for example adjacent to the free margins thereof. Selective fiber-reinforcement of the membrane in this manner may serve to augment the resiliency of the occluder towards its membrane-spaced EDPS configuration while keeping the membrane thicknesses generally uniform and thin.

Metallic elements such as thin nitinol wires may also be embedded within polymer membranes during the fabrication process. For example, such elements can serve as structural elements (i.e., occluder membranes overmolded to frame 8020), or can serve as biasing members (similar to 6450; FIG. 98B). Repelling magnets may also be embedded within the free margins of opposing occluder membranes to urge the occluder from the closed diastolic configuration to the membrane-spaced EDPS configuration.

Polymeric materials also open the possibilities for incorporating movable membranes with elastomeric material properties wherein the occluder cavity 8049 may stretch to enlarge when exposed to systolic pressures and as such resolve a larger regurgitant orifice MRO than a non-elastic, flexible membrane.

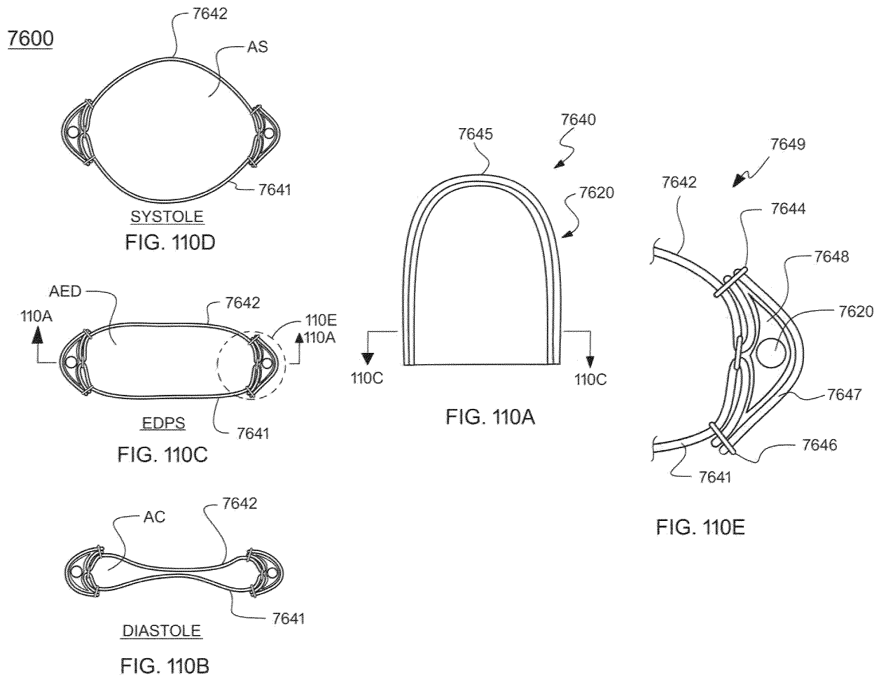

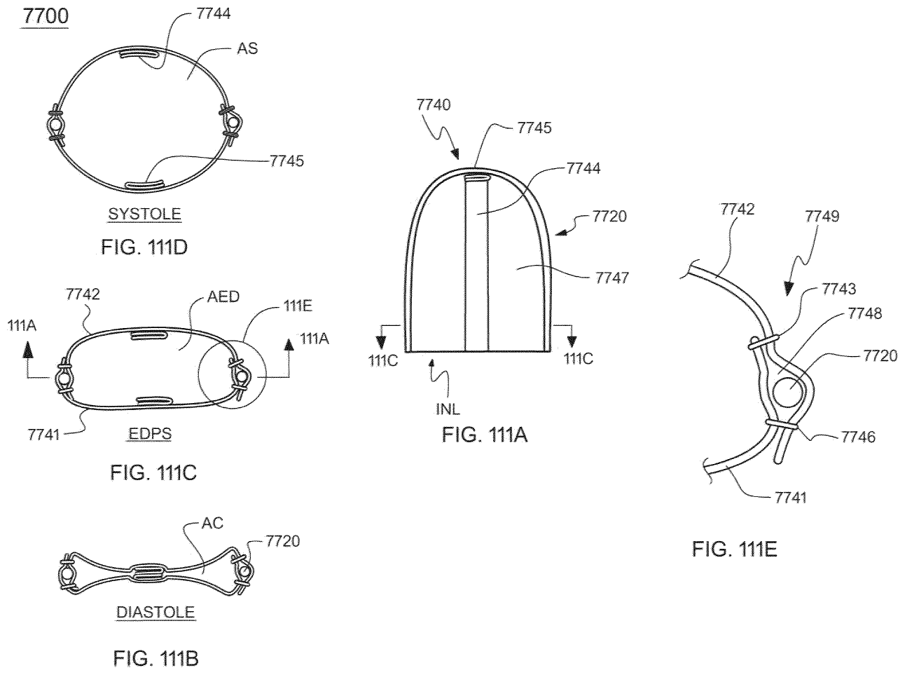

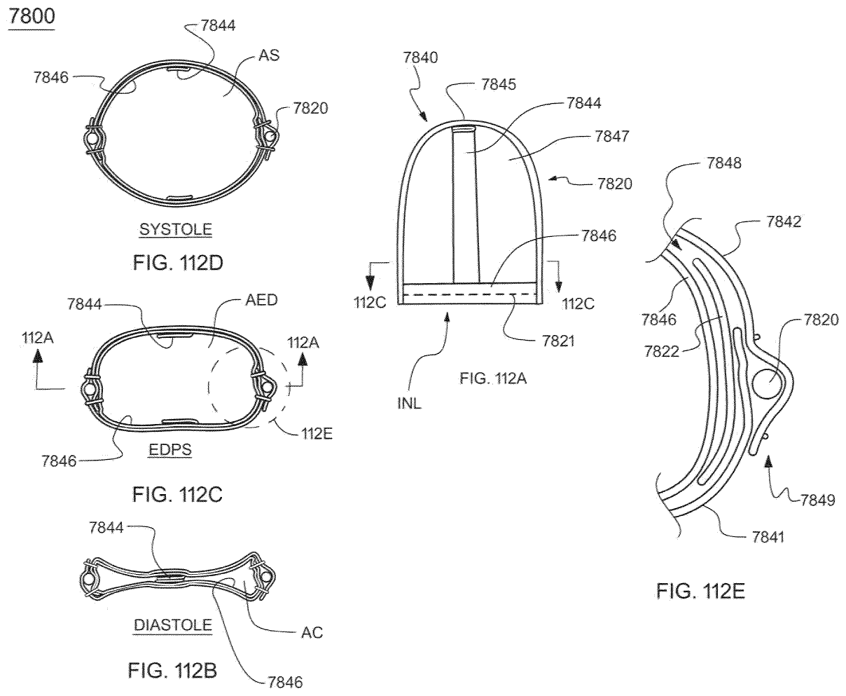

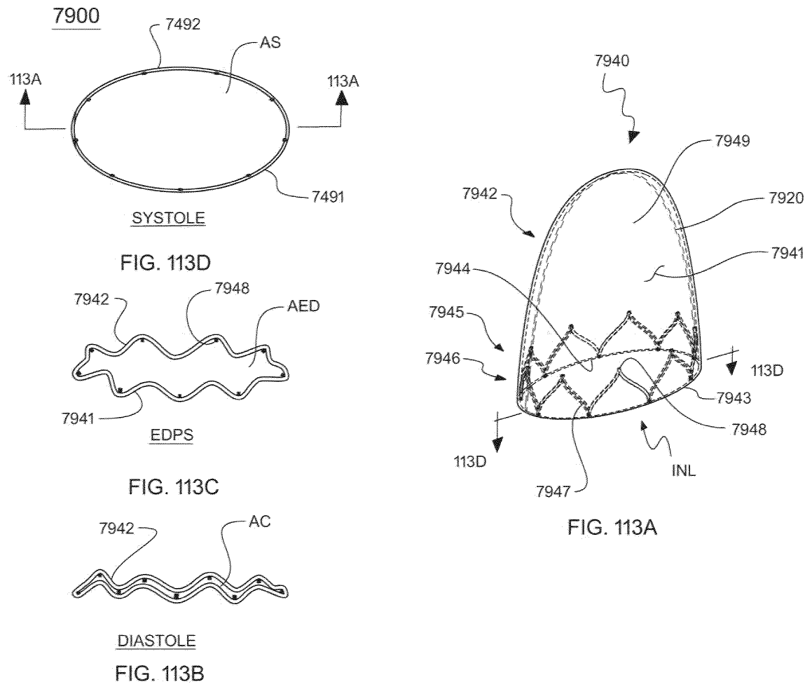

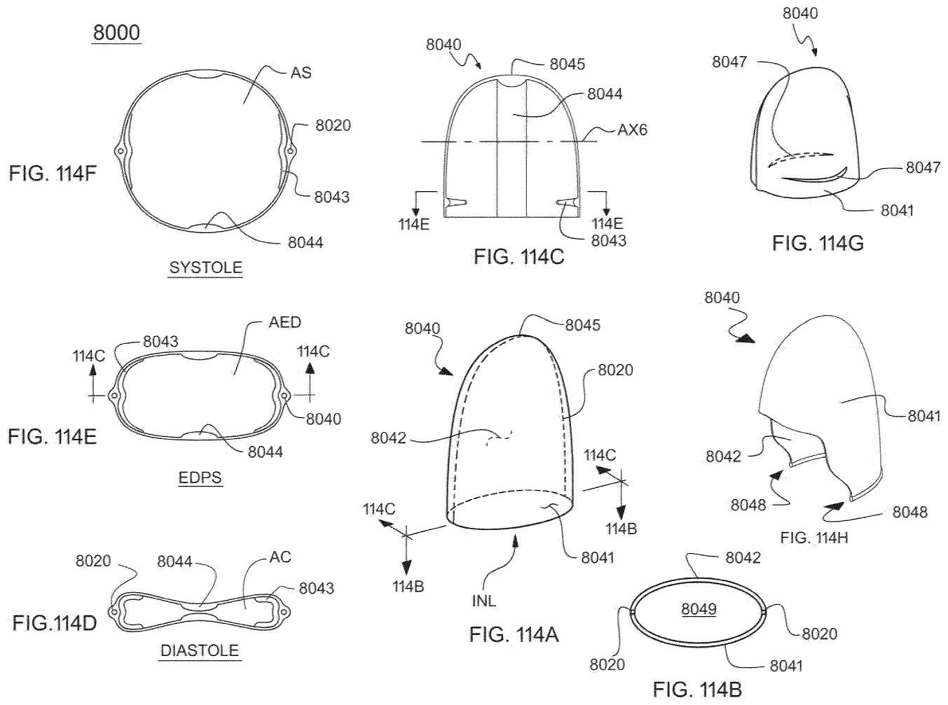

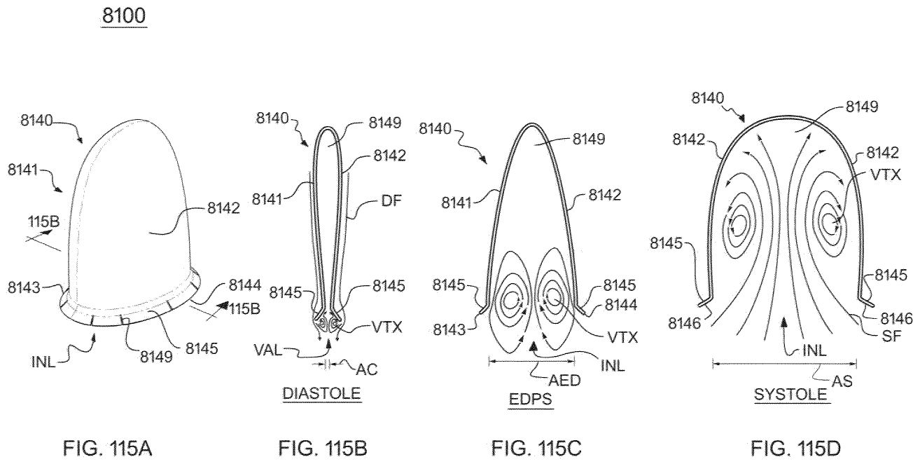

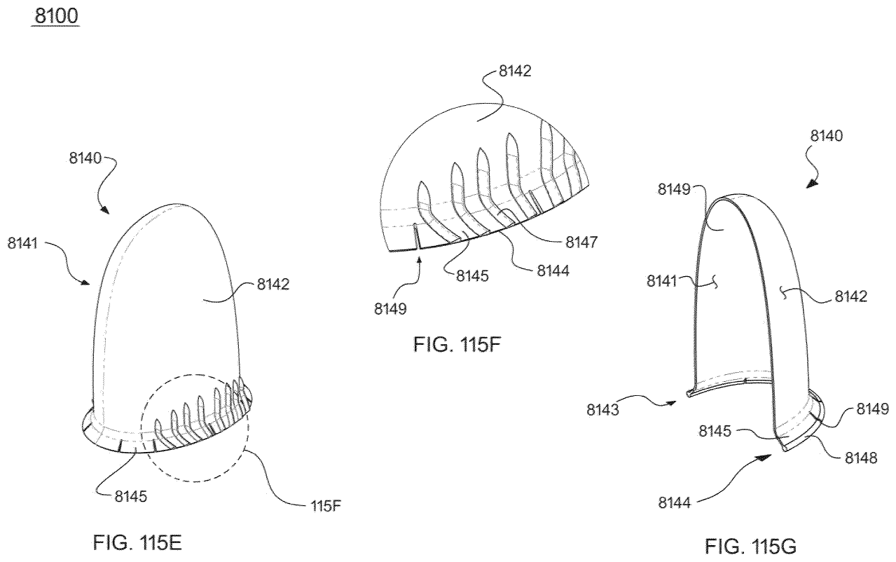

With reference to FIGS. 115A-115G, movable membrane SOD 8100 is described, wherein the membrane spacing is implemented with a vortex-generating element/feature disposed proximal to the free margin of a movable occluder membrane.

Occluder 8140 is preferably produced from polymeric material (i.e., similar to occluder 8040 described in FIG. 114A), and includes two movable membranes 8141, 8142 having, respectively, membrane free margins 8143, 8144.

The membranes are produced or shaped with a trailing-edge configuration including one or more flow diverters or raised-edge spoilers 8145. The spoilers are preferably disposed on both movable membranes at a location where the opposed membrane free margins 8143, 8144 come into close proximity to one another during diastole (i.e., occluder diastolic configuration). In diastole, the opposing spoilers define a generally concave, inverted V-shaped valley VAL defined as such by the cavity-facing, inner surfaces 8146 of the spoilers (FIG. 115B). The spoilers cause the diastolic flow DF to trip over the membrane free margins initiating a rotational vortex VTX behind the free margin within the valley VAL. The rotating vortex VTX impinges on the inner surfaces 8146 of the opposing membranes, transferring its energy thereto, thus causing the membranes to start moving apart locally at the VAL location. A favorable pressure gradient across the spoiler surface may also assist in aiding the separation.

The vortex VTX gains rotational energy that persists even after the diastolic flow has stopped, thereby spreading the opposing membranes 8141, 8142 further apart as best shown in FIG. 115C (i.e., at the EDPS point in the cardiac cycle).

As illustrated, spoilers 5145 are disposed around the entire occluder inlet INL with adjacent spoilers separated by slits 8149. Adjacent spoilers may be of varying stiffness, thus permitting each spoiler to act and flex independently based on the magnitude of the flow and the energy stored within the locally shed vortex. The selective placement of individually tuned spoilers may further augment the favorable opening of the occluder and ensuring an inlet area AED is obtained to receive the systolic blood flow SF. During systole, the vortices VTX will migrate deeper within cavity 8149 and mix with the incoming systolic flow SF resulting in the occluder assuming the systolic configuration (i.e., occluder inlet area AS).

Alternatively, the occluder may be configured with a single spoiler that extends around the entire perimeter of inlet INL, but having a variable trailing-edge length at different locations along the perimeter, or even the uniform length.

As illustrated in FIGS. 115E-115F, spoiler 8145 may be configured with a series of ridges or directional vanes 8147 that serve multiple purposes. The vanes may be oriented in a manner to deflect or direct the diastolic flow towards a zone along the occluder inlet where the membrane separation is most desirable to have or likely to be maximized. The vanes may initiate the rotational vortex VTX flow before the diastolic flow reaches the membrane free margin 8144, thus maximizing the rotational energy of vortex VTX that is formed. Finally, the vanes may add stiffness to the spoiler 8145 in a manner that optimizes its deflection during contact with the diastolic flow DF.

As illustrated in FIG. 115G, spoiler 8145 may be configured with a raised, rounded lip 8148 along the membrane free margins 8144, 8143 in order to promote the formation of a vortex VTX within the valley VAL by tripping the diastolic flow DF as it passes thereover. The rounded profile of lip 8148 directs the tripped flow DF smoothly towards the vortex VTX, thus minimizing parasitic energy losses during the vortex formation.

In the embodiments of movable membrane SODs described above, when the occluder assumes the membrane spaced, intermediate EDPS configuration, the occluder inlet area AED ensures that the dynamic forces in systole act on the inside surfaces of the occluder membranes (i.e., on the occluder internal cavity or volume) rather than exclusively on the outside surfaces of the occluder membranes which would otherwise result in the occluder membranes staying collapsed and occluder remaining closed between diastole and systole (i.e., not providing the required sealing engagement with native leaflets to resolve mitral regurgitation). Any of the implementations of the membrane spacing element may be used with any of the flexible membrane SOD embodiments described herein.

With reference to FIGS. 116A-116D, a method for implanting a clip CL and SOD 8200 as part of an integrated procedure (i.e., a destination therapy) is described. As schematically shown, the method will be described in the context of a transeptal approach into the left atrium LA provided by an access cannula disposed in the atrial septum. A steerable guide catheter SGC is positioned through the access cannula and directs subsequent interventional catheters towards the mitral valve MV.

In this method embodiment, the clip CL and SOD 8200 are pre-coupled to one another prior to being delivered to the left atrium LA. Clip CL and SOD 8200 are delivered into the left atrium by a delivery catheter C1.

A movable membrane occluder 8240 is retained in a collapsed occluder configuration within a lumen of catheter C1, while clip CL extending from catheter C1 is deployed in a valve clipping Transcatheter Edge-to-Edge Repair (“TEER”) procedure (FIG. 116A). With clip CL engaged to the native leaflets of the mitral valve, the catheter C1 is retracted through catheter SGC thereby releasing occluder 8240 from the lumen of catheter C1 and allowing occluder 8240 to assume a non-collapsed occluder configuration (FIG. 116B). With catheter C1 retracted from the left atrium, the clip CL remains engaged to a catheter C2 and SOD 8200 remains engaged to a catheter C3 (i.e., adjacent ventricular connector 8290). At this point in the procedure, with medical imaging, catheter C3 is displaced or rotated relative to catheter C2 so as to position and orient occluder 8240 within a flow control portion FCP1 of the mitral valve in order to determine a favorable position in which to secure the SOD 8200, in order to reduce the mitral regurgitation. C2 and C3 are configured with sufficient structural stiffness to resist the dynamic forces on occluder 8240 during the positioning manipulations. Once a suitable position for securing the SOD 8200 is determined, the ventricular connector 8290 is engaged to a ventricular tissue through catheter C3 (e.g., by retracting sheath S1 of catheter C3 to expose and engage a tissue anchor in an integrated procedure wherein the occluder 8240 and ventricular connector 8290 are deployed through the same catheter C1). If required, the ventricular connector is repositioned with catheter C3. Once the SOD 8200 is satisfactorily secured at both the ventricular connector and at the clip CL, catheters C2 and C3 decoupled and withdrawn.

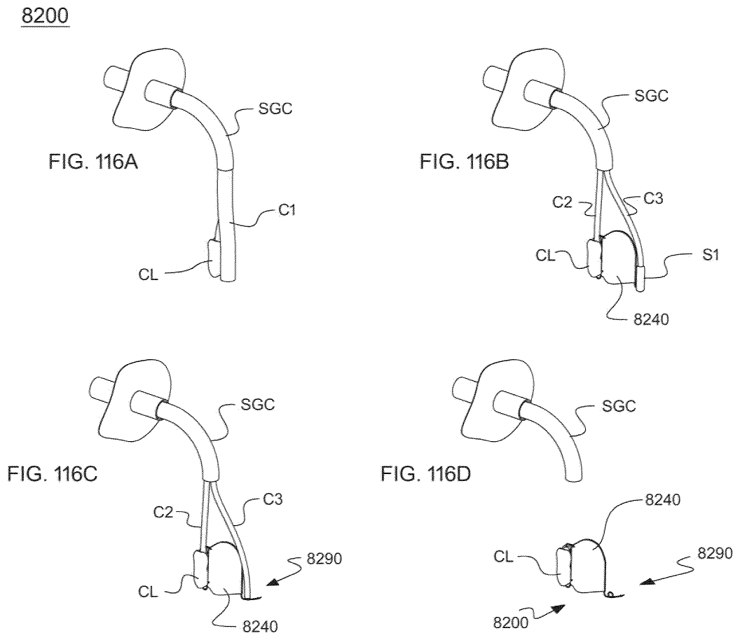

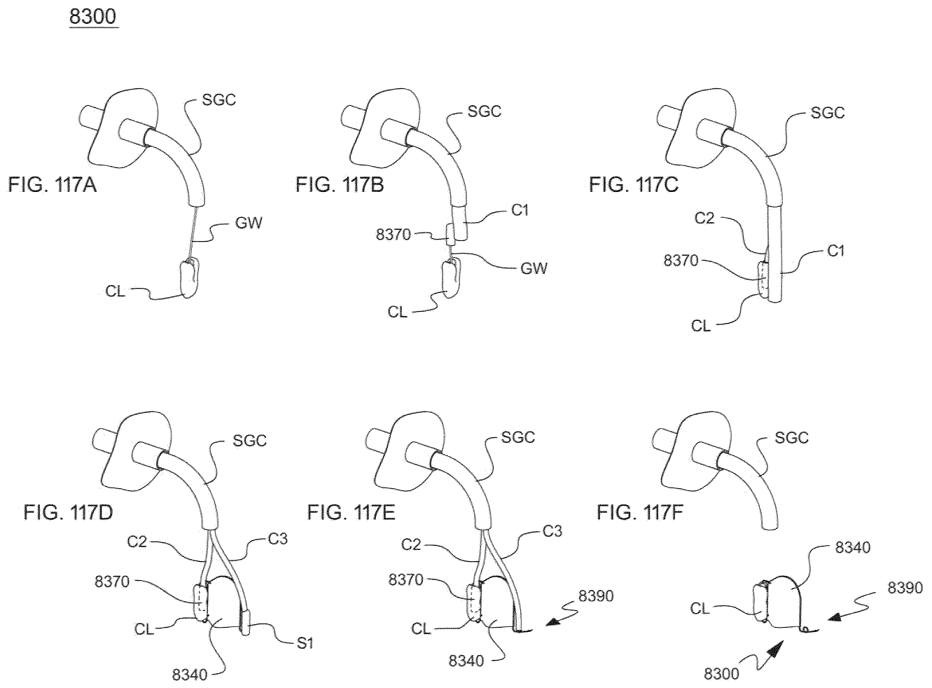

With reference to FIGS. 117A-117F, a method for implanting a SOD 8300 sequentially after a clip CL has been firstly deployed during a same intervention, or after a separate prior procedure, is described.

In this method embodiment, a guide wire GW is left attached to the firstly deployed clip CL during a same intervention (FIG. 117A), or a separate guide wire GW is engaged to the previously deployed clip CL through a delivery catheter (not shown) according to one of the previously described methods (i.e., lassoing a clip CL).

Clip connector 8370 of SOD 8300 is engaged to guide wire GW extracorporeally.

SOD 8200 is delivered into the left atrium LA by a delivery catheter C1 with clip connector 8370 riding over guide wire GW (FIG. 117B).

A movable membrane occluder 8340 is retained in a collapsed occluder configuration within a lumen of catheter C1, while clip connector 8370 extending from catheter C1 is coupled to clip CL (e.g., at a docking orifice in the clip spacer SP) (FIG. 117C).

With clip connector 8370 engaged to clip CL, the catheter C1 is retracted through catheter SGC thereby releasing occluder 8340 from the lumen of catheter C1 and allowing occluder 8340 to assume a non-collapsed occluder configuration (FIG. 117D). With catheter C1 retracted from the left atrium, the clip connector 8370 (and also clip CL) remains engaged to a catheter C2 and SOD 8300 remains engaged to a catheter C3 (i.e., adjacent ventricular connector 8390). At this point in the procedure, with medical imaging, catheter C3 is displaced or rotated relative to catheter C2 so as to position and orient occluder 8340 within a flow control portion FCP1 of the mitral valve in order to determine a favorable position in which to secure the SOD 8300, in order to reduce the mitral regurgitation. C2 and C3 are configured with sufficient structural stiffness to resist the dynamic forces on occluder 8340 during the positioning manipulations. Once a suitable position for securing the SOD 8300 is determined, the ventricular connector 8390 is engaged to a ventricular tissue through catheter C3 (e.g., by retracting sheath S1 of catheter C3 to expose and engage a tissue anchor). If required, the ventricular connector is repositioned with catheter C3. With the SOD 8300 satisfactorily secured at both the ventricular connector 8390 and at the clip CL through clip connector 8370, catheters C2 and C3 decoupled and withdrawn.

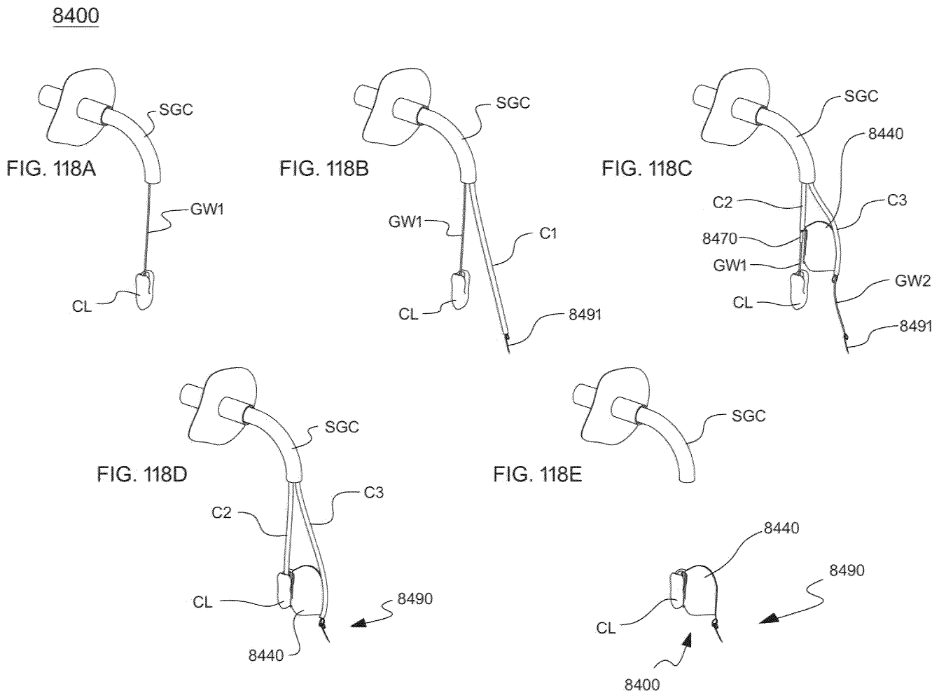

With reference to FIGS. 118A-118E, a method for implanting a SOD 8400 using two guide wires after a clip CL has been firstly deployed during a same intervention is described.

In this method embodiment, a guide wire GW1 is left attached to the firstly deployed clip CL during a same intervention (FIG. 118A).

A ventricular anchor 8491 is delivered to the left ventricle LV by a delivery catheter C1 and anchored at a suitable location to ventricular tissue (FIG. 118B). Catheter C1 is then retracted leaving behind a guide wire GW2 attached to the wall of the left ventricle and extending through steerable guide catheter SGC similarly to guide wire GW1.

Clip connector 8470 of SOD 8400 is engaged to guide wire GW1 and ventricular connector 8490 is engaged to guide wire GW2, extracorporeally.

SOD 8400 is then delivered to the left atrium LA with clip connector 8470 connected to catheter C2 (riding over guide wire GW1) and ventricular connector 8490 connected to catheter C3 (riding over guide wire GW2) (FIG. 118C). With catheters C2 and catheter C3, SOD 8400 is advanced over guide wires GW1 and GW2 to engage clip connector 8470 to clip CL. Ventricular connector 8490 is then secured to ventricular anchor 8491 using catheter C3 (e.g., with a securing collet or crimping element). With the SOD 8400 is satisfactorily secured at both the ventricular connector 8490 and at the clip CL through clip connector 8470, catheters C2 and C3 decoupled and withdrawn.

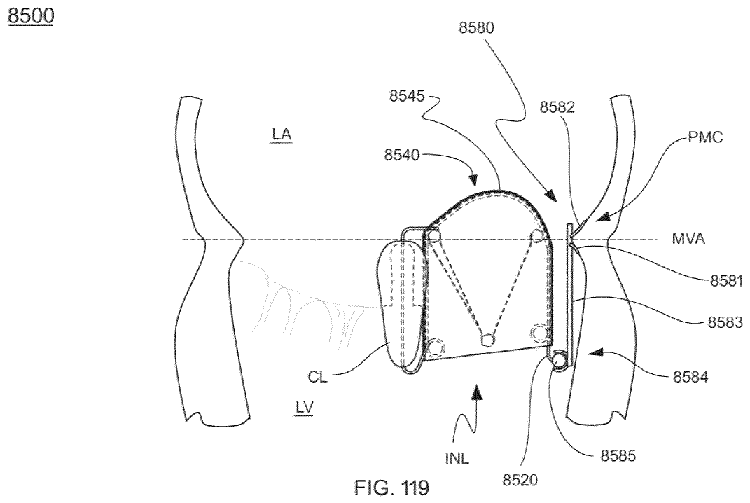

In an alternative embodiment, as shown in FIGS. 119, SOD 8500 includes an annulus connector 8580, which in this embodiment includes a frame post extending into the left ventricle LV for pivotably engaging occluder 8540 which is similar to previously described occluder 6440 of SOD 6400 (FIG. 98B).

Annulus connector 8580 is similar to the previously described annulus connectors, except that the annulus connector engages with cardiac tissue proximal or adjacent to a valve commissure location (i.e., a commissure connector). In this embodiment, annulus connector includes upper and lower connector elements 8582, 8581 configured and sized to capture annulus tissue therebetween adjacent to a commissure location (e.g., commissure PMC as shown) of the mitral valve. Frame post 8583 is rigidly connected to at least one of the connector elements and, in use, extends below the MVA into the left ventricle LV. Occluder 8540 is pivotably engaged to the terminal end of frame post 8583 at support frame 8520 through a movable joint 8584, which in this embodiment is implemented as a pivoting socket joint 8585. Frame post 8583 extends sufficiently deep within the LV so that the socket joint 8585 is located below the occluder inlet INL. As such, occluder 8540 may pivot about an occluder pivoting axis OPX (FIG. 96A) and the device operability benefits previously described in reference to SOD 6200 (FIGS. 96B-96C) also apply to SOD 8500. In an alternative embodiment (not illustrated), a frame post 8583′ may extend above the MVA into the left atrium LA, and occluder 8540′ may be engaged to terminal end thereof by an occluder frame member extending from occluder apex 8545 to the terminal end of frame post 8583′. The joints between the occluder 8540′ and apex frame member and between apex frame member and frame post 8583′ may be movable pivoting joints or static rigid joints or a combination of both types of joints.

In an alternative embodiment, as shown in FIG. 120, SOD 8600 includes a prosthetic valve occluder 8640, a ventricular connector 8690, and a clip connector 8670.

In this embodiment, prosthetic valve occluder 8640 is similar to some of the prosthetic valves described above, except that the prosthetic valve occluder 8640 is configured and adapted (i.e., relative to commercially available TAVI valves for example) with the elements to allow its deployment together with clip CL (i.e., destination therapy). Prosthetic valve 8640 includes a ventricular connector 8690, similar to ventricular connector 6490 of SOD 6400, and a clip connector 8670 that are each connected to one or several structural frame elements (e.g., to a stent frame web) of the prosthetic valve outer body 8620. The clip connector 8670 is coupled to the clip CL either extracorporeally, or during one of the steps in the surgical procedure. Prosthetic valve 8640 may be implanted according to one of the methods described above in reference to FIGS. 116A-118E.

Prosthetic valve occluder 8640 includes an outer body sheath or static membrane 8641 to engage native leaflets AL, PL during diastole and systole. A seal member 8642 extending radially outward from outer body 8620 toward the annulus of the mitral valve MVA is configured and appropriately sized to seal residual openings or leaks between the native leaflets and the outer body 8620. For example, seal member 8642 may be made from a compressible foam or deformable polymer flap or weir and disposed over a section of the outer body 8620 that is adjacent to a commissure to seal a commissural leak.

Alternatively, seal member 8642 can be configured as an inflatable cuff, a deformable bladder member.

Optionally, prosthetic valve occluder 8640 may also include an annulus connector to further secure its attachment to the native valve annulus.

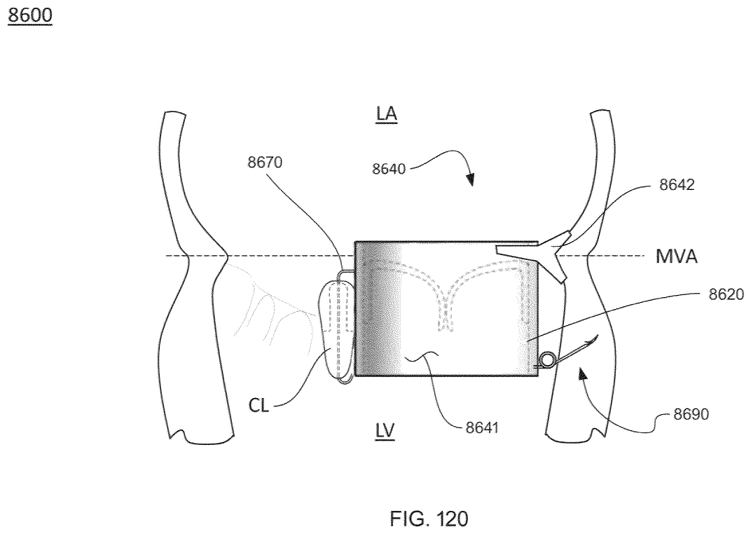

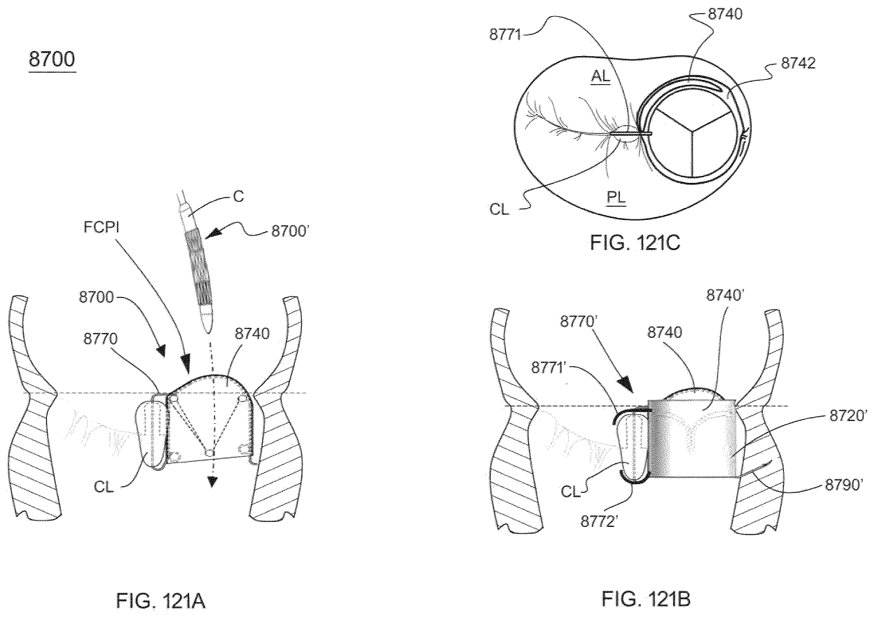

FIGS. 121A-121C illustrate a rescue SOD 8700′ including a prosthetic valve occluder or prosthetic valve 8740′ for implanting in a mitral valve MV that has been previously implanted with an SOD 8700, which in this embodiment includes a movable membrane occluder 8740.

Over time, SOD 8700 or the native mitral valve may have deteriorated to the point that mitral regurgitation has returned. Occluder 8740 may no longer be functioning well to resolve the leakage across the mitral valve leaflets. For example, the movable occluder membranes may have lost some mobility or the valve disease may have progressed to a point that the sealing engagement between the native leaflets and occluder membranes has been compromised. In patients where removing the SOD 8700 is not an option, a salvage prosthetic valve 8740′ may be implanted by a delivery catheter C.

As shown in FIG. 121A, delivery catheter C is directed and positioned within a flow control portion FCP1 of the mitral valve, in the space between the previously implanted occluder 8740 and one of the leaflets (i.e., anterior AL or posterior PL leaflets). The collapsed SOD 8700′ is then deployed and the previously implanted occluder 8740 is displaced towards the leaflet that is not in direct contact with the delivery catheter C. As shown in FIG. 121C, delivery catheter C is directed in the FCP1 in the space between the occluder 8740 and the posterior leaflet PL and the occluder is displaced toward the anterior leaflet AL and anterior valve annulus.

During deployment, prosthetic valve 8740′ is coupled to clip CL of the previously deployed SOD 8700. The coupling of the prosthetic valve to the clip CL may be achieved through a variety of methods and with similar coupling connectors as previously described in embodiments 300, 2200, 2300, 2400, 2500, 2600, or 2700. As shown in FIG. 121B, a clip connector 8770′ is engaged to the outer body 8720′ of the prosthetic valve 8740′. Clip connector 8770′ includes a first clamping member 8771′ and a second clamping members 8772′. The clamping members cooperate to securely engage the clip CL one from each side of the flow control portion FCP1 (i.e., clamping member 8771′ from the LA side and clamping member 8772′ from the LV side). Clip connector 8770′ is preferably integral with the prosthetic valve 8740′ but may also be a separately deployed discrete element adapted to couple to both the clip and the prosthetic valve.

Prosthetic valve 8740′ is also configured with a ventricular connector 8790′, similar to ventricular connector 8690 of SOD 8600, to further secure the prosthetic valve to the left ventricle LV. Optionally, an annulus connector may also be provided.

Prosthetic valve 8740′ may be an existing commercially available valve or, alternatively, a version thereof that may be optimized for the above salvage procedure to include a predetermined clip connector and optionally either an annulus connector, or ventricular connector, or even an additional clip CL provided with the prosthetic valve, or a combination thereof. A seal member 8742′ similar to seal member 8642 may also be provided to seal a potential leakage between the displaced occluder 8740, the native leaflets and the prosthetic valve outer body 8720′.

Some patients develop severe MR months or years after undergoing a TEER procedure. Frequently the left ventricle enlarges and distracts the margins of the anterior and posterior leaflets and prevents them from meeting, thus causing blood to leak or regurgitate into the atrium during systole. Alternatively, leaking or regurgitation can result when chordal supports weaken and allow leaflets to prolapse or flail. With a clip in place, there are few options for a patient who develops recurrent MR. These patients are frequently old and frail and cannot tolerate an open surgical procedure.

In this situation, it can be useful to solve the recurrent MR by anchoring an SOD to the existing, previously placed clip. The SOD can be any of those previously described herein. Another alternative under development by Edwards Lifesciences would involve using the Sapien M3 valve for mitral replacement. In this procedure, a helical anchor is placed around the perimeter of the mitral annulus and a modified transcatheter aortic valve (a tubular stent valve with three leaflets inside and with the addition of a fabric cover around the outside and over the outflow perimeter of the valve) is delivered inside the helix. Such a valve could be very appropriate to anchor to a clip on the side of an MR leak. Or two such valves could be applied-one on each side of the clip. One advantage of using such a modified valve is that the valve has already been developed and benchtop, animal and human clinical testing have all been completed. This reduces the cost of development and the risk for the first patients who receive such a valve as it has already been implanted in many patients. A TAVR type valve that is unaltered or that includes modifications such as those used by Edwards in their Sapien M3 device are good options to use in conjunction with a clip to stop MR leaks in conjunction with a clip. SODs (such as those disclosed herein) may also benefit from an attachment or anchoring not just at the clip, but also to the heart at the region of the commissure or to the left atrial or left ventricular tissue in the region of the commissure—which is approximately opposite to the location of the clip. Such a second anchor would help to stabilize, retain and prevent rocking of the SOD. The second anchor could be spaced from the commissure and located deeper in the left ventricle or in the left atrium.

To retain the SOD, lassos or snares can be applied around the clip and the SOD can be attached to the lassos or snares to hold it in place. To help guide and engage snares, modifications to enhance attachment could be made to the clip. One option would be to add channels or grooves in the clip to help retain the snares. Channels or grooves could potentially be deeper at the bottom (or other convenient location) and with a wider orifice at the bottom to help start engagement with a snare.

Clamps could be used to engage the clip. The clamps could surround the clip or engage from side to side or top to bottom.

The clip could also have a recess or indentation or channel that could be cannulated to facilitate attachment and anchoring. The recess could have retention features such as a friction lock to hold the valve or occluder in place. A recess or indentation or channel could also be useful to facilitate an anchoring procedure. A funnel-shaped opening may make the recess easier to cannulate.

It could also be beneficial to help guide engagement and attachment on a clip after it has been previously applied. An operator could identify the engagement elements, and then working from a distance, and with a catheter, manipulate the engagement elements in a moving and beating heart. Devices, systems and methods to help this engagement would also be valuable. The clip could also be modified with features to help guide and anchor an SOD. The clip could have hooks or circles or loops that could be used to attach an SOD. The attachment could be anywhere on the clip-on the left atrial side, the left ventricular side, on a lateral side or between the jaws. Radiopaque markers could be added or construction features of the clip which appear on fluoroscopy can also be used to guide a procedure.

Sometimes when a TEER procedure is performed, there is significant MR remaining. So adding an SOD could be accomplished as part of the initial procedure. Further, some patients are denied treatment with TEER because it is expected too much MR will remain. Such patients have few options for treatment under these circumstances. If an SOD could be delivered as part of a TEER procedure, many patients could be treated with TEER who would otherwise have few good choices.

Attachment guides or mechanisms for attachment of valve structures to the clip could be added or removed to or from the clip prior to implantation so the user could decide if they preferred adding this feature.

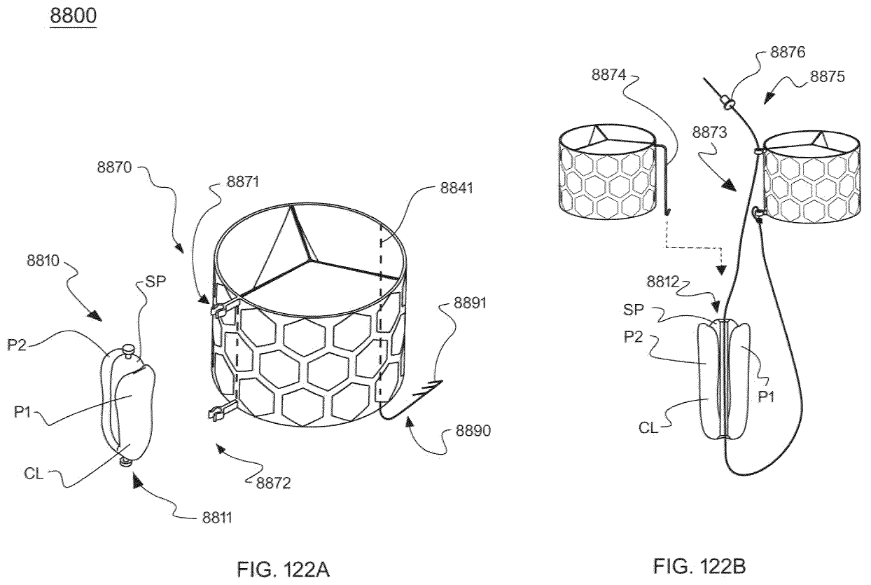

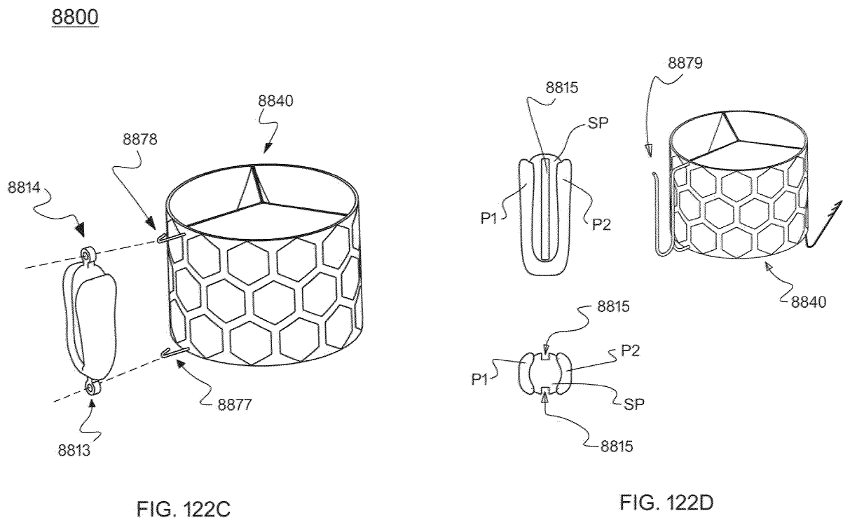

As illustrated in FIGS. 122A-122D, a clip CL for a valve leaflet clipping procedure (i.e., TEER repair) is advantageously configured with predetermined clip features or clip elements to facilitate engagement with a SOD 8800, at a future time through a separate rescue procedure, in the event that the TEER repair fails, or disease progresses and the MR reoccurs.

In this embodiment, SOD 8800 includes a prosthetic valve occluder or prosthetic valve 8840.

As shown in FIG. 122A, clip CL is configured with an upstanding post 8810, extending above the clip spacer SP into the left atrium LA, and a similar post element 8811 extending below the clip paddles P1, P2 into the left ventricle LV. Post elements 8810, 8811 may advantageously serve as coupling elements or anchoring interfaces for a clip connector 8870 provided on the “rescue” prosthetic valve 8840. For example, clip connector 8870 includes a first clamping element 8871 and a second clamping element 8872 for coupling, respectively, to clip post elements 8810, 8811. Clamping elements 8871 or other similar elements may be implemented on the stent frame or outer body 8841 of the prosthetic valve. Alternatively, the post elements 8810, 8811 may be engaged with a clip connector, such as a snare, suture loop, or lasso element, that is deployed during the procedure to approximate and secure the prosthetic valve 8840 to the clip CL. The lasso element may engage the prosthetic valve at a suitable location on the stent frame, or if so provided, on similar post elements to 8810, 8811 to facilitate the procedure. A ventricular connector 8890 is provided with a tissue anchoring barb 8891 to secure the prosthetic valve to the wall of the left ventricle LV. Alternatively, an annulus connector may be provided instead of the ventricular connector or in addition thereto.

As shown in FIG. 122B, clip CL is configured with a channel 8812 in spacer SP, communicating between the LA and LV, and sized to facilitate the passage of a catheter-delivered suture, lasso, snare, or other type of flexible wire element serving as a clip connector 8873 to couple the prosthetic valve 8840 to clip CL through eyelets provided on the outer body of the prosthetic valve. For example, a snare element may be inserted through the channel 8812 from the top of the clip into the left ventricle, to be retrieved by a cooperating catheter into the lumen a delivery cannula, and coupled to lower eyelet 8873 of a collapsed prosthetic valve. The proximal end of the snare element is inserted through the upper eyelet 8875 of the collapsed prosthetic valve. The prosthetic valve may then be delivered in place, adjacent clip CL, in a flow control portion of the clipped mitral valve, by retracting the snare element through the spacer channel 8812. A locking collet or crimp element 8876 secures the coupling arrangement. The prosthetic valve can then be released or radially expanded from its collapsed configuration to resolve the MR. Alternatively, in a variant clip connector configuration, channel 8812 may serve as a docking port for an insertable post element 8874 provided on prosthetic valve 8840.

As shown in FIG. 122C, clip CL is configured with eyelet or hoop members 8814, 8813 extending from the top of spacer SP and below the clip paddles P1, P2, respectively. The hoop members are appropriately sized to receive and engage with elastically deformable retaining hooks 8878, 8877 provided on the outer body of rescue valve 8840.

As shown in FIG. 122D, clip CL is configured with a pair of grooves 8815 on spacer SP (shown in a side view and a top view) suitable to engage with a U-shaped clip connector 8879 provided on prosthetic valve 8840 (i.e., clip connector inserted from below the clip CL) or alternatively with an inverted U-shaped clip connector (i.e., clip connector inserted from above the clip CL; not shown). In an alternative clip connector configuration, groove 8815 may also serve to retain a suture loop or surgical snare engaged with the prosthetic valve, and used to approximate and secure the prosthetic valve to the clip CL. Other predetermined clip features or clip elements are also possible from the examples described above. It is understood that the prosthetic valve SOD embodiments described above in reference to FIGS. 122A-122D may also apply to static membrane of movable membrane SOD in the context of a rescue procedure.

Clip CL may be configured with a predetermined fracture point designed to split the clip and release the captured native leaflets when the rescue valve 8840 is deployed within FCP1. This allows for larger diameter valve to be deployed in an unclipped mitral valve. Alternatively, a balloon valvuloplasty can be performed in a flow control portion of the mitral valve to split the clip CL at the predetermined fracture point prior to deploying a larger diameter valve than would otherwise be possible.

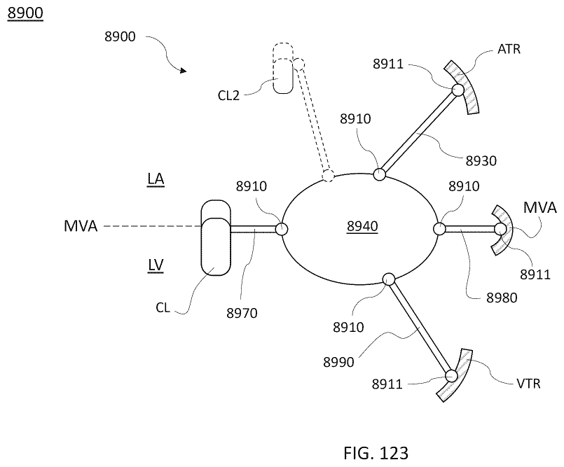

There are many different structures for supporting an SOD in a native valve (mitral, tricuspid, or other valve). FIG. 123 schematically illustrates the various types of structures or connectors that may be used to secure an occluder of an SOD to various cardiac tissues and to a clip CL. These connectors include: a clip connector 8970 to couple occluder 8940 to clip CL; an annulus or commissure connector 8980 to secure occluder 8940 to a valve annulus MVA; a ventricular connector 8990 to secure occluder 8940 to a ventricular tissue VTR; and an atrial connector 8930 to secure occluder 8940 to an atrial tissue ATR. In some embodiments, the occluder may be secured to the native valve leaflets through a second clip CL2 and second clip connector (both shown in dashed line). The connectors are engaged to the occluder each at a respective mechanical joint 8910, and to the respective cardiac tissue at a tissue-anchoring joint 8911. The connectors may be substantially rigid of fixed configuration (i.e., a frame member), flexible and movable (i.e., a flexible tether or wire), or semi-rigid and elastically deformable (i.e., able to bend a predetermined amount such as a cable element). The mechanical joints 8910 may be disposed on the occluder at any suitable location thereon and may be either rigid, static joints, pivoting joints, or rotating joints. The tissue-anchoring joints 8911 may be engaged with the appropriate cardiac tissue at any suitable location and are configured according to the type of cardiac tissue to be engaged (i.e., MVA, ATR, VTR). Anchoring joints 8911 may be rigid, static joints (to the extent that the cardiac tissue permits), pivoting joints, or rotating joints. Any one, two, three, or all four of such types of connectors, according to any of the embodiments disclosed herein, may be used with any of the SOD embodiments disclosed herein. Said another way, any SOD may be supported in operative position in the native valve by engagement with any one or more of the different parts of the heart tissue (leaflets via the clip, atrial tissue, annulus tissue, and/or ventricle tissue). In addition, any type of connector may be engaged with the SOD at any one or more locations on the SOD disclosed herein for any connector.

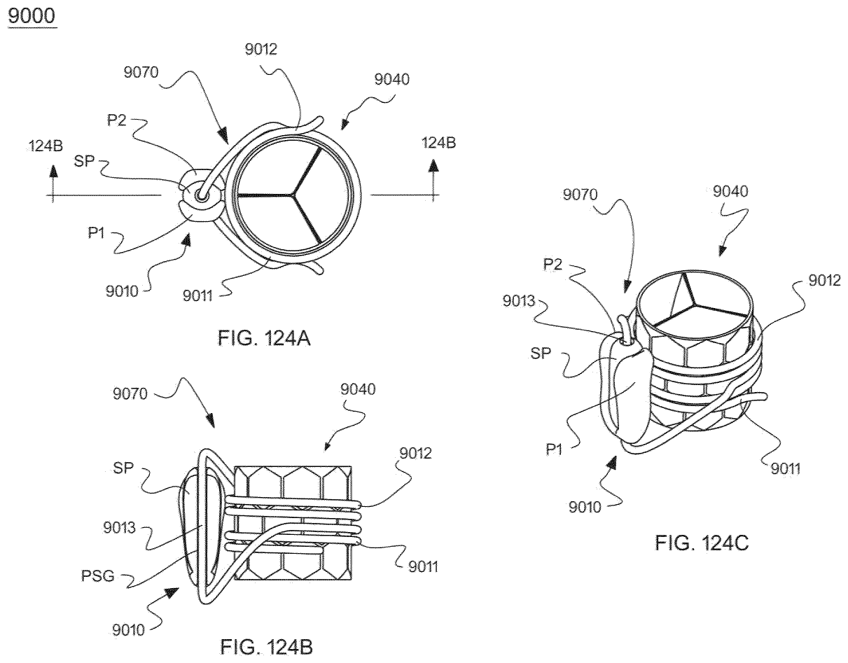

In an alternative embodiment as shown in FIGS. 124A-124C, SOD 9000 includes a prosthetic valve occluder 9040 and a clip connector 9070, which in this embodiment is implemented as a helical docking frame or helical anchor 9010. Prosthetic valve occluder is similar to some of the commercially available TAVR prosthetic valves, or to the Edwards Lifesciences Sapien M3 prosthetic mitral valve. The anchoring or securing of a TAVR-type prosthetic valve to the mitral valve with a helical anchor is described in U.S. Pat. No. 10,945,837 (“the '837 patent”), the disclosure of which is incorporated by reference herein. SOD 9000 adapts the anchoring mechanism of the '837 patent to a mitral valve with clipped native leaflets, wherein a helical anchor is engaged with a clip CL, and a prosthetic valve is disposed in a flow control passage. The helical anchor 9010 has a flexural stiffness that is sufficient to securely retain the prosthetic valve in position when the prosthetic valve 9040 is exposed to the dynamic loads during the cardiac cycle, yet sufficiently flexible to allow deployment of the helical anchor through a delivery catheter without permanent deformation. The helical anchor is configured with a ventricular helix 9011 and an atrial helix 9012. The ventricular helix, which is deployed through a catheter (not shown, but as described in the '837 patent), is wound between the chordae on the ventricular side of the mitral valve leaflets. The atrial helix is deployed on the atrial side of the mitral valve leaflets. As such, the mitral valve leaflets are trapped between the atrial helix and the ventricular helix. Helical anchor 9010 is provided with a helix spacer section 9013 that connects atrial helix 9012 to the ventricular helix 9011. Clip spacer SP is configured with a through-passage PSG appropriately sized to retain therein the helix spacer section 9013 thereby coupling the clip CL with the helical anchor 9010. Once the helical anchor 9010 is fully deployed and coupled to clip CL, the prosthetic valve 9040 is delivered through a separate delivery catheter and deployed within the helical anchor as shown in FIG. 124C.

It is contemplated that for a movable membrane occluder, that is transitioning from the EDPS to the systolic occluder configuration, the arcuate support frame (e.g., frame 6420) may flex inwardly at its terminal ends, due to the effect of fluid inertial forces and/or systolic pressure within the occluder cavity which places the free margins of the occluder membranes in under tension to support the resulting hoop stress. This effect may not be accurately reflected in some of the schematic representations.

In diastole, it is understood that to reduce mitral stenosis, the occluder membranes are generally collapsed and in proximity to one another, and not just the membrane free margins as shown in the schematic illustrations of the occluder in the diastolic configuration.

Some of the movable membrane SOD embodiments described above include occluders that may advantageously assume a membrane-spaced EDPS configuration due to the membrane spacing element provided in the occluder. It is understood that some of the embodiments of a movable membrane SOD may also be provided without this membrane spacing element and still achieve a degree of functionality in use.

Some of the movable membrane SOD embodiments described above include occluders that may advantageously assume a membrane-spaced EDPS configuration due to the membrane spacing element provided in the occluder between two movable occluder membranes. It is understood that some of these embodiments may also function with only one of the occluder membranes being movable and able to move relative to the opposing occluder membrane which itself is relatively static.

Many different embodiments of SODs have been disclosed herein. As noted, any movable membrane occluder disclosed herein may incorporate any of the disclosed membrane spacing element embodiments. For an SOD with more than one occluder, any one or more of the occluders may be movable membrane occluders, and any one or more of the movable membrane occluders may include any of the membrane spacing element embodiments, and different movable membrane occluders in the same SOD may include different membrane spacing element embodiments.

In some of the movable membrane SOD embodiments described above, the occluder free margins are illustrated as being generally aligned and in register with one another, especially in the closed, diastolic configuration of the occluder. It is understood that opposing occluder membranes may also have free margins that are intentionally not aligned with one another, in order to advantageously urge the occluder to transition from the closed, diastolic configuration to the intermediate EDPS configuration. For instance, the free margin of one movable occluder membrane may extend deeper into the left ventricle LV than the free margin of the opposing movable occluder membrane. In another example, a membrane free margin may extend deeper into the LV at its mid-span location relative to its margin terminal end location (i.e., adjacent the support frame), or extend less into the LV at its mid-span location relative to its margin terminal end locations, while the free margin of the opposing membrane may be generally linear between the margin terminal end locations and at the mid-span location (i.e., extending equally within the LV throughout its free margin length). In another example, when the movable occluder membranes are each attached to a first and second spaced apart frame member, the length of the free margins between first and second frame members may be different for each of the occluder membranes. Alternatively, at a first frame member, a first membrane free margin may be attached higher up towards the left atrium and a second opposing membrane free margin may be attached deeper within the left ventricle, with the inverse opposite free margin arrangement at the second frame member. Many other combinations are possible to set the opposing membrane free margins in a generally non-aligned configuration relative to one another, especially in the closed, diastolic configuration of the movable membrane occluder.

While various embodiments have been described herein, textually and/or graphically, it should be understood that they have been presented by way of example only, and not limitation. Likewise, it should be understood that the specific terminology used herein is for the purpose of describing particular embodiments and/or features or components thereof and is not intended to be limiting. Various modifications, changes, enhancements, and/or variations in form and/or detail may be made without departing from the scope of the disclosure and/or without altering the function and/or advantages thereof unless expressly stated otherwise. Functionally equivalent embodiments, implementations, and/or methods, in addition to those enumerated herein, will be apparent to those skilled in the art from the foregoing descriptions and are intended to fall within the scope of the disclosure.

For example, while the prosthetic valves are described herein as being used with particular native valves and clip configurations, it should be understood that they have been presented by way of example only and not limitation. The embodiments and/or devices described herein are not intended to be limited to any specific implementation unless expressly stated otherwise.

Where schematics, embodiments, and/or implementations described above indicate certain components arranged and/or configured in certain orientations or positions, the arrangement of components may be modified, adjusted, optimized, etc. The specific size and/or specific shape of the various components can be different from the embodiments shown and/or can be otherwise modified, while still providing the functions as described herein. More specifically, the size and shape of the various components can be specifically selected for a desired or intended usage. Thus, it should be understood that the size, shape, and/or arrangement of the embodiments and/or components thereof can be adapted for a given use unless the context explicitly states otherwise. By way of example, in some implementations, a treatment device intended to provide treatment to an adult user may have a first size and/or shape, while a treatment device intended to provide treatment to a pediatric user may have a second size and/or shape smaller than the first size and/or shape. Moreover, the smaller size and/or shape of, for example, a pediatric treatment device may result in certain components being moved, reoriented, and/or rearranged while maintaining the desired function of the device.

Although various embodiments have been described as having particular characteristics, functions, components, elements, and/or features, other embodiments are possible having any combination and/or sub-combination of the characteristics, functions, components, elements, and/or features from any of the embodiments described herein, except mutually exclusive combinations or when clearly stated otherwise. Moreover, unless otherwise clearly indicated herein, any particular combination of components, functions, features, elements, etc. can be separated and/or segregated into independent components, functions, features, elements, etc. or can integrated into a single or unitary component, function, feature, element, etc.

Where methods described above indicate certain events occurring in certain order, the ordering of certain events may be modified. Additionally, certain of the events may be performed concurrently in a parallel process when possible, as well as performed sequentially as described above. While methods have been described as having particular steps and/or combinations of steps, other methods are possible having a combination of any steps from any of methods described herein, except mutually exclusive combinations and/or unless the context clearly states otherwise.

1. Apparatus comprising:
an occluder configured to be disposable between a first native leaflet and a second native leaflet of a native heart valve, in a flow control portion of the native valve formed between the native leaflets, a commissure of the native leaflets, and a clip securing the margins of the native leaflets together, the occluder configured to selectively permit blood to flow in a first direction between the native leaflets and the occluder and to inhibit blood from flowing in a second, opposite direction between the native leaflets and the occluder, the occluder formed at least in part from a first flexible membrane having a first free margin and a second flexible membrane having a second free margin, the flexible membranes movable in response to the flow of blood adjacent thereto,
at least one of the first flexible membrane movable away from the first native leaflet and the second flexible membrane movable away from the second native leaflet, to a closed configuration of the occluder in which the first free margin and the second free margin are adjacent, in response to blood flow in the first direction,
at least one of the first flexible membrane and the second flexible membrane movable toward the first native leaflet and the second native leaflet, respectively, and the first flexible membrane and the second flexible membrane coaptable with the first native leaflet and the second native leaflet, respectively, in an open configuration in which the first free margin and the second free margin are fully spaced apart, in response to blood flow in the second direction, and
the first flexible membrane and the second flexible membrane disposable in an intermediate configuration in which the first free margin and the second free margin are partially spaced, less than in the open configuration;
a support frame coupled to the occluder and coupleable to the clip to secure the support frame and the occluder in the native valve with the occluder disposed in the flow control portion of the native valve; and
a membrane spacing element configured to dispose the flexible membranes in the intermediate configuration in response to a substantial absence of blood flow between the native leaflets, the membrane spacing element configured to permit the flexible membranes to move from the intermediate configuration to the closed configuration in response to blood flow in the first direction and to move from the intermediate configuration to the open configuration in response to blood flow in the second direction.

2. The apparatus of claim 1, wherein the membrane spacing element is disposed between the first flexible membrane and the second flexible membrane, and is configured to urge the first free margin and the second free margin away from each other towards the intermediate configuration.

3. The apparatus of claim 2, wherein the membrane spacing element includes an elastically-deformable biasing element coupled to at least a portion of both the first and the second flexible membranes, the biasing element assuming a predetermined, intermediate element configuration during the substantial absence of blood flow between the native leaflets thereby disposing the flexible membranes in the intermediate configuration, the biasing element elastically deformable from the intermediate element configuration toward a first element configuration in response to blood flow in the first direction as the occluder assumes the closed configuration, the biasing element elastically deformable from the intermediate configuration toward a second, opposite element configuration in response to blood flow in the second direction as the occluder assumes the open configuration.

4. The apparatus of claim 2, wherein the membrane spacing element includes a first rib stiffener formed on the first flexible membrane and a second rib stiffener portion formed on the second flexible membrane.

5. The apparatus of claim 4, wherein the flexible membranes are formed of polymer material, and the rib stiffeners are integrally formed therewith.

6. The apparatus of claim 1, wherein the native valve is an atrioventricular valve having an annulus disposed between an atrium of the heart above the annulus and a ventricle of the heart below the annulus, the frame has a first portion couplable to the clip proximate to a first end of the occluder and second portion, spaced from the first portion, proximate to a second end of the occluder, and further comprising a ventricular connector coupled to the second portion of the frame, the ventricular connector including a tissue anchor configured to penetratingly engage with tissue of the ventricle, the occluder being securable in operative position in the native valve, and resistant to displacement from the operative position by the flow of blood in the first direction and the second direction between the atrium and the ventricle, by engagement of the frame with the clip and with the ventricular connector.

7. The apparatus of claim 6, wherein the apparatus, when disposed in the native valve with the first portion of the frame coupled to the clip and with the ventricular connector coupled to ventricular tissue of the heart by the tissue anchor, has an occluder pivoting axis defined between a location at which the clip is engaged with native leaflets and a location at which the tissue anchor is engaged with the ventricular tissue, the apparatus configured so that the occluder can pivot about the occluder pivot axis, the occluder configured such that when in the open configuration in the heart, the occluder has a center of pressure formed by higher pressure blood on the ventricle side of the occluder and lower pressure blood on an atrial side of the occluder, the center of pressure being positioned above the occluder pivot axis.

8. The apparatus of claim 6, wherein the apparatus has an occluder pivoting axis defined between a point on the first portion of the frame configured to engage the clip and the tissue anchor, the apparatus configured so that the occluder can pivot about the occluder pivot axis, the tissue anchor being disposed below at least a portion of the lower edge of at least one of the first free margin and the second free margin.

9. The apparatus of claim 1 further comprising the clip, wherein the occluder is a first occluder, the flow control portion is a first flow control portion, and the commissure is a first commissure, and further comprising a second occluder configured to be disposable between the first native leaflet and the second native leaflet, in a second flow control portion of the native valve formed between the native leaflets, a second commissure of the native leaflets, and the clip. 10. The apparatus of claim 1, further comprising the clip.

10-11. (canceled)

12. The apparatus of claim 6, wherein the tissue anchor is one of a barb, helically wound wire, clip, and multi-pronged anchor.

13. The apparatus of claim 6, wherein the tissue anchor includes a flexible tether coupled to the support frame and having a tissue penetrating barb element at a distal end therefore, the tissue anchor configured to be engaged with a papillary muscle of the ventricle.

14. (canceled)

15. Apparatus comprising:
an occluder configured to be disposable between a first native leaflet and a second native leaflet of a native heart valve, in a flow control portion of the native valve formed between the native leaflets, a commissure of the native leaflets, and a clip securing the margins of the native leaflets together, the occluder configured to selectively permit blood to flow in a first direction between the native leaflets and the occluder and to inhibit blood from flowing in a second, opposite direction between the native leaflets and the occluder; and
a support frame coupled to the occluder and coupleable to the clip to secure the support frame and the occluder in the native valve with the occluder disposed in the flow control portion of the native valve, the support frame configured to be adjustable to conform to the geometry of the clipped native valve.

16. The apparatus of claim 15, wherein the support frame includes an occluder arm coupleable to the clip at a first end thereof, the occluder coupled to the occluder arm for movement along the occluder arm toward and away from the first end of the occluder arm.

17. (canceled)

18. The apparatus of claim 15, wherein the support frame includes an occluder arm coupled to the occluder, and an occluder arm pivot coupleable to the clip and coupled to the occluder arm, the occluder arm pivot being pivotably adjustable relative to the clip to dispose the occluder at a variable angular orientation relative to the clip.

19. (canceled)

20. The apparatus of claim 18, wherein the flow control portion is a first flow control portion, the commissure is a first commissure, the occluder is a first occluder, and the occluder arm is a first occluder arm, the native heart valve having a second flow control portion formed between the native leaflets, the clip, and a second commissure of the native leaflets, further comprising:
a second occluder arm coupled to the occluder arm pivot, and
a second occluder coupled to the second occlude arm and configured to be disposable in the second flow control portion and to selectively permit blood to flow in the first direction through the second flow control portion between the native leaflets and the second occluder and to inhibit blood from flowing in the second direction through the second flow control portion between the native leaflets and the second occluder, the occluder arm pivot being further adjustable relative to the clip to dispose the second occluder at a variable angular orientation relative to the clip,
the first and second occluder coupled to the first and second occluder arm, respectively, for movement along the first and second occluder arm, respectively, toward and away from the occluder arm pivot.

21. The apparatus of claim 18, wherein the occluder arm pivot is pivotably adjustable relative to the clip to tilt the occluder laterally relative to a vertical axis of the clip.

22. The apparatus of claim 18, wherein the occluder arm pivot is pivotably adjustable relative to the clip to tilt the occluder longitudinally relative to a vertical axis of the clip, to dispose the occluder higher or lower relative to the clip.

23. The apparatus of claim 18, wherein the support frame is coupleable to the clip for selective axial positioning relative to the clip to set a height of the support frame relative to the clip, and thereby the height of the occluder in the flow control portion.

24. (canceled)

25. A clip for a leaflet clipping procedure on a native heart valve having native leaflets, comprising:
a body portion;
a first paddle configured to engage a first native leaflet between the paddle and the body portion;
a second paddle configured to engage a second native leaflet between the paddle and the body portion; and
one or more elements coupled to the body portion and configured to be engaged by an occluder that can be disposed between the native leaflets when the leaflets are engaged by the clip, the occluder configured to selectively permit blood to flow in a first direction between the native leaflets and the occluder and to inhibit blood from flowing in a second, opposite direction between the native leaflets and the occluder.

26. (canceled)

27. A system including the clip of claim 25 and the occluder coupled to the clip via the one or more elements, and wherein the one or more elements include one or more of a channel, a groove, a loop, a circle, and a surface configured to be engaged by the occluder.
